(19) United States
(12) Patent Application Publication
(10) Pub. No.: US 2003/0228585 A1
(43) Pub. Date: Dec. 11, 2003

(54) Kit and method for determining hla type

(76) Inventors: Hidetoshi Inoko, Yokohama-shi (JP); Taeko Kagiya, Naka-gun (JP); Tatsuo Ichihara, Chiba-shi (JP); Yoshiyuki Matsumura, Chiba-shi (JP); Shogo Moriya, Chiba-shi (JP); Michio Nishida, Chigasaki-shi (JP)

Correspondence Address: KNOBBE MARTENS OLSON & BEAR LLP, 2040 MAIN STREET, FOURTEENTH FLOOR, IRVINE, CA, 92614, US

(21) Appl. No.: 10/297,068

(22) PCT Filed: Jun. 1, 2001
(86) PCT No.: PCT/JP01/04662

(51) Int. Cl. C12Q 1/68; C12M 1/34

(52) U.S. Cl. 435/6; 435/287.2

(57) ABSTRACT

HLA genotype of a test specimen is determined by hybridization of a nucleic acid sequence derived from the test specimen by using a substrate on which oligonucleotides of 10-24 nucleotide length derived from sequences of a group of genes belonging to HLA class I or class II antigen on a human genome and including polymorphism of gene as alloantigens in the sequences are immobilized through covalent bonds. There are provided a typing kit and typing method that are suitable for processing of a large number of specimens and enable high accuracy typing by one test.

### TECHNICAL FIELD

The present invention relates to a typing kit and typing method for determining HLA genotypes. HLA typing is carried out to judge possibility of transplantation between immunogenetically different individuals. Specifically, its purpose is to provide genetic information to determine transplantation compatibility for transplantation of an organ or tissue such as bone marrow, kidney, liver, pancreas, islets of Langerhans or cornea. The kit of the present invention can also be applied to susceptibility test for a specific disease that is caused by a hereditary factor, or individual identification.

The present invention further relates to PCR primers used for the aforementioned kit and method and a method for producing the same.

### BACKGROUND ART

1. Human Leukocyte Antigen (HLA) Types and Importance of Histocompatibility

In autogenous transplantation or transplantation between immunogenetically homologous individuals such as identical twins of a tissue or cells, a graft is taken without inducing rejection reactions. On the other hand, in transplantation between immunogenetically different individuals such as other family members or unrelated individuals (allogenic transplantation) or transplantation between different species such as human being and other primates (interspecific transplantation), transplantation immunity is established and a rejection reaction is induced against a graft. There are antigens inducing particularly intense transplantation immunity as targets in this reaction, which are called major histocompatibility antigens. These antigens are controlled by a group of genes called MHC (Major Histcompatibility Complex). MHC is classified into classes I, II and III. class I and class II genes code for HLA (Human Leukocytic Antigen), which are transplantation antigens. HLA antigens are proteins responsible for discriminating self and non-self in the immune mechanism. Since a large number of alleles exist on their gene loci, the genes coding for these antigens are known to be highly polymorphic.

Determination of genotypes (typing) of HLA provides information for determination of the possibility of transplantation between immunogenetically different individuals. Specifically, this is employed for transplantation of an organ or tissue such as bone marrow, kidney, liver, pancreas, islets of Langerhans or cornea. The HLA genotypes are considered to be associated with certain refractory diseases and can also be utilized as supplementary means for diagnosing chronic rheumatoid arthritis, IDDM, insulin autoimmune syndrome and so forth. Besides, HLA typing is applied in a paternity test or individual identification because of the genetic polymorphism of HLA antigens.

2. Genes Involved in HLA Typing and Polymorphism

Human MHC is an HLA gene complex located on the short arm of the 6th chromosome. HLA antigens encoded by these genes include class I antigens controlled by HLA-A, -B, -C loci and class II antigens controlled by the HLA-D region (DR, DQ and DP).

class I antigens have a double-stranded structure composed of L-chain called â2 microglobulin having a molecular weight of 12 kDa and H-chain having a molecular weight of 45 kDa. These antigens are involved in cellular immunity mediated by T cells, determine antigen specificity, and become target antigens in transplantation immunity. class II antigens have a double-stranded structure of two chains having molecular weights of 34 kDa and 29 kDa, respectively, determine antigen recognition and are involved in antigen presentation in humoral immunity. DP, DQ and DR in the HLA-D region are further subtyped according to polymorphism based on the amino acid or nucleotide sequence. DR is classified into about 20 subtypes, DQ is classified into about 10 subtypes, and DP is classified into a few subtypes. This large number of subtypes is attributed to the facts that the HLA antigens are composed of the á-chain and the â-chain each having a different molecular weight and that only the â-chain serves as an alloantigenic determinant except for the DQ antigens. Moreover, the subtypes are further subdivided based on the amino acid sequences or the corresponding nucleotide sequences of the antigenic determinants.

3. Means for HLA Typing

Serological test utilizing antiserum reactions and cytological methods utilizing blast transformation of lymphocytes have been generally used. However, not only time and labor are required for examination and operation is complicated, but also accuracy of obtainable results are not so high. Therefore, DNA-based typing methods utilizing PCR are being developed to overcome these drawbacks and put into practical use. Examples thereof include the PCR-SSP method (2), PCR-RFLP method (3), PCR-SSOP method (4), PCR-SSCP method (5) and so forth.

In the PCR-SSP method, a gene sequence is confirmed by amplifying the hypervariable region of target HLA antigen to determine the HLA antigen type. In this method, a large number of specific primers must be prepared to enable high accuracy typing and an enormous number of times of PCR also must be performed. Thus, this method is not necessarily practical.

In the PCR-RFLP (PCR-restriction fragment length polymorphism) method, a nucleotide sequence is amplified by PCR so as to contain a region involved in HLA antigen typing and the obtained amplification product is digested with restriction enzymes. The nucleotide sequence of the amplification product is digested by using restriction enzymes cleaving different sites depending on the HLA type and separated by electrophoresis. Then, the type is determined based on the migration pattern. This method is characterized in that results can be rapidly obtained. However, the cleavage sites of the restriction enzymes may not necessarily exist in the sequence. Further, the amplification product must be digested with multiple kinds of restriction enzymes for one typing. Thus, this method is not practical to process a large amount of specimen.

In the PCR-SSOP (PCR-sequence specific oligonucleotide probe) method, a nylon membrane on which DNA amplified by primers specific to HLA gene is immobilized is prepared and respective HLA type specific oligonucleotide probes are hybridized for typing. In a modified method of PCR-SSOP, a membrane on which respective HLA type specific oligonucleotides are immobilized is prepared and DNA amplified by using HLA gene specific primers are hybridized. In the method of immobilizing amplified DNA, a membrane must be prepared for a probe of each type for hybridization and therefore much labor is required for multiple specimen typing. Since the substrate on which amplified DNA or oligonucleotides are immobilized is a membrane, a membrane having a large area must be prepared to perform high accuracy typing with one membrane. Therefore, the typing pattern may be complicated.

In the PCR-SSCP (PCR-single strand conformation polymorphism) method, DNA amplified by PCR is denatured to obtain single strands and changes in the high-order structure due to the difference in nucleotide sequences are detected by electrophoresis. In this method, all DNA of known types must be electrophoresed on the same gel. Thus, practical multiple specimen processing is almost impossible.

4. Importance of High Accuracy HLA Typing

To achieve ideal transplantation, it is important that HLA types of donor and recipient are compatible with high accuracy. Therefore, a tissue or individual information typed in high accuracy must be clarified in advance. In this respect, HLA antigens of the donor and the recipient must be compared with all the types currently discovered for determination before transplantation. Even if no donor having completely compatible HLA types is found, a high success rate can be expected by performing transplantation between individuals having similar types of which high accuracy HLA types are determined. Further, it is also expected that load of recipient can also be relieved in immune responses such as a rejection reaction occurring after transplantation.

When high accuracy typing is carried out in personal identification, one completely compatible individual is found out of tens or hundreds of thousands of individuals, but such an individual can be identified in high accuracy. Furthermore, if high accuracy typing is performed for each gene locus of HLA antigens, the accuracy of the personal identification can be further improved.

In a medical scene requiring HLA type information or a situation requiring a personal identification, provision of highly reliable HLA type information is being required. It is important that typing of a large number of specimens can be easily and conveniently performed by HLA typing methods therefor. Currently, various methods are being developed and improved, but there remain many drawbacks that operation is complicated, multiple specimen processing is not possible and so forth.

### DISCLOSURE OF THE INVENTION

To solve these problems, an object of the present invention is to provide a kit and method that are suitable for processing of a large number of specimens and enable a large number of high accuracy typing tests by one test for one specimen.

The present invention provides a typing kit for determining HLA genotype of a test specimen by hybridization between a nucleic acid sequence derived from the test specimen and oligonucleotides, which comprises a substrate on which the oligonucleotides are immobilized through covalent bonds, wherein the oligonucleotides are of 10-24 nucleotide length and are derived from sequences of a group of genes belonging to HLA class I or class II antigen on a human genome and each of the oligonucleotides includes polymorphism of each gene as alloantigen in the sequence.

The present invention also provides a method for determining HLA genotype of a test specimen, which comprises allowing hybridization of the oligonucleotides on the substrate of the aforementioned typing kit with a nucleic acid sequence derived from the specimen and detecting occurrence of hybridization of the oligonucleotides and the nucleic acid sequence derived from the specimen.

The present invention also provides the aforementioned typing kit, wherein class I antigen is an antigen controlled by a gene locus coding for any of HLA-A, HLA-B, HLA-C, HLA-D, HLA-E, HLA-F and HLA-G or a null gene and class II antigen is an antigen controlled by a gene locus coding for any of HLA-DQ, HLA-DR or HLA-DP or a pseudogene;

The aforementioned typing kit, wherein HLA-DQ is an antigen derived from any of DQA1, DQA2, DQB1 and DQB2 gene loci or a pseudogene;

the aforementioned typing kit, wherein HLA-DR is an antigen derived from any of DRA, DRB1, DRB3, DRB4 and DRB5 gene loci or a pseudogene; and

the aforementioned typing kit, wherein HLA-DP is an antigen derived from any of DPA1, DPA2, DPB1 and DPB2 gene loci or a pseudogene.

The present invention also provides an oligonucleotide, which comprises a nucleotide sequence based on any of the aforementioned oligonucleotides of 10-24 nucleotide length comprising a nucleotide sequence specific to each type of HLA gene sequences on the human genome and obtained by extending or shortening one of the oligonucleotides at 5′ or 3′ end or at both ends, and in which the sequence specific to each HLA type is not substituted.

The present invention also provides the aforementioned typing kit, wherein at least one of the aforementioned oligonucleotides is replaced with an oligonucleotide of 8-24 nucleotide length obtained by extending or shortening any of the nucleic acid sequences of SEQ ID NOS: 1-397, 456-503, 507-589, 594-898, 908-1072 or 1080-1298 for a gene sequence on the genome at 5′ or 3′ end or at both ends without eliminating or changing nucleotides associated with the gene polymorphism to have optimized binding affinity for the aforementioned hybridization.

The present invention further provides the aforementioned typing kit, wherein at least one of the oligonucleotides is an oligonucleotide whose binding affinity for the hybridization is reduced by replacing an arbitrary nucleotide not involved in the gene polymorphism with a spacer compound, for example, a spacer compound having a nucleic acid frame that does not form a hydrogen bond with any nucleotide.

Furthermore, peptide nucleic acids (PNA) can be used instead of these oligonucleotides.

The present invention further provides a method for determining HLA genotype of a specimen, which comprises a step of carrying out low accuracy typing of HLA genotype of the specimen by performing first PCR amplification using primers for low accuracy genotyping and a nucleic acid sequence derived from the. specimen as a template, allowing hybridization of the amplification product with oligonucleotides contained in the aforementioned typing kit, and detecting occurrence of hybridization of the nucleic acid sequence derived from the specimen and each of the oligonucleotides; and

a step of carrying out high accuracy HLA genotyping of the specimen by performing second PCR amplification based on the above determination result using primers appropriately selected from primers for high accuracy genotyping and a nucleic acid sequence derived from the specimen as a template, allowing hybridization of the amplification product with the oligonucleotides contained in the aforementioned typing kit, and detecting occurrence of hybridization of the nucleic acid sequence derived from the specimen and each of the oligonucleotides.

The present invention provides primers for low accuracy typing used for preparation of a probe for low accuracy typing, which consist of any oligonucleotide pair or an arbitrary combination of oligonucleotide pairs selected from a pair of oligonucleotides having nucleotide sequences of SEQ ID NOS: 398 and 400 for amplifying DQB1;

a pair of oligonucleotides having nucleotide sequences of SEQ ID NOS: 399 and 400 for amplifying DQB1;

a pair of oligonucleotides having nucleotide sequences of SEQ ID NOS: 401 and 403 for amplifying DRB1; and

a pair of oligonucleotides having nucleotide sequences of SEQ ID NOS: 402 and 403 for amplifying DRB1.

The present invention further provides primers for high accuracy typing used for preparation of a probe for high accuracy typing based on result of low accuracy typing, which consist of any oligonucleotides pair or an arbitrary combination of oligonucleotides pairs selected from:

a pair of oligonucleotides having nucleotide sequences of SEQ ID NOS: 404 and 405 for amplifying DQB1;

a pair of oligonucleotides having nucleotide sequences of SEQ ID NOS: 404 and 406 for amplifying DQB1;

a pair of oligonucleotides having nucleotide sequences of SEQ ID NOS: 407 and 409 for amplifying DQB1;

a pair of oligonucleotides having nucleotide sequences of SEQ ID NOS: 408 and 409 for amplifying DQB1;

a pair of oligonucleotides having nucleotide sequences of SEQ ID NOS: 410 and 412 for amplifying DQB1;

a pair of oligonucleotides having nucleotide sequences of SEQ ID NOS: 411 and 412 for amplifying DQB1;

a pair of oligonucleotides having nucleotide sequences of SEQ ID NOS: 413 and 417 for amplifying DRB1;

a pair of oligonucleotides having nucleotide sequences of SEQ ID NOS: 414 and 417 for amplifying DRB1;

a pair of oligonucleotides having nucleotide sequences of SEQ ID NOS: 415 and 417 for amplifying DRB1; and

a pair of oligonucleotides having nucleotide sequences of SEQ ID NOS: 416 and 417 for amplifying DRB1.

The present invention further provides primers for high accuracy typing used for preparation of a probe for high accuracy typing, which consist of any oligonucleotide pair or an arbitrary combination of oligonucleotides pair selected from:

a pair of oligonucleotides having nucleotide sequences of SEQ ID NOS: 418 and 420 for amplifying DQA1;

a pair of oligonucleotides having nucleotide sequences of SEQ ID NOS: 419 and 420 for amplifying DQA1;

a pair of oligonucleotides having nucleotide sequences of SEQ ID NOS: 421 and 422 for amplifying DQB1;

a pair of oligonucleotides having nucleotide sequences of SEQ ID NOS: 423 and 424 for amplifying DRA1;

a pair of oligonucleotides having nucleotide sequences of SEQ ID NOS: 425 and 428 for amplifying DRB1;

a pair of oligonucleotides having nucleotide sequences of SEQ ID NOS: 426 and 428 for amplifying DRB1;

a pair of oligonucleotides having nucleotide sequences of SEQ ID NOS: 427 and 428 for amplifying DRB1;

a pair of oligonucleotides having nucleotide sequences of SEQ ID NOS: 429 and 431 for amplifying DRB3;

a pair of oligonucleotides having nucleotide sequences of SEQ ID NOS: 430 and 431 for amplifying DRB3;

a pair of oligonucleotides having nucleotide sequences of SEQ ID NOS: 432 and 433 for amplifying DRB4;

a pair of oligonucleotides having nucleotide sequences of SEQ ID NOS: 434 and 435 for amplifying DRB4;

a pair of oligonucleotides having nucleotide sequences of SEQ ID NOS: 436 and 437 for amplifying DRB5;

a pair of oligonucleotides having nucleotide, sequences of SEQ ID NOS: 438 and 439 for amplifying DRB6;

a pair of oligonucleotides having nucleotide sequences of SEQ ID NOS: 438 and 440 for amplifying DRB6;

a pair of oligonucleotides having nucleotide sequences of SEQ ID NOS: 441 and 442 for amplifying DRB7;

a pair of oligonucleotides having nucleotide sequences of SEQ ID NOS: 443 and 444 for amplifying DPA1;

a pair of oligonucleotides having nucleotide sequences of SEQ ID NOS: 445 and 446 for amplifying DPB1; and

a pair of oligonucleotides having nucleotide sequences of SEQ ID NOS: 445 and 447 for amplifying DPB1.

The term “class” used for HLA classes I and II in the present specification means classification based on the roles played in an organism. That is, the present specification is also according to this classification. class I antigen is a generic designation of HLA-A, HLA-B, HLA-C, HLA-D, HLA-E, HLA-F and HLA-G antigens. class II antigen is a generic designation of HLA-DQ, HLA-DR and HLA-DP antigens. Alloantigens are antigens characterizing individuals that belong to the same animal species but have genetic differences. Genetic differences mean differences in nucleotide sequences. Further, each of alloantigens classified based on genetic differences is designated as an alloantigen type.

In the present specification, the term “polymorphism” means a characteristic of having the same function but a different primary structure in a protein or DNA coding therefor.

In the present specification, the term “gene locus” means a position occupied by each HLA gene having polymorphism on a chromosome. As for gene loci of DR, DQ and DP antigens, gene loci of the DR antigens include DRA, DRB1, DRB3, DRB4 and DRB5, gene loci of the DQ antigens include DQA1, DQA2, DQB1 and DQB2, and gene loci of the DP antigens include DPA1, DPA2, DPB1 and DPB2.

In the present specification, the term “antigen derived from a gene locus” means an antigen translated from a gene present on the gene locus.

In the present specification, the term “nucleotides involved in gene polymorphism” means nucleotides having different primary sequences in genes coding for each alloantigen.

In the present specification, the term “test specimen” means a sample to be typed. In the present specification, it means chromosome DNA or RNA extracted from a tissue or somatic cell or a reactant enzymatically produced by using any of these as a template.

In the present specification, the term “homopolymer of thymidine residue” means a polymer obtained by repeatedly synthesizing deoxythymidine at 5′ or 3′ end of nucleotide sequence portion involved in typing. This synthesis of oligonucleotide can be performed by a commercially available DNA synthesizer.

Hereafter, a constitution of the kit of the present invention and a method of manufacturing the same, HLA typing method using the kit of the present invention, oligonucleotides for use in these kit and methods, a nucleic acid target derived from a test specimen human genome, which is a sample for use in the HLA typing using the kit of the present invention, and a method of manufacturing the same and primers for preparing the nucleic acid target will be explained.

<1> Synthetic Oligonucleotide for Use in the Present Invention

The synthetic oligonucleotides for use in the present invention are derived from human genomic genes involved in HLA typing and synthesized based on nucleotide sequences obtained from these genes. Hereafter, these oligonucleotides are also referred to as capture oligos.

Capture oligos are designed so that they should contain a type-specific nucleotide sequence region present in HLA gene of each type. The essential characteristic of the present invention is that the HLA typing is performed by identifying whether a test human genome has these regions.

Typing of a type-specific nucleotide sequence in genes involved in HLA typing can be carried out by allowing hybridization of each type-specific capture oligo with a nucleic acid fragment target (also referred to as “nucleic acid target”) having a part of nucleotide sequence of the genes involved in HLA typing of human chromosome DNA and determining with which capture oligo the fragment hybridizes.

The capture oligos can be prepared as synthetic oligonucleotides having a nucleotide sequence consisting of 10-24 nucleotides containing a nucleotide sequence specific to each type in a nucleotide sequence of gene involved in HLA typing. It is generally preferred that the capture oligos are designed so that the type-specific nucleotide sequence should be positioned in the center portions of the capture oligos. If the capture oligos are too short, it becomes difficult to detect hybridization. On the other hand, if the capture oligo is too long, inhibition of hybridization by a type-specific sequence does not occur. Therefore, the range of 10-24 nucleotides is preferred. However, this optimization of the capture oligo length primarily depends on characteristics of the sequence (content of specific nucleotide and repetition of the same nucleotide), and it has been confirmed by experiments according to the present invention that even a short chain can be used so long as it shows good binding property. In the process of hybrid formation of a capture oligo and a target, if there is an obstruction in the secondary structure negatively affecting the hybridization, a spacer or a nucleotide forming no hydrogen bond with any nucleotide can be introduced into the oligonucleotide sequence to obviate the obstruction. Further, the obstruction in the secondary structures of the target and capture oligo can also be obviated by extending or shortening the gene sequence on genome corresponding to the capture oligo at 5′ or 3′ end or at both ends of the capture oligo so that a type-specific sequence portion should be included.

DNA is usually used as the capture oligos, but a peptide nucleic acid (PNA) may also be used. The hybrids of a nucleic acid derived from a test human genome with a peptide nucleic acid have a higher Tm (melting temperature) compared with those of oligonucleotides and therefore they are expected to provide stable hybridization signals. The peptide nucleic acids can be readily synthesized by using a common peptide synthesizer.

Examples of capture oligo nucleotide sequences are shown as SEQ ID NOS: 1-397, 456-503, 507-589, 594-898, 908-1078 and 1080-1298. These capture oligos are designed based on nucleotide sequence data obtained from publications and the home page of the Japanese Society for Histocompatibility and Immunogenetics (http://square.umin.ac.jp/JSHI/frame.html) and cover the whole HLA types.

Typing of a specific HLA type can be carried out by appropriately selecting a capture oligo from them and using it in a test.

The capture oligo length is, in general, in a range of 14-22 nucleotides. From the viewpoint of hybridization, 16-17 nucleotides is a typical size. Besides the nucleotide sequences of SEQ ID NOS: 1-397, oligonucleotides having a nucleotide sequence obtained by extending or shortening any of the HLA type gene sequences at 5′ or 3′ end or at both ends of the nucleotide sequences can be used as a capture oligo.

The nucleotide sequences of SEQ ID NOS: 1-397, 456-503, 507-589, 594-898, 908-1078 and 1080-1298 may also be extended at either 5′ or 3′ end and shortened at the other end, but the length of a capture oligo is in a range of 10-24 nucleotides in any case.

In the present invention, the designed capture oligos well reflect the results of studies and researches before the present application. However, when additional information about nucleotide sequnences of novel HLA types is obtained thereafter, novel capture oligos will be designed based on the method described in the present specification and they will fall within the claimed scope of the present application.

Synthesis of oligonucleotides, preparation of chromosome DNA, hybridization and PCR can be performed by usual methods known to those skilled in the art (see Maniatis, T. et al., “Molecular Cloning, A Laboratory Manual, second edition”, Cold Spring Harbor Laboratory Press (1989)). The oligonucleotides can also be synthesized by using a commercially available DNA synthesizer.

<2> Preparation of Substrate for Use in Present Invention

Material of a substrate on which oligonucleotides are immobilized is not particularly limited so long as the oligonucleotides can be stably immobilized. For example, there can be mentioned glass, synthetic resins such as polycarbonates and plastics. The form of the substrate is not particularly limited, but there can be mentioned a form of plate or film. A uniform and flat surface is suitable as a surface of the substrate.

The oligonucleotides can be immobilized on a substrate by using a method employed in usual hybridization such as physical absorption, electric bond and molecular covalent bond. In the examples of the present specification, substrates coated with carbodiimide groups or isocyanate groups on the surface thereof (Japanese Patent Laid-Open (Kokai) No. 08-023975) were used. To prepare the substrates coated with a material having carbodiimide groups or isocyanate groups on the surface thereof, employed was a method in which the substrate surfaces were coated with a polymer compound containing carbodiimide groups or isocyanate groups and irradiated with ultraviolet ray so that oligonucleotides can be covalently bonded and immobilized thereon. This is because marked improvement of immobilization efficiency was successfully attained by irradiating a substrate having carbodiimide groups or isocyanate groups on the surface thereof with ultraviolet ray. As a linker for binding carbodiimide group or isocyanate group and an oligonucleotide, a compound having an amino group or imino group highly reactive to the carbodiimide group or isocyanate group is used. In case of the imino group, carbodiimide group or isocyanate group can be bound by polymerizing thymine at either end of the capture oligo.

When oligonucleotides are spotted, if the amount of spotted oligonucleotides is too small, sufficient reactivity between the oligonucleotides and the nucleic acid target may not be ensured and therefore typing may become difficult. Further, high-density spotting invites technical problems as well as high cost. In addition, a more precise and expensive detecting device (typically, a scanner) is required for determination of occurrence of hybridization utilizing fluorescent labeling of the nucleic acid target, chemical color development or the like. Therefore, the oligonucleotides are preferably immobilized on the substrate surface in a diameter of 10-1,000 μm. The oligonucleotides can be immobilized by spotting an oligonucleotide solution on a substrate by using, for example, a spotting machine. It is usually preferable that the oligonucleotide solution is spotted substantially in a circular shape.

Each oligonucleotide is spotted at a plurality of sites on one single substrate, and the spots are preferably arranged in a grid pattern. When the spot size is 1000 μm in diameter, the total number of the spots is preferably 1600 or less per cm², and when such spots are spotted in a square shape, the square dimension is preferably 40×40 or less. When the spot size is 10 μm in diameter, the total number of spots is preferably 400 or less per cm² , and when such spots are spotted in a square shape, the square dimension is preferably 20×20 or less. If the longitudinal and transverse sizes are different, the numbers along longitudinal and transverse directions can be adjusted depending on the shape.

<3> Arrangement of Oligonucleotides on Substrate

To facilitate typing of each HLA type, oligonucleotides are preferably arranged on a substrate so that oligonucleotides to be used for determining each HLA type should be placed in one partition, in a line, or the like. Oligonucleotides suitable for low accuracy genotyping and those suitable for high accuracy genotyping may be immobilized on the same substrate to simultaneously carry out low accuracy genotyping and high accuracy genotyping. To perform low accuracy genotyping and high accuracy genotyping stepwise, a substrate on which oligonucleotides suitable for low accuracy genotyping are immobilized and a substrate on which oligonucleotides suitable for high accuracy genotyping are immobilized may be separately prepared.

Alternatively, the typing may be performed by preparing alignment of gene sequence of each HLA, setting a nucleotide sequence in which at least two nucleotide polymorphisms or sequence polymorphisms involving the HLA typing are observed in a nucleotide sequence consisting of 1-10 nucleotides as a patchwork segment, and finding the patchworks in gene sequences of all of the HLA genes and, performing typing by combination of the patchworks. To perform advanced typing, the typing may be performed by setting a nucleotide sequence in which at least one nucleotide polymorphism or sequence polymorphism are observed in a nucleotide sequence consisting of 1-10 nucleotides based on the alignment of the nucleotide sequences of HLA genes as a satellite segment, and finding the satellites in gene sequences of all of the HLA genes and, performing typing by combination of the satellites, whereby a type which cannot be discriminated by the patchworks may be discriminated. Typing using patchworks and satellites may make it possible to judge whether each of HLA typing of the test specimen is homozygote or heterozygote and determine the types of HLA simultaneously. The oligonucleotides may be prepared to be allocated so that the HLA types are determined by developing progressively the patchworks and satellites in a test specimen.

Spots on the substrate are usually arranged within an area of 1 cm². FIGS. 1-7 and 11-17 show examples of preferred positions of oligonucleotides immobilized on the substrate. One square in the figure represents a spot of each oligonucleotide. The indicated numbers represent sequence numbers of the oligonucleotides.

<4> Preparation of Nucleic Acid from Test Sample and Preparation of Nucleic Acid Probe

Nucleic acid can be prepared from a test sample by the same way as a usual method of preparing nucleic acids from animal cells. For example, DNA can be prepared by a method described in Maniatis, T. et al., “Molecular Cloning, A Laboratory Manual, second edition”, Cold Spring Harbor Laboratory Press (1989). DNA can also be extracted from cells obtained by culture in a similar manner. This method is a standard test method, but there are many alternative methods and any of these methods may be employed.

A nucleic acid target for use in HLA typing is prepared by using the obtained DNA. The nucleic acid target can be prepared by amplifying a nucleic acid by using primers designed so as to correspond to a nucleotide sequence of capture oligo. While DNA is usually used as the nucleic acid target, RNA may also be used. As a nucleic acid amplification method, there can be mentioned, for example, a method of amplifying the nucleic acid as DNA by PCR (polymerize chain reaction) or as RNA by in vitro transcription.

Primers used for PCR are designed so that a nucleic acid target should include a complementary nucleotide sequence of a capture oligo except for a sequence region specific to each HLA type. A nucleic acid target may be longer or shorter than a capture oligo, so long as it is capable of hybridization. To increase specificity of amplification reactions, initial amplification may be carried out by using preliminary primers for amplifying a region larger than the target nucleic acid probe and then nucleic acid amplification may be carried out by using the amplified DNA as a template and primers for obtaining the target nucleic acid probe.

There may be a plurality of type-specific nucleotide sequence regions involved in typing depending on HLA types. In such a case, nucleic acid targets corresponding to each specific region can be prepared.

Furthermore, typing of DQB and DRB is carried out by selecting primers for high accuracy typing based on results obtained by using a nucleic acid sequence amplified by primers for low accuracy typing and then carrying out high accuracy typing, or allowing hybridization of nucleic acid sequences amplified by using all of the low accuracy primers for DQB and DRB and nucleic acid sequences amplified by using all of the high accuracy primers for DQB and DRB in separate blocks on which different captures are immobilized and determining low accuracy or high accuracy type from the obtained respective results. For typing of DQA, DRA, DPA and DPB, high accuracy typing is performed by using nucleic acid sequences amplified by using primers for high accuracy typing.

If a primer for use in final nucleic acid amplification is labeled in advance, a labeled nucleic acid target can be obtained. The nucleic acid target may be labeled during or after nucleic acid amplification. As the label, those labeling substances similar to those for a probe for use in usual hybridization, such as fluorescent substances or haptens, can be used. Specifically, as the fluorescent substances, there can be mentioned, for example, fluoresceine (FITC), rhodamine, phycoerythrin (PE), Texas Red, cyanine fluorescent dyes and so forth. As the haptens, there can be mentioned biotin, digoxigenin (Dig), dinitrophenyl (DNP) and so forth.

Primers for preparing a nucleic acid target can be included in an HLA typing kit together with a substrate on which oligonucleotides are immobilized.

<5> Hybridization of Oligonucleotides on Substrate and Nucleic Acid Target

Hybridization can be performed in the same way as usual nucleic acid hybridization. A specific procedure will be exemplified below.

A nucleic acid target is added to a fusion solution comprising a salt solution such as standard saline citrate (SSC), a blocking solution such as sodium dodecyl sulfate (SDS) or bovine serum albumin (BSA) and an additive for promoting fusion reactions. When the target is double-stranded, denaturation is performed by heating or the like. After a few μL of a nucleic acid target solution is added onto a substrate, heat treatment is performed for a few hours (usually at 37-70° C.) so that a hybrid should be formed between an oligonucleotide immobilized on the substrate and the nucleic acid target.

5× SSC or 3 M tetramethylammonium chloride is added onto the substrate and heated (usually at 37-50° C.). Then, nonspecific nucleic acids that do not form hybrids are removed from the substrate and only specific hybrids are selectively maintained on the substrate.

To detect a hybrid, a fluorescent substance or hapten introduced into the nucleic acid target is used. When a hapten is used, a solution containing a conjugate of a protein recognizing the hapten or a protein that binds to the hapten and alkaline phosphatase, horseradish peroxidase or the like (enzyme conjugate) is added onto the substrate and allowed to react at room temperature for several tens of minutes. A nonspecific adsorption reaction of the enzyme conjugate and the substrate can be prevented by completely coating regions on the substrate other than the regions on which oligonucleotides are immobilized with a protein such as casein before this binding reaction of the hapten and the enzyme conjugate is performed. This treatment can be carried out by, after the oligonucleotides are immobilized, adding a solution of a protein such as casein onto the substrate and leaving it at room temperature for a several tens of minutes.

After the binding reaction of the enzyme conjugate and the hapten of the nucleic acid target is completed, the substrate is washed with an appropriate buffer containing a surfactant to remove enzyme conjugates that have not bound to the hapten. Thus, only enzyme conjugates bound to the hapten in the nucleic acid target should remain on the substrate.

To visualize the hybrid, a compound that becomes insoluble only in the presence of a hapten and an enzyme conjugate is added. The generation of such an insoluble compound is amplified by enzymatic reaction, and hence the hybrid is visualized. As the compound used for this purpose, when the enzyme in the enzyme conjugate is alkaline phosphatase, there can be used nitroblue tetrazolium chloride (NBT) and 5-bromo-4-chloro-3-indolylphosphate p-toluidine salt (BCIP). When the enzyme is horseradish peroxidase, 3,3′,5,5′-tetramethylbenzidine (TMB) or the like can be used.

HLA typing based on the obtained results of hybridization is carried out by observing pigmentation or fluorescence at positions where capture oligos are immobilized. That is, the positions showing pigmentation or fluorescence indicate a corresponding gene. When all of oligonucleotides belonging to each type show color development, the type is determined. For example, in FIGS. 8-10 referred to in the examples, hybridization signals were obtained only for oligonucleotides of SEQ ID NOS: 141 and 143 out of SEQ ID NOS: 141-144, and thus the test specimen is typed as DRA*0101. Correspondence of hybridization signals obtained at positions of capture oligos of SEQ ID NOS: 1-397 and HLA types are summarized in Tables 1-20.

TABLE 1
Correspondence of DQA1 types and high accuracy typing capture oligos

| Type | Corresponding capture oligos |
|---|---|
| DQA1*0101 | 1, 4, 7, 10, 12, 14, 16, 18, 22, 24, 27, 31, 35, 39, 42, 47, 51, 53 |
| DQA1*01021 | 1, 4, 7, 10, 12, 14, 16, 22, 24, 27, 31, 35, 39, 42, 47, 51, 53 |
| DQA1*01022 | 1, 4, 7, 10, 12, 14, 16, 20, 22, 24, 2731, 35, 39, 42, 47, 51, 53 |
| DQA1*0103 | 2, 4, 7, 10, 15, 20, 22, 26, 27, 31, 35, 39, 42, 47, 51, 53 |
| DQA1*0104 | 1, 4, 7, 10, 12, 14, 16, 18, 22, 24, 27, 31, 35, 39, 42, 47, 51, 53 |
| DQA1*0105 | 1, 4, 7, 10, 12, 14, 16, 18, 22, 24, 27, 31, 35, 39, 42, 47, 51, 53 |
| DQA1*0201 | 1, 5, 8, 10, 15, 19, 23, 24, 28, 32, 37, 39, 42, 47, 52, 53 |
| DQA1*03011 | 2, 5, 9, 13, 17, 19, 23, 24, 29, 33, 37, 41, 43, 48, 49, 52, 53 |
| DQA1*0302 | 2, 5, 9, 13, 17, 19, 23, 24, 29, 33, 36, 41, 43, 49, 52, 53 |
| DQA1*0303 | 1, 5, 9, 12, 17, 19, 23, 24, 29, 33, 36, 41, 43, 49, 52, 53 |
| DQA1*0401 | 2, 6, 8, 10, 12, 14, 16, 21, 22, 25, 30, 34, 38, 41, 44, 48, 52, 54 |
| DQA1*05011 | 3, 6, 8, 10, 12, 14, 16, 21, 22, 25, 30, 34, 38, 41, 45, 50, 52, 53 |
| DQA1*05012 | 1, 6, 8, 10, 12, 14, 16, 20, 22, 25, 30, 34, 38, 41, 45, 50, 52, 53 |
| DQA1*05013 | 3, 6, 8, 10, 12, 14, 16, 20, 22, 25, 30, 34, 38, 41, 45, 50, 52, 53 |
| DQA1*0502 | 8, 10, 12, 14, 16, 20, 22, 25, 30, 34, 38, 40, 45, 50, 52, 53 |
| DQA1*0503 | 3, 6, 8, 10, 12, 14, 16, 20, 22, 25, 30, 34, 38, 41, 45, 50, 52, 53 |
| DQA1*0504 | 3, 6, 8, 11, 14, 16, 20, 22, 25, 30, 34, 38, 41, 45, 50, 52, 53 |
| DQA1*06011 | 2, 6, 8, 10, 15, 21, 22, 25, 30, 34, 38, 41, 46, 48, 52, 53 |
| DQA1*06012 | 8, 10, 15, 21, 22, 25, 30, 34, 38, 41, 46, 48, 52, 53 |

TABLE 2
Correspondence of DQB types and low
accuracy typing capture oligos

| | Corresponding |
|---|---|
| Type | capture oligos |
| DQB1*0501 | 56 |
| DQB1*0502 | 56 |
| DQB1*05031 | 56 |
| DQB1*06011 | 55 |
| DQB1*06013 | 55 |
| DQB1*0602 | 55 |
| DQB1*0603 | 55 |
| DQB1*0604 | 56 |
| DQB1*06051 | 56 |
| DQB1*0609 | 56 |
| DQB1*0610 | 55 |
| DQB1*0611 | 55 |
| DQB1*0612 | 56 |
| DQB1*0614 | 55 |
| DQB1*0201 | 57, 58 |
| DQB1*0202 | 57, 58 |
| DQB1*0203 | 57, 58 |
| DQB1*03011 | 57 |
| DQB1*03012 | 57 |
| DQB1*0302 | 57 |
| DQB1*03032 | 57 |
| DQB1*0304 | 57 |
| DQB1*0305 | 57 |
| DQB1*0306 | 57 |
| DQB1*0307 | 57 |
| DQB1*0308 | 57 |
| DQB1*0401 | 57 |

TABLE 3
Correspondence of DQB types and high accuracy typing capture oligos

| Type | Corresponding capture oligos |
|---|---|
| DQB1*0501 | 62, 65, 68, 70, 74, 79, 82, 86, 89, 92, 97, 99, 103, 114, 116, 118, 126, 131, 137, 138 |
| DQB1*0502 | 62, 65, 68, 70, 74, 79, 82, 86, 89, 93, 97, 99, 104, 114, 116, 118, 126, 130, 137, 138 |
| DQB1*05031 | 62, 65, 68, 70, 74, 79, 82, 86, 94, 97, 99, 106, 114, 116, 118, 126, 130, 137, 138 |
| DQB1*05032 | 74, 79, 82, 86, 94, 97, 99, 105, 114, 116 |
| DQB1*0504 | 65, 68, 70, 74, 80, 83, 86, 89, 93, 97, 99, 104, 114, 117, 119, 126, 131 |
| DQB1*06011 | 63, 66, 69, 75, 80, 84, 94, 97, 99, 106, 114, 117, 120, 124, 127, 132, 135, 139 |
| DQB1*06012 | 66, 68, 70, 75, 80, 84, 94, 98, 99, 106, 114, 117, 120, 124, 127, 132, 135, 139 |
| DQB1*06013 | 63, 66, 69, 75, 80, 84, 94, 97, 99, 106, 114, 117, 120, 124, 127, 132, 135, 139 |
| DQB1*0602 | 64, 67, 68, 70, 76, 80, 87, 89, 95, 97, 99, 105, 114, 116, 121, 125, 127, 132, 135, 139 |
| DQB1*0603 | 62, 67, 68, 70, 77, 79, 87, 89, 95, 97, 99, 105, 114, 116, 121, 125, 127, 132, 135, 139 |
| DQB1*0604 | 62, 67, 68, 70, 77, 79, 87, 89, 93, 97, 99, 103, 114, 116, 122, 125, 127, 132, 134, 138 |
| DQB1*06051 | 62, 65, 68, 70, 77, 8087, 89, 93, 97, 99, 103, 114, 116, 122, 125, 127, 132, 134, 138 |
| DQB1*06052 | 77, 80, 87, 89, 93, 97, 99107, 114, 116, 122, 125, 127, 132 |
| DQB1*0606 | 77, 80, 87, 89, 93, 97, 99, 103, 114, 116, 122, 128, 129, 131 |
| DQB1*0607 | 67, 68, 70, 77, 79, 87, 89, 95, 97, 99, 105, 114, 116, 122, 125, 127, 132 |
| DQB1*0608 | 67, 68, 70, 77, 79, 87, 89, 95, 97, 99, 103, 114, 116, 121, 127, 132 |
| DQB1*0609 | 62, 67, 68, 7077, 80, 87, 89, 93, 97, 99, 103, 114, 116, 122, 125, 127, 132, 134, 138 |
| DQB1*0610 | 64, 67, 68, 70, 76, 80, 87, 89, 95, 97, 99, 104, 114, 116, 121, 125, 127, 132, 135, 139 |
| DQB1*0611 | 67, 68, 70, 76, 80, 87, 89, 95, 97, 99, 105, 114, 116, 121, 125, 127, 132, 135, 139 |
| DQB1*0612 | 62, 67, 68, 70, 77, 80, 87, 89, 93, 97, 99, 103, 114, 116, 121, 125, 127, 132, 134, 138 |
| DQB1*0613 | 64, 67, 68, 70, 76, 80, 87, 89, 95, 97, 99, 103, 114, 116, 121, 125, 127, 132, 135 |
| DQB1*0614 | 67, 68, 70, 77, 79, 87, 89, 95, 97, 99, 105, 114, 116, 121, 125, 127, 132, 135, 139 |
| DQB1*0201 | 60, 61, 62, 67, 71, 78, 81, 85, 90, 100, 108, 114, 116, 123, 128, 129, 131, 136, 140 |
| DQB1*0202 | 59, 60, 61, 62, 67, 71, 78, 81, 85, 90, 100, 108, 114, 117, 123, 128, 129, 131, 136, 140 |
| DQB1*0203 | 62, 67, 71, 78, 81, 85, 90, 100, 109, 114, 116, 117, 123, 128, 129, 131, 136, 140 |
| DQB1*03011 | 62, 66, 68, 70, 75, 80, 88, 91, 93, 101, 110, 114, 116, 122, 125, 127, 132, 136, 140 |
| DQB1*03012 | 66, 68, 70, 75, 80, 87, 91, 93, 101, 110, 114, 116, 122, 125, 127, 132, 136, 140 |
| DQB1*0302 | 62, 67, 68, 70, 76, 80, 88, 94, 101, 111, 114, 116, 122, 125, 127, 132, 136, 140 |
| DQB1*03032 | 62, 67, 68, 70, 76, 80, 88, 94, 101, 110, 114, 116, 122, 125, 127, 132, 136, 140 |
| DQB1*0304 | 62, 66, 68, 70, 75, 80, 88, 91, 93, 101, 111, 114, 116, 122, 125, 127, 132, 136, 140 |
| DQB1*0305 | 62, 67, 72, 74, 80, 87, 94, 101, 111, 114, 116, 122, 125, 127, 132, 136, 140 |
| DQB1*0306 | 62, 67, 68, 70, 76, 80, 88, 94, 101, 110, 115, 117, 119, 126, 133, 136, 140 |
| DQB1*0307 | 62, 67, 68, 70, 76, 80, 88, 96, 101, 111, 114, 116, 122, 125, 127, 132, 136, 140 |
| DQB1*0308 | 62, 67, 68, 70, 76, 80, 88, 94, 101, 111, 114, 116, 121, 125, 127, 132, 136, 140 |
| DQB1*0401 | 64, 67, 73, 74, 80, 87, 94, 102, 112, 113, 115, 117, 119, 126, 133, 136, 140 |
| DQB1*0402 | 64, 67, 72, 74, 80, 87, 94, 102, 112, 113, 115, 117, 119, 126, 133, 136, 140 |

TABLE 4
Correspondence of DRA types and high
accuracy typing capture oligos

| | Corresponding |
|---|---|
| Type | capture oligos |
| DRA*0101 | 141, 143 |
| DRA*0102 | 142, 144 |

TABLE 5
Correspondence of DRB1 types and low accuracy
and high accuracy typing capture oligos (1), alleles
whose serological antigen type is DR1

| | Corresponding capture oligos | |
|---|---|---|
| Type | low accuracy | High accuracy |
| DRB1*0101 | 145 | 166, 173, 187, 213, 246, 250 |
| DRB1*01021 | 145 | 166, 173, 187, 213, 232, 236, 250 |
| DRB1*01022 | 145 | 166, 173, 187, 213, 228, 232, 236, 250 |
| DRB1*0103 | 145 | 166, 173, 187, 211, 215, 246, 250 |
| DRB1*0104 | 145 | 173, 187, 213, 230, 250 |
| DRB1*0105 | 145 | 173, 187, 192, 213, 246, 250 |
| DRB1*0106 | 145 | |

TABLE 6
Correspondence of DRB1 types and low accuracy and high accuracy typing
capture oligos (2), alleles whose serological antigen type is DR2

Corresponding capture oligos

| Type | low accuracy | high accuracy |
|---|---|---|
| DRB1*15011 | 149 | 157, 187, 194, 201, 211, 212, 234, 247 |
| DRB1*15012 | 149 | 157, 187, 194, 201, 211, 212, 233, 247 |
| DRB1*15021 | 149 | 157, 18, 7194, 201, 211, 212, 246, 247 |
| DRB1*15022 | | 187, 194, 211, 212, 247, 250 |
| DRB1*15023 | 149 | 157, 181, 187, 194, 201, 211, 212 |
| DRB1*1503 | 149 | 157, 178, 187, 194, 201, 211, 212, 234, 247 |
| DRB1*1504 | 149 | 157, 187, 194, 201, 210, 212, 234, 247 |
| DRB1*1505 | 149 | 157, 187, 194, 201, 212, 234, 247 |
| DRB1*1506 | 149 | 187, 194, 196 201, 211, 212, 247 |
| DRB1*1507 | 149 | 157, 187, 201, 211, 212, 247 |
| DRB1*1508 | 149 | 157, 187, 194 201 212, 246, 247 |
| DRB1*1509 | 149 | |
| DRB1*1510 | 149 | |
| DRB1*16011 | 149 | 157, 187 201, 210 220, 246, 247 |
| DRB1*16012 | 149 | 157, 187, 20, 210, 246, 247 |
| DRB1*16021 | 149 | 157, 187, 201, 220, 246, 247 |
| DRB1*16022 | 149 | 187, 201, 247 |
| DRB1*1603 | 149 | 157, 187 201, 210, 221, 246, 247 |
| DRB1*1604 | 149 | 187, 201, 210, 222, 247 |
| DRB1*1605 | 149 | 187, 201, 211, 220, 247 |
| DRB1*1607 | 149 | 157, 187, 201, 211, 220, 241, 246, 247 |
| DRB1*1608 | 149 | 157, 184 201, 210, 220, 246, 247 |

TABLE 7
Correspondence of DRB1 Types and low accuracy and high accuracy typing
capture oligos (3), alleles whose serological antigen type is DR3

| | Corresponding capture oligos | |
|---|---|---|
| | low | |
| Type | accuracy | high accuracy |
| DRB1*03011 | 146 | 171, 179, 184, 194, 216, 226, 229, 234, 229, 234, 248, 250 |
| DRB1*03012 | 146 | 171, 179, 184, 194, 216, 226, 230, 234, 230, 234, 248, 250 |
| DRB1*03021 | 146 | 174, 184, 216 226, 229, 246, 248, 250 |
| DRB1*03022 | 146 | 174, 184, 216, 226, 230, 246, 248, 250 |
| DRB1*0303 | 146 | 174, 184, 216, 226, 229, 234, 248, 250 |
| DRB1*0304 | 146 | 171, 179, 187, 194, 216, 226, 229, 248, 250 |
| DRB1*0305 | 146 | 171, 179, 184, 194, 216, 226, 229, 246, 248, 250 |
| DRB1*0306 | 146 | 171, 179, 184, 216, 229, 234, 248, 250 |
| DRB1*0307 | 146 | 179, 184, 194, 216, 226, 229, 234, 248, 250 |
| DRB1*0308 | 146 | 171, 179, 184, 194, 206, 216, 226, 229, 234, 248 |
| DRB1*0309 | 146 | 171, 182, 184, 194, 216, 226. 229, 246, 250 |
| DRB1*0310 | 146 | 171, 179, 184, 194, 200, 216, 226, 229, 234, 244, 248 |
| DRB1*0311 | 146 | 171, 179, 184, 194, 216, 227, 229, 234, 248, 250 |
| DRB1*0312 | 146 | 171, 179, 184, 194, 199, 216, 226, 229, 248 |
| DRB1*0313 | 146 | |
| DRB1*0314 | 146 | |
| DRB1*0315 | 146 | |
| DRB1*0316 | 146 | |
| DRB1*0317 | 146 | |

TABLE 8
Correspondence of DRB1 types and low accuracy and high accuracy typing
capture oligos (4), alleles whose serological antigen type is DR4

Corresponding capture oligos

| Type | low accuracy | high accuracy |
|---|---|---|
| DRB1*04011 | 147 | 160, 169, 180, 216, 246, 249, 250 |
| DRB1*04012 | 147 | 160, 169, 183, 216, 246, 249, 250 |
| DRB1*0402 | 147 | 160, 169, 180, 211, 215, 234, 249, 250 |
| DRB1*0403 | | 160, 169, 180, 213, 223, 234, 249, 250 |
| DRB1*04031 | 147 | |
| DRB1*04032 | 147 | 161, 169, 180, 201, 213, 223, 234, 249 |
| DRB1*0404 | 147 | 160, 169, 180, 213, 234, 249, 250 |
| DRB1*04051 | 147 | 160, 169, 180, 199, 213, 246, 249 |
| DRB1*04052 | 147 | 160, 169, 180, 193, 199, 213, 246 |
| DRB1*0406 | 147 | 160, 169, 180, 187, 213, 223, 234, 250 |
| DRB1*0407 | 147 | 160, 169, 180, 213, 223, 246, 249, 250 |
| DRB1*0408 | 147 | 160, 169, 180, 213, 246, 249, 250 |
| DRB1*0409 | | 169, 180, 199, 216, 249 |
| DRB1*0410 | 147 | 160, 169, 180, 199, 213, 234, 249 |
| DRB1*0411 | 147 | 160, 169, 180, 199, 213, 223, 234, 249 |
| DRB1*0412 | 147 | 160, 169, 199, 211, 222, 234, 249 |
| DRB1*0413 | | 169, 180, 216, 234, 249, 250 |
| DRB1*0414 | | 169, 180, 211, 215, 249, 250 |
| DRB1*0415 | 147 | 160, 169, 180, 206, 210, 249 |
| DRB1*0416 | | 169, 180, 207, 216, 249 |
| DRB1*0417 | | 169, 180, 199, 213, 223, 249 |
| DRB1*0418 | | 180, 211, 222, 234, 249, 250 |
| DRB1*0419 | | 169, 180, 187, 213, 246, 250 |
| DRB1*0420 | | 169, 180, 187, 213, 223, 250 |
| DRB1*0421 | 147 | 160, 169, 180, 187, 216, 246, 250 |
| DRB1*0422 | 147 | 160, 169, 180, 216, 226, 229, 234, 249, 250 |
| DRB1*0423 | 147 | 160, 169, 180, 213, 245, 249, 250 |
| DRB1*0424 | 147 | 160, 169, 180, 199, 214, 246, 249 |
| DRB1*0425 | 147 | 160, 169, 180, 210, 222, 249, 250 |
| DRB1*0426 | 147 | 160, 169, 180, 209, 216, 249 |
| DRB1*0427 | 147 | 160, 169, 118, 213, 223, 236, 249, 250 |
| DRB1*0428 | 147 | 160, 169, 180, 194, 199, 213, 246, 249 |
| DRB1*0429 | 147 | 160, 169, 180, 197, 199, 213, 246, 249 |
| DRB1*0430 | 147 | 160, 169, 180, 195, 213, 246, 249 |
| DRB1*0431 | 147 | 160, 169, 180, 213, 222, 246, 249, 250 |
| DRB1*0432 | 147 | 160, 169, 180, 219, 249, 250 |
| DRB1*0433 | 147 | |
| DRB1*0434 | 147 | |

TABLE 9
Correspondence of DRB1 types and low accuracy and high
accuracy typing capture oligos (5), alleles whose serological
antigen type is DR5 and whose subtype is DR11

| | Corresponding capture oligos | |
|---|---|---|
| Type | low accuracy | high accuracy |
| DRB1*11011 | 146 | 194, 206, 210, 246, 249 |
| DRB1*11012 | 146 | 194, 206, 210, 239, 246, 249 |
| DRB1*11013 | 146 | 194, 206, 210, 220, 246, 249 |
| DRB1*1102 | 146 | 194, 206, 211, 215, 234, 249 |
| DRB1*1103 | 146 | 194, 206, 210, 220, 246, 249 |
| DRB1*11041 | 146 | 194, 206, 210, 234, 249 |
| DRB1*11042 | 146 | 194, 206, 210, 234, 239, 249 |
| DRB1*1105 | 146 | 194, 206, 210, 246, 249 |
| DRB1*1106 | 146 | 194, 206, 210, 236, 249 |
| DRB1*1107 | 146 | 194, 206, 216, 226, 229, 234, 249 |
| DRB1*11081 | | 194, 206, 246, 249 |
| DRB1*11082 | | 194, 206, 246, 249 |
| DRB1*1109 | | 179, 184, 194, 206, 210, 246, 248 |
| DRB1*1110 | | 179, 188, 194, 206, 210, 248 |
| DRB1*1111 | | 194, 206, 210, 215, 246, 249 |
| DRB1*1112 | | 188, 194, 206, 210 |
| DRB1*1113 | 146 | 179, 188, 194, 206, 214, 232, 234, 248 |
| DRB1*1114 | 146 | 194, 206, 211, 215, 246, 249 |
| DRB1*1115 | 146 | 186, 194, 206, 210, 246 |
| DRB1*1116 | 146 | 179, 184, 194, 206, 211, 215, 248 |
| DRB1*1117 | 146 | 179, 188, 206, 214, 223, 232, 234, 248 |
| DRB1*1118 | 146 | 194, 206, 211, 234, 249 |
| DRB1*1119 | 146 | 194, 206, 211, 246, 249 |
| DRB1*1120 | 146 | 179, 184, 194, 206, 211, 215, 246, 248 |
| DRB1*1121 | 146 | 194, 206, 211, 215, 249 |
| DRB1*1122 | 147 | 162, 194, 206 210, 246, 247, 249 |
| DRB1*1123 | 146 | 194, 206, 210, 222, 249 |
| DRB1*1124 | 146 | 185, 194, 206, 210 |
| DRB1*1125 | 146 | 194, 206, 210, 222, 234, 249 |
| DRB1*1126 | 146 | 194, 206, 213, 234, 249 |
| DRB1*1127 | | 194, 206, 210, 230, 249 |
| DRB1*11271 | 146 | |
| DRB1*11272 | 146 | |
| DRB1*1128 | 146 | 184, 194 206 |
| DRB1*1129 | 146 | 187, 194, 206, 210, 246 |
| DRB1*1130 | 154 | 165, 194, 206, 210, 246, 249 |
| DRB1*1131 | 146 | 194, 208, 211, 246, 249 |
| DRB1*1132 | 146 | 194, 206, 210, 224, 246, 249 |
| DRB1*1133 | 146 | 194, 210, 242, 246, 249, 250 |
| DRB1*1134 | 146 | 194, 206, 213, 234, 249 |
| DRB1*1135 | 146 | 194, 210, 234, 242, 249, 250 |
| DRB1*1136 | 146 | |
| DRB1*1137 | 146 | |

TABLE 10
Correspondence of DRB1 types and low accuracy and high
accuracy typing capture oligos (6), alleles whose serological
antigen type is DR5 and whose subtype is DR12

| | Corresponding capture oligos | |
|---|---|---|
| Type | low accuracy | high accuracy |
| DRB1*1201 | 146 | 158, 172, 191, 194, 202, 211, 220, 232, 236, 248 |
| DRB1*12021 | 146 | 158, 172, 191, 194, 202, 210, 220, 232, 246, 248 |
| DRB1*12022 | 146 | 158, 172, 191, 194, 202, 210, 220, 248 |
| DRB1*12032 | 146 | 158, 172, 191, 194, 202, 211, 220, 234, 248 |
| DRB1*1204 | 146 | 158, 172, 191, 194, 206, 211, 220, 232, 248 |
| DRB1*1205 | 146 | 158, 172, 190, 194, 202, 211, 220, 232, 236, 248 |
| DRB1*1206 | 146 | |

TABLE 11
Correspondence of DRB1 types and low accuracy and high
accuracy typing capture oligos (7), alleles whose serological
antigen type is DR6 and whose subtype is DR13

| | Corresponding capture oligos | |
|---|---|---|
| Type | low accuracy | high accuracy |
| DRB1*1301 | 146 | 179, 184, 194, 211, 215, 234, 248, 250 |
| DRB1*1302 | | 179, 184, 194 211, 215, 246, 248, 250 |
| DRB1*13021 | 146 | |
| DRB1*13022 | 146 | |
| DRB1*12031 | 146 | 199, 211 218, 239, 246, 249 |
| DRB1*13032 | | 199, 211 218, 239, 246, 249 |
| DRB1*13032 | 146 | |
| DRB1*1304 | 146 | 194, 199, 211, 215, 234, 249 |
| DRB1*1305 | 146 | 179, 184, 194, 210, 246, 248, 250 |
| DRB1*1306 | 146 | 179, 184, 194, 211, 234, 248, 250 |
| DRB1*13071 | 146 | 210, 246, 249, 250 |
| DRB1*13072 | 146 | 201, 210, 246, 249 |
| DRB1*1308 | 146 | 179, 188, 211, 215, 234, 248, 250 |
| DRB1*1309 | 146 | 179, 184, 194, 211, 212, 234, 248, 250 |
| DRB1*1310 | 146 | 179, 184, 194, 211, 218, 234, 248, 250 |
| DRB1*1311 | 146 | 194, 210, 249, 250 |
| DRB1*1312 | 146 | 199, 211, 246, 249 |
| DRB1*1313 | 146 | 199, 211, 222, 246, 249 |
| DRB1*1314 | | 194, 210, 246, 249, 250 |
| DRB1*13142 | 146 | |
| DRB1*1315 | 146 | 174, 184, 194, 211, 215, 234, 248, 250 |
| DRB1*1316 | 146 | 179, 184, 194, 211, 215, 235, 248, 250 |
| DRB1*1317 | 146 | 158, 194, 211, 215, 234, 249, 250 |
| DRB1*1318 | 146 | 179, 184, 194, 210, 222, 234, 248, 250 |
| DRB1*1319 | 146 | 174, 188, 211, 215, 222, 234, 248, 250 |
| DRB1*1320 | 146 | 179, 184, 194, 215, 234, 248, 250 |
| DRB1*1321 | 146 | 194, 199, 210, 246, 249 |
| DRB1*1322 | 146 | 194, 211, 215, 234, 249, 250 |
| DRB1*1323 | 146 | 194, 211, 215, 239, 246, 249, 250 |
| DRB1*1324 | 146 | 194, 210, 215, 234, 249, 250 |
| DRB1*1325 | 146 | 194, 249, 250 |
| DRB1*1326 | 146 | 174, 184, 201, 210, 220, 248 |
| DRB1*1327 | 146 | 171, 179, 184, 194, 211, 215, 220, 234, 248, 250 |
| DRB1*1328 | 146 | 179, 184, 194, 211 215, 237, 248, 250 |
| DRB1*1329 | 146 | 179, 184, 194, 215, 246, 248, 250 |
| DRB1*1330 | 146 | 194, 199 211, 246, 249 |
| DRB1*1331 | 146 | 179, 184 194 211 215, 246, 248, 250 |
| DRB1*1332 | 146 | 179, 184, 199, 211, 215, 234, 248 |
| DRB1*1333 | 146 | 199, 211, 218, 225, 229, 249 |
| DRB1*1334 | 146 | 179, 189, 194, 211, 215, 240, 246, 248, 250 |
| DRB1*1335 | 146 | |
| DRB1*1336 | 146 | |
| DRB1*1337 | 146 | |
| DRB1*1338 | 146 | |
| DRB1*1339 | 146 | |
| DRB1*1340 | 146 | |
| DRB1*1341 | 146 | |

TABLE 12
Correspondence of DRB1 types and low accuracy and high
accuracy typing capture oligos (8), alleles whose serological
antigen type is DR6 and whose subtype is DR14

Corresponding capture oligos

| Type | low accuracy | high accuracy |
|---|---|---|
| DRB1*1401 | 146 | 179, 188, 200, 214, 223, 232, 234, 244, 248 |
| DRB1*1402 | 146 | 174, 184, 213, 246, 248, 250 |
| DRB1*1403 | 146 | 174, 184, 222, 246, 248, 250 |
| DRB1*1404 | 146 | 158, 179, 188, 200, 214, 223, 232, 234, 244, 248 |
| DRB1*1405 | 146 | 159, 179, 188, 205, 214, 223, 232, 234, 248 |
| DRB1*1406 | 146 | 174, 184, 213, 234, 248, 250 |
| DRB1*1407 | 146 | 179, 188, 200, 214, 223, 232, 244, 248 |
| DRB1*1408 | 146 | 179, 188, 203, 214, 223, 232, 234, 244, 248 |
| DRB1*1409 | 146 | 179, 184, 213, 246, 248, 250 |
| DRB1*1410 | 147 | 160, 179, 188, 200, 214, 223, 232, 234, 244, 248 |
| DRB1*1411 | 146 | 158, 179, 188, 206, 214, 223, 232, 248 |
| DRB1*1412 | | 174, 184, 222, 248, 250 |
| DRB1*1413 | 146 | 174, 184, 199, 213, 248 |
| DRB1*1414 | 146 | 179, 188, 214, 223, 232, 246, 248, 250 |
| DRB1*1415 | | 158, 179, 188, 210, 222, 234, 248, 250 |
| DRB1*1416 | 146 | 179, 188, 200, 211, 215, 234, 244, 248 |
| DRB1*1417 | 146 | 179, 184, 194, 213, 234, 248, 250 |
| DRB1*1418 | 146 | 174, 184, 205, 214, 223, 232, 234, 248 |
| DRB1*1419 | 146 | 174, 184, 216, 246, 248, 250 |
| DRB1*1420 | 146 | 174, 188, 213, 248, 250 |
| DRB1*1421 | 146 | 179, 184, 194, 216, 248, 250 |
| DRB1*1422 | 146 | 179, 188 200, 210, 244, 246, 248 |
| DRB1*1423 | 146 | 179, 188, 214, 223, 232, 234, 248, 250 |
| DRB1*1424 | 146 | 174, 184, 211 212, 246, 248, 250 |
| DRB1*1425 | 146 | 200, 210, 244, 249 |
| DRB1*1426 | 146 | 170, 179, 188, 200, 214, 223, 232, 234, 244, 248 |
| DRB1*1427 | 146 | 174, 184 210, 222, 246, 248, 250 |
| DRB1*1428 | 146 | 158, 179, 188, 200, 214, 223, 232, 244, 248 |
| DRB1*1429 | 146 | 174, 184, 213, 236, 248, 250 |
| DRB1*1430 | 146 | 179, 184 194, 213, 246, 248, 250 |
| DRB1*1431 | 146 | 158, 179, 188, 200, 214, 232, 234, 244, 2480 |
| DRB1*1432 | 146 | |
| DRB1*1433 | 146 | 179, 184, 194, 213, 223, 234, 248, 250 |
| DRB1*1434 | 146 | |
| DRB1*1435 | 146 | |
| DRB1*1436 | 146 | |

TABLE 13
Correspondence of DRB1 types and low accuracy and high accuracy typing capture
oligos (9), alleles whose serological antigen type is DR7, DR8, DR9 or DR10

| | Corresponding capture oligos | |
|---|---|---|
| Type | low accuracy | high accuracy |
| DRB1*0701 | | 164, 169, 170, 176, 188, 198, 202, 211, 217, 227, 231, 246, 247 |
| DRB1*07011 | 148 | |
| DRB1*07012 | 148 | |
| DRB1*0703 | 148 | 164, 169, 170, 177, 188, 198, 202, 211, 217, 227, 231, 247 |
| DRB1*0704 | 148 | 164, 169, 170, 176, 188, 198, 202, 211, 217, 227, 230, 247 |
| DRB1*0801 | 146 | 158, 199, 210, 222, 239, 246, 249 |
| DRB1*08021 | 146 | 158, 210, 222, 246, 249, 250 |
| DRB1*08022 | 146 | 158, 210, 222, 246, 249, 250 |
| DRB1*08032 | 146 | 158, 199, 211, 222, 246, 249 |
| DRB1*08041 | 146 | 158, 210, 222, 234, 249, 250 |
| DRB1*08042 | | 158, 210, 222, 238, 239, 249, 250 |
| DRB1*08043 | 146 | 158, 210, 222, 238, 239, 249, 250 |
| DRB1*0805 | 146 | 158, 199, 210, 249 |
| DRB1*0806 | 146 | 158, 199, 210, 222, 234, 249 |
| DRB1*0807 | 146 | 158, 210, 222, 239, 246, 249, 250 |
| DRB1*0808 | 146 | 158, 200, 210, 222, 244, 249 |
| DRB1*0809 | | 168, 179, 188, 210, 222, 239, 246, 248, 250 |
| DRB1*0810 | 146 | 158, 199, 211, 222, 234, 249 |
| DRB1*0811 | 146 | 158, 210, 222, 239, 246, 249, 250 |
| DRB1*0812 | 146 | 158, 199, 211, 222, 249 |
| DRB1*0813 | 146 | 158, 222, 249, 250 |
| DRB1*0814 | 146 | 167, 199, 211 222, 2467, 249 |
| DRB1*0815 | 146 | 158203211222, 244, 246, 249 |
| DRB1*0816 | 146 | 158, 185, 199, 210, 222 |
| DRB1*0817 | 146 | 158, 194, 199, 210, 222, 249 |
| DRB1*0818 | 146 | 158, 199, 211, 246, 249 |
| DRB1*0819 | 146 | 58, 211 222, 246, 249, 250 |
| DRB1*0820 | 146 | |
| DRB1*0821 | 146 | 168, 179, 188, 210, 222, 239, 246, 248, 250 |
| DRB1*09012 | 150 | 163, 169, 175, 184, 202, 210, 214, 223, 231, 243 |
| DRB1*10011 | 151 | |
| DRB1*10012 | 151 | |

TABLE 14
Correspondence of DRB3 types and low accuracy
typing and high accuracy typing capture oligos

| | Corresponding capture oligos | |
|---|---|---|
| | low | |
| Type | accuracy | High accuracy |
| DRB3*01011 | 152 | 252, 255, 258, 260, 265, 267, 271, 273, 275, 277, 279 |
| DRB3*01012 | 152 | 252, 255, 258, 260, 265, 267, 271, 273, 275, 277, 279 |
| DRB3*01013 | 152 | 252, 255, 258, 260, 265, 268, 271, 273, 275, 277, 279 |
| DRB3*01014 | 152 | 252, 255, 258, 260, 265, 267, 271, 273, 275, 278, 279 |
| DRB3*0102 | 153 | 253, 255, 258, 260, 265, 267, 271, 273, 275, 277, 279 |
| DRB3*0103 | 152 | 252, 255, 258, 260, 265, 267, 271, 273, 275, 277, 279 |
| DRB3*0104 | 152 | 252, 255, 258, 260, 265, 267, 271, 273, 275, 277, 279 |
| DRB3*0105 | 152 | 252, 256, 258, 260, 265, 267, 271, 273, 275, 277, 279 |
| DRB3*0106 | 152 | 252, 255, 258, 261, 265, 267, 271, 273, 275, 277, 279 |
| DRB3*0107 | 152 | 252, 255, 258, 262, 266, 269, 272, 273, 275, 277, 279 |
| DRB3*0201 | 154 | 254, 257, 259, 262, 266, 269, 272, 273, 275, 277, 280 |
| DRB3*02021 | 154 | 254, 257, 259, 262, 266, 269, 272, 273, 275, 277, 279 |
| DRB3*02022 | 154 | 254, 257, 259, 262, 266, 269, 272, 274, 275, 277, 279 |
| DRB3*02023 | 154 | 254, 257, 259, 262, 266, 269, 272, 273, 276, 277, 279 |
| DRB3*0203 | 154 | 254, 257, 259, 263, 266, 269, 272, 273, 275, 277, 279 |
| DRB3*0204 | 154 | 254, 257, 259, 262, 266, 269, 271, 274, 275, 277, 280 |
| DRB3*0205 | 154 | 254, 257, 258, 262, 266, 269, 272, 273, 275, 277, 279 |
| DRB3*0206 | 154 | 254, 257, 259, 264, 266, 269, 272, 273, 275, 277, 279 |
| DRB3*0207 | 154 | 254, 257, 259, 262, 266, 267, 272, 273, 275, 277, 279 |
| DRB3*0208 | 154 | 254, 257, 259, 262, 266, 270, 272, 273, 275, 277, 279 |
| DRB3*0210 | 154 | 254, 257, 259, 262, 265, 269, 272, 273, 275, 277, 279 |
| DRB3*0211 | 154 | 254, 257, 259, 262, 266, 269, 272, 273, 275, 277, 279 |
| DRB3*0212 | 154 | 254, 257, 259, 262, 266, 269, 272, 273, 275, 277, 279 |
| DRB3*0301 | 154 | 254, 257, 258, 261, 265, 267, 272, 273, 275, 277, 280 |
| DRB3*0302 | 154 | 254, 257, 259, 261, 265, 267, 272, 273, 275, 277, 280 |
| DRB3*0303 | 154 | 254, 257, 258, 261, 265, 267, 271, 273, 275, 277, 279 |

TABLE 15
Correspondence of DRB4 types and low accuracy
typing and high accuracy typing capture oligos

Corresponding capture oligos

| Type | low accuracy | high accuracy |
|---|---|---|
| DRB4*01011 | 155 | 281, 284, 286, 288 |
| DRB4*0102 | | 282, 284, 287 |
| DRB4*0103101 | 155 | 281, 284, 287, 288 |
| DRB4*0103102N | | 287 |
| DRB4*01032 | 155 | 281, 284, 287, 289 |
| DRB4*0104 | 155 | 283, 284 |
| DRB4*0105 | 155 | 281, 285 |
| DRB4*0201N | 155 | 281, 284, 286 |
| DRB4*0301N | | 286 |

TABLE 16
Correspondence of DRB5 types and low accuracy
typing and high accuracy typing capture oligos

| | Corresponding capture oligos | |
|---|---|---|
| Type | low accuracy | high accuracy |
| DRB5*01011 | 156 | 290, 292, 295, 297, 302, 304, 306 |
| DRB5*01012 | 156 | 290, 292, 295, 297, 300, 305, 306 |
| DRB5*0102 | 156 | 291, 293, 295, 297, 302, 304, 306 |
| DRB5*0103 | 156 | 291, 293, 295, 298, 304, 306 |
| DRB5*0104 | 156 | 290, 292, 295, 297, 303, 306 |
| DRB5*0105 | 156 | 290, 294, 295, 297, 302, 304, 306 |
| DRB5*0106 | 156 | 290, 292, 296, 299, 301, 305, 307 |
| DRB5*0107 | 156 | 290, 292, 296, 297, 302, 305, 307 |
| DRB5*0108N | 156 | 291, 293, 295, 297, 302, 304, 306 |
| DRB5*0109 | 156 | 290, 292, 295, 297, 302, 304, 306 |
| DRB5*0110N | 156 | 291, 293, 295, 297, 302, 304, 306 |
| DRB5*0202 | 156 | 291, 293, 296, 299, 301, 305, 306 |
| DRB5*0203 | 156 | 291, 293, 296, 299, 301, 305, 306 |
| DRB5*0204 | 156 | 291, 293, 295, 299, 301, 305, 307 |

TABLE 17
Correspondence of DRB2, DRB6 and DRB7 types and low accuracy
typing and high accuracy typing capture oligos

Corresponding capture oligos

| Type | low accuracy | high accuracy |
|---|---|---|
| DRB2*0101 | | 251 |
| DRB6*0101 | 155 | 308, 310, 312, 314, 316, 318 |
| DRB6*0201 | 155 | 309, 311, 313, 315, 317, 319 |
| DRB6*0202 | | 309, 311, 313, 315, 317, 320 |
| DRB7*01011 | | 321 |
| DRB7*01012 | | 322 |
| DRB7*01011 | 155 | 323 |

TABLE 18
Correspondence of DPA types and high
accuracy typing capture oligos

| Type | Corresponding capture oligos |
|---|---|
| DPA1*0103 | 324, 326, 330, 331, 333, 336, 338, 341, 343, 345, 347 |
| DPA1*0104 | 324, 326, 330, 331, 334, 336, 338, 341, 343, 345, 347 |
| DPA1*0105 | 324, 326, 330, 331, 333, 336, 338, 341, 343, 345, 348 |
| DPA1*0106 | 324, 326, 330, 331, 333, 337, 339, 341, 343, 345, 347 |
| DPA1*02011 | 324, 327, 330, 331, 333, 337, 339, 342, 343, 345, 348 |
| DPA1*02012 | 324, 326, 330, 331, 333, 337, 339, 342, 343, 345, 348 |
| DPA1*02013 | 324, 326, 330, 331, 333, 337, 338, 342, 343, 345 |
| DPA1*02014 | 324, 330, 331, 333, 337, 339, 342, 343, 345, 348 |
| DPA1*02021 | 325, 327, 329, 331, 333, 337, 340, 342, 343, 345, 348 |
| DPA1*02022 | 325, 327, 329, 331, 333, 337, 338, 342, 343, 345, 348 |
| DPA1*0203 | 324, 327, 330, 331, 333, 336, 338, 342, 343, 345, 347 |
| DPA1*0301 | 325, 327, 330, 331, 333, 336, 338, 341, 344, 345, 347 |
| DPA1*0302 | 325, 327, 330, 331, 333, 336, 338, 341, 343, 345, 347 |
| DPA1*0401 | 324, 326, 328, 332, 335, 336, 338, 342, 343, 346, 348 |

TABLE 19
Correspondence of DPB high accuracy typing capture (1)

| Type | Corresponding capture oligos |
|---|---|
| DPB1*01011 | 349, 254, 358, 361, 366, 367, 376 |
| DPB1*01012 | 350, 355, 358, 364, 365, 368, 373, 376 |
| DPB1*02011 | 350, 354, 358, 364, 365, 368, 373, 376 |
| DPB1*02012 | 350, 354, 358, 364, 365, 369, 376 |
| DPB1*02013 | 350, 354, 358, 362, 365, 371, 376 |
| DPB1*0202 | 349, 356, 358, 364, 365, 370, 374, 377 |
| DPB1*0301 | 350, 354, 358, 363, 365, 367, 376 |
| DPB1*0401 | 350, 354, 358, 364, 365, 368, 373, 376 |
| DPB1*0402 | 350, 354, 358, 362, 365, 371, 376 |
| DPB1*0501 | 349, 356, 358, 364, 365, 370, 374, 377 |
| DPB1*0601 | 350, 354, 358, 364, 365, 368, 373, 376 |
| DPB1*0801 | 351, 356, 358, 364, 365, 370, 374, 376 |
| DPB1*0901 | 351, 356, 358, 364, 365, 368, 373, 376, 386, 388, 390, 393, 396 |
| DPB1*1001 | 349, 356, 360, 361, 366, 367, 377, 384, 385, 388, 391, 393, 396 |
| DPB1*11011 | 349, 356, 359, 361, 366, 367, 377, 384, 386, 388, 391, 393, 396 |
| DPB1*11012 | 349, 356, 358, 361, 365, 367, 376, 386, 388, 392, 393, 396 |
| DPB1*1301 | 351, 356, 358, 364, 365, 370, 374, 377, 382, 386, 388, 390, 393, 396 |
| DPB1*1401 | 349, 354, 360, 361, 366, 367, 377, 384, 386, 388, 391, 395, 396 |
| DPB1*1501 | 350, 354, 358, 364, 365, 368, 373, 376, 386, 388, 391, 393, 396 |
| DPB1*1601 | 351, 356, 358, 364, 365, 370, 374, 376, 386, 388, 391, 393, 396 |
| DPB1*1701 | 349, 354, 358, 364, 365, 368, 373, 376, 382, 386, 388, 391, 395, 396 |
| DPB1*1801 | 350, 354, 358, 364, 365, 371, 376, 386, 388, 392, 393, 396 |
| DPB1*1901 | 349, 356, 358, 364, 365, 370, 374, 377, 382, 386, 388, 391, 393, 396 |
| DPB1*20011 | 349, 356, 358, 364, 365, 370, 374, 377, 382, 386, 388, 391, 393, 396 |
| DPB1*20012 | 349, 356, 358, 362, 365, 371, 376, 386, 388, 391, 393, 396 |
| DPB1*2101 | 350, 354, 358, 362, 365, 371, 376, 386, 388, 391, 393, 396 |
| DPB1*2201 | 350, 354, 358, 364, 365, 367, 376, 382, 386, 388, 391, 394, 396 |
| DPB1*2301 | 350, 354, 358, 363, 365, 371, 376, 382, 386, 388, 391, 394, 396 |
| DPB1*2401 | 349, 356, 358, 364, 365, 368, 373, 377, 382, 386, 388, 390, 393, 396 |
| DPB1*2501 | 349, 356, 358, 361, 366, 367, 376, 382, 386, 388, 393, 396 |
| DPB1*26011 | 349, 356, 358, 361, 365, 367, 376, 382, 386, 388, 390, 393, 396 |
| DPB1*26012 | 349, 356, 358, 361, 365, 367, 376, 382, 386, 388, 391, 393, 396 |
| DPB1*2701 | 350, 354, 368, 363, 365, 368, 373, 377, 382, 386, 388, 391, 395, 396 |
| DPB1*2801 | 349, 356, 358, 364, 365, 370, 374, 377, 386, 388, 390, 393, 396 |
| DPB1*2901 | 351, 356, 358, 364, 365, 371, 376, 386, 388, 391, 393, 396 |
| DPB1*3001 | 350, 354, 358, 363, 365, 367, 377, 382, 387, 391, 393, 396 |
| DPB1*3101 | 350, 354, 358, 364, 365, 372, 375, 376, 386, 388, 391, 394, 396 |
| DPB1*3201 | 350, 354, 358, 363, 365, 367, 376, 386, 388, 391, 394, 396 |
| DPB1*3301 | 350, 354, 358, 362, 365, 367, 377, 382, 387, 391, 395, 396 |
| DPB1*3401 | 351, 356, 358, 364, 365, 370, 374, 376, 382, 386, 388, 390, 393, 396 |
| DPB1*3501 | 349, 356, 358, 362, 365, 371, 376, 382, 386, 388, 391, 393, 396 |
| DPB1*3601 | 349, 356, 358, 364, 365, 368, 373, 376, 386, 388, 390, 393, 396 |
| DPB1*3701 | 350, 354, 358, 362, 365, 371, 376, 382, 386, 388, 391, 393, 397 |
| DPB1*3801 | 350, 354, 358, 361, 365, 367, 376, 382, 386, 388, 391, 394, 396 |
| DPB1*3901 | 350, 354, 358, 361, 365, 367, 376, 382, 386, 388, 391, 395, 396 |
| DPB1*4001 | 350, 354, 358, 364, 365, 368, 373, 378, 386, 388, 391, 394, 396 |

TABLE 20
Correspondence of DPB types and high accuracy typing capture oligos (2)

| Type | Corresponding capture oligos |
|---|---|
| DPB1*4101 | 349, 356, 358, 362, 365, 370, 374, 377, 386, 388, 390, 393, 396 |
| DPB1*4401 | 351, 356, 358, 364, 365, 368, 373, 377, 382, 386, 388, 390, 393, 396 |
| DPB1*4501 | 350, 354, 358, 364, 365, 370, 374, 376, 386, 388, 391, 394, 396 |
| DPB1*4601 | 350, 354, 358, 364, 365, 371, 376, 386, 388, 391, 394, 396 |
| DPB1*4701 | 350, 354, 358, 362, 365, 368, 373, 376, 386, 388, 391, 394, 396 |
| DPB1*4801 | 350, 354, 358, 361, 365, 368, 373, 376, 382, 386, 388, 391, 394, 396 |
| DPB1*4901 | 349, 354, 358, 364, 365, 370, 374, 377, 382, 386, 388, 390, 393, 396 |
| DPB1*5001 | 350, 354, 358, 363, 365, 368, 373, 376, 382, 386, 388, 391, 394, 396 |
| DPB1*5101 | 349, 356, 358, 364, 365, 367, 377, 382, 386, 388, 390, 393, 396 |
| DPB1*5201 | 350, 354, 358, 361, 365, 368, 373, 376, 382, 386, 388, 391, 395, 396 |
| DPB1*5301 | 351, 356, 358, 364, 365, 371, 376, 386, 388, 390, 393, 396 |
| DPB1*5401 | 351, 356, 358, 364, 365, 367, 376, 386, 388, 391, 393, 396 |
| DPB1*5501 | 349, 356, 358, 363, 365, 367, 377, 382, 386, 388, 390, 393, 396 |
| DPB1*5601 | 350, 354, 358, 364, 365, 370, 374, 377, 382, 386, 388, 390, 393, 396 |
| DPB1*5701 | 351, 356, 358, 362, 365, 367, 376, 386, 388, 391, 393, 396 |
| DPB1*5801 | 350, 354, 358, 364, 365, 368, 373, 377, 382, 386, 388, 391, 394, 396 |
| DPB1*5901 | 350, 354, 358, 364, 365, 368, 373, 379, 382, 386, 388, 391, 394, 396 |
| DPB1*6001 | 349, 356, 358, 364, 365, 370, 374, 380, 381, 385, 386, 388, 390, 393 |
| DPB1*6101N | 350, 354, 358, 362, 365, 367, 376, 382, 386, 388, 391, 395, 396 |
| DPB1*6201 | 350, 354, 358, 362, 365, 367, 376, 382, 386, 388, 391, 393, 396 |
| DPB1*6301 | 352, 356, 358, 364, 365, 370, 374, 377, 386, 388, 391, 393, 396 |
| DPB1*6401N | 350, 354, 358, 361, 365, 367, 376, 382, 386, 388, 390, 393, 396 |
| DPB1*6501 | 351, 356, 358, 363, 365, 367, 376, 382, 386, 388, 391, 394, 396 |
| DPB1*6601 | 351, 356, 358, 364, 365, 367, 377, 382, 386, 388, 390, 393, 396 |
| DPB1*6701 | 350, 354, 358, 364, 365, 368, 373, 376, 382, 386, 388, 390, 393, 396 |
| DPB1*6801 | 349, 356, 358, 364, 365, 370, 374, 377, 384, 386, 388, 391, 393, 396 |
| DPB1*6901 | 353, 356, 358, 364, 365, 370, 374, 377, 382, 386, 388, 390, 393, 396 |
| DPB1*7001 | 350, 354, 358, 364, 365, 367, 376, 386, 388, 391, 394, 396 |
| DPB1*7101 | 350, 354, 358, 363, 365, 367, 377, 382, 386, 388, 391, 394, 396 |
| DPB1*7201 | 350, 354, 358, 364, 365, 368, 373, 377, 382, 386, 388, 390, 394, 396 |
| DPB1*7301 | 349, 356, 359, 364, 366, 367, 377, 386, 388, 391, 395, 396 |
| DPB1*7401 | 350, 354, 358, 364, 365, 368, 373, 376, 382, 386, 388, 390, 394, 396 |
| DPB1*7501 | 351, 356, 358, 363, 365, 368, 373, 377, 382, 386, 388, 390, 393, 396 |
| DPB1*7601 | 350, 357, 358, 361, 365, 368, 373, 376, 382, 386, 388, 391, 394, 396 |
| DPB1*7701 | 349, 356, 358, 364, 365, 370, 374, 377, 382, 389, 390, 393, 396 |
| DPB1*7801 | 349, 356, 358, 364, 365, 368, 373, 376, 382, 386, 388, 390, 393, 396 |
| DPB1*7901 | 349, 356, 358, 364, 365, 368, 373, 376, 382, 386, 388, 390, 393, 396 |

The kit of the present invention includes a substrate on which capture oligos are immobilized. The kit of the present invention can include primers for preparing a nucleic acid target or reagents for hybridization such as a labeled nucleic acid target, buffer and enzyme conjugate recognizing a hapten.

### BRIEF DESCRIPTION OF THE DRAWINGS

FIG. 1 shows positions of capture oligos (for HLA-DQA1) immobilized on a substrate. Numbers in the figure represent sequence numbers (the same shall apply in FIGS. 2-7).

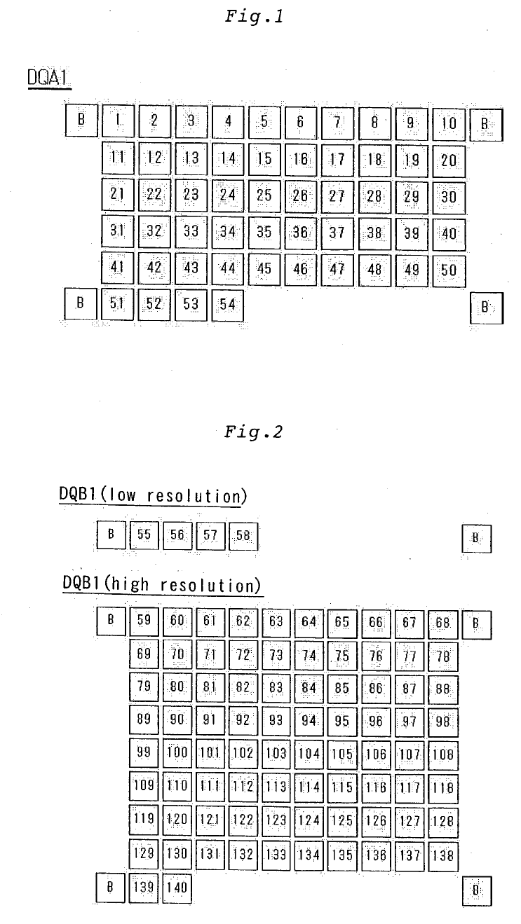

FIG. 2 shows positions of capture oligos (for HLA-DQB1) immobilized on a substrate.

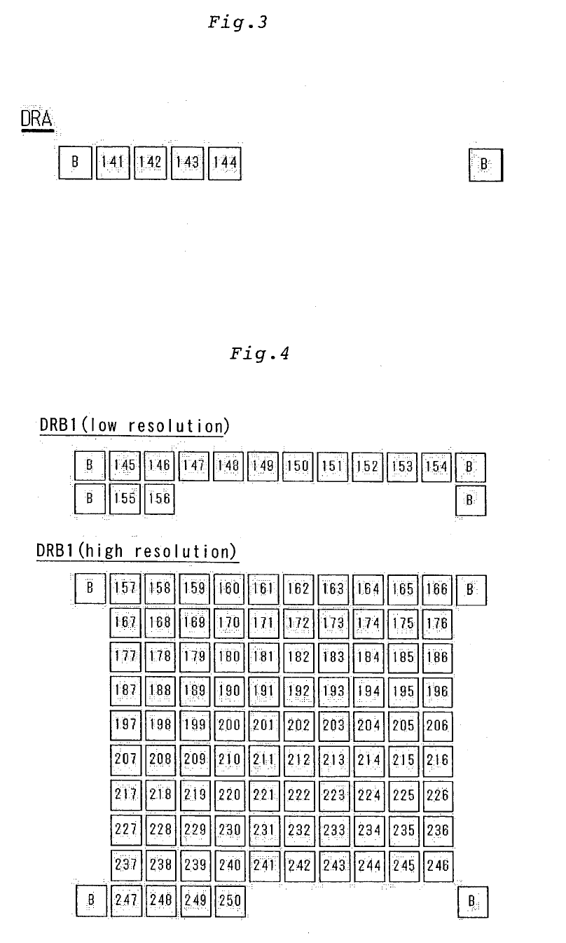

FIG. 3 shows positions of capture oligos (for HLA-DRA) immobilized on a substrate.

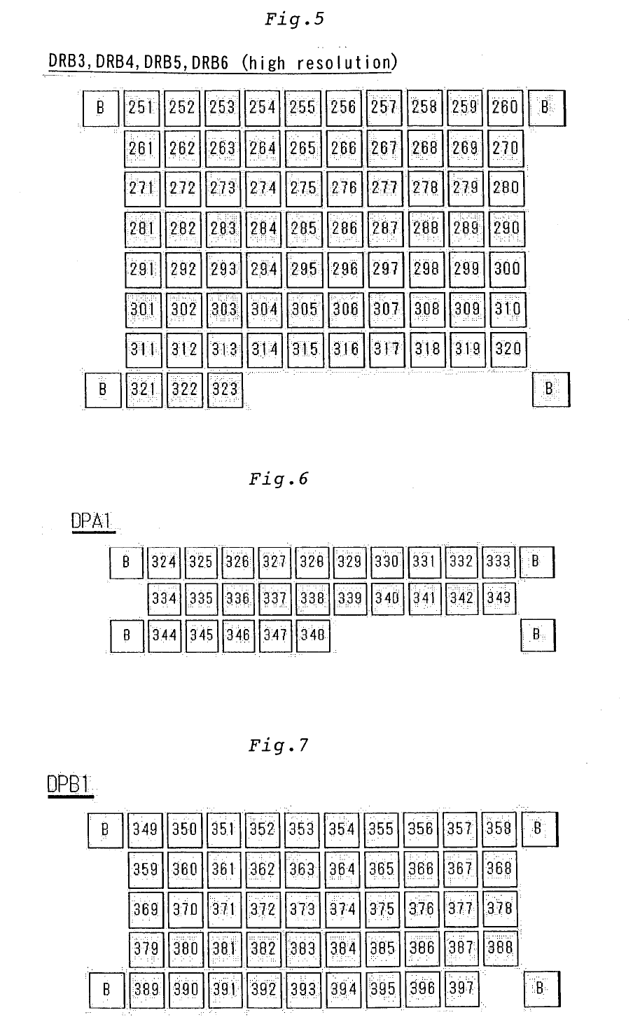

FIG. 4 shows positions of capture oligos (for HLA-DRB1) immobilized on a substrate.

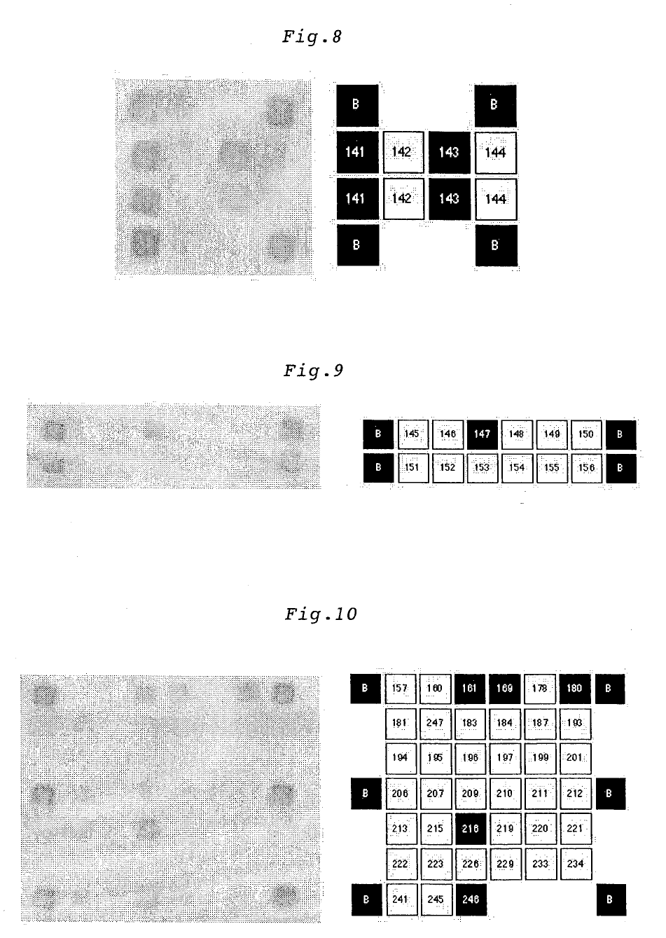

FIG. 5 shows positions of capture oligos (for HLA-DRB3, HLA-DRB4, HLA-DRB5 and HLA-DRB6,) immobilized on a substrate.

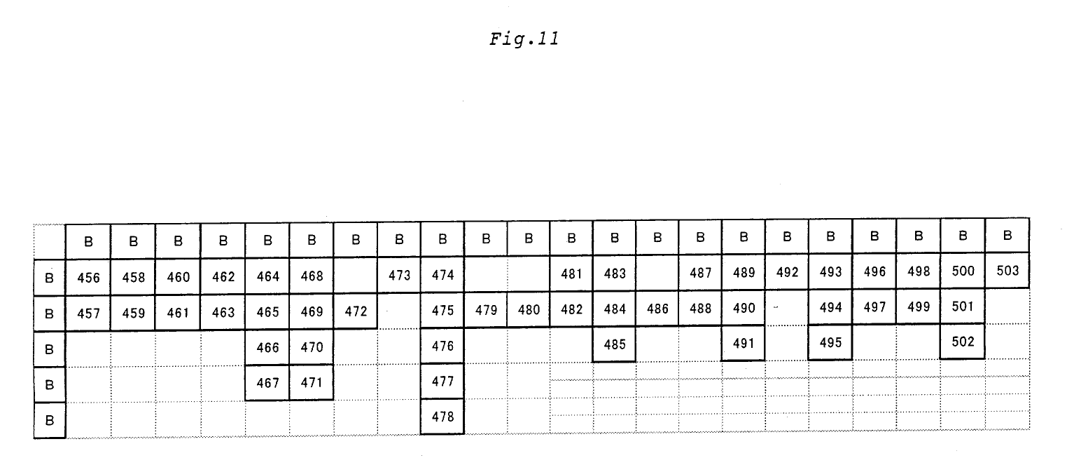

FIG. 6 shows positions of capture oligos (for HLA-DPA1) immobilized on a substrate.

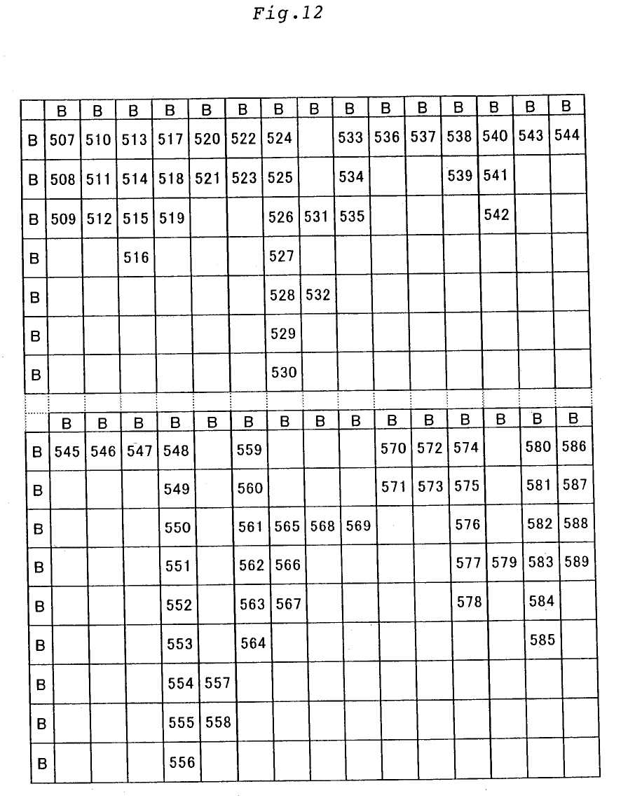

FIG. 7 shows positions of capture oligos (for HLA-DPB1) immobilized on a substrate.

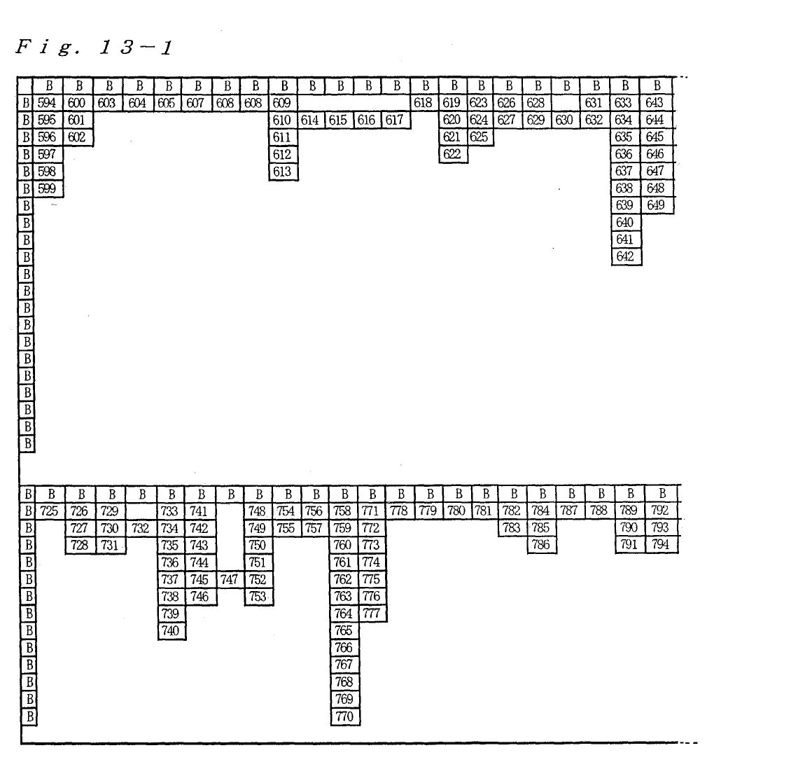

FIG. 8 shows a schematic view of a kit of the present invention and a detection result of DRA typing using the same (detection result typed as DRA*1010).

Numbers in the figure represent sequence numbers. B indicates that an oligonucleotide in which biotin is introduced at 5′ end is immobilized. indicates that hybridization is not obtained. indicates that a hybridization signal is obtained (the same shall apply in FIGS. 9 and 10).

FIG. 9 shows a schematic view of the kit of the present invention and a detection result of DRB low resolution typing using the same (detection result typed as DR4).

FIG. 10 shows a schematic view of the kit of the present invention and a detection result of DRB1 high resolution typing using the same (detection result typed as DRB*04011).

FIG. 11 shows positions of capture oligos (for HLA-DPB1) immobilized on a substrate.

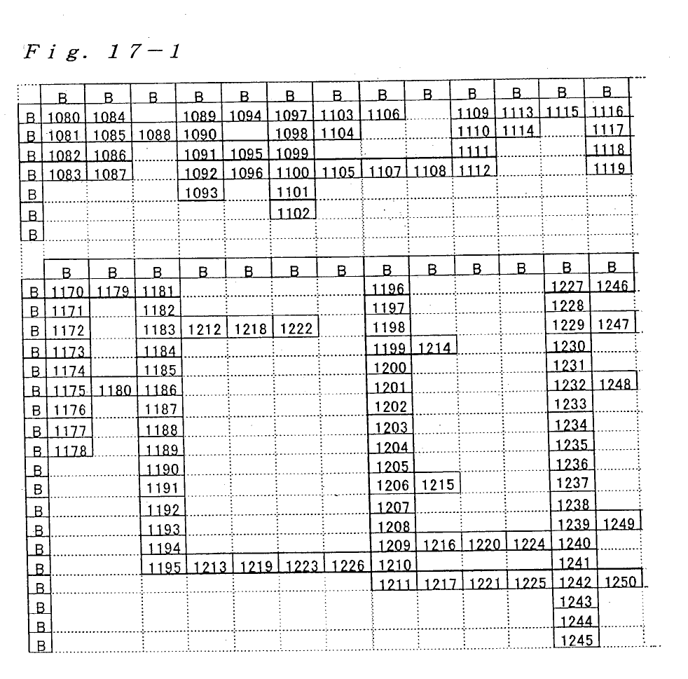

FIG. 12 shows positions of capture oligos (for HLA-DQB1) immobilized on a substrate.

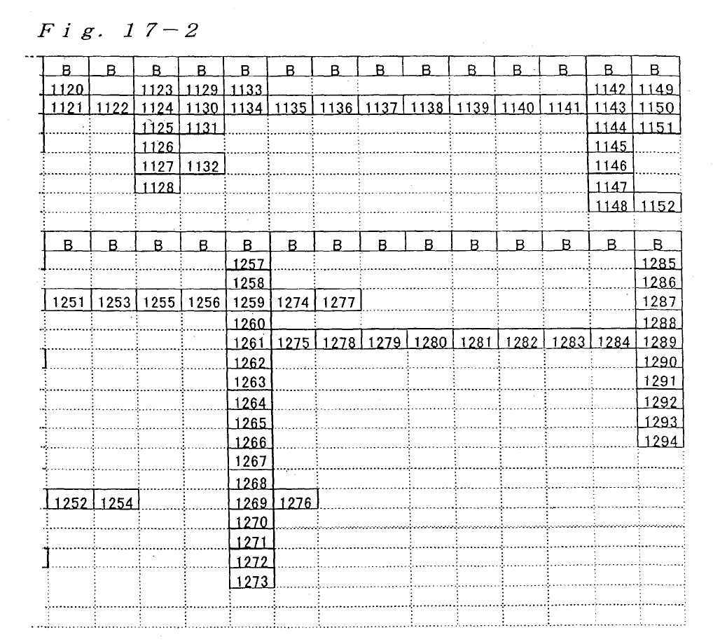

FIG. 13 shows positions of capture oligos (for HLA-DRB1) immobilized on a substrate.

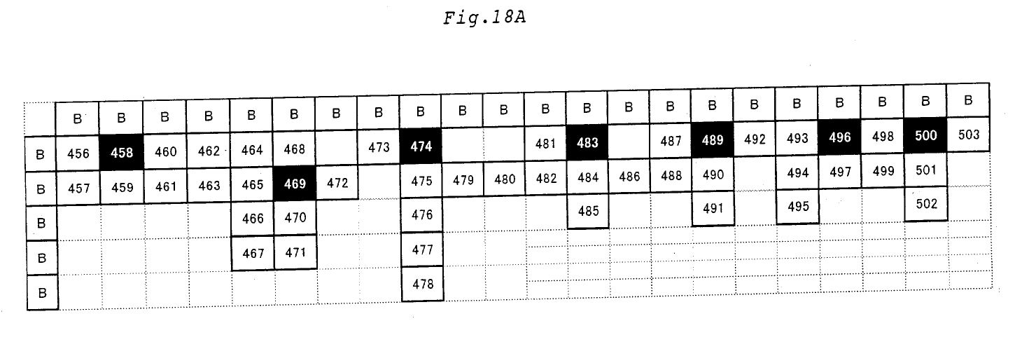

FIG. 14 shows positions of capture oligos (for HLA-DRB pseudogene) immobilized on a substrate.

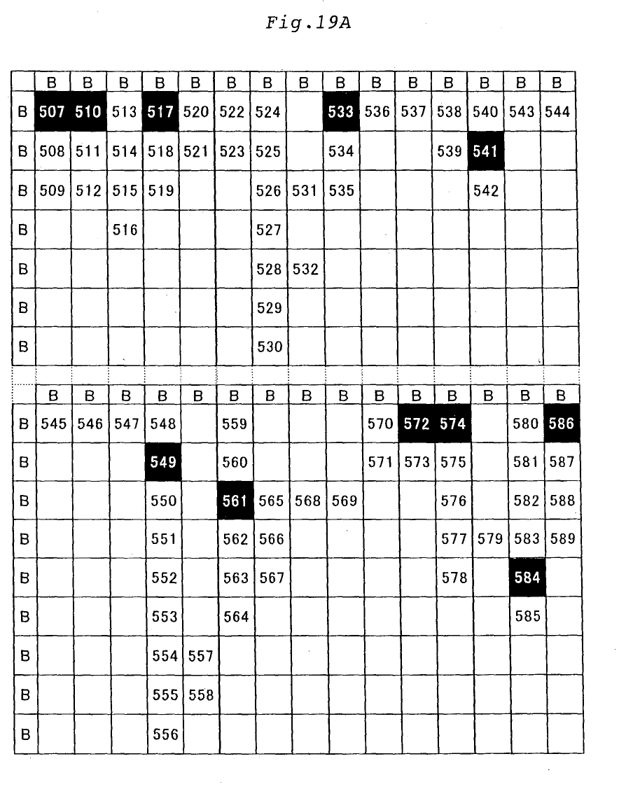

FIG. 15 shows positions of capture oligos (for HLA-DPB3, 4, 5) immobilized on a substrate.

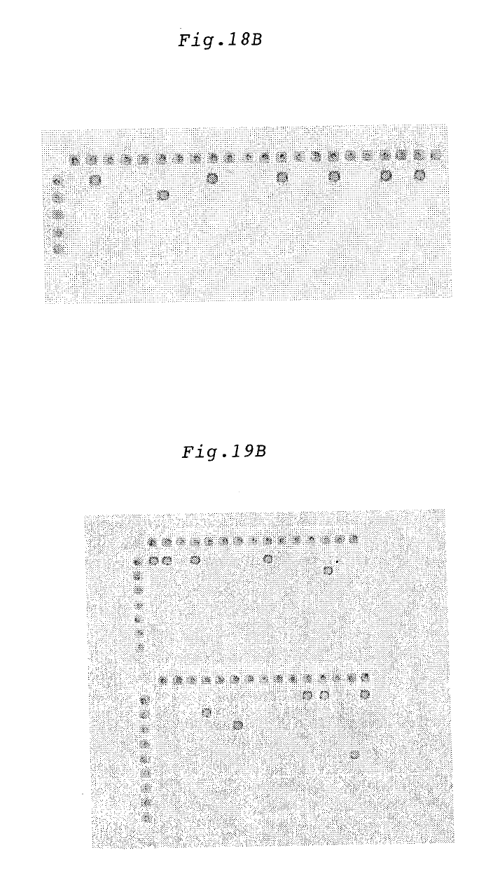

FIG. 16 shows positions of capture oligos (for HLA-A) immobilized on a substrate.

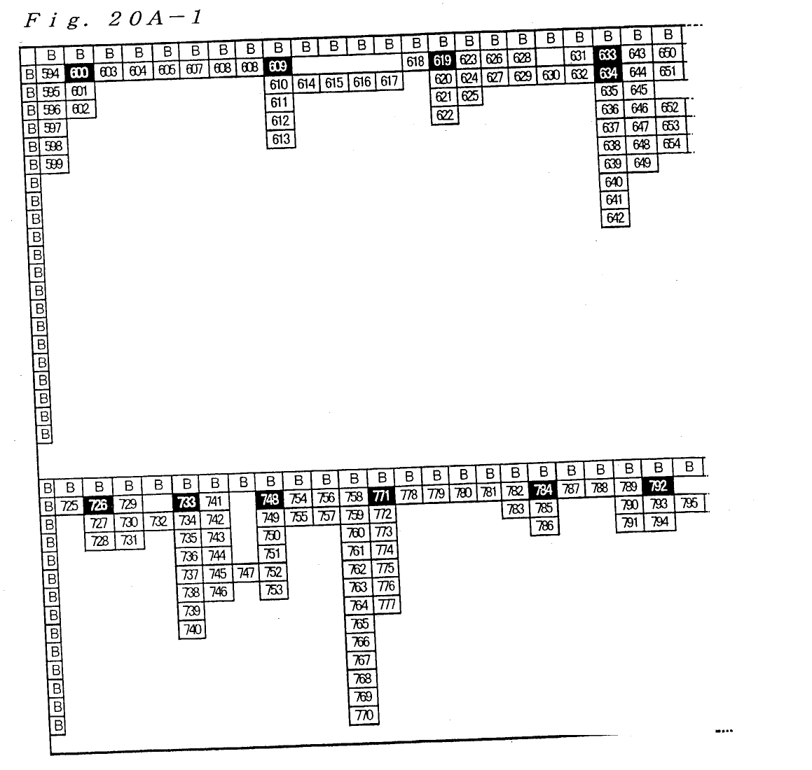

FIG. 17 shows positions of capture oligos (for HLA-B) immobilized on a substrate.

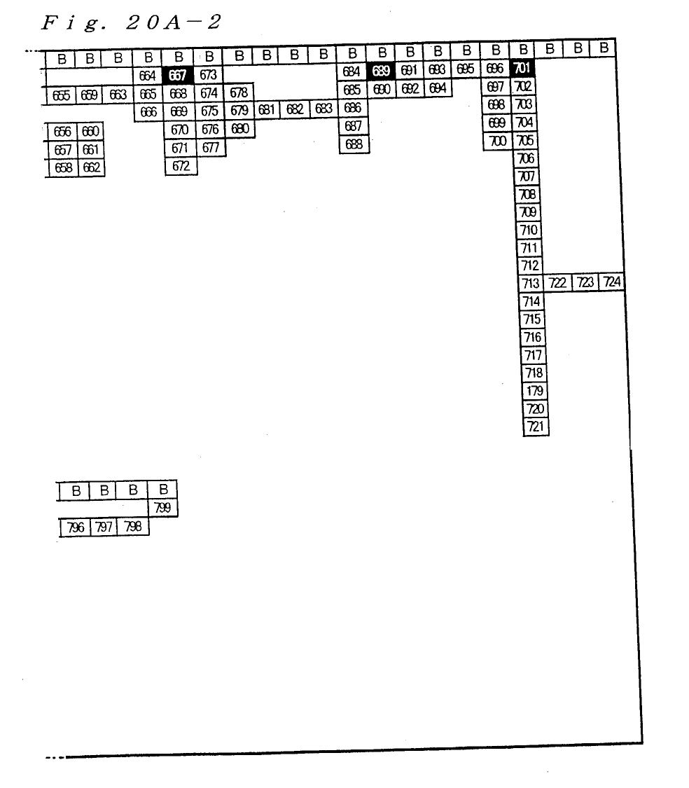

FIG. 18 shows a schematic view of the kit of the present invention (A) and a photograph showing detection result of DPB1 typing using the same (detection result typed as DPB1*01011) (B).

FIG. 19 shows a schematic view of the kit of the present invention (A) and a photograph showing detection result of DQB1 typing using the same (detection result typed as DQB1*05031) (B).

FIG. 20 shows a schematic view of the kit of the present invention (A) and a photograph showing detection result of DRB1 typing using the same (detection result typed as DRB1*0101) (B).

FIG. 21 shows a schematic view of the kit of the present invention (A) and a photograph showing detection result of DRB9 typing using the same (detection result typed as DRB9 0101) (B).

FIG. 22 shows a schematic view of the kit of the present invention (A) and a photograph showing detection result of DRB3 typing using the same (detection result typed as DRB3 0301) (B).

FIG. 23 shows a schematic view of the kit of the present invention (A) and a photograph showing detection result of A typing using the same (detection result typed as A 01011) (B).

FIG. 24 shows a schematic view of. the kit of the present invention (A) and a photograph showing detection result of B typing using the same (detection result typed as B 0724) (B).

### BEST MODE FOR CARRYING OUT THE INVENTION

Hereafter, the present invention will be explained more specifically with reference to the following examples.

### EXAMPLE 1

### Synthesis of Oligonucleotides

According to a usual method, oligonucleotides having an amino group or hydroxyl group at 5′ end were synthesized by using an oligonucleotide synthesizer (Perkin-Elmer Applied Biosystems), deprotected and dried. These dried oligonucleotides were dissolved in a buffer of 10 mM Tris-HCl (pH 7.5) and 1 mM EDTA to prepare 100 pmol/μL solutions of oligonucleotides. This synthesis method can be employed both for oligonucleotides used as capture oligos and those used as primers. The nucleotide sequences of the synthesized oligonucleotides are listed in Sequence Listing as SEQ ID NOS: 1-447. Among these, SEQ ID NOS: 1-397 are capture oligos and SEQ ID NOS: 398-447 are primers.

### EXAMPLE 2

### Spotting of Capture Oligos on Substrate (Utilizing Oligonucleotide Having Amino Group at 5′ End)

In an amount of 10 μL of a microspotting solution (TeleChem International Inc.) was mixed with 10 μL of a solution of oligonucleotide having an amino group at 5′ end and added onto a microtiter plate (Greiner Laboratory Inc.). Silanized slide glass (Matsunami Glass Ind. Ltd.) was placed at a predetermined position in a. spotting machine and then the spotting machine was operated. After the spotting was completed, the slide glass was exposed to vapor from hot water for a few seconds and then irradiated with ultraviolet ray of 30 mJ. After exposed to vapor again for a few seconds, the slide glass was placed on a hot plate to remove moisture. The slide glass was rinsed with 0.1% sodium dodecyl sulfate aqueous solution and then with distilled water. The slide glass was blocked by immersing it in 100 mM Tris-HCl (pH 7.5), 100 mM NaCl and 0.1% Triton X-100 containing 3% bovine serum albumin (BSA) at room temperature for 30 minutes. Then, after dried at room temperature, the slide glass was washed with a buffer of 10 mM Tris-HCl (pH 7.5) and 1 mM EDTA. The slide glass was dried at room temperature again and maintained in a dry state in a dark cold place until use.

### EXAMPLE 3

### Spotting of Capture Oligos on Substrate (Utilizing Oligonucleotide Having Hydroxyl Group at 5′ End)

In an amount of 10 μL of a microspotting solution (2 M sodium chloride aqueous solution) was mixed with 10 μL of a solution of oligonucleotide having a hydroxyl group at 5′ at end and added onto a microtiter plate (Greiner Laboratory Inc.). Silanized slide glass (Matsunami Glass Ind. Ltd.) was placed at a predetermined position in a spotting machine and then the spotting machine was operated. After the spotting was completed, the slide glass was placed in a drier at 37° C. for 30 minutes. The slide glass was blocked by immersing it in 100 mM Tris-HCl (pH 7.5), 100 mM NaCl and 0.1% Triton X-100 containing 3% bovine serum albumin (BSA) at room temperature for 30 minutes. Then, after dried at room temperature, the slide glass was washed with a buffer of 10 mM Tris-HCl (pH 7.5) and 1 mM EDTA. The slide glass was dried at room temperature again and maintained in a dry state in a dark cold place until use.

### EXAMPLE 4

### Preparation of Nucleic Acid Target

Nucleated cells were collected from peripheral blood, cultured cells or the like, placed in a microtube and washed 3 times with physiological saline. The cells were added and mixed with 800 μl of 0.1 M Tris-HCl (pH 8.0), 0.2 M NaCl, 0.04 M EDTA and 2% SDS, then added with 120 μg of proteinase K and incubated overnight at 50° C. The mixture was added with 800 μl of 1 M Tris-HCl saturated phenol was added and rotated on a rotator for 1 hour or longer, and extracted 3 times with phenol to remove proteins. The aqueous layer was added with 2 ml of 2-propanol and 60 μl of 5 M NaCl and gently mixed, and then the precipitated DNA was quickly sucked up with a micropipette. The DNA was washed 3 times by centrifugation with addition of 70% cold ethanol. The DNA was dried under reduced pressure and dissolved in 70 μl of sterilized water to prepare a PCR template solution.

The composition for PCR amplification consisted of 2 units of Taq polymerase, 25 pmol each of primers , 5 μl of reaction buffer, 10 nmol each of dNTP, 0.5 μl of template DNA solution and sterilized water giving a total volume of 50 μl. The solution placed in a tube was set on a thermal cycler. A program was operated such that, out of a cycle of (1) 95° C., 3 minutes, (2) 95° C., 30 seconds, (3) 71° C., 30 seconds and (4) 72° C., 3 minutes, (2) and (3) should be repeated 30 times.

In this example, agarose gel electrophoresis described below was performed as a confirmation test, but is not required in identification in an actual clinical case. In an amount of 1 μl of the PCR reaction mixture was taken and mixed with 1 μl of 6× migration pigment (30% glycerol, 0.25% Bromophenol Blue, 0.25% xylene cyanol) and 4 μl of distilled water. After electrophoresis was performed on 2% agarose gel for 90 minutes at 100 V, the gel was immersed in distilled water containing 0.5 μg/ml of ethidium bromide for 30 minutes and photographed under ultraviolet ray irradiation by using a CCD camera.

### EXAMPLE 5

### Hybridization

In an amount of 2 μl of the nucleic acid target prepared in Example 4 was taken and mixed with 8 μl of ArrayIt Unihyb Hybridization Solution (TeleChem International Inc.), subjected to a heat treatment at 100° C. for 10 minutes, and then immersed in ice for 5 minutes. In an amount of 5 μl of this nucleic acid target solution was taken and placed on a substrate on which the capture oligos prepared in Example 2 or 3 were immobilized and then cover glass was placed thereon. This substrate was placed in a moisture box, further placed in an incubator set at 42° C. and left for 60 minutes. The substrate was taken out from the incubator and quickly immersed in 5× SSC (0.083 M NaCl and 0.083 M sodium citrate) at 4° C. to remove the cover glass. Immersion of the substrate in 5× SSC at 4° C. for 10 minutes was repeated twice and then the substrate was rinsed twice with 3 M tetramethylammonium chloride aqueous solution at room temperature. Immersion of the substrate in 3 M tetramethylammonium chloride aqueous solution at 45° C. for 20 minutes was repeated twice. Finally, the substrate was immersed in 2× SSC (0.033 M NaCl and 0.033 M sodium citrate).

### EXAMPLE 6

### Detection of Chemical Color Development

After hybridization was completed in Example 5, the substrate was taken out from 2× SSC. Block Ace (Dainippon Pharmaceutical Co., Ltd.) stock solution (4% aqueous solution) was placed on the substrate in an amount of 70 μl per cm² and left at room temperature for 20 minutes, and then the solution was removed. Subsequently, a mixed solution obtained by adding 2 drops each of avidin DH (Vector Laboratories, Inc.) and biotinylated horseradish peroxidase H (Vector Laboratories, Inc.) to 5 ml of TBST (0.05 M Tris-HCl (pH 7.6), 0.15 M NaCl, 0.05% Tween 20) and mixing them was placed on the substrate in an amount of 70 μl per cm² and left at room temperature for 30 minutes, and then the solution was removed. After immersion of the substrate in TBST at room temperature for 5 minutes was repeated twice, the substrate was taken out from the buffer and the moisture was sufficiently removed with paper towel. A TMB (3,3′,5,5′-tetramethylbenzidine) color development substrate kit (Vector Laboratories, Inc.) was placed on the substrate in amount of 70 μl per cm² and left at room temperature for 10 minutes, and then the solution was removed. The substrate was immersed in deionized water to stop the enzymatic reaction.

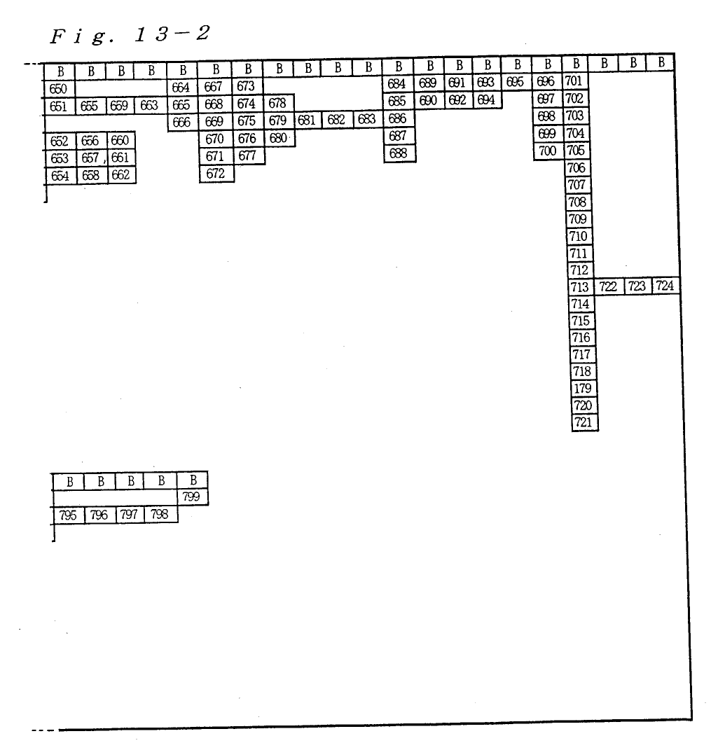

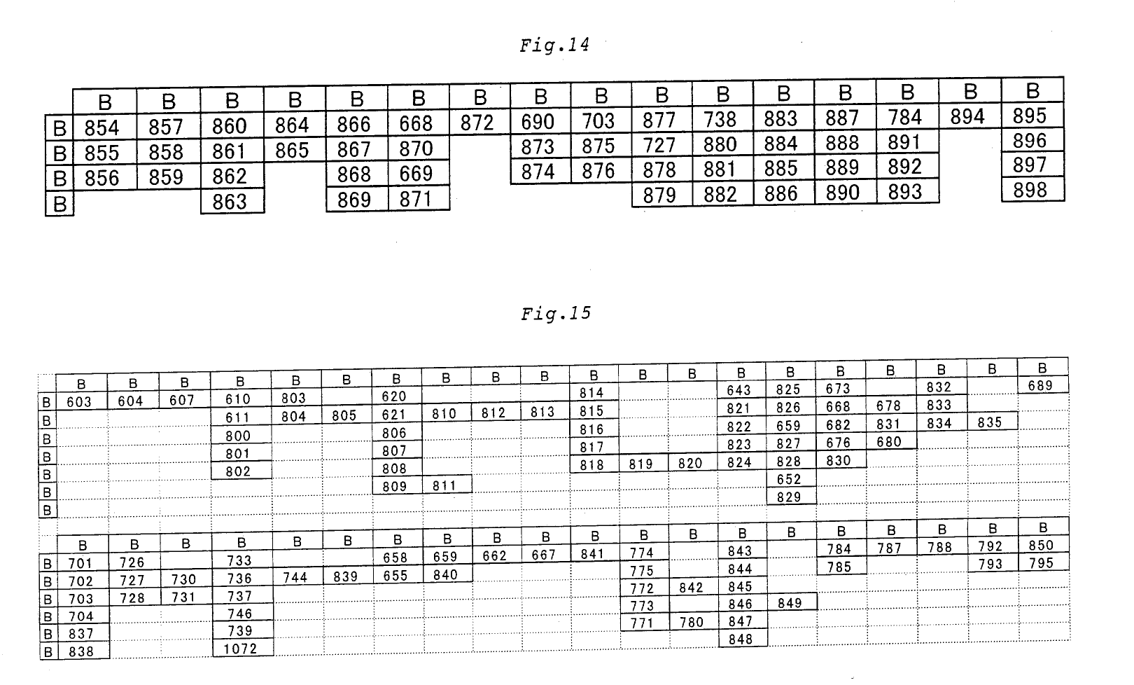

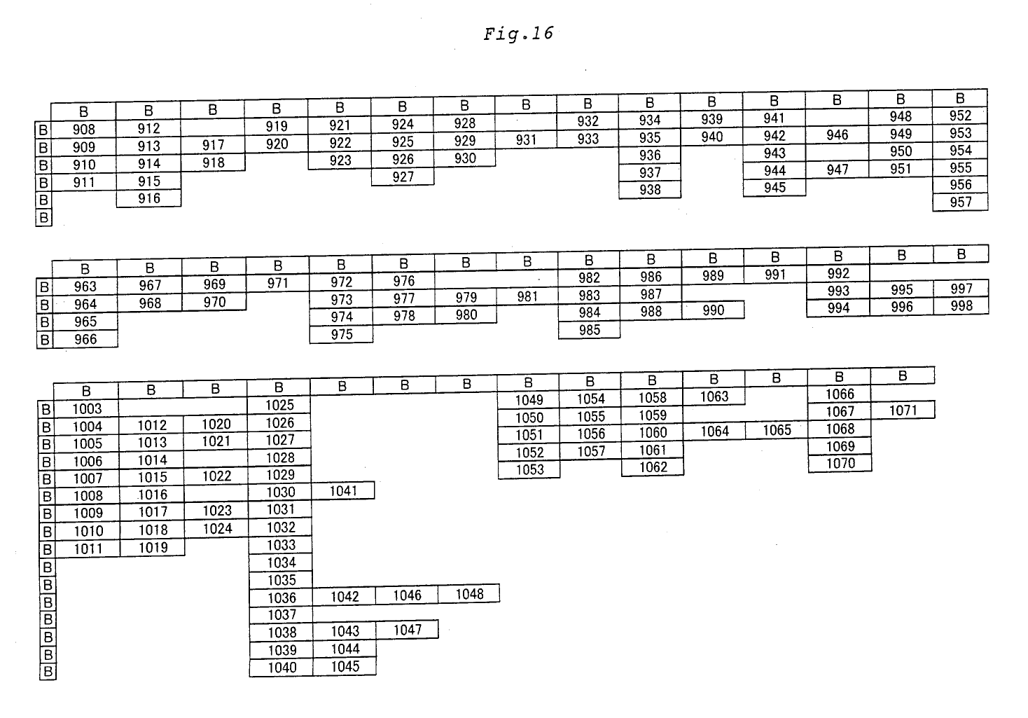

The results of the above examples are shown in FIGS. 8-10. It is evident that HLA typing can be carried out by the method of the present invention.

According to the present invention, there are provided a kit and method that enable multiple specimen processing and a highly precise HLA typing with one time of test as well as oligonucleotides and primers for use in the same.

### EXAMPLE 7

### Typing Using Patchworks

Oligonucleotides used for preparation of arrays were synthesized in a similar manner as Example 1. Arrays were prepared in a similar manner as Example 2 or 3. Correspondence of the sequences of oligonucleotides used in arrays for typing HLA and HLA types are shown in Tables 21-32. For amplifying targets, combinations of primers shown in Table 33 were used. Typing was performed in a similar manner as Examples 4-6.

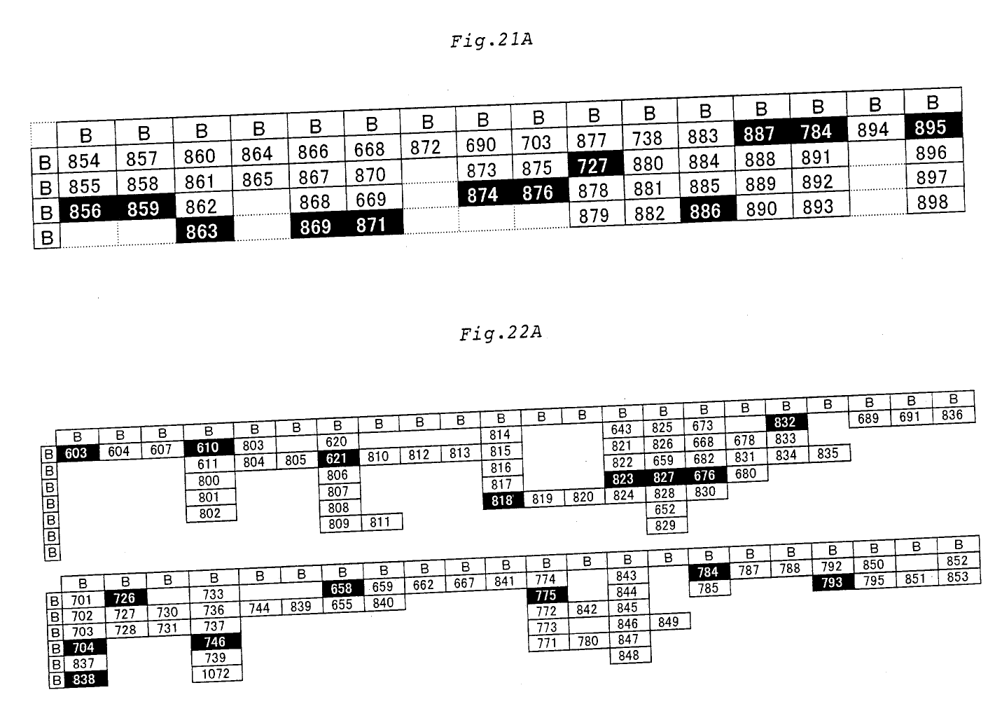

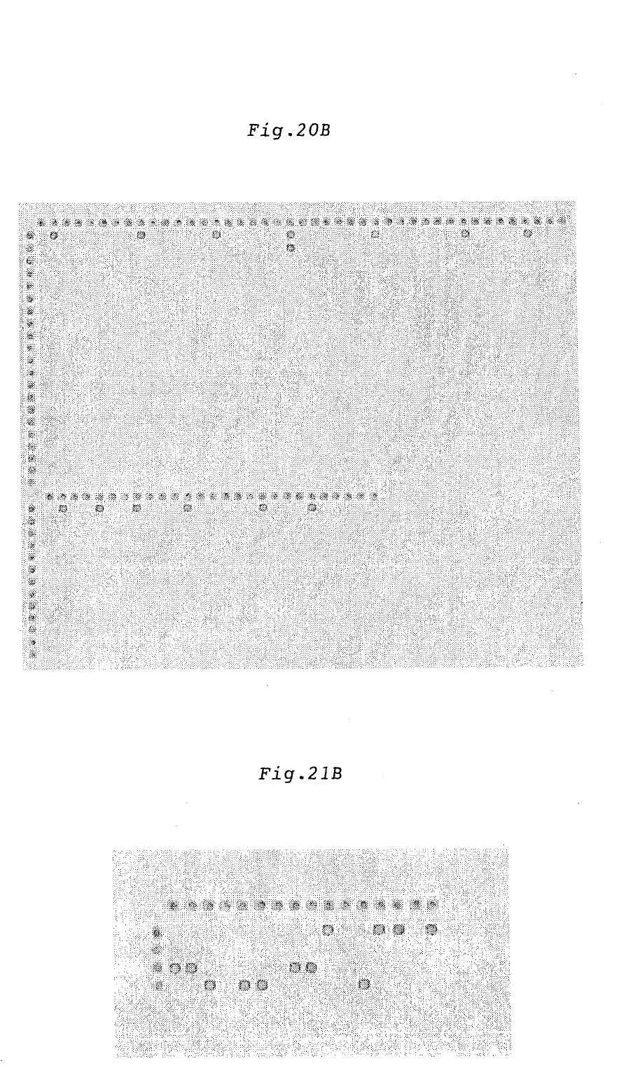

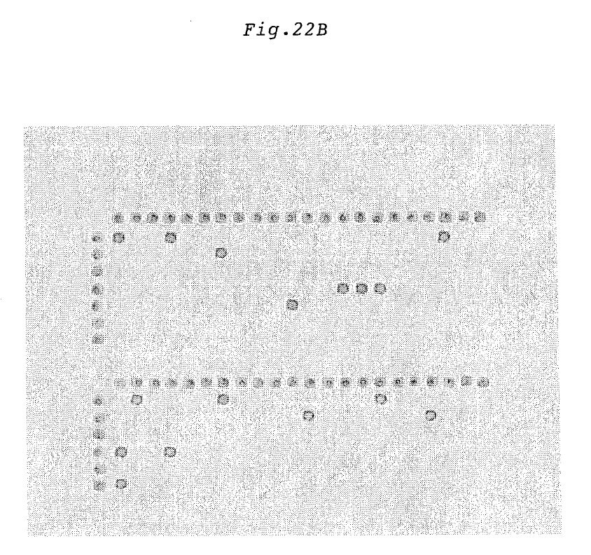

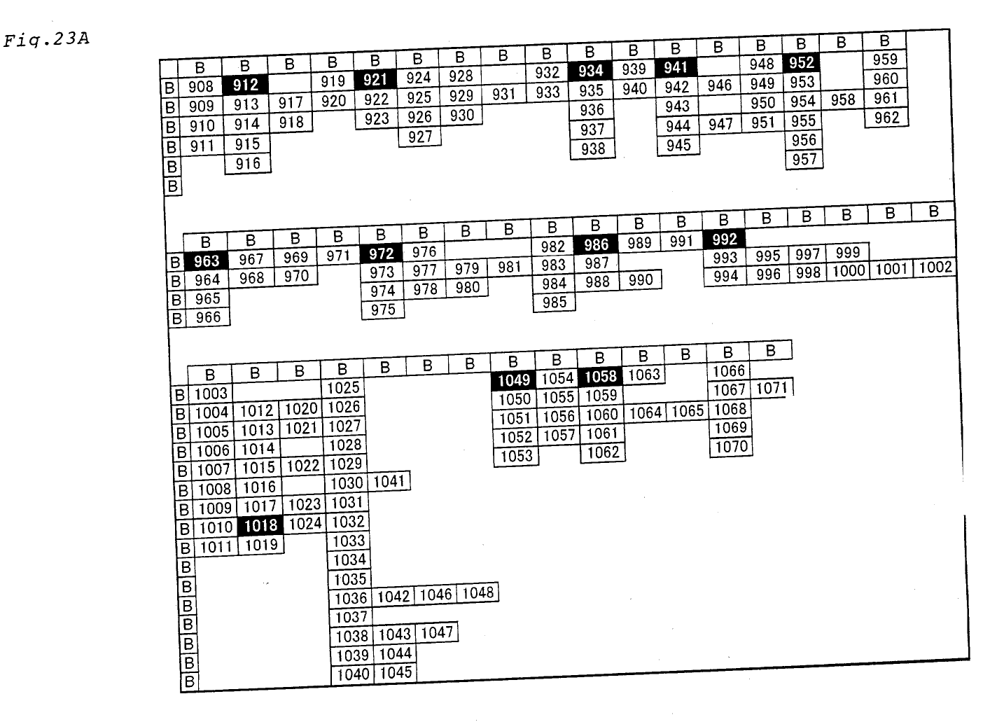

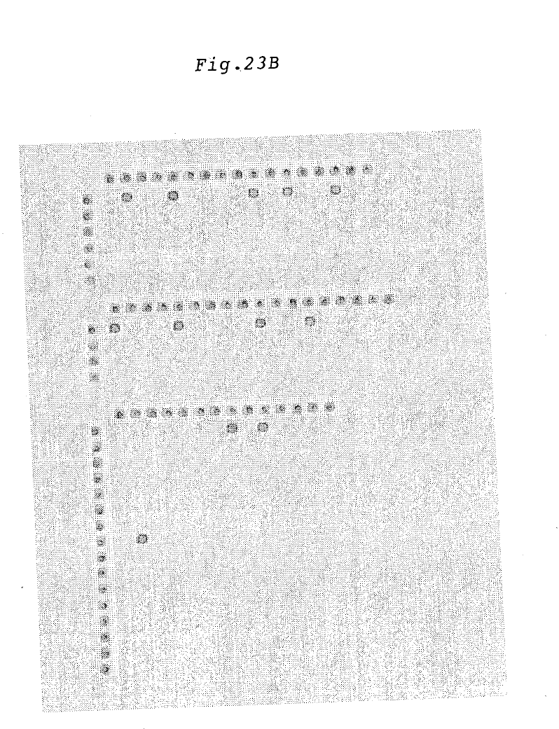

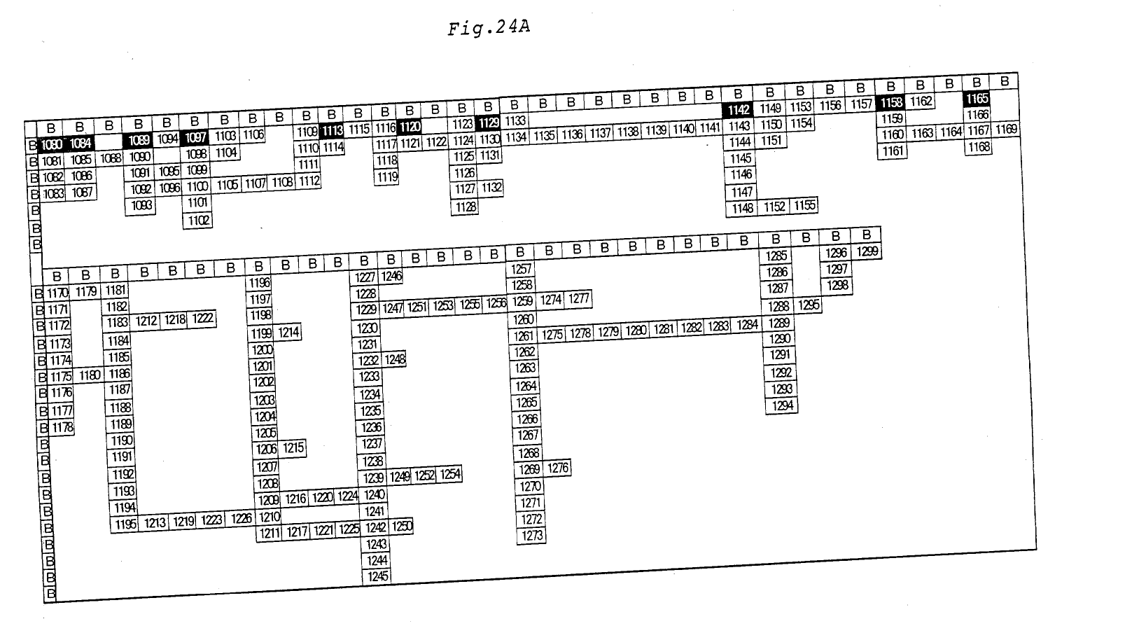

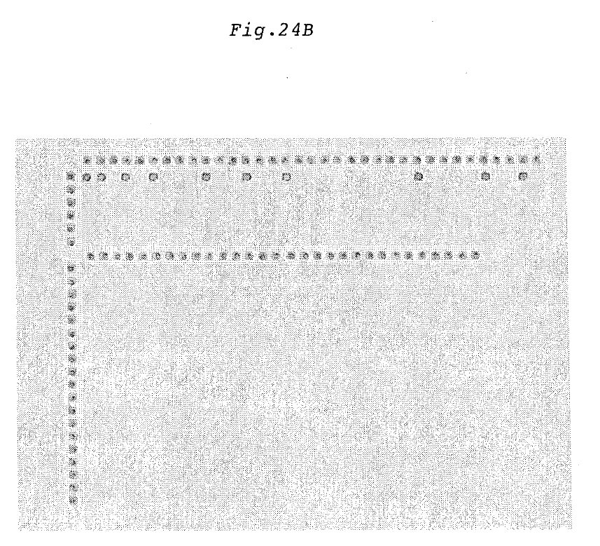

The results are shown in FIGS. 18-24. Thus, HLA typing of the test specimen can be performed with the arrays which were prepared using oligonucleotides designed according to patchworks and satellites.

TABLE 21
Correspondence of the patchwork regions and satellite regions for types of DPB1

| | patchwork region | | | | | | | satellite region | | | |
|---|---|---|---|---|---|---|---|---|---|---|---|
| DPB1*01011 | DPB1 | DPB4 | DPB7 | DPB12 | DPB15 | DPB18 | DPB21 | | | | |
| DPB1*6501 | DPB1 | DPB4 | DPB7 | DPB12 | DPB15 | DPB18 | DPB21 | | | | |
| DPB1*01012 | DPB1 | DPB4 | DPB7 | DPB12 | DPB15 | DPB18 | DPB21 | 473 | | | |
| DPB1*8901 | DPB1 | DPB4 | DPB7 | DPB12 | DPB15 | DPB19 | DPB21 | | | | |
| DPB1*1301 | DPB2 | DPB4 | DPB7 | DPB12 | DPB16 | DPB20 | DPB21 | | | | |
| DPB1*26011 | DPB2 | DPB4 | DPB7 | DPB12 | DPB15 | DPB18 | DPB21 | 473 | 495 | | |
| DPB1*26012 | DPB2 | DPB4 | DPB7 | DPB12 | DPB15 | DPB18 | DPB21 | | | | |
| DPB1*2701 | DPB2 | DPB4 | DPB7 | DPB12 | DPB15 | DPB19 | DPB21 | | | | |
| DPB1*8501 | DPB2 | DPB4 | DPB7 | DPB12 | DPB15 | DPB19 | DPB21 | | | | 456 |
| DPB1*3901 | DPB1 | DPB4 | DPB7 | DPB12 | DPB15 | DPB19 | DPB22 | | | | |
| DPB1*4001 | DPB1 | DPB4 | DPB7 | DPB12 | DPB15 | DPB19 | DPB23 | | | | |
| DPB1*0401 | DPB1 | DPB3 | DPB7 | DPB12 | DPB15 | DPB19 | DPB22 | | | | |
| DPB1*3301 | DPB1 | DPB3 | DPB7 | DPB12 | DPB16 | DPB19 | DPB22 | | | | |
| DPB1*6601 | DPB2 | DPB3 | DPB7 | DPB12 | DPB15 | DPB19 | DPB22 | | | | 456 |
| DPB1*2301 | DPB1 | DPB5 | DPB7 | DPB12 | DPB15 | DPB19 | DPB22 | | | | |
| DPB1*7101 | DPB1 | DPB5 | DPB7 | DPB12 | DPB16 | DPB19 | DPB22 | | | | |
| DPB1*5501 | DPB2 | DPB5 | DPB7 | DPB12 | DPB16 | DPB19 | DPB21 | | | | 456 |
| DPB1*5801 | DPB2 | DPB6 | DPB7 | DPB12 | DPB16 | DPB19 | DPB21 | | | | 456 |
| DPB1*6301 | DPB1 | DPB6 | DPB7 | DPB12 | DPB15 | DPB19 | DPB21 | | | | |
| DPB1*6201 | DPB1 | DPB6 | DPB7 | DPB12 | DPB15 | DPB19 | DPB23 | | | | |
| DPB1*1601 | DPB1 | DPB4 | DPB8 | DPB12 | DPB16 | DPB19 | DPB21 | | | | |
| DPB1*4901 | DPB1 | DPB4 | DPB8 | DPB12 | DPB15 | DPB19 | DPB22 | | | | |
| DPB1*5301 | DPB1 | DPB4 | DPB8 | DPB12 | DPB15 | DPB19 | DPB23 | | | | |
| DPB1*0801 | DPB1 | DPB5 | DPB8 | DPB12 | DPB16 | DPB18 | DPB21 | | | | |
| DPB1*6801 | DPB1 | DPB5 | DPB8 | DPB12 | DPB15 | DPB18 | DPB21 | | | | |
| DPB1*7501 | DPB1 | DPB5 | DPB8 | DPB12 | DPB15 | DPB18 | DPB22 | | | | |
| DPB1*02012 | DPB1 | DPB5 | DPB8 | DPB12 | DPB16 | DPB19 | DPB22 | | | | |
| DPB1*3201 | DPB1 | DPB5 | DPB8 | DPB12 | DPB16 | DPB19 | DPB22 | 481 | | | |
| DPB1*4601 | DPB1 | DPB5 | DPB8 | DPB12 | DPB16 | DPB19 | DPB22 | 482 | | | |
| DPB1*6001 | DPB1 | DPB5 | DPB8 | DPB12 | DPB15 | DPB19 | DPB22 | 487 | | | |
| DPB1*7701 | DPB1 | DPB5 | DPB8 | DPB12 | DPB15 | DPB19 | DPB22 | 464 | | | |
| DPB1*8001 | DPB1 | DPB5 | DPB8 | DPB12 | DPB15 | DPB19 | DPB22 | | | | |
| DPB1*8201 | DPB1 | DPB5 | DPB8 | DPB12 | DPB15 | DPB19 | DPB22 | | | | |
| DPB1*0901 | DPB2 | DPB5 | DPB8 | DPB12 | DPB16 | DPB18 | DPB21 | 482 | | | 456 |
| DPB1*1001 | DPB2 | DPB5 | DPB8 | DPB12 | DPB16 | DPB18 | DPB21 | | | | 456 |
| DPB1*3701 | DPB2 | DPB5 | DPB8 | DPB12 | DPB16 | DPB18 | DPB21 | | | | |
| DPB1*3501 | DPB2 | DPB5 | DPB8 | DPB12 | DPB15 | DPB18 | DPB21 | 482 | | | 456 |
| DPB1*7901 | DPB2 | DPB5 | DPB8 | DPB12 | DPB15 | DPB18 | DPB21 | | | | |
| DPB1*8801 | DPB2 | DPB5 | DPB8 | DPB12 | DPB16 | DPB18 | DPB21 | 482 | | | |
| DPB1*1701 | DPB2 | DPB5 | DPB8 | DPB12 | DPB16 | DPB19 | DPB21 | 482 | | | 456 |
| DPB1*0402 | DPB2 | DPB5 | DPB8 | DPB12 | DPB15 | DPB19 | DPB22 | | | | |
| DPB1*1801 | DPB1 | DPB5 | DPB8 | DPB12 | DPB15 | DPB19 | DPB23 | | | | |
| DPB1*4801 | DPB1 | DPB6 | DPB8 | DPB12 | DPB16 | DPB19 | DPB22 | | | | |
| DPB1*5101 | DPB1 | DPB3 | DPB8 | DPB12 | DPB15 | DPB19 | DPB22 | | | | |
| DPB1*8101 | DPB1 | DPB3 | DPB8 | DPB12 | DPB16 | DPB19 | DPB22 | | | | |
| DPB1*02013 | DPB1 | DPB5 | DPB9 | DPB12 | DPB16 | DPB19 | DPB22 | | | | |
| DPB1*4701 | DPB1 | DPB5 | DPB10 | DPB12 | DPB16 | DPB19 | DPB22 | | | | |
| DPB1*1901 | DPB1 | DPB5 | DPB10 | DPB12 | DPB16 | DPB20 | DPB21 | | | | |
| DPB1*3001 | DPB2 | DPB5 | DPB10 | DPB12 | DPB16 | DPB19 | DPB21 | | | | 456 |
| DPB1*5401 | DPB2 | DPB5 | DPB10 | DPB12 | DPB16 | DPB18 | DPB21 | | | | 456 |
| DPB1*0202 | DPB1 | DPB6 | DPB10 | DPB12 | DPB16 | DPB19 | DPB22 | | | | |
| DPB1*2201 | DPB1 | DPB6 | DPB10 | DPB12 | DPB16 | DPB19 | DPB21 | | | | |
| DPB1*0501 | DPB1 | DPB6 | DPB10 | DPB12 | DPB15 | DPB19 | DPB21 | | | | |
| DPB1*3801 | DPB1 | DPB6 | DPB10 | DPB12 | DPB15 | DPB19 | DPB21 | 465 | | | |
| DPB1*2101 | DPB2 | DPB6 | DPB10 | DPB12 | DPB16 | DPB19 | DPB21 | | | | |
| DPB1*3601 | DPB2 | DPB6 | DPB10 | DPB12 | DPB15 | DPB19 | DPB21 | | | | |
| DPB1*2401 | DPB1 | DPB3 | DPB10 | DPB12 | DPB15 | DPB19 | DPB22 | | | | |
| DPB1*8401 | DPB1 | DPB5 | DPB11 | DPB12 | DPB15 | DPB18 | DPB22 | | | | |
| DPB1*11011 | DPB2 | DPB4 | DPB7 | DPB13 | DPB17 | DPB19 | DPB21 | 467 | 473 | | |
| DPB1*11012 | DPB2 | DPB4 | DPB7 | DPB13 | DPB17 | DPB19 | DPB21 | 466 | 467 | 473 | |
| DPB1*1501 | DPB1 | DPB4 | DPB7 | DPB13 | DPB17 | DPB19 | DPB23 | 467 | 473 | | |
| DPB1*7401 | DPB2 | DPB4 | DPB7 | DPB13 | DPB17 | DPB19 | DPB23 | 467 | 473 | | |
| DPB1*3101 | DPB1 | DPB3 | DPB7 | DPB13 | DPB15 | DPB19 | DPB21 | | | | |
| DPB1*5601 | DPB2 | DPB3 | DPB7 | DPB13 | DPB15 | DPB18 | DPB21 | | | | |
| DPB1*7201 | DPB1 | DPB3 | DPB7 | DPB13 | DPB15 | DPB19 | DPB22 | | | | |
| DPB1*3401 | DPB1 | DPB6 | DPB7 | DPB13 | DPB15 | DPB19 | DPB23 | 493 | | | |
| DPB1*5201 | DPB2 | DPB5 | DPB7 | DPB13 | DPB15 | DPB18 | DPB21 | | | | |
| DPB1*03012 | DPB2 | DPB5 | DPB8 | DPB13 | DPB15 | DPB18 | DPB21 | 482 | 503 | | |
| DPB1*1401 | DPB2 | DPB5 | DPB8 | DPB13 | DPB15 | DPB18 | DPB21 | 482 | | | 456 |
| DPB1*6101N | DPB2 | DPB5 | DPB8 | DPB13 | DPB15 | DPB18 | DPB21 | 482 | | | |
| DPB1*7001 | DPB2 | DPB5 | DPB8 | DPB13 | DPB15 | DPB18 | DPB21 | 482 | | | 457 |
| DPB1*2501 | DPB2 | DPB5 | DPB8 | DPB13 | DPB15 | DPB18 | DPB21 | | | | |
| DPB1*4501 | DPB2 | DPB5 | DPB8 | DPB13 | DPB15 | DPB18 | DPB21 | | | | 456 |
| DPB1*6701 | DPB2 | DPB5 | DPB7 | DPB13 | DPB15 | DPB18 | DPB21 | | | | 456 |
| DPB1*7801 | DPB2 | DPB5 | DPB8 | DPB13 | DPB15 | DPB18 | DPB21 | 482 | | | |
| DPB1*2901 | DPB2 | DPB5 | DPB8 | DPB13 | DPB16 | DPB18 | DPB21 | 482 | | | |
| DPB1*0601 | DPB2 | DPB5 | DPB8 | DPB13 | DPB16 | DPB19 | DPB21 | 482 | | | |
| DPB1*20011 | DPB2 | DPB5 | DPB8 | DPB13 | DPB15 | DPB19 | DPB21 | 482 | | | |
| DPB1*20012 | DPB2 | DPB5 | DPB8 | DPB13 | DPB15 | DPB19 | DPB21 | 482 | | | |
| DPB1*6401N | DPB2 | DPB5 | DPB8 | DPB13 | DPB16 | DPB19 | DPB21 | | | | |
| DPB1*6901 | DPB2 | DPB5 | DPB8 | DPB13 | DPB17 | DPB19 | DPB21 | 482 | | | |
| DPB1*5001 | DPB1 | DPB5 | DPB8 | DPB13 | DPB15 | DPB18 | DPB21 | 482 | | | |
| DPB1*5701 | DPB1 | DPB5 | DPB8 | DPB13 | DPB15 | DPB18 | DPB21 | 482 | | | |
| DPB1*5901 | DPB1 | DPB5 | DPB8 | DPB13 | DPB15 | DPB19 | DPB22 | | | | |
| DPB1*7301 | DPB1 | DPB5 | DPB8 | DPB13 | DPB15 | DPB18 | DPB22 | | | | |
| DPB1*8701 | DPB2 | DPB5 | DPB7 | DPB13 | DPB15 | DPB19 | DPB22 | | | | |
| DPB1*2801 | DPB1 | DPB3 | DPB8 | DPB13 | DPB15 | DPB19 | DPB23 | | | | |
| DPB1*7601 | DPB2 | DPB3 | DPB8 | DPB13 | DPB15 | DPB18 | DPB21 | 482 | | | 456 |
| DPB1*4401 | DPB2 | DPB6 | DPB8 | DPB13 | DPB16 | DPB18 | DPB21 | 482 | | | |
| DPB1*03011 | DPB2 | DPB5 | DPB10 | DPB13 | DPB15 | DPB18 | DPB21 | 482 | | | |
| DPB1*4101 | DPB1 | DPB5 | DPB8 | DPB14 | DPB16 | DPB19 | DPB22 | | | | |
| DPB1*8301 | DPB1 | DPB5 | DPB8 | DPB14 | DPB15 | DPB19 | DPB22 | | | | |
| DPB1*8601 | DPB2 | DPB5 | DPB8 | DPB9 | DPB16 | DPB19 | DPB22 | 482 | | | |

TABLE 22
Correspondence of Patchwork
DPB1 and SEQ ID NOS. for DPB1

| Patchwork | SEQ ID NOS. | | |
|---|---|---|---|
| F | 458 | 460 | 462 |
| DPB2 | 459 | 461 | 463 |
| DPB3 | 468 | | |
| DPB4 | 469 | 472 | |
| DPB5 | 470 | | |
| DPB6 | 471 | | |
| DPB7 | 474 | | |
| DPB8 | 475 | 479 | 480 |
| DPB9 | 476 | | |
| DPB10 | 477 | | |
| DPB11 | 478 | | |
| DPB12 | 483 | | |
| DPB13 | 484 | 486 | |
| DPB14 | 485 | | |
| DPB15 | 489 | 92 | |
| DPB16 | 490 | | |
| DPB17 | 491 | | |
| DPB18 | 496 | 498 | |
| DPB19 | 497 | | |
| DPB20 | 499 | | |
| DPB21 | 500 | | |
| DPB22 | 501 | | |
| DPB23 | 502 | | |

TABLE 23
Correspondence of the patchwork regions and satellite regions for types of DQB1

| | patchwork region | | | | | | | | | | |
|---|---|---|---|---|---|---|---|---|---|---|---|
| DQB1*0201 | DQB1 | DQB5 | DQB8 | DQB10 | DQB15 | DQB19 | DQB23 | DQB26 | DQB30 | DQB34 | |
| DQB1*0202 | DQB1 | DQB5 | DQB8 | DQB10 | DQB15 | DQB19 | DQB23 | DQB26 | DQB30 | DQB34 | |
| DQB1*0203 | DQB1 | DQB5 | DQB8 | DQB10 | DQB15 | DQB18 | DQB23 | DQB26 | DQB30 | DQB33 | DQB34 |
| DQB1*05031 | DQB1 | DQB4 | DQB7 | DQB10 | DQB14 | DQB18 | DQB22 | DQB24 | DQB29 | | |
| DQB1*03032 | DQB1 | DQB5 | DQB8 | DQB11 | DQB14 | DQB18 | DQB22 | DQB27 | DQB30 | DQB35 | |
| DQB1*0310 | DQB1 | DQB6 | DQB9 | DQB11 | DQB14 | DQB18 | DQB22 | DQB27 | DQB30 | DQB35 | |
| DQB1*03033 | DQB1 | DQB5 | DQB8 | DQB12 | DQB14 | DQB18 | DQB22 | DQB27 | DQB30 | DQB35 | |
| DQB1*06011 | DQB3 | DQB6 | DQB9 | DQB10 | DQB14 | DQB18 | DQB23 | DQB27 | DQB31 | | |
| DQB1*06013 | DQB6 | DQB9 | DQB10 | DQB14 | DQB18 | DQB23 | DQB27 | DQB31 | | | |
| DQB1*06012 | DQB3 | DQB6 | DQB9 | DQB10 | DQB14 | DQB18 | DQB23 | DQB27 | | | |
| DQB1*0306 | DQB1 | DQB5 | DQB8 | DQB11 | DQB14 | DQB18 | DQB23 | DQB25 | DQB30 | DQB35 | |
| DQB1*0401 | DQB2 | DQB5 | DQB7 | DQB12 | DQB14 | DQB18 | DQB23 | DQB25 | DQB30 | | |
| DQB1*0402 | DQB2 | DQB5 | DQB7 | DQB12 | DQB14 | DQB18 | DQB23 | DQB25 | DQB30 | | |
| DQB1*05032 | DQB7 | DQB10 | DQB14 | DQB20 | DQB22 | | | | | | |
| DQB1*0302 | DQB1 | DQB5 | DQB8 | DQB11 | DQB14 | DQB19 | DQB22 | DQB27 | DQB30 | DQB33 | |
| DQB1*0307 | DQB1 | DQB5 | DQB8 | DQB11 | DQB14 | DQB19 | DQB22 | DQB27 | DQB30 | DQB35 | |
| DQB1*0308 | DQB1 | DQB5 | DQB8 | DQB11 | DQB14 | DQB19 | DQB22 | DQB28 | DQB30 | DQB35 | |
| DQB1*0309 | DQB1 | DQB5 | DQB7 | DQB12 | DQB14 | DQB19 | DQB22 | DQB27 | DQB30 | DQB35 | |
| DQB1*0309 | DQB1 | DQB6 | DQB9 | DQB11 | DQB13 | DQB18 | DQB22 | DQB27 | DQB30 | DQB35 | |
| DQB1*03011 | DQB1 | DQB6 | DQB9 | DQB11 | DQB13 | DQB18 | DQB22 | DQB27 | DQB30 | DQB35 | |
| DQB1*03012 | DQB1 | DQB6 | DQB9 | DQB12 | DQB13 | DQB18 | DQB22 | DQB27 | DQB30 | DQB35 | |
| DQB1*05011 | DQB1 | DQB4 | DQB7 | DQB10 | DQB13 | DQB16 | DQB22 | DQB24 | DQB29 | | |
| DQB1*05012 | DQB1 | DQB4 | DQB7 | DQB10 | DQB13 | DQB16 | DQB22 | DQB24 | DQB29 | | |
| DQB1*06041 | DQB1 | DQB5 | DQB8 | DQB12 | DQB13 | DQB16 | DQB22 | DQB27 | DQB32 | | |
| DQB1*06042 | DQB1 | DQB5 | DQB8 | DQB12 | DQB13 | DQB16 | DQB22 | DQB27 | DQB32 | | |
| DQB1*0606 | DQB1 | DQB4 | DQB8 | DQB12 | DQB13 | DQB16 | DQB22 | DQB27 | | | |
| DQB1*0609 | DQB8 | DQB12 | DQB13 | DQB16 | DQB22 | DQB27 | DQB32 | | | | |
| DQB1*06051 | DQB1 | DQB5 | DQB8 | DQB12 | DQB13 | DQB16 | DQB22 | DQB27 | DQB32 | | |
| DQB1*0608 | DQB1 | DQB5 | DQB8 | DQB12 | DQB13 | DQB16 | DQB22 | DQB28 | DQB31 | | |
| DQB1*0613 | DQB1 | DQB5 | DQB8 | DQB12 | DQB13 | DQB16 | DQB22 | DQB28 | DQB31 | | |
| DQB1*0612 | DQB2 | DQB5 | DQB8 | DQB12 | DQB13 | DQB16 | DQB22 | DQB28 | DQB32 | | |
| DQB1*0617 | DQB1 | DQB5 | DQB8 | DQB12 | DQB13 | DQB16 | DQB22 | DQB24 | DQB32 | | |
| DQB1*0502 | DQB1 | DQB4 | DQB7 | DQB10 | DQB13 | DQB17 | DQB22 | DQB24 | DQB29 | | |
| DQB1*0504 | DQB1 | DQB4 | DQB7 | DQB10 | DQB13 | DQB17 | DQB23 | DQB25 | DQB33 | | |
| DQB1*0610 | DQB2 | DQB5 | DQB8 | DQB12 | DQB13 | DQB17 | DQB22 | DQB28 | DQB31 | | |
| DQB1*06111 | DQB2 | DQB5 | DQB8 | DQB12 | DQB13 | DQB20 | DQB22 | DQB28 | DQB31 | | |
| DQB1*06112 | DQB1 | DQB5 | DQB8 | DQB12 | DQB13 | DQB20 | DQB22 | DQB28 | DQB31 | | |
| DQB1*0603 | DQB1 | DQB5 | DQB8 | DQB12 | DQB13 | DQB20 | DQB22 | DQB28 | DQB31 | | |
| DQB1*0602 | DQB1 | DQB5 | DQB8 | DQB12 | DQB13 | DQB20 | DQB22 | DQB28 | DQB31 | | |
| DQB1*0616 | DQB2 | DQB5 | DQB8 | DQB12 | DQB13 | DQB20 | DQB22 | DQB28 | DQB31 | | |
| DQB1*0614 | DQB2 | DQB5 | DQB8 | DQB12 | DQB13 | DQB20 | DQB22 | DQB28 | DQB31 | | |
| DQB1*0615 | DQB1 | DQB5 | DQB8 | DQB12 | DQB13 | DQB20 | DQB22 | DQB27 | DQB32 | | |
| DQB1*0607 | DQB2 | DQB5 | DQB8 | DQB12 | DQB13 | DQB20 | DQB22 | DQB27 | DQB32 | | |
| DQB1*0304 | DQB1 | DQB6 | DQB9 | DQB11 | DQB13 | DQB19 | DQB22 | DQB27 | DQB30 | DQB35 | |
| DQB1*06052 | DQB8 | DQB12 | DQB13 | DQB21 | DQB22 | DQB27 | | | | | |

satellite region

| | | | | | | | | | |
|---|---|---|---|---|---|---|---|---|---|
| DQB1*0201 | 514 | 525 | 527 | 539 | 549 | 552 | 553 | 581 | |
| DQB1*0202 | 514 | 525 | 527 | 539 | 549 | 552 | 553 | 581 | |
| DQB1*0203 | 514 | 525 | 527 | 529 | 539 | 549 | 552 | 553 | 581 |
| DQB1*05031 | 549 | 584 | | | | | | | |
| DQB1*03032 | 526 | 549 | 553 | 582 | 583 | | | | |
| DQB1*0310 | 526 | 549 | 582 | 583 | | | | | |
| DQB1*03033 | 526 | 553 | 582 | 583 | | | | | |
| DQB1*06011 | 513 | 526 | 530 | 549 | 580 | 582 | 583 | | |
| DQB1*06013 | 513 | 526 | 530 | 549 | 580 | 582 | 583 | | |
| DQB1*06012 | 526 | 530 | 549 | 551 | 580 | 582 | 583 | | |
| DQB1*0306 | 515 | 526 | 549 | 553 | 571 | 582 | 583 | | |
| DQB1*0401 | 515 | 516 | 526 | 549 | 553 | 556 | 571 | 585 | |
| DQB1*0402 | 515 | 526 | 549 | 553 | 556 | 571 | 585 | | |
| DQB1*05032 | 549 | | | | | | | | |
| DQB1*0302 | 526 | 549 | 553 | 582 | 583 | | | | |
| DQB1*0307 | 526 | 550 | 553 | 582 | 583 | | | | |
| DQB1*0308 | 526 | 549 | 553 | 582 | 583 | | | | |
| DQB1*0309 | 515 | 526 | 549 | 553 | 582 | 583 | | | |
| DQB1*0309 | 526 | 538 | 549 | 553 | 582 | 583 | | | |
| DQB1*03011 | 526 | 538 | 549 | 553 | 582 | 583 | | | |
| DQB1*03012 | 526 | 538 | 549 | 553 | 582 | 583 | | | |
| DQB1*05011 | | | | | | | | | |
| DQB1*05012 | 549 | 584 | | | | | | | |
| DQB1*06041 | 524 | 549 | 582 | 583 | | | | | |
| DQB1*06042 | 524 | 548 | 549 | 582 | 583 | | | | |
| DQB1*0606 | 524 | 526 | 549 | 581 | | | | | |
| DQB1*0609 | 524 | 526 | 549 | 582 | 583 | | | | |
| DQB1*06051 | 524 | 526 | 549 | 582 | 583 | | | | |
| DQB1*0608 | 524 | 548 | 549 | 582 | 583 | | | | |
| DQB1*0613 | 526 | 548 | 549 | 582 | 583 | | | | |
| DQB1*0612 | 524 | 526 | 549 | 582 | 583 | | | | |
| DQB1*0617 | 524 | 549 | 582 | 583 | | | | | |
| DQB1*0502 | 549 | 584 | | | | | | | |
| DQB1*0504 | 526 | 549 | | | | | | | |
| DQB1*0610 | 526 | 548 | 549 | 582 | 583 | | | | |
| DQB1*06111 | 526 | 548 | 549 | 582 | 583 | | | | |
| DQB1*06112 | 524 | 526 | 548 | 549 | 582 | 583 | | | |
| DQB1*0603 | 524 | 548 | 549 | 582 | 583 | | | | |
| DQB1*0602 | 526 | 548 | 549 | 582 | 583 | | | | |
| DQB1*0616 | 526 | 548 | 549 | 570 | 582 | 583 | | | |
| DQB1*0614 | 524 | 548 | 549 | 582 | 583 | | | | |
| DQB1*0615 | 526 | 548 | 549 | 582 | 583 | | | | |
| DQB1*0607 | 524 | 548 | 549 | 582 | 583 | | | | |
| DQB1*0304 | 526 | 538 | 549 | 582 | 583 | | | | |
| DQB1*06052 | 524 | 526 | 549 | 582 | 583 | | | | |

TABLE 24
Correspondence of the patchworks and SEQ ID Nos for DQB1

| | |
|---|---|
| patchwork | SEQ ID NO: |

| | | | | | | |
|---|---|---|---|---|---|---|
| DQB1 | 507 | | | | | |
| DQB2 | 508 | | | | | |
| DQB3 | 509 | | | | | |
| DQB4 | 510 | | | | | |
| DQB5 | 511 | | | | | |
| DQB6 | 512 | | | | | |
| DQB7 | 517 | 520 | 522 | | | |
| DQB8 | 518 | 521 | 523 | | | |
| DQB9 | 519 | | | | | |
| DQB10 | 533 | 536 | 537 | | | |
| DQB11 | 534 | | | | | |
| DQB12 | 535 | | | | | |
| DQB13 | 540 | 543 | 544 | 545 | 546 | 547 |
| DQB14 | 541 | | | | | |
| DQB15 | 542 | | | | | |
| DQB16 | 559 | | | | | |
| DQB17 | 560 | | | | | |
| DQB18 | 561 | 565 | 568 | 569 | | |
| DQB19 | 562 | 566 | | | | |
| DQB20 | 563 | 567 | | | | |
| DQB21 | 564 | | | | | |
| DQB22 | 572 | | | | | |
| DQB23 | 573 | | | | | |
| DQB24 | 574 | | | | | |
| DQB25 | 575 | | | | | |
| DQB26 | 576 | | | | | |
| DQB27 | 577 | 579 | | | | |
| DQB28 | 578 | | | | | |
| DQB29 | 586 | | | | | |
| DQB30 | 587 | | | | | |
| DQB31 | 588 | | | | | |
| DQB32 | 589 | | | | | |
| DQB33 | 528 | 532 | | | | |
| DQB34 | 554 | 557 | | | | |
| DQB35 | 555 | 558 | | | | |

TABLE 25
Correspondence of the patchwork regions and satellite regions for types of DRB1

| | patchwork region | | | | | | | | | | | | | | | satellite region | |
|---|---|---|---|---|---|---|---|---|---|---|---|---|---|---|---|---|---|
| DRB1*0101 | D1 | D4 | DD11 | D13 | D18 | D23 | D29 | D31 | D48 | D51 | D59 | D63 | D70 | D73 | | | |
| DRB1*0419 | D1 | D5 | D10 | D16 | D23 | D29 | D31 | D48 | D51 | D59 | D63 | D70 | D73 | | | | |
| DRB1*0105 | D1 | D4 | DD11 | D13 | D18 | D23 | D29 | D31 | D48 | D51 | D59 | D63 | D70 | D73 | | 688 | |
| DRB1*01021 | D1 | D4 | DD11 | D13 | D18 | D23 | D29 | D31 | D48 | D51 | D59 | D63 | D71 | D74 | | 791 | |
| DRB1*0104 | D1 | D4 | DD11 | D13 | D18 | D23 | D29 | D31 | D48 | D51 | D59 | D63 | D70 | D74 | | 783 | |
| DRB1*01022 | D1 | D4 | DD11 | D13 | D18 | D23 | D29 | D31 | D48 | D51 | D59 | D69 | D71 | D74 | | 791 | |
| DRB1*0406 | D1 | D5 | D10 | D16 | D23 | D29 | D31 | D48 | D51 | D59 | D65 | D70 | D74 | | | | |
| DRB1*0420 | D1 | D5 | D10 | D16 | D23 | D29 | D31 | D48 | D51 | D59 | D65 | D70 | D73 | | | | |
| DRB1*0106 | D1 | D4 | DD11 | D13 | D18 | D23 | D29 | D31 | D48 | D57 | D59 | D63 | D70 | D74 | | | |
| DRB1*0304 | D1 | D6 | D10 | D14 | D19 | D23 | D30 | D31 | D48 | D56 | D59 | D66 | D70 | D74 | | 782 | |
| DRB1*0421 | D1 | D5 | D10 | D16 | D23 | D29 | D31 | D48 | D56 | D59 | D63 | D70 | D73 | | | | |
| DRB1*03021 | D1 | D5 | D11 | D14 | D19 | D24 | D29 | D31 | D48 | D56 | D59 | D66 | D70 | D73 | | 782 | |
| DRB1*0303 | D1 | D5 | D11 | D14 | D19 | D24 | D29 | D31 | D48 | D56 | D59 | D66 | D70 | D74 | | 782 | |
| DRB1*0306 | D1 | D6 | D10 | D14 | D19 | D24 | D29 | D31 | D48 | D56 | D59 | D66 | D70 | D74 | | 782 | |
| DRB1*0305 | D1 | D6 | D10 | D14 | D19 | D24 | D30 | D31 | D48 | D56 | D59 | D66 | D70 | D73 | | 782 | |
| DRB1*0309 | D1 | D6 | D10 | D14 | D21 | D24 | D30 | D31 | D48 | D56 | D59 | D66 | D70 | D73 | | 782 | |
| DRB1*0307 | D1 | D5 | D10 | D14 | D19 | D24 | D30 | D31 | D48 | D56 | D59 | D66 | D70 | D74 | | 782 | |
| DRB1*03011 | D1 | D6 | D10 | D14 | D19 | D24 | D30 | D31 | D48 | D56 | D59 | D66 | D70 | D74 | | 782 | |
| DRB1*0311 | D1 | D6 | D10 | D14 | D19 | D24 | D30 | D31 | D48 | D56 | D59 | D67 | D70 | D74 | | 782 | |
| DRB1*03012 | D1 | D6 | D10 | D14 | D19 | D24 | D30 | D31 | D48 | D56 | D59 | D66 | D70 | D74 | | 783 | |
| DRB1*0314 | D1 | D6 | D10 | D14 | D19 | D24 | D30 | D31 | D48 | D56 | D59 | D66 | D70 | D73 | | | |
| DRB1*0316 | D1 | D6 | D10 | D14 | D19 | D24 | D30 | D31 | D48 | D56 | D59 | D66 | D70 | D74 | | 782 | 699 |
| DRB1*0315 | D1 | D6 | D10 | D14 | D19 | D24 | D30 | D31 | D48 | D56 | D59 | D66 | D70 | D74 | | | |
| DRB1*1421 | D1 | D5 | D10 | D14 | D19 | D24 | D30 | D31 | D48 | D56 | D59 | D63 | D70 | D74 | | | |
| DRB1*03022 | D1 | D5 | D11 | D14 | D19 | D24 | D29 | D31 | D48 | D56 | D59 | D66 | D70 | D73 | | 783 | |
| DRB1*1419 | D1 | D5 | D11 | D14 | D19 | D24 | D29 | D31 | D48 | D56 | D59 | D63 | D70 | D73 | | | |
| DRB1*1320 | D1 | D5 | D10 | D14 | D19 | D24 | D30 | D31 | D48 | D52 | D59 | D63 | D70 | D74 | | | |
| DRB1*1329 | D1 | D5 | D10 | D14 | D19 | D24 | D30 | D31 | D48 | D52 | D59 | D63 | D70 | D73 | | | |
| DRB1*1412 | D1 | D5 | D11 | D14 | D19 | D24 | D29 | D31 | D48 | D54 | D59 | D64 | D70 | D74 | | | |
| DRB1*1403 | D1 | D5 | D11 | D14 | D19 | D24 | D29 | D31 | D48 | D54 | D59 | D64 | D70 | D73 | | | |
| DRB1*1406 | D1 | D5 | D11 | D14 | D19 | D24 | D29 | D31 | D48 | D51 | D59 | D63 | D70 | D74 | | | |
| DRB1*1417 | D1 | D5 | D10 | D14 | D19 | D24 | D30 | D31 | D48 | D51 | D59 | D63 | D70 | D74 | | | |
| DRB1*1433 | D1 | D5 | D10 | D14 | D19 | D24 | D30 | D31 | D48 | D51 | D59 | D65 | D70 | D74 | | | |
| DRB1*1430 | D1 | D5 | D10 | D14 | D19 | D24 | D30 | D31 | D48 | D51 | D59 | D63 | D70 | D73 | | | |
| DRB1*1429 | D1 | D5 | D11 | D14 | D19 | D24 | D29 | D31 | D48 | D51 | D59 | D63 | D70 | D74 | | 791 | |
| DRB1*1402 | D1 | D5 | D11 | D14 | D19 | D24 | D29 | D31 | D48 | D51 | D59 | D63 | D70 | D73 | | | |
| DRB1*1409 | D1 | D5 | D10 | D14 | D19 | D24 | D29 | D31 | D48 | D51 | D59 | D63 | D70 | D73 | | | |
| DRB1*04031 | D1 | D5 | D10 | D16 | D25 | D29 | D31 | D48 | D51 | D59 | D65 | D70 | D74 | | | | |
| DRB1*0407 | D1 | D5 | D10 | D16 | D25 | D29 | D31 | D48 | D51 | D59 | D65 | D70 | D73 | | | | |
| DRB1*0427 | D1 | D5 | D10 | D16 | D25 | D29 | D31 | D48 | D51 | D59 | D65 | D70 | D74 | | | 791 | |
| DRB1*0404 | D1 | D5 | D10 | D16 | D25 | D29 | D31 | D48 | D51 | D59 | D63 | D70 | D74 | | | | |
| DRB1*0408 | D1 | D5 | D10 | D16 | D25 | D29 | D31 | D48 | D51 | D59 | D63 | D70 | D73 | | | | |
| DRB1*0423 | D1 | D5 | D10 | D16 | D25 | D29 | D31 | D48 | D51 | D59 | D63 | D70 | D74 | | | 799 | |
| DRB1*0432 | D1 | D5 | D10 | D16 | D25 | D29 | D31 | D48 | D51 | D59 | D63 | D70 | D74 | | | 751 | |
| DRB1*0431 | D1 | D5 | D10 | D16 | D25 | D29 | D31 | D48 | D51 | D59 | D64 | D70 | D73 | | | | |
| DRB1*1344 | D1 | D5 | D10 | D13 | D18 | D25 | D30 | D31 | D48 | D51 | D59 | D63 | D70 | D74 | | | |
| DRB1*0317 | D1 | D5 | D10 | D13 | D18 | D25 | D29 | D31 | D48 | D56 | D59 | D67 | D70 | D73 | | 699 | 783 |
| DRB1*04011 | D1 | D5 | D10 | D16 | D25 | D29 | D31 | D48 | D56 | D59 | D63 | D70 | D73 | | | | |
| DRB1*04012 | D1 | D5 | D10 | D16 | D25 | D29 | D31 | D48 | D56 | D59 | D63 | D70 | D73 | | | 665 | |
| DRB1*0413 | D1 | D5 | D10 | D16 | D25 | D29 | D31 | D48 | D56 | D59 | D63 | D70 | D74 | | | | |
| DRB1*0422 | D1 | D5 | D10 | D16 | D25 | D29 | D31 | D48 | D56 | D59 | D66 | D70 | D74 | | | 782 | |
| DRB1*0433 | D1 | D5 | D10 | D16 | D25 | D29 | D31 | D48 | D56 | D59 | D63 | D70 | D73 | | | 684 | |
| DRB1*0434 | D1 | D5 | D10 | D16 | D25 | D29 | D31 | D48 | D56 | D59 | D63 | D70 | D73 | | | | |
| DRB1*0435 | D1 | D5 | D10 | D16 | D25 | D30 | D31 | D48 | D56 | D59 | D63 | D70 | D73 | | | | |
| DRB1*0813 | D2 | D5 | D10 | D13 | D18 | D25 | D29 | D31 | D48 | D54 | D59 | D64 | D70 | D73 | | | |
| DRB1*1325 | D1 | D5 | D10 | D13 | D18 | D25 | D30 | D31 | D48 | D54 | D59 | D63 | D70 | D73 | | | |
| DRB1*10011 | D1 | D4 | DD11 | D17 | D22 | D25 | D29 | D31 | D48 | D55 | D62 | D63 | D70 | D73 | | 752 | |
| DRB1*10012 | D1 | D4 | DD11 | D17 | D22 | D25 | D29 | D31 | D48 | D55 | D60 | D63 | D70 | D73 | | 753 | |
| DRB1*1414 | D1 | D5 | D10 | D14 | D19 | D26 | D29 | D31 | D48 | D55 | D59 | D65 | D71 | D73 | | | |
| DRB1*1436 | D1 | D5 | D10 | D14 | D19 | D26 | D29 | D31 | D48 | D55 | D59 | D65 | D71 | D73 | | 686 | |
| DRB1*1423 | D1 | D5 | D10 | D14 | D19 | D26 | D29 | D31 | D48 | D55 | D59 | D65 | D71 | D74 | | | |
| DRB1*1420 | D1 | D5 | D11 | D14 | D19 | D26 | D29 | D31 | D48 | D51 | D59 | D63 | D70 | D74 | | | |
| DRB1*0103 | D1 | D4 | DD11 | D13 | D18 | D23 | D29 | D31 | D49 | D52 | D59 | D63 | D70 | D73 | | | |
| DRB1*15022 | D1 | D5 | D10 | D15 | D20 | D23 | D30 | D31 | D49 | D57 | D59 | D63 | D70 | D73 | | | |
| DRB1*1301 | D1 | D5 | D10 | D14 | D19 | D24 | D30 | D31 | D49 | D52 | D59 | D63 | D70 | D74 | | | |
| DRB1*1327 | D1 | D6 | D10 | D14 | D19 | D24 | D30 | D31 | D49 | D52 | D59 | D63 | D70 | D74 | | | |
| DRB1*1315 | D1 | D5 | D11 | D14 | D19 | D24 | D30 | D31 | D49 | D52 | D59 | D63 | D70 | D74 | | | |
| DRB1*1328 | D1 | D5 | D10 | D14 | D19 | D24 | D30 | D31 | D49 | D52 | D59 | D63 | D70 | D74 | | 789 | |
| DRB1*1340 | D1 | D5 | D10 | D14 | D19 | D24 | D29 | D31 | D49 | D52 | D59 | D63 | D70 | D74 | | | |
| DRB1*13021 | D1 | D5 | D10 | D14 | D19 | D24 | D30 | D31 | D49 | D52 | D59 | D63 | D70 | D73 | | | |
| DRB1*13022 | D1 | D5 | D10 | D14 | D19 | D24 | D30 | D31 | D49 | D52 | D60 | D63 | D70 | D73 | | | |
| DRB1*1334 | D1 | D5 | D10 | D14 | D19 | D24 | D30 | D31 | D49 | D52 | D59 | D63 | D70 | D73 | | 664 | |
| DRB1*1335 | D1 | D5 | D10 | D14 | D19 | D24 | D30 | D31 | D49 | D52 | D59 | D63 | D70 | D73 | | 687 | |
| DRB1*1336 | D1 | D5 | D10 | D14 | D19 | D24 | D29 | D31 | D49 | D52 | D59 | D63 | D70 | D73 | | | |
| DRB1*1341 | D1 | D6 | D10 | D14 | D19 | D24 | D30 | D31 | D49 | D52 | D59 | D63 | D70 | D73 | | | |
| DRB1*1316 | D1 | D5 | D10 | D14 | D19 | D24 | D30 | D31 | D49 | D52 | D59 | D63 | D70 | D75 | | | |
| DRB1*1306 | D1 | D5 | D10 | D14 | D19 | D24 | D30 | D31 | D49 | D54 | D59 | D63 | D70 | D74 | | | |
| DRB1*1310 | D1 | D5 | D10 | D14 | D19 | D24 | D30 | D31 | D49 | D53 | D59 | D63 | D70 | D74 | | | |
| DRB1*1424 | D1 | D5 | D11 | D14 | D19 | D24 | D29 | D31 | D49 | D57 | D59 | D63 | D70 | D73 | | | |
| DRB1*1309 | D1 | D5 | D10 | D14 | D19 | D24 | D30 | D31 | D49 | D57 | D59 | D63 | D70 | D74 | | | |
| DRB1*0402 | D1 | D5 | D10 | D16 | D25 | D29 | D31 | D49 | D52 | D59 | D63 | D70 | D74 | | | | |
| DRB1*0414 | D1 | D5 | D10 | D16 | D25 | D29 | D31 | D49 | D52 | D59 | D63 | D70 | D73 | | | | |
| DRB1*1317 | D2 | D5 | D10 | D13 | D18 | D25 | D30 | D31 | D49 | D52 | D59 | D63 | D70 | D74 | | | |
| DRB1*1322 | D1 | D5 | D10 | D13 | D18 | D25 | D30 | D31 | D49 | D52 | D59 | D63 | D70 | D74 | | | |
| DRB1*1323 | D1 | D5 | D10 | D13 | D18 | D25 | D30 | D31 | D49 | D52 | D59 | D63 | D70 | D73 | | | |
| DRB1*0418 | D1 | D5 | D10 | D16 | D25 | D29 | D31 | D49 | D54 | D59 | D64 | D70 | D74 | | | | |
| DRB1*1337 | D1 | D5 | D10 | D13 | D18 | D25 | D29 | D31 | D49 | D53 | D59 | D63 | D70 | D73 | | | |
| DRB1*1308 | D1 | D5 | D10 | D14 | D19 | D26 | D29 | D31 | D49 | D52 | D59 | D63 | D70 | D74 | | | |
| DRB1*1319 | D1 | D5 | D11 | D14 | D19 | D26 | D29 | D31 | D49 | D52 | D59 | D63 | D70 | D74 | | | |
| DRB1*0425 | D1 | D5 | D10 | D16 | D25 | D29 | D31 | D50 | D54 | D59 | D64 | D70 | D74 | | | | |
| DRB1*0436 | D1 | D5 | D10 | D16 | D25 | D29 | D31 | D50 | D54 | D59 | D63 | D70 | D74 | | | | |
| DRB1*08021 | D2 | D5 | D10 | D13 | D18 | D25 | D29 | D31 | D50 | D54 | D59 | D64 | D70 | D73 | | | |
| DRB1*08022 | D2 | D5 | D10 | D13 | D18 | D25 | D29 | D31 | D50 | D54 | D59 | D64 | D70 | D73 | | | |
| DRB1*08041 | D2 | D5 | D10 | D13 | D18 | D25 | D29 | D31 | D50 | D54 | D59 | D64 | D70 | D74 | | | |
| DRB1*08042 | * | D5 | D10 | D13 | D18 | D25 | D29 | D31 | D50 | D54 | D59 | D64 | D70 | D75 | | | |
| DRB1*08043 | D2 | D5 | D10 | D13 | D18 | D25 | D29 | D31 | D50 | D54 | D59 | D64 | D70 | D75 | | | |
| DRB1*0820 | D1 | D5 | D10 | D13 | D18 | D25 | D29 | D31 | D50 | D54 | D59 | D64 | D70 | D74 | | | |
| DRB1*1311 | D1 | D5 | D10 | D13 | D18 | D25 | D30 | D31 | D50 | D54 | D59 | D63 | D70 | D74 | | | |
| DRB1*13141 | D1 | D5 | D10 | D13 | D18 | D25 | D30 | D31 | D50 | D54 | D59 | D63 | D70 | D73 | | | |
| DRB1*1324 | D1 | D5 | D10 | D13 | D18 | D25 | D30 | D31 | D50 | D52 | D59 | D63 | D70 | D74 | | | |
| DRB1*13071 | D1 | D5 | D10 | D13 | D18 | D25 | D29 | D31 | D50 | D53 | D59 | D63 | D70 | D73 | | | |
| DRB1*0809 | D2 | D5 | D10 | D14 | D19 | D26 | D29 | D31 | D50 | D54 | D59 | D64 | D70 | D73 | | | |
| DRB1*0821 | D2 | D5 | D10 | D14 | D19 | D26 | D29 | D31 | D50 | D54 | D59 | D64 | D70 | D73 | | | |
| DRB1*1415 | D2 | D5 | D10 | D14 | D19 | D26 | D29 | D31 | D50 | D54 | D59 | D64 | D70 | D74 | | | |
| DRB1*1318 | D1 | D5 | D10 | D14 | D19 | D24 | D30 | D31 | D50 | D54 | D59 | D64 | D70 | D74 | | | |
| DRB1*1342 | D1 | D5 | D10 | D14 | D19 | D24 | D30 | D31 | D50 | D54 | D59 | D63 | D70 | D74 | | | |
| DRB1*1427 | D1 | D5 | D11 | D14 | D19 | D24 | D29 | D31 | D50 | D54 | D59 | D64 | D70 | D73 | | | |
| DRB1*1305 | D1 | D5 | D10 | D14 | D19 | D24 | D30 | D31 | D50 | D53 | D59 | D63 | D70 | D73 | | | |
| DRB1*0312 | D1 | D6 | D10 | D14 | D19 | D24 | D30 | D32 | D48 | D56 | D59 | D66 | D70 | D74 | | 782 | |
| DRB1*0411 | D1 | D5 | D10 | D16 | D25 | D29 | D32 | D48 | D51 | D59 | D59 | D70 | D74 | | | | |
| DRB1*0417 | D1 | D5 | D10 | D16 | D25 | D29 | D32 | D48 | D51 | D59 | D65 | D70 | D73 | | | | |
| DRB1*0410 | D1 | D5 | D10 | D16 | D25 | D29 | D32 | D48 | D51 | D59 | D63 | D70 | D74 | | | | |
| DRB1*04051 | D1 | D5 | D10 | D16 | D25 | D29 | D32 | D48 | D51 | D59 | D63 | D70 | D73 | | | | |
| DRB1*04052 | D1 | D5 | D10 | D16 | D25 | D29 | D32 | D48 | D51 | D59 | D63 | D70 | D73 | | | 685 | |
| DRB1*0429 | D1 | D5 | D10 | D16 | D25 | D29 | D32 | D48 | D51 | D59 | D63 | D70 | D73 | | | 700 | |
| DRB1*0430 | D1 | D5 | D10 | D16 | D25 | D29 | D32 | D48 | D51 | D59 | D63 | D70 | D73 | | | 697 | |
| DRB1*0428 | D1 | D5 | D10 | D16 | D25 | D30 | D32 | D48 | D51 | D59 | D63 | D70 | D73 | | | | |
| DRB1*0409 | D1 | D5 | D10 | D16 | D25 | D29 | D32 | D48 | D56 | D59 | D63 | D70 | D73 | | | | |
| DRB1*0412 | D1 | D5 | D10 | D16 | D25 | D29 | D32 | D49 | D54 | D59 | D64 | D70 | D74 | | | | |
| DRB1*0810 | D2 | D5 | D10 | D13 | D18 | D25 | D29 | D32 | D49 | D54 | D59 | D64 | D70 | D74 | | | |
| DRB1*0812 | D2 | D5 | D10 | D13 | D18 | D25 | D29 | D32 | D49 | D54 | D59 | D64 | D70 | D74 | | 791 | |
| DRB1*08032 | D2 | D5 | D10 | D13 | D18 | D25 | D29 | D32 | D49 | D54 | D59 | D64 | D70 | D73 | | | |
| DRB1*0814 | D2 | D5 | D10 | D13 | D18 | D25 | D29 | D32 | D49 | D54 | D59 | D64 | D70 | D73 | | | |
| DRB1*1313 | D1 | D5 | D10 | D13 | D18 | D25 | D29 | D32 | D49 | D54 | D59 | D64 | D70 | D73 | | | |
| DRB1*0818 | D2 | D5 | D10 | D13 | D18 | D25 | D29 | D32 | D49 | D54 | D59 | D63 | D70 | D73 | | | |
| DRB1*1312 | D1 | D5 | D10 | D13 | D18 | D25 | D29 | D32 | D49 | D54 | D59 | D63 | D70 | D73 | | | |
| DRB1*1330 | D1 | D5 | D10 | D13 | D18 | D25 | D30 | D32 | D49 | D54 | D59 | D63 | D70 | D73 | | | |
| DRB1*13031 | D1 | D5 | D10 | D13 | D18 | D25 | D29 | D32 | D49 | D53 | D59 | D63 | D70 | D73 | | | |
| DRB1*13032 | D1 | D5 | D10 | D13 | D18 | D25 | D29 | D32 | D49 | D53 | D59 | D63 | D70 | D73 | | | |
| DRB1*1333 | D1 | D5 | D10 | D13 | D18 | D25 | D29 | D32 | D49 | D53 | D59 | D63 | D70 | D73 | | 782 | |
| DRB1*1304 | D1 | D5 | D10 | D13 | D18 | D25 | D30 | D32 | D49 | D52 | D59 | D63 | D70 | D74 | | | |
| DRB1*1338 | D1 | D5 | D10 | D13 | D18 | D25 | D29 | D32 | D49 | D52 | D59 | D63 | D70 | D73 | | | |
| DRB1*1332 | D1 | D5 | D10 | D14 | D19 | D24 | D29 | D32 | D49 | D52 | D59 | D63 | D70 | D74 | | | |
| DRB1*0801 | D2 | D5 | D10 | D13 | D18 | D25 | D29 | D32 | D50 | D54 | D59 | D64 | D70 | D73 | | | |
| DRB1*0817 | D2 | D5 | D10 | D13 | D18 | D25 | D30 | D32 | D50 | D54 | D59 | D64 | D70 | D73 | | | |
| DRB1*0816 | D2 | D5 | D10 | D13 | D18 | D27 | D29 | D32 | D50 | D54 | D59 | D64 | D70 | D73 | | | |
| DRB1*0806 | D2 | D5 | D10 | D13 | D18 | D25 | D29 | D32 | D50 | D54 | D59 | D64 | D70 | D74 | | | |
| DRB1*0822 | D2 | D5 | D10 | D13 | D18 | D25 | D29 | D32 | D50 | D54 | D59 | D64 | D70 | D74 | | 791 | |
| DRB1*0805 | D2 | D5 | D10 | D13 | D18 | D25 | D29 | D32 | D50 | D54 | D59 | D63 | D70 | D73 | | | |
| DRB1*1321 | D1 | D5 | D10 | D13 | D18 | D25 | D30 | D32 | D50 | D54 | D59 | D63 | D70 | D73 | | | |
| DRB1*1413 | D1 | D5 | D11 | D14 | D19 | D24 | D29 | D32 | D48 | D51 | D59 | D63 | D70 | D73 | | | |
| DRB1*0308 | D1 | D6 | D10 | D14 | D19 | D24 | D30 | D43 | D48 | D56 | D59 | D66 | D70 | D74 | | 782 | |
| DRB1*1107 | D1 | D5 | D10 | D13 | D18 | D25 | D30 | D43 | D48 | D56 | D59 | D66 | D70 | D74 | | 782 | |
| DRB1*11081 | D1 | D5 | D10 | D13 | D18 | D25 | D30 | D43 | D48 | D54 | D59 | D63 | D70 | D73 | | | |
| DRB1*11082 | D1 | D5 | D10 | D13 | D18 | D25 | D30 | D43 | D48 | D54 | D59 | D63 | D70 | D73 | | | |
| DRB1*1136 | D1 | D5 | D10 | D13 | D18 | D25 | D30 | D43 | D48 | D52 | D59 | D63 | D70 | D74 | | | |
| DRB1*1126 | D1 | D5 | D10 | D13 | D18 | D25 | D30 | D43 | D48 | D51 | D59 | D63 | D70 | D73 | | | |
| DRB1*1134 | D1 | D5 | D10 | D13 | D18 | D25 | D30 | D43 | D48 | D51 | D59 | D63 | D70 | D74 | | | |
| DRB1*1113 | D1 | D5 | D10 | D14 | D19 | D26 | D30 | D43 | D48 | D55 | D59 | D63 | D71 | D74 | | | |
| DRB1*1117 | D1 | D5 | D10 | D14 | D19 | D26 | D29 | D43 | D48 | D55 | D59 | D65 | D71 | D74 | | | |
| DRB1*11011 | D1 | D5 | D10 | D13 | D18 | D25 | D30 | D43 | D50 | D54 | D59 | D63 | D70 | D73 | | | |
| DRB1*11012 | D1 | D5 | D10 | D13 | D18 | D25 | D30 | D43 | D50 | D54 | D59 | D63 | D70 | D73 | | | |
| DRB1*11013 | D1 | D5 | D10 | D13 | D18 | D25 | D30 | D43 | D50 | D54 | D60 | D63 | D70 | D73 | | | |
| DRB1*11271 | D1 | D5 | D10 | D13 | D18 | D25 | D30 | D43 | D50 | D54 | D59 | D63 | D70 | D73 | | 783 | |
| DRB1*11272 | D1 | D5 | D10 | D13 | D18 | D25 | D30 | D43 | D50 | D54 | D59 | D63 | D70 | D73 | | 782 | |
| DRB1*1130 | D1 | D5 | D10 | D13 | D18 | D25 | D30 | D43 | D50 | D54 | D59 | D63 | D70 | D73 | | 594 | |
| DRB1*1105 | D1 | D5 | D10 | D13 | D18 | D25 | D30 | D43 | D50 | D54 | D59 | D63 | D70 | D73 | | 597 | |
| DRB1*1137 | D1 | D5 | D10 | D13 | D18 | D25 | D29 | D43 | D50 | D54 | D59 | D63 | D70 | D73 | | | |
| DRB1*0415 | D1 | D5 | D10 | D16 | D25 | D29 | D43 | D50 | D54 | D59 | D63 | D70 | D74 | | | | |
| DRB1*1122 | D1 | D5 | D10 | D15 | D20 | D25 | D30 | D43 | D50 | D54 | D59 | D63 | D70 | D73 | | | |
| DRB1*1139 | D1 | D5 | D10 | D13 | D18 | D25 | D30 | D43 | D50 | D54 | D59 | D63 | D70 | D73 | | 699 | |
| DRB1*1123 | D1 | D5 | D10 | D13 | D18 | D25 | D30 | D43 | D50 | D54 | D59 | D64 | D70 | D73 | | | |
| DRB1*1132 | D1 | D5 | D10 | D13 | D18 | D25 | D30 | D43 | D50 | D54 | D59 | D68 | D70 | D73 | | | |
| DRB1*1125 | D1 | D5 | D10 | D13 | D18 | D25 | D30 | D43 | D50 | D54 | D59 | D64 | D70 | D74 | | | |
| DRB1*1106 | D1 | D5 | D10 | D13 | D18 | D25 | D30 | D43 | D50 | D54 | D59 | D63 | D70 | D74 | | 791 | |
| DRB1*11041 | D1 | D5 | D10 | D13 | D18 | D25 | D30 | D43 | D50 | D54 | D59 | D63 | D70 | D74 | | | |
| DRB1*11042 | D1 | D5 | D10 | D13 | D18 | D25 | D30 | D43 | D50 | D54 | D59 | D63 | D70 | D74 | | | |
| DRB1*1111 | D1 | D5 | D10 | D13 | D18 | D25 | D30 | D43 | D50 | D52 | D59 | D63 | D70 | D73 | | | |
| DRB1*1141 | D1 | D5 | D10 | D13 | D18 | D25 | D29 | D43 | D50 | D52 | D59 | D63 | D70 | D74 | | | |
| DRB1*1103 | D1 | D5 | D10 | D13 | D18 | D25 | D30 | D43 | D50 | D53 | D59 | D63 | D70 | D74 | | | |
| DRB1*1109 | D1 | D5 | D10 | D14 | D19 | D24 | D30 | D43 | D50 | D54 | D59 | D63 | D70 | D73 | | | |
| DRB1*1128 | D1 | D5 | D10 | D13 | D18 | D24 | D30 | D43 | D50 | D54 | D59 | D63 | D70 | D73 | | | |
| DRB1*1110 | D1 | D5 | D10 | D14 | D19 | D26 | D30 | D43 | D50 | D54 | D59 | D63 | D70 | D73 | | | |
| DRB1*11121 | D1 | D5 | D10 | D13 | D18 | D26 | D30 | D43 | D50 | D54 | D59 | D63 | D70 | D73 | | | |
| DRB1*1115 | D1 | D5 | D10 | D13 | D18 | D27 | D30 | D43 | D50 | D54 | D59 | D63 | D70 | D73 | | 666 | |
| DRB1*1124 | D1 | D5 | D10 | D13 | D18 | D27 | D30 | D43 | D50 | D54 | D59 | D63 | D70 | D73 | | | |
| DRB1*1129 | D1 | D5 | D10 | D13 | D18 | D23 | D30 | D43 | D50 | D54 | D59 | D63 | D70 | D73 | | | |
| DRB1*1119 | D1 | D5 | D10 | D13 | D18 | D25 | D30 | D43 | D49 | D54 | D59 | D63 | D70 | D73 | | | |
| DRB1*1118 | D1 | D5 | D10 | D13 | D18 | D25 | D30 | D43 | D49 | D54 | D59 | D63 | D70 | D74 | | | |
| DRB1*1114 | D1 | D5 | D10 | D13 | D18 | D25 | D30 | D43 | D49 | D52 | D59 | D63 | D70 | D73 | | | |
| DRB1*1102 | D1 | D5 | D10 | D13 | D18 | D25 | D30 | D43 | D49 | D52 | D59 | D63 | D70 | D74 | | | |
| DRB1*1121 | D1 | D5 | D10 | D13 | D18 | D25 | D30 | D43 | D49 | D52 | D59 | D63 | D70 | D74 | | 791 | |
| DRB1*1120 | D1 | D5 | D10 | D14 | D19 | D24 | D30 | D43 | D49 | D52 | D59 | D63 | D70 | D73 | | | |
| DRB1*1116 | D1 | D5 | D10 | D14 | D19 | D24 | D30 | D43 | D49 | D52 | D59 | D63 | D70 | D74 | | | |
| DRB1*1204 | D2 | D8 | D11 | D14 | D19 | D28 | D30 | D43 | D49 | D54 | D60 | D63 | D71 | D74 | D76 | 791 | |
| DRB1*1131 | D1 | D5 | D10 | D13 | D18 | D25 | D30 | D43 | D49 | D54 | D59 | D63 | D70 | D73 | | 718 | |
| DRB1*1133 | D1 | D5 | D10 | D13 | D18 | D25 | D30 | D43 | D50 | D54 | D59 | D63 | D70 | D73 | | 716 | |
| DRB1*1135 | D1 | D5 | D10 | D13 | D18 | D25 | D30 | D43 | D50 | D54 | D59 | D63 | D70 | D74 | | 716 | |
| DRB1*1138 | D1 | D5 | D10 | D13 | D18 | D25 | D30 | D43 | D50 | D54 | D59 | D63 | D70 | D74 | | 715 | |
| DRB1*1201 | D2 | D8 | D11 | D14 | D19 | D28 | D30 | D45 | D49 | D54 | D60 | D63 | D71 | D74 | D76 | 791 | |
| DRB1*1206 | D2 | D8 | D11 | D14 | D19 | D28 | D30 | D45 | D49 | D54 | D60 | D63 | D71 | D74 | D76 | 791 | |
| DRB1*12032 | D2 | D8 | D11 | D14 | D19 | D28 | D30 | D45 | D49 | D54 | D60 | D63 | D70 | D74 | D76 | | |
| DRB1*1205 | D2 | D8 | D11 | D14 | D19 | D26 | D30 | D45 | D49 | D54 | D60 | D63 | D71 | D74 | D76 | 791 | |
| DRB1*12021 | D2 | D8 | D11 | D14 | D19 | D28 | D30 | D45 | D50 | D54 | D60 | D63 | D71 | D74 | D76 | 791 | |
| DRB1*12022 | D2 | D8 | D11 | D14 | D19 | D28 | D30 | D45 | D50 | D54 | D60 | D63 | D70 | D74 | D76 | 791 | |
| DRB1*07011 | D1 | D5 | DD11 | D15 | D20 | D26 | D29 | D45 | D49 | D58 | D59 | D67 | D72 | D73 | | | |
| DRB1*07012 | D1 | D5 | DD11 | D15 | D20 | D26 | D29 | D45 | D49 | D58 | D59 | D67 | D72 | D73 | | 595 | |
| DRB1*0703 | D1 | D5 | DD11 | D15 | D20 | D26 | D29 | D45 | D49 | D58 | D59 | D67 | D72 | D73 | | 631 | |
| DRB1*0704 | D1 | D5 | DD11 | D15 | D20 | D26 | D29 | D45 | D49 | D58 | D59 | D67 | D70 | D73 | | 783 | |
| DRB1*04032 | D1 | D5 | D10 | D16 | D25 | D29 | D33 | D48 | D51 | D59 | D65 | D70 | D74 | | | | |
| DRB1*1505 | D1 | D5 | D10 | D15 | D20 | D23 | D30 | D33 | D48 | D57 | D59 | D63 | D70 | D74 | | | |
| DRB1*1506 | D1 | D5 | D10 | D15 | D20 | D23 | D30 | D33 | D48 | D57 | D59 | D63 | D70 | D74 | | 698 | |
| DRB1*16021 | D1 | D5 | D10 | D15 | D20 | D23 | D29 | D33 | D48 | D54 | D60 | D63 | D70 | D73 | | | |
| DRB1*16022 | D1 | D5 | D10 | D15 | D20 | D23 | D29 | D33 | D48 | D54 | D59 | D63 | D70 | D73 | | | |
| DRB1*13072 | D1 | D5 | D10 | D13 | D18 | D25 | D29 | D33 | D50 | D53 | D59 | D63 | D70 | D73 | | | |
| DRB1*13142 | D1 | D5 | D10 | D13 | D18 | D25 | D30 | D33 | D50 | D54 | D59 | D63 | D70 | D73 | | | |
| DRB1*1326 | D1 | D5 | D10 | D14 | D19 | D24 | D29 | D33 | D50 | D54 | D60 | D63 | D70 | D73 | | | |
| DRB1*1608 | D1 | D5 | D10 | D15 | D20 | D24 | D29 | D33 | D50 | D54 | D60 | D63 | D70 | D73 | | | |
| DRB1*16011 | D1 | D5 | D10 | D15 | D20 | D23 | D29 | D33 | D50 | D54 | D60 | D63 | D70 | D73 | | | |
| DRB1*16012 | D1 | D5 | D10 | D15 | D20 | D23 | D29 | D33 | D50 | D54 | D59 | D63 | D70 | D73 | | | |
| DRB1*1603 | D1 | D5 | D10 | D15 | D20 | D23 | D29 | D33 | D50 | D54 | D61 | D63 | D70 | D73 | | | |
| DRB1*1604 | D1 | D5 | D10 | D15 | D20 | D23 | D29 | D33 | D50 | D54 | D59 | D64 | D70 | D73 | | | |
| DRB1*1504 | D1 | D5 | D10 | D15 | D20 | D23 | D30 | D33 | D50 | D57 | D59 | D63 | D70 | D74 | | | |
| DRB1*15011 | D1 | D5 | D10 | D15 | D20 | D23 | D30 | D33 | D49 | D57 | D59 | D63 | D70 | D74 | | | |
| DRB1*15012 | D1 | D5 | D10 | D15 | D20 | D23 | D30 | D33 | D49 | D57 | D59 | D63 | D70 | D74 | | 790 | |
| DRB1*1503 | D1 | D5 | D10 | D15 | D20 | D23 | D30 | D33 | D49 | D57 | D59 | D63 | D70 | D74 | | 632 | |
| DRB1*1509 | D1 | D5 | D10 | D15 | D20 | D23 | D30 | D33 | D49 | D57 | D59 | D63 | D70 | D74 | | 696 | |
| DRB1*1507 | D1 | D5 | D10 | D15 | D20 | D23 | D29 | D33 | D49 | D57 | D59 | D63 | D70 | D74 | | | |
| DRB1*15021 | D1 | D5 | D10 | D15 | D20 | D23 | D30 | D33 | D49 | D57 | D59 | D63 | D70 | D73 | | | |
| DRB1*15023 | D1 | D5 | D10 | D15 | D20 | D23 | D30 | D33 | D49 | D57 | D59 | D63 | D70 | D73 | | | |
| DRB1*1508 | D1 | D5 | D10 | D15 | D20 | D23 | D30 | D33 | D49 | D57 | D59 | D63 | D70 | D73 | | 725 | |
| DRB1*1110 | D1 | D5 | D10 | D15 | D20 | D23 | D30 | D33 | D49 | D52 | D59 | D63 | D70 | D74 | | | |
| DRB1*1605 | D1 | D5 | D10 | D15 | D20 | D23 | D29 | D33 | D49 | D54 | D60 | D63 | D70 | D73 | | | |
| DRB1*1607 | D1 | D5 | D10 | D15 | D20 | D23 | D29 | D33 | D49 | D54 | D60 | D63 | D70 | D73 | | 618 | |
| DRB1*0416 | D1 | D5 | D10 | D16 | D25 | D29 | D44 | D48 | D56 | D59 | D63 | D70 | D73 | | | | |
| DRB1*0426 | D1 | D5 | D10 | D16 | D25 | D29 | D41 | D48 | D56 | D59 | D63 | D70 | D73 | | | | |
| DRB1*0807 | D2 | D5 | D10 | D13 | D18 | D25 | D29 | D38 | D50 | D54 | D59 | D64 | D70 | D73 | | | |
| DRB1*0811 | D2 | D5 | D10 | D13 | D18 | D25 | D29 | D38 | D50 | D54 | D59 | D64 | D70 | D73 | | | |
| DRB1*0815 | D2 | D5 | D10 | D13 | D18 | D25 | D29 | D42 | D49 | D54 | D59 | D64 | D70 | D73 | | 717 | |
| DRB1*0819 | D2 | D5 | D10 | D13 | D18 | D25 | D29 | D40 | D49 | D54 | D59 | D64 | D70 | D73 | | | |
| DRB1*09012 | D1 | D7 | D12 | D13 | D18 | D24 | D29 | D38 | D50 | D55 | D59 | D65 | D72 | D73 | | | |
| DRB1*0808 | D2 | D5 | D10 | D13 | D18 | D25 | D29 | D36 | D50 | D54 | D59 | D64 | D70 | D73 | | | |
| DRB1*14011 | D1 | D5 | D10 | D14 | D19 | D26 | D29 | D36 | D48 | D55 | D59 | D65 | D71 | D74 | | | |
| DRB1*1407 | D1 | D5 | D10 | D14 | D19 | D26 | D29 | D36 | D48 | D55 | D59 | D65 | D71 | D73 | | | |
| DRB1*1410 | D1 | D5 | D10 | D14 | D19 | D26 | D29 | D36 | D48 | D55 | D59 | D65 | D71 | D74 | | 596 | |
| DRB1*1426 | D1 | D5 | D10 | D14 | D19 | D26 | D29 | D36 | D48 | D55 | D59 | D65 | D71 | D74 | | 608 | |
| DRB1*1404 | D2 | D5 | D10 | D14 | D19 | D26 | D29 | D36 | D48 | D55 | D59 | D65 | D71 | D74 | | | |
| DRB1*1428 | D2 | D5 | D10 | D14 | D19 | D26 | D29 | D36 | D48 | D55 | D59 | D65 | D71 | D74 | | 791 | |
| DRB1*1431 | D2 | D5 | D10 | D14 | D19 | D26 | D29 | D36 | D48 | D55 | D59 | D63 | D71 | D74 | | | |
| DRB1*1432 | D1 | D5 | D10 | D14 | D19 | D26 | D29 | D36 | D48 | D55 | D59 | D63 | D70 | D74 | | | |
| DRB1*1435 | D1 | D5 | D10 | D14 | D19 | D26 | D30 | D36 | D48 | D55 | D59 | D65 | D71 | D74 | | | |
| DRB1*1343 | D1 | D5 | D10 | D14 | D19 | D24 | D30 | D36 | D48 | D52 | D59 | D63 | D70 | D74 | | | |
| DRB1*1345 | D1 | D5 | D10 | D13 | D18 | D25 | D30 | D36 | D49 | D52 | D59 | D63 | D70 | D73 | | | |
| DRB1*1416 | D1 | D5 | D10 | D14 | D19 | D26 | D29 | D36 | D49 | D52 | D59 | D63 | D70 | D74 | | | |
| DRB1*0310 | D1 | D6 | D10 | D14 | D19 | D24 | D30 | D36 | D48 | D56 | D59 | D66 | D70 | D74 | | 782 | |
| DRB1*1422 | D1 | D5 | D10 | D14 | D19 | D26 | D29 | D36 | D50 | D54 | D59 | D63 | D70 | D73 | | | |
| DRB1*1425 | D1 | D5 | D10 | D13 | D18 | D25 | D29 | D36 | D50 | D54 | D59 | D63 | D70 | D73 | | | |
| DRB1*1331 | D1 | D5 | D10 | D14 | D19 | D24 | D30 | D34 | D49 | D52 | D59 | D63 | D70 | D73 | | | |
| DRB1*1339 | D1 | D5 | D10 | D14 | D19 | D24 | D30 | D35 | D49 | D52 | D59 | D63 | D70 | D73 | | | |
| DRB1*0313 | D1 | D6 | D10 | D14 | D19 | D24 | D30 | D35 | D48 | D56 | D59 | D66 | D70 | D74 | | 782 | |
| DRB1*1408 | D1 | D5 | D10 | D14 | D19 | D26 | D29 | D46 | D48 | D55 | D59 | D65 | D71 | D74 | | | |
| DRB1*1411 | D2 | D5 | D10 | D14 | D19 | D26 | D29 | D47 | D48 | D55 | D59 | D65 | D71 | D74 | | | |
| DRB1*1418 | D1 | D5 | D11 | D14 | D19 | D24 | D29 | D37 | D48 | D55 | D59 | D65 | D71 | D74 | | | |
| DRB1*1405 | D3 | D5 | D10 | D14 | D19 | D26 | D29 | D37 | D48 | D55 | D59 | D65 | D71 | D74 | | | |
| DRB1*1437 | D3 | D5 | D10 | D14 | D19 | D26 | D29 | D37 | D49 | D57 | D59 | D63 | D70 | D74 | | | |
| DRB1*1434 | D1 | D5 | D10 | D14 | D19 | D26 | D29 | D46 | D48 | D55 | D59 | D63 | D71 | D74 | | | |

TABLE 26
Correspondence of the patchworks and SEQ ID Nos for DRB1

| Patchwork | SEQ ID NO: | | | | | | | | | | | | | | |
|---|---|---|---|---|---|---|---|---|---|---|---|---|---|---|---|
| D1 | 600 | 603 | 604 | 605 | 606 | 607 | | | | | | | | | |
| D2 | 601 | | | | | | | | | | | | | | |
| D3 | 602 | | | | | | | | | | | | | | |
| D4 | 609 | | | | | | | | | | | | | | |
| D5 | 610 | 614 | 615 | 616 | 617 | | | | | | | | | | |
| D6 | 611 | | | | | | | | | | | | | | |
| D7 | 612 | | | | | | | | | | | | | | |
| D8 | 613 | | | | | | | | | | | | | | |
| D9 | 619 | 623 | 626 | 627 | | | | | | | | | | | |
| D10 | 620 | 624 | 627 | 629 | 630 | | | | | | | | | | |
| D11 | 621 | 625 | | | | | | | | | | | | | |
| D12 | 622 | | | | | | | | | | | | | | |
| D13 | 633 | 643 | 650 | | | | | | | | | | | | |
| D14 | 635 | 645 | | | | | | | | | | | | | |
| D15 | 637 | 647 | 653 | 657 | 661 | | | | | | | | | | |
| D16 | 639 | 649 | | | | | | | | | | | | | |
| D17 | 641 | | | | | | | | | | | | | | |
| D18 | 634 | 644 | 651 | 655 | 659 | 663 | | | | | | | | | |
| D19 | 636 | 646 | 652 | 656 | 660 | | | | | | | | | | |
| D20 | 638 | 648 | 654 | 658 | 662 | | | | | | | | | | |
| D21 | 640 | | | | | | | | | | | | | | |
| D22 | 642 | | | | | | | | | | | | | | |
| D23 | 667 | 673 | | | | | | | | | | | | | |
| D24 | 668 | 674 | 678 | | | | | | | | | | | | |
| D25 | 669 | 675 | 679 | 681 | 682 | 683 | | | | | | | | | |
| D26 | 670 | 676 | 680 | | | | | | | | | | | | |
| D27 | 671 | 677 | | | | | | | | | | | | | |
| D28 | 672 | | | | | | | | | | | | | | |
| D29 | 689 | 691 | 693 | 695 | | | | | | | | | | | |
| D30 | 690 | 692 | 694 | | | | | | | | | | | | |
| D31 | 701 | | | | | | | | | | | | | | |
| D32 | 702 | | | | | | | | | | | | | | |
| D33 | 703 | | | | | | | | | | | | | | |
| D34 | 704 | | | | | | | | | | | | | | |
| D35 | 705 | | | | | | | | | | | | | | |
| D36 | 706 | | | | | | | | | | | | | | |
| D37 | 707 | | | | | | | | | | | | | | |
| D38 | 708 | | | | | | | | | | | | | | |
| D39 | 709 | | | | | | | | | | | | | | |
| D40 | 710 | | | | | | | | | | | | | | |
| D41 | 711 | | | | | | | | | | | | | | |
| D42 | 712 | | | | | | | | | | | | | | |
| D43 | 713 | 722 | 723 | 724 | | | | | | | | | | | |
| D44 | 714 | | | | | | | | | | | | | | |
| D45 | 719 | | | | | | | | | | | | | | |
| D46 | 720 | | | | | | | | | | | | | | |
| D47 | 721 | | | | | | | | | | | | | | |
| D48 | 726 | 729 | | | | | | | | | | | | | |
| D49 | 727 | 730 | 732 | | | | | | | | | | | | |
| D50 | 728 | 731 | | | | | | | | | | | | | |
| D51 | 733 | 741 | | | | | | | | | | | | | |
| D52 | 734 | 742 | | | | | | | | | | | | | |
| D53 | 735 | 743 | | | | | | | | | | | | | |
| D54 | 736 | 744 | | | | | | | | | | | | | |
| D55 | 737 | 745 | 747 | | | | | | | | | | | | |
| D56 | 738 | 746 | | | | | | | | | | | | | |
| D57 | 739 | | | | | | | | | | | | | | |
| D58 | 740 | | | | | | | | | | | | | | |
| D59 | 748 | 754 | 756 | 758 | 759 | 760 | 761 | 762 | 763 | 764 | 765 | 767 | 768 | 769 | 770 |
| D60 | 749 | 755 | 757 | | | | | | | | | | | | |
| D61 | 750 | | | | | | | | | | | | | | |
| D62 | 752 | | | | | | | | | | | | | | |
| D63 | 771 | 778 | 779 | 780 | 781 | | | | | | | | | | |
| D64 | 772 | | | | | | | | | | | | | | |
| D65 | 773 | | | | | | | | | | | | | | |
| D66 | 774 | | | | | | | | | | | | | | |
| D67 | 775 | | | | | | | | | | | | | | |
| D68 | 776 | | | | | | | | | | | | | | |
| D69 | 777 | | | | | | | | | | | | | | |
| D70 | 784 | 787 | 788 | | | | | | | | | | | | |
| D71 | 785 | | | | | | | | | | | | | | |
| D72 | 786 | | | | | | | | | | | | | | |
| D73 | 792 | | | | | | | | | | | | | | |
| D74 | 793 | 795 | 796 | 797 | 798 | | | | | | | | | | |
| D75 | 794 | | | | | | | | | | | | | | |
| D76 | 597 | 598 | 599 | | | | | | | | | | | | |

TABLE 27
Correspondence of the patchwork regions and satellite regions for types of DRB 3-5

| | patchwork | | | | | | | | | | | | | | | | satellite | | |
|---|---|---|---|---|---|---|---|---|---|---|---|---|---|---|---|---|---|---|---|
| DRB3*01011 | R1 | R3 | R7 | R17 | R19 | R21 | R25 | R27 | R31 | R32 | R34 | R40 | R43 | R45 | R50 | R52 | 832 | | |
| DRB3*0101201 | R1 | R3 | R7 | R17 | R19 | R21 | R25 | R27 | R31 | R32 | R34 | R40 | R43 | R45 | R50 | R52 | 832 | | |
| DRB3*0101202 | R1 | R3 | R7 | R17 | R19 | R21 | R25 | R27 | R31 | R32 | R34 | R40 | R43 | R45 | R50 | R52 | 832 | | |
| DRB3*01014 | R1 | R3 | R7 | R17 | R19 | R21 | R25 | R27 | R31 | R32 | R34 | R40 | R43 | R45 | R50 | R52 | 832 | 847 | |
| DRB3*0102 | R1 | R3 | R7 | R17 | R19 | R21 | R25 | R27 | R31 | R32 | R34 | R40 | R43 | R45 | R50 | R52 | 832 | 853 | |
| DRB3*0103 | R1 | R3 | R8 | R17 | R19 | R21 | R25 | R27 | R31 | R32 | R34 | R40 | R43 | R45 | R50 | R52 | 832 | | |
| DRB3*0104 | R1 | R3 | R7 | R17 | R19 | R21 | R25 | R27 | R31 | R32 | R34 | R40 | R43 | R45 | R50 | R52 | 832 | 852 | |
| DRB3*0105 | R1 | R3 | R10 | R17 | R19 | R21 | R25 | R27 | R31 | R32 | R34 | R40 | R43 | R45 | R50 | R52 | 832 | | |
| DRB3*0106 | R1 | R3 | R7 | R17 | R19 | R21 | R25 | R27 | R31 | R32 | R34 | R40 | R43 | R45 | R50 | R52 | | | |
| DRB3*0209 | R1 | R2 | R8 | R16 | R19 | R21 | R24 | R27 | R31 | R32 | R34 | R40 | R43 | R46 | R50 | R52 | 832 | | |
| DRB3*0301 | R1 | R2 | R8 | R17 | R19 | R21 | R25 | R27 | R31 | R32 | R34 | R40 | R43 | R46 | R50 | R53 | | | |
| DRB3*03012 | R1 | R2 | R8 | R17 | R19 | R21 | R25 | R27 | R31 | R32 | R34 | R40 | R43 | R46 | R50 | R53 | 832 | | |
| DRB3*0302 | R1 | R2 | R8 | R16 | R19 | R21 | R25 | R27 | R31 | R32 | R34 | R40 | R43 | R46 | R50 | R53 | | | |
| DRB3*0303 | R1 | R2 | R8 | R17 | R19 | R21 | R25 | R27 | R31 | R32 | R34 | R40 | R43 | R45 | R50 | R52 | | | |
| DRB3*01013 | R1 | R3 | R7 | R17 | R19 | R21 | R25 | R27 | R32 | R33 | R34 | R40 | R43 | R45 | R50 | R52 | 832 | | |
| DRB3*0107 | R1 | R3 | R7 | R17 | R19 | R21 | R24 | R27 | R28 | R34 | R40 | R43 | R46 | R50 | R52 | | 834 | 847 | |
| DRB3*02022 | R1 | R2 | R8 | R16 | R19 | R21 | R24 | R27 | R28 | R34 | R40 | R43 | R46 | R50 | R52 | | 834 | 847 | |
| DRB3*02010 | R1 | R2 | R8 | R16 | R19 | R21 | R24 | R27 | R28 | R34 | R40 | R43 | R46 | R50 | R52 | | 832 | | |
| DRB3*02012 | R1 | R2 | R8 | R16 | R19 | R21 | R24 | R27 | R28 | R34 | R40 | R43 | R46 | R50 | R52 | | 832 | 847 | |
| DRB3*0205 | R1 | R2 | R8 | R17 | R19 | R21 | R24 | R27 | R28 | R34 | R40 | R43 | R46 | R50 | R52 | | 832 | 847 | |
| DRB3*02021 | R1 | R2 | R8 | R16 | R19 | R21 | R24 | R27 | R28 | R34 | R40 | R43 | R46 | R50 | R52 | | 834 | 847 | |
| DRB3*02011 | R1 | R2 | R8 | R16 | R19 | R21 | R24 | R27 | R28 | R35 | R40 | R43 | R46 | R50 | R52 | | 832 | 847 | |
| DRB3*02023 | R1 | R2 | R8 | R16 | R19 | R21 | R24 | R27 | R28 | R34 | R40 | R43 | R46 | R50 | R52 | | 834 | 847 | 845 |
| DRB3*0201 | R1 | R2 | R8 | R16 | R19 | R21 | R24 | R27 | R28 | R34 | R40 | R43 | R46 | R50 | R53 | | 834 | 847 | |
| DRB3*0203 | R1 | R2 | R8 | R16 | R19 | R21 | R22 | R27 | R28 | R34 | R40 | R43 | R46 | R50 | R52 | | 847 | | |
| DRB3*0206 | R1 | R2 | R8 | R16 | R19 | R21 | R23 | R27 | R28 | R34 | R40 | R43 | R46 | R50 | R52 | | 832 | 847 | |
| DRB3*0204 | R1 | R2 | R8 | R16 | R19 | R21 | R24 | R27 | R28 | R34 | R40 | R43 | R45 | R50 | R52 | | 832 | 847 | |
| DRB3*02013 | R1 | R4 | R8 | R16 | R19 | R21 | R24 | R27 | R28 | R34 | R40 | R43 | R46 | R50 | R52 | | 832 | 847 | |
| DRB3*0207 | R1 | R2 | R8 | R16 | R19 | R21 | R24 | R27 | R31 | R34 | R40 | R43 | R46 | R50 | R52 | | 832 | 847 | |
| DRB3*0208 | R1 | R2 | R8 | R16 | R19 | R21 | R24 | R27 | R29 | R34 | R40 | R43 | R46 | R50 | R52 | | 832 | 847 | |
| DRB4*0103101 | R1 | R5 | R10 | R14 | R18 | R20 | R24 | R27 | R30 | R34 | R39 | R43 | R48 | R50 | R53 | | | | |
| DRB4*01032 | R1 | R5 | R10 | R14 | R18 | R20 | R24 | R27 | R30 | R34 | R39 | R43 | R48 | R50 | R53 | | | | |
| DRB4*01011 | R1 | R5 | R10 | R14 | R18 | R20 | R24 | R27 | R30 | R34 | R39 | R43 | R48 | R50 | R53 | | | | |
| DRB4*01033 | R1 | R5 | R10 | R14 | R18 | R20 | R24 | R27 | R30 | R34 | R39 | R43 | R48 | R51 | R53 | | | | |
| DRB4*0102 | | R5 | R10 | R14 | R18 | R20 | R24 | R27 | R30 | R34 | R39 | R43 | R48 | R50 | R53 | | 843 | | |
| DRB4*0104 | R1 | R5 | R10 | R14 | R18 | R20 | R24 | R27 | R30 | R34 | R39 | R43 | R48 | R50 | R53 | | 844 | | |
| DRB4*0105 | R1 | R5 | R10 | R14 | R18 | R20 | R24 | R27 | R30 | R34 | R39 | R43 | R48 | R50 | R53 | | | | |
| DRB4*0201N | R1 | R6 | R11 | R14 | R18 | R20 | R24 | R27 | R30 | R34 | R39 | R43 | R48 | R50 | R53 | | | | |
| DRB5*01011 | R1 | R2 | R12 | R15 | R18 | R20 | R26 | R27 | R30 | R36 | R38 | R44 | R49 | R50 | R52 | | 833 | | |
| DRB5*01012 | R1 | R2 | R12 | R15 | R18 | R20 | R26 | R27 | R30 | R36 | R38 | R43 | R49 | R50 | R52 | | 833 | | |
| DRB5*0105 | R1 | R2 | R12 | R15 | R18 | R20 | R26 | R27 | R30 | R36 | R38 | R44 | R49 | R50 | R52 | | | | |
| DRB5*0104 | R1 | R2 | R12 | R15 | R18 | R20 | R26 | R27 | R30 | R36 | R38 | R43 | R47 | R50 | R52 | | | | |
| DRB5*0106 | R1 | R2 | R12 | R15 | R18 | R20 | R26 | R27 | R30 | R35 | R41 | R43 | R49 | R50 | R53 | | | | |
| DRB5*0107 | R1 | R2 | R12 | R15 | R18 | R20 | R26 | R27 | R30 | R35 | R38 | R44 | R49 | R50 | R52 | | | | |
| DRB5*0109 | R1 | R2 | R12 | R15 | R18 | R20 | R26 | R27 | R30 | R36 | R42 | R44 | R49 | R50 | R52 | | | | |
| DRB5*0108N | R1 | R2 | R12 | R13 | R18 | R20 | R23 | R27 | R30 | R36 | R38 | R44 | R49 | R50 | R52 | | | | |
| DRB5*0102 | R1 | R2 | R12 | R13 | R18 | R20 | R23 | R27 | R30 | R36 | R38 | R44 | R49 | R50 | R52 | | | | |
| DRB5*0110N | R1 | R2 | R12 | R13 | R18 | R20 | R23 | R27 | R30 | R36 | R38 | R44 | R49 | R50 | R52 | | | | |
| DRB5*0103 | R1 | R2 | R12 | R13 | R18 | R20 | R23 | R27 | R30 | R36 | R31 | R44 | R49 | R50 | R52 | | | | |
| DRB5*0202 | R1 | R2 | R12 | R13 | R18 | R20 | R23 | R27 | R30 | R35 | R41 | R43 | R49 | R50 | R53 | | | | |
| DRB5*0203 | R1 | R2 | R12 | R13 | R18 | R20 | R23 | R27 | R30 | R35 | R41 | R43 | R49 | R50 | R52 | | | | |
| DRB5*0204 | R1 | R2 | R12 | R13 | R18 | R20 | R23 | R27 | R30 | R36 | R41 | R43 | R49 | R50 | R53 | | 848 | | |
| DRB5*0205 | R1 | R2 | R12 | R13 | R18 | R20 | R23 | R27 | R30 | R34 | R37 | R43 | R49 | R50 | R53 | | 848 | | |

TABLE 28
Correspondence of the patchworks and SEQ ID NOs for DRB 3-5

| Patchwork | SEQ ID NO: | | | | |
|---|---|---|---|---|---|
| R1 | 603 | 604 | 607 | | |
| R2 | 610 | 803 | | | |
| R3 | 611 | 804 | 805 | | |
| R4 | 800 | | | | |
| R5 | 801 | | | | |
| R6 | 802 | | | | |
| R7 | 620 | | | | |
| R8 | 621 | 810 | 812 | 813 | |
| R9 | 806 | | | | |
| R10 | 807 | | | | |
| R11 | 808 | | | | |
| R12 | 809 | 811 | | | |
| R13 | 814 | | | | |
| R14 | 815 | | | | |
| R15 | 816 | | | | |
| R16 | 817 | | | | |
| R17 | 818 | 819 | 820 | | |
| R18 | 643 | 821 | 822 | | |
| R19 | 823 | 824 | | | |
| R20 | 825 | 826 | 659 | | |
| R21 | 827 | 828 | 652 | 829 | |
| R22 | 673 | | | | |
| R23 | 668 | 678 | | | |
| R24 | 682 | 831 | | | |
| R25 | 676 | 680 | | | |
| R26 | 830 | | | | |
| R27 | 689 | 691 | | | |
| R28 | 701 | | | | |
| R29 | 702 | | | | |
| R30 | 703 | | | | |
| R31 | 704 | | | | |
| R32 | 837 | | | | |
| R33 | 838 | | | | |
| R34 | 726 | | | | |
| R35 | 727 | 730 | | | |
| R36 | 728 | 731 | | | |
| R37 | 733 | | | | |
| R38 | 736 | 744 | 839 | | |
| R39 | 737 | | | | |
| R40 | 746 | | | | |
| R41 | 739 | | | | |
| R42 | 1072 | | | | |
| R43 | 658 | 659 | 662 | 667 | 841 |
| R44 | 655 | 840 | | | |
| R45 | 774 | | | | |
| R46 | 775 | | | | |
| R47 | 772 | 842 | | | |
| R48 | 773 | | | | |
| R49 | 771 | 780 | | | |
| R50 | 784 | 787 | 788 | | |
| R51 | 785 | | | | |
| R52 | 792 | 850 | | | |
| R53 | 793 | 795 | 851 | | |

TABLE 29
Correspondence of the patchwork regions and satellite regions for types of A

| | patchwork region | | | | | | | | | | | | | | | | |
|---|---|---|---|---|---|---|---|---|---|---|---|---|---|---|---|---|---|
| A*01011 | 1A | 6A | 9A | 14A | 19A | 25A | 29A | 33A | 36A | 45A | 47A | 52A | | | | | |
| A*0106 | 1A | 6A | 9A | 14A | 19A | 25A | 29A | 33A | 36A | 45A | 48A | 52A | | | | | |
| A*0108 | 1A | 6A | 9A | 14A | 19A | 25A | 29A | 33A | 36A | 45A | 47A | 53A | | | | | |
| A*3601 | 1A | 6A | 9A | 14A | 19A | 25A | 29A | 33A | 36A | 45A | 47A | 54A | 64A | | | | |
| A*3602 | 1A | 6A | 9A | 14A | 19A | 25A | 29A | 33A | 36A | 45A | 47A | 54A | 64A | | | | |
| A*01012 | 1A | 7A | 9A | 14A | 19A | 25A | 29A | 33A | 36A | 45A | 47A | 52A | | | | | |
| A*0102 | 3A | 7A | 9A | 14A | 19A | 25A | 29A | 33A | 36A | 45A | 47A | 52A | | | | | |
| A*0107 | 1A | 6A | 10A | 14A | 20A | 25A | 30A | 33A | 36A | 45A | 47A | 52A | | | | | |
| A*3002 | 3A | 7A | 10A | 14A | 20A | 25A | 30A | 33A | 36A | 44A | 48A | 54A | 60A | 63A | 64A | 58A | |
| A*3009 | 3A | 7A | 10A | 14A | 20A | 25A | 30A | 33A | 36A | 44A | 48A | 54A | 60A | 61A | 64A | 58A | |
| A*3003 | 3A | 7A | 9A | 14A | 20A | 25A | 30A | 33A | 36A | 44A | 48A | 54A | 60A | 63A | 64A | 58A | |
| A*3004 | 3A | 7A | 10A | 14A | 20A | 25A | 30A | 33A | 36A | 44A | 49A | 54A | 61A | 64A | | 58A | |
| A*3006 | 3A | 7A | 10A | 14A | 20A | 25A | 30A | 33A | 36A | 44A | 49A | 54A | 61A | 64A | | 58A | |
| A*03011 | 1A | 6A | 9A | 14A | 20A | 25A | 31A | 33A | 36A | 45A | 48A | 54A | 62A | 64A | | 58A | |
| A*0304 | 1A | 6A | 9A | 14A | 20A | 25A | 31A | 33A | 36A | 45A | 48A | 54A | 62A | 64A | | 58A | |
| A*0308 | 1A | 6A | 9A | 14A | 20A | 25A | 31A | 33A | 36A | 45A | 48A | 54A | 62A | 64A | 58A | | |
| A*0305 | 1A | 6A | 9A | 14A | 20A | 25A | 31A | 33A | 36A | 45A | 48A | 54A | 62A | 64A | 58A | | |
| A*0306 | 1A | 6A | 9A | 14A | 20A | 25A | 31A | 33A | 36A | 45A | 48A | 54A | 62A | 64A | 58A | | |
| A*0307 | 1A | 6A | 9A | 14A | 20A | 25A | 31A | 33A | 36A | 45A | 48A | 54A | 61A | 64A | 58A | | |
| A*03013 | 1A | 6A | 11A | 14A | 20A | 25A | 31A | 33A | 36A | 45A | 48A | 54A | 62A | 64A | 58A | | |
| A*3103 | 4A | 6A | 10A | 14A | 20A | 25A | 31A | 33A | 36A | 45A | 48A | 54A | 60A | 61A | 64A | 58A | |
| A*3104 | 4A | 6A | 10A | 14A | 20A | 25A | 31A | 33A | 36A | 45A | 48A | 54A | 60A | 61A | 64A | 58A | |
| A*3001 | 3A | 7A | 10A | 14A | 20A | 25A | 31A | 33A | 36A | 44A | 48A | 54A | 60A | 63A | 64A | 58A | |
| A*3008 | 2A | 7A | 10A | 14A | 20A | 25A | 31A | 33A | 36A | 44A | 48A | 54A | 60A | 63A | 64A | 58A | |
| A*03012 | 1A | 6A | 9A | 14A | 20A | 25A | 31A | 33A | 36A | 45A | 51A | 54A | 62A | 64A | 58A | | |
| A*0302 | 1A | 6A | 9A | 14A | 20A | 25A | 31A | 33A | 36A | 45A | 50A | 54A | 62A | 64A | 58A | | |
| A*11011 | 2A | 6A | 9A | 14A | 20A | 25A | 31A | 33A | 36A | 45A | 50A | 52A | 64A | | | | |
| A*11012 | 2A | 6A | 9A | 14A | 20A | 25A | 31A | 33A | 36A | 45A | 50A | 52A | 64A | | | | |
| A*1102 | 2A | 6A | 9A | 14A | 20A | 25A | 31A | 33A | 36A | 45A | 50A | 52A | 64A | | | | |
| A*1103 | 2A | 6A | 9A | 14A | 20A | 25A | 31A | 33A | 36A | 45A | 50A | 52A | 60A | 62A | 64A | | |
| A*1105 | 2A | 6A | 9A | 14A | 20A | 25A | 31A | 33A | 36A | 45A | 50A | 52A | 64A | | | | |
| A*1106 | 2A | 6A | 9A | 14A | 20A | 26A | 31A | 33A | 36A | 45A | 50A | 52A | 64A | | | | |
| A*1107 | 2A | 6A | 9A | 14A | 20A | 25A | 31A | 33A | 36A | 45A | 50A | 52A | 64A | | | | |
| A*1109 | 2A | 6A | 9A | 14A | 20A | 25A | 31A | 33A | 36A | 45A | 50A | 52A | 64A | | | | |
| A*1104 | 2A | 6A | 9A | 14A | 20A | 25A | 31A | 33A | 36A | 45A | 50A | 54A | 64A | | | | |
| A*1108 | 2A | 6A | 9A | 14A | 20A | 25A | 31A | 33A | 36A | 45A | 47A | 52A | 62A | 64A | | | |
| A*3204 | 1A | 6A | 9A | 14A | 20A | 25A | 32A | 33A | 36A | 45A | 48A | 54A | 62A | 64A | 58A | | |
| A*0103 | 1A | 6A | 9A | 14A | 19A | 25A | 29A | 33A | 37A | 45A | 47A | 52A | | | | | |
| A*3203 | 1A | 6A | 9A | 14A | 20A | 25A | 30A | 33A | 37A | 41A | 48A | 54A | 60A | 61A | 64A | | |
| A*31012 | 4A | 6A | 10A | 14A | 20A | 25A | 31A | 33A | 37A | 41A | 48A | 54A | 60A | 61A | 64A | 58A | |
| A*3105 | 4A | 6A | 10A | 14A | 20A | 25A | 31A | 33A | 37A | 41A | 48A | 54A | 60A | 61A | 58A | | |
| A*3102 | 4A | 6A | 10A | 14A | 21A | 25A | 31A | 33A | 37A | 41A | 48A | 54A | 60A | 61A | 64A | 58A | |
| A*7401 | 1A | 6A | 9A | 14A | 20A | 25A | 31A | 33A | 37A | 41A | 48A | 54A | 60A | 61A | 64A | | |
| A*7402 | 1A | 6A | 9A | 14A | 20A | 25A | 31A | 33A | 37A | 41A | 48A | 54A | 60A | 61A | 64A | | |
| A*7403 | 1A | 6A | 9A | 14A | 20A | 25A | 31A | 33A | 37A | 41A | 48A | 54A | 60A | 61A | 64A | | |
| A*7405 | 1A | 6A | 9A | 14A | 20A | 25A | 31A | 33A | 37A | 41A | 48A | 54A | 60A | 61A | 64A | | |
| A*3202 | 1A | 6A | 9A | 14A | 20A | 25A | 32A | 33A | 37A | 41A | 50A | 54A | 61A | 64A | | | |
| A*3201 | 1A | 6A | 9A | 14A | 20A | 25A | 32A | 33A | 37A | 41A | 48A | 54A | 60A | 61A | 64A | | |
| A*3206 | 1A | 6A | 9A | 14A | 20A | 25A | 32A | 33A | 37A | 41A | 48A | 54A | 61A | 64A | | | |
| A*3205 | 1A | 6A | 9A | 14A | 21A | 25A | 32A | 33A | 37A | 41A | 48A | 54A | 60A | 61A | 64A | | |
| A*0245 | 1A | 7A | 11A | 14A | 21A | 26A | 31A | 34A | 38A | 39A | 48A | 54A | 61A | 64A | | | |
| A*0204 | 1A | 7A | 11A | 15A | 21A | 26A | 31A | 34A | 37A | 39A | 48A | 54A | 61A | 64A | | | |
| A*02171 | 1A | 7A | 11A | 15A | 21A | 26A | 31A | 35A | 37A | 39A | 48A | 54A | 61A | 64A | | | |
| A*02172 | 1A | 7A | 11A | 15A | 21A | 26A | 31A | 35A | 37A | 39A | 48A | 54A | 61A | 64A | | | |
| A*7404 | 1A | 6A | 11A | 15A | 21A | 25A | 31A | 33A | 37A | 41A | 48A | 54A | 60A | 61A | 64A | | |
| A*2408 | 3A | 6A | 9A | 15A | 21A | 25A | 30A | 35A | 37A | 39A | 50A | 54A | 61A | 58A | | | |
| A*02012 | 1A | 7A | 11A | 15A | 21A | 26A | 31A | 34A | 38A | 39A | 48A | 54A | 61A | 64A | | | |
| A*02013 | 1A | 7A | 11A | 15A | 21A | 27A | 31A | 34A | 38A | 39A | 48A | 54A | 61A | 64A | | | |
| A*02014 | 1A | 7A | 11A | 15A | 21A | 26A | 31A | 34A | 38A | 39A | 48A | 54A | 61A | 64A | | | |
| A*02015 | 1A | 7A | 11A | 15A | 21A | 26A | 31A | 34A | 38A | 39A | 48A | 54A | 61A | 64A | | | |
| A*0209 | 1A | 7A | 11A | 15A | 21A | 26A | 31A | 34A | 38A | 39A | 48A | 54A | 61A | 64A | | | |
| A*0224 | 1A | 7A | 11A | 15A | 21A | 26A | 31A | 34A | 38A | 39A | 48A | 54A | 61A | 64A | | | |
| A*0236 | 1A | 7A | 11A | 15A | 21A | 26A | 31A | 34A | 38A | 39A | 48A | 54A | 61A | | | | |
| A*02016 | 1A | 7A | 11A | 15A | 21A | 26A | 31A | 34A | 38A | 39A | 48A | 54A | 61A | 64A | | | |
| A*0207 | 1A | 7A | 11A | 15A | 21A | 26A | 31A | 34A | 38A | 39A | 48A | 54A | 61A | 64A | | | |
| A*0218 | 1A | 7A | 11A | 15A | 21A | 26A | 31A | 34A | 38A | 39A | 48A | 54A | 61A | 64A | | | |
| A*0225 | 1A | 7A | 11A | 15A | 21A | 26A | 31A | 34A | 38A | 39A | 48A | 54A | 61A | 64A | | | |
| A*0226 | 1A | 7A | 11A | 15A | 21A | 26A | 31A | 34A | 38A | 39A | 48A | 54A | 62A | 64A | | | |
| A*0230 | 1A | 7A | 11A | 15A | 21A | 26A | 31A | 34A | 38A | 39A | 48A | 54A | 61A | 64A | | | |
| A*0231 | 1A | 7A | 11A | 15A | 21A | 26A | 31A | 34A | 38A | 39A | 48A | 54A | 61A | 64A | | | |
| A*0233 | 1A | 7A | 11A | 15A | 21A | 26A | 31A | 34A | 38A | 39A | 48A | 54A | 61A | 64A | | | |
| A*0234 | 1A | 7A | 11A | 15A | 21A | 26A | 31A | 34A | 38A | 39A | 48A | 54A | 61A | 64A | | | |
| A*0239 | 1A | 7A | 11A | 15A | 21A | 26A | 31A | 34A | 38A | 39A | 48A | 54A | 61A | 64A | | | |
| A*0240 | 1A | 7A | 11A | 15A | 21A | 26A | 31A | 34A | 38A | 39A | 48A | 54A | 60A | 61A | 64A | | |
| A*0211 | 1A | 7A | 11A | 15A | 21A | 25A | 31A | 34A | 38A | 39A | 48A | 54A | 61A | 64A | | | |
| A*02201 | 1A | 7A | 11A | 15A | 20A | 26A | 31A | 34A | 38A | 39A | 48A | 54A | 61A | 64A | | | |
| A*02202 | 1A | 7A | 11A | 15A | 22A | 26A | 31A | 34A | 38A | 39A | 48A | 54A | 61A | 64A | | | |
| A*0229 | 1A | 7A | 11A | 15A | 23A | 26A | 31A | 34A | 38A | 39A | 48A | 54A | 61A | 64A | | | |
| A*0235 | 1A | 7A | 11A | 15A | 21A | 25A | 31A | 34A | 38A | 39A | 48A | 54A | 61A | 64A | | | |
| A*02011 | 1A | 7A | 11A | 15A | 21A | 26A | 31A | 34A | 38A | 39A | 47A | 54A | 61A | 64A | | | |
| A*0242 | 1A | 8A | 11A | 15A | 21A | 26A | 31A | 34A | 38A | 39A | 48A | 54A | 61A | 64A | | | |
| A*0216 | 1A | 7A | 11A | 15A | 21A | 26A | 31A | 34A | 38A | 39A | 48A | 55A | 61A | 64A | | | |
| A*0206 | 2A | 7A | 11A | 15A | 21A | 26A | 31A | 34A | 38A | 39A | 48A | 54A | 61A | 64A | | | |
| A*0241 | 2A | 7A | 11A | 15A | 21A | 26A | 31A | 34A | 38A | 39A | 48A | 54A | 61A | 64A | | | |
| A*0210 | 2A | 7A | 11A | 15A | 21A | 26A | 31A | 34A | 38A | 39A | 48A | 54A | 61A | 64A | | | |
| A*0221 | 2A | 7A | 11A | 15A | 21A | 26A | 31A | 34A | 38A | 39A | 48A | 54A | 61A | 64A | | | |
| A*0244 | 2A | 7A | 11A | 15A | 21A | 26A | 31A | 34A | 38A | 39A | 50A | 54A | 61A | 64A | | | |
| A*0228 | 2A | 7A | 12A | 15A | 21A | 26A | 31A | 34A | 38A | 39A | 48A | 54A | 61A | 64A | | | |
| A*0214 | 2A | 7A | 11A | 15A | 21A | 26A | 31A | 35A | 38A | 39A | 48A | 54A | 61A | 64A | | | |
| A*0205 | 2A | 7A | 11A | 15A | 21A | 26A | 31A | 35A | 38A | 39A | 49A | 54A | 61A | 64A | | | |
| A*0208 | 2A | 7A | 11A | 15A | 20A | 26A | 31A | 35A | 38A | 39A | 49A | 54A | 61A | 64A | | | |
| A*0202 | 1A | 7A | 11A | 15A | 21A | 26A | 31A | 35A | 38A | 39A | 49A | 54A | 61A | 64A | | | |
| A*0247 | 1A | 7A | 11a | 15A | 21A | 26A | 31A | 35A | 38A | 39A | 49A | 54A | 61A | 64A | | | |
| A*0203 | 1A | 7A | 11A | 15A | 21A | 26A | 31A | 34A | 38A | 39A | 49A | 54A | 62A | 64A | | | |
| A*0222 | 1A | 7A | 11A | 15A | 21A | 26A | 31A | 34A | 38A | 39A | 49A | 54A | 61A | 64A | | | |
| A*0212 | 1A | 7A | 11A | 15A | 21A | 26A | 31A | 34A | 38A | 39A | 50A | 54A | 61A | 64A | | | |
| A*0213 | 1A | 7A | 11A | 15A | 21A | 26A | 31A | 34A | 38A | 39A | 50A | 54A | 62A | 64A | | | |
| A*0219 | 1A | 7A | 11A | 15A | 21A | 26A | 31A | 34A | 38A | 39A | 50A | 54A | 61A | | | | |
| A*0227 | 1A | 7A | 11A | 15A | 21A | 26A | 31A | 34A | 38A | 39A | 50A | 54A | 64A | | | | |
| A*0237 | 1A | 7A | 11A | 15A | 21A | 26A | 31A | 34A | 38A | 39A | 50A | 54A | 61A | | | | |
| A*0238 | 1A | 7A | 11A | 15A | 21A | 26A | 31A | 34A | 38A | 39A | 50A | 52A | 62A | 64A | | | |
| A*2607 | 2A | 6A | 9A | 15A | 21A | 25A | 29A | 33A | 38A | 41A | 49A | 52A | 62A | 64A | | | |
| A*3007 | 3A | 7A | 10A | 16A | 21A | 25A | 30A | 33A | 36A | 44A | 48A | 54A | 57A | 58A | 60A | 63A | 64A |
| A*8001 | 1A | 7A | 13A | 16A | 20A | 28A | 29A | 33A | 36A | 45A | 49A | 55A | 58A | 60A | 63A | | |
| A*2404 | 3A | 6A | 9A | 16A | 21A | 25A | 29A | 35A | 37A | 39A | 50A | 54A | 57A | 58A | 61A | | |
| A*2301 | 3A | 6A | 9A | 16A | 21A | 25A | 30A | 35A | 37A | 39A | 48A | 54A | 57A | 58A | 60A | 61A | |
| A*2303 | 3A | 6A | 9A | 16A | 21A | 25A | 30A | 35A | 37A | 39A | 48A | 54A | 57A | 58A | 60A | 61A | |
| A*2304 | 3A | 6A | 9A | 16A | 21A | 25A | 30A | 35A | 37A | 39A | 48A | 54A | 57A | 58A | 60A | 61A | 64A |
| A*2305 | 3A | 6A | 9A | 16A | 21A | 25A | 30A | 35A | 37A | 39A | 48A | 54A | 57A | 58A | 60A | 61A | |
| A*2306 | 3A | 6A | 9A | 16A | 21A | 25A | 30A | 35A | 37A | 39A | 48A | 54A | 57A | 58A | 60A | 61A | |
| A*2413 | 3A | 6A | 9A | 16A | 21A | 25A | 30A | 35A | 37A | 39A | 48A | 54A | 57A | 58A | 61A | | |
| A*2418 | 3A | 6A | 9A | 16A | 21A | 25A | 30A | 35A | 37A | 39A | 48A | 54A | 57A | 58A | 62A | 64A | |
| A*2302 | 3A | 6A | 9A | 16A | 21A | 25A | 30A | 35A | 37A | 39A | 49A | 54A | 57A | 58A | 60A | 61A | |
| A*2406 | 3A | 6A | 9A | 16A | 21A | 25A | 30A | 35A | 37A | 39A | 49A | 54A | 57A | 58A | 61A | | |
| A*2422 | 3A | 6A | 9A | 16A | 21A | 25A | 30A | 35A | 37A | 39A | 49A | 54A | 57A | 58A | 61A | 64A | |
| A*2402101 | 3A | 6A | 9A | 16A | 21A | 25A | 30A | 35A | 37A | 39A | 50A | 54A | 57A | 58A | 61A | | |
| A*2402102L | 3A | 6A | 9A | 16A | 21A | 25A | 30A | 35A | 37A | 39A | 50A | 54A | 57A | 58A | 61A | | |
| A*24031 | 3A | 6A | 9A | 16A | 21A | 25A | 30A | 35A | 37A | 39A | 50A | 54A | 57A | 58A | 61A | 64A | |
| A*24022 | 3A | 6A | 9A | 16A | 21A | 25A | 30A | 35A | 37A | 39A | 50A | 54A | 57A | 58A | 61A | | |
| A*2405 | 3A | 6A | 9A | 16A | 21A | 25A | 30A | 35A | 37A | 39A | 50A | 54A | 57A | 58A | 61A | | |
| A*2407 | 3A | 6A | 9A | 16A | 21A | 25A | 30A | 35A | 37A | 39A | 50A | 54A | 57A | 58A | 61A | | |
| A*2415 | 3A | 6A | 9A | 16A | 21A | 25A | 30A | 35A | 37A | 39A | 50A | 54A | 57A | 61A | | | |
| A*2417 | 3A | 6A | 9A | 16A | 21A | 25A | 30A | 35A | 37A | 45A | 50A | 54A | 57A | 58A | 61A | | |
| A*2420 | 3A | 6A | 9A | 16A | 21A | 25A | 30A | 35A | 37A | 39A | 50A | 54A | 57A | 58A | 61A | | |
| A*2421 | 3A | 6A | 9A | 16A | 21A | 25A | 30A | 35A | 37A | 39A | 50A | 54A | 57A | 58A | 61A | | |
| A*2425 | 3A | 6A | 9A | 16A | 21A | 25A | 30A | 35A | 37A | 39A | 50A | 54A | 57A | 58A | 61A | | |
| A*2426 | 3A | 6A | 9A | 16A | 21A | 25A | 30A | 35A | 37A | 39A | 50A | 54A | 57A | 58A | 61A | | |
| A*2427 | 3A | 6A | 9A | 16A | 21A | 25A | 30A | 35A | 37A | 39A | 50A | 54A | 57A | 58A | 59A | 61A | |
| A*2429 | 3A | 6A | 9A | 16A | 21A | 25A | 30A | 35A | 37A | 39A | 50A | 54A | 58A | 61A | | | |
| A*2431 | 3A | 6A | 9A | 16A | 21A | 25A | 30A | 35A | 37A | 39A | 50A | 54A | 57A | 58A | 61A | | |
| A*24032 | 3A | 6A | 9A | 16A | 21A | 25A | 30A | 35A | 37A | 39A | 50A | 54A | 57A | 58A | 61A | 64A | |
| A*2423 | 3A | 6A | 9A | 16A | 21A | 25A | 30A | 35A | 37A | 39A | 50A | 54A | 57A | 58A | 61A | 64A | |
| A*2410 | 3A | 6A | 9A | 16A | 21A | 25A | 30A | 35A | 37A | 39A | 50A | 52A | 57A | 58A | 61A | 64A | |
| A*2430 | 3A | 6A | 9A | 16A | 21A | 26A | 30A | 35A | 37A | 39A | 50A | 54A | 57A | 58A | 61A | | |
| A*2416 | 4A | 6A | 9A | 16A | 21A | 25A | 30A | 33A | 37A | 41A | 48A | 54A | 57A | 58A | 60A | 61A | 64A |
| A*2419 | 3A | 6A | 9A | 16A | 21A | 25A | 31A | 35A | 37A | 39A | 50A | 54A | 57A | 58A | 61A | | |
| A*2428 | 3A | 6A | 9A | 16A | 21A | 26A | 31A | 35A | 37A | 39A | 50A | 54A | 57A | 58A | 61A | | |
| A*6810 | 2A | 6A | 9A | 16A | 20A | 25A | 31A | 33A | 37A | 45A | 49A | 54A | 58A | 61A | 64A | | |
| A*6814 | 2A | 6A | 9A | 16A | 20A | 25A | 31A | 33A | 37A | 45A | 49A | 54A | 58A | 61A | 64A | | |
| A*0246 | 1A | 7A | 11A | 16A | 21A | 26A | 31A | 34A | 38A | 39A | 48A | 54A | 57A | 61A | 64A | | |
| A*0248 | 1A | 7A | 9A | 16A | 21A | 25A | 31A | 34A | 38A | 39A | 48A | 54A | 57A | 61A | 64A | | |
| A*2414 | 3A | 6A | 9A | 16A | 21A | 25A | 30A | 34A | 38A | 39A | 50A | 54A | 57A | 58A | 61A | | |
| A*3402 | 2A | 6A | 9A | 17A | 20A | 25A | 31A | 33A | 36A | 45A | 48A | 54A | 58A | 62A | 64A | | |
| A*3403 | 2A | 6A | 9A | 17A | 20A | 25A | 31A | 33A | 36A | 45A | 48A | 54A | 58A | 62A | 64A | | |
| A*2424 | 3A | 6A | 9A | 17A | 20A | 25A | 30A | 35A | 37A | 39A | 48A | 54A | 58A | 60A | 61A | | |
| A*3301 | 4A | 6A | 9A | 17A | 20A | 25A | 31A | 33A | 37A | 41A | 48A | 54A | 58A | 60A | 61A | 64A | |
| A*3303 | 4A | 6A | 9A | 17A | 20A | 25A | 31A | 33A | 37A | 41A | 48A | 54A | 58A | 60A | 61A | 64A | |
| A*3304 | 4A | 6A | 9A | 17A | 20A | 25A | 31A | 33A | 37A | 41A | 48A | 54A | 58A | 60A | 61A | 64A | |
| A*3305 | 4A | 6A | 9A | 17A | 20A | 25A | 31A | 33A | 37A | 41A | 48A | 54A | 58A | 60A | 61A | 64A | |
| A*3306 | 4A | 6A | 9A | 17A | 20A | 25A | 31A | 33A | 37A | 41A | 48A | 54A | 58A | 60A | 61A | 64A | |
| A*68011 | 2A | 6A | 9A | 17A | 20A | 25A | 31A | 33A | 37A | 45A | 49A | 54A | 58A | 61A | 64A | | |
| A*6804 | 2A | 6A | 9A | 17A | 20A | 25A | 31A | 33A | 37A | 45A | 49A | 54A | 58A | 61A | 64A | | |
| A*6813 | 2A | 6A | 9A | 17A | 20A | 25A | 31A | 33A | 37A | 45A | 49A | 54A | 58A | 61A | 64A | | |
| A*6808 | 2A | 6A | 9A | 17A | 20A | 25A | 31A | 33A | 37A | 45A | 51A | 54A | 58A | 61A | 64A | | |
| A*68012 | 5A | 6A | 9A | 17A | 20A | 25A | 31A | 33A | 37A | 45A | 49A | 54A | 58A | 61A | 64A | | |
| A*6812 | 5A | 6A | 9A | 17A | 20A | 25A | 31A | 33A | 37A | 45A | 49A | 54A | 58A | 61A | 64A | | |
| A*6816 | 5A | 6A | 9A | 17A | 20A | 25A | 31A | 33A | 37A | 45A | 49A | 54A | 58A | 61A | 64A | | |
| A*6817 | 5A | 6A | 9A | 17A | 20A | 25A | 31A | 33A | 37A | 45A | 49A | 54A | 58A | 61A | 64A | | |
| A*6819 | 5A | 6A | 9A | 17A | 20A | 25A | 31A | 33A | 37A | 45A | 49A | 54A | 58A | 61A | 64A | | |
| A*68031 | 5A | 6A | 9A | 17A | 20A | 25A | 31A | 33A | 37A | 45A | 49A | 54A | 58A | 61A | 64A | | |
| A*68032 | 5A | 6A | 9A | 17A | 20A | 25A | 31A | 33A | 37A | 45A | 49A | 54A | 58A | 61A | 64A | | |
| A*6806 | 5A | 6A | 9A | 17A | 20A | 25A | 31A | 33A | 37A | 44A | 49A | 54A | 58A | 61A | 64A | | |
| A*6807 | 5A | 6A | 9A | 17A | 20A | 25A | 31A | 33A | 37A | 46A | 49A | 54A | 58A | 61A | 64A | | |
| A*6805 | 5A | 6A | 9A | 17A | 20A | 26A | 31A | 33A | 37A | 45A | 49A | 54A | 58A | 61A | 64A | | |
| A*6809 | 5A | 6A | 9A | 17A | 20A | 25A | 31A | 33A | 37A | 45A | 50A | 54A | 58A | 61A | 64A | | |
| A*2501 | 2A | 6A | 9A | 17A | 20A | 25A | 32A | 33A | 38A | 41A | 49A | 52A | 62A | 64A | | | |
| A*2502 | 2A | 6A | 9A | 17A | 20A | 25A | 32A | 33A | 38A | 41A | 49A | 52A | 62A | 64A | | | |
| A*2503 | 1A | 6A | 9A | 17A | 20A | 25A | 32A | 33A | 38A | 41A | 49A | 52A | 62A | 64A | | | |
| A*2601 | 2A | 6A | 9A | 17A | 20A | 25A | 29A | 33A | 38A | 41A | 49A | 52A | 62A | 64A | | | |
| A*2610 | 2A | 6A | 9A | 17A | 20A | 25A | 29A | 33A | 38A | 41A | 49A | 52A | 62A | 64A | | | |
| A*2612 | 2A | 6A | 9A | 17A | 20A | 25A | 29A | 33A | 38A | 41A | 49A | 52A | 61A | 64A | | | |
| A*2613 | 2A | 6A | 9A | 17A | 20A | 25A | 29A | 33A | 38A | 41A | 49A | 52A | 62A | 64A | | | |
| A*2614 | 2A | 6A | 9A | 17A | 20A | 25A | 29A | 33A | 38A | 41A | 49A | 52A | 62A | 64A | | | |
| A*2615 | 2A | 6A | 9A | 17A | 20A | 25A | 29A | 33A | 38A | 41A | 49A | 52A | 62A | 64A | | | |
| A*2617 | 2A | 6A | 9A | 17A | 20A | 25A | 29A | 33A | 38A | 41A | 49A | 52A | 62A | 64A | | | |
| A*2608 | 2A | 6A | 9A | 17A | 20A | 25A | 29A | 33A | 38A | 41A | 50A | 52A | 62A | 64A | | | |
| A*2616 | 3A | 6A | 9A | 17A | 20A | 25A | 29A | 33A | 38A | 41A | 49A | 52A | 62A | 64A | | | |
| A*2602 | 2A | 6A | 9A | 17A | 20A | 25A | 29A | 33A | 38A | 44A | 49A | 52A | 62A | 64A | | | |
| A*2604 | 2A | 6A | 9A | 17A | 20A | 25A | 29A | 33A | 38A | 41A | 49A | 56A | 62A | 64A | | | |
| A*2609 | 2A | 6A | 9A | 17A | 20A | 25A | 29A | 33A | 38A | 41A | 49A | 54A | 62A | 64A | | | |
| A*2605 | 2A | 6A | 9A | 17A | 20A | 25A | 30A | 33A | 38A | 41A | 49A | 52A | 62A | 64A | | | |
| A*2603 | 2A | 6A | 9A | 17A | 20A | 26A | 31A | 33A | 38A | 41A | 49A | 52A | 62A | 64A | | | |
| A*2606 | 2A | 6A | 9A | 17A | 20A | 26A | 31A | 33A | 38A | 45A | 49A | 52A | 62A | 64A | | | |
| A*6601 | 2A | 6A | 9A | 17A | 20A | 25A | 31A | 33A | 38A | 41A | 49A | 52A | 62A | 64A | | | |
| A*6604 | 2A | 6A | 9A | 17A | 20A | 25A | 31A | 33A | 38A | 41A | 49A | 52A | 62A | 64A | | | |
| A*3401 | 2A | 6A | 9A | 17A | 21A | 25A | 31A | 33A | 38A | 41A | 49A | 54A | 62A | 64A | | | |
| A*6602 | 2A | 6A | 9A | 17A | 20A | 25A | 31A | 33A | 38A | 41A | 49A | 55A | 62A | 64A | | | |
| A*6603 | 2A | 6A | 9A | 17A | 20A | 25A | 31A | 33A | 38A | 41A | 49A | 55A | 62A | 64A | | | |
| A*6802 | 2A | 6A | 9A | 17A | 20A | 25A | 31A | 33A | 38A | 39A | 49A | 54A | 61A | 64A | | | |
| A*6815 | 2A | 6A | 9A | 17A | 20A | 26A | 31A | 33A | 38A | 39A | 49A | 54A | 61A | 64A | | | |
| A*6901 | 2A | 6A | 9A | 17A | 20A | 25A | 31A | 34A | 38A | 39A | 48A | 54A | 58A | 61A | 64A | | |
| A*2901 | 4A | 6A | 9A | 18A | 20A | 25A | 29A | 33A | 37A | 45A | 48A | 54A | 58A | 60A | 61A | 64A | |
| A*2902 | 4A | 6A | 9A | 18A | 20A | 25A | 29A | 33A | 37A | 45A | 48A | 54A | 58A | 60A | 61A | 64A | |
| A*2903 | 4A | 6A | 9A | 18A | 20A | 25A | 29A | 33A | 37A | 45A | 48A | 54A | 58A | 60A | 61A | | |
| A*2904 | 4A | 6A | 9A | 18A | 24A | 25A | 29A | 33A | 37A | 45A | 48A | 54A | 58A | 60A | 61A | 64A | |
| A*4301 | 2A | 6A | 9A | 18A | 20A | 25A | 29A | 33A | 38A | 41A | 49A | 52A | 64A | | | | |

satellite region

| | | | | | | | | | |
|---|---|---|---|---|---|---|---|---|---|
| A*01011 | | | | | | | | | |
| A*0106 | | | | | | | | | |
| A*0108 | | | | | | | | | |
| A*3601 | 1066 | | | | | | | | |
| A*3602 | 1056 | 1066 | | | | | | | |
| A*01012 | | | | | | | | | |
| A*0102 | 919 | | | | | | | | |
| A*0107 | 984 | | | | | | | | |
| A*3002 | 919 | 984 | 1011 | 1031 | 1033 | 1066 | | | |
| A*3009 | 919 | 984 | 1011 | 1031 | 1033 | 1066 | | | |
| A*3003 | 919 | 984 | 1011 | 1031 | 1033 | 1066 | | | |
| A*3004 | 919 | 984 | 1011 | 1031 | 1033 | 1066 | | | |
| A*3006 | 919 | 984 | 1011 | 1031 | 1033 | 1066 | | | |
| A*03011 | 959 | 984 | 1031 | 1056 | 1066 | | | | |
| A*0304 | 959 | 984 | 1031 | 1056 | 1066 | 1069 | | | |
| A*0308 | 984 | 1031 | 1056 | 1066 | | | | | |
| A*0305 | 959 | 984 | 1031 | 1066 | | | | | |
| A*0306 | 959 | 984 | 1031 | 1056 | 1057 | 1066 | | | |
| A*0307 | 959 | 984 | 1031 | 1056 | 1066 | | | | |
| A*03013 | 959 | 984 | 1031 | 1056 | 1066 | | | | |
| A*3103 | 961 | 1031 | 1033 | 1066 | | | | | |
| A*3104 | 961 | 984 | 1031 | 1033 | 1066 | | | | |
| A*3001 | 919 | 959 | 984 | 1011 | 1031 | 1033 | 1066 | | |
| A*3008 | 908 | 919 | 959 | 984 | 1011 | 1031 | 1033 | 1066 | |
| A*03012 | 959 | 984 | 1031 | 1056 | 1066 | | | | |
| A*0302 | 959 | 984 | 1031 | 1056 | 1066 | | | | |
| A*11011 | 959 | 1066 | | | | | | | |
| A*11012 | 959 | 1029 | 1066 | | | | | | |
| A*1102 | 920 | 959 | 1066 | | | | | | |
| A*1103 | 959 | 1066 | | | | | | | |
| A*1105 | 959 | 1034 | 1066 | | | | | | |
| A*1106 | 959 | 1066 | | | | | | | |
| A*1107 | 959 | 1009 | 1066 | | | | | | |
| A*1109 | 959 | 1055 | 1066 | | | | | | |
| A*1104 | 959 | 1066 | | | | | | | |
| A*1108 | 959 | 1066 | | | | | | | |
| A*3204 | 984 | 1031 | 1056 | 1066 | | | | | |
| A*0103 | | | | | | | | | |
| A*3203 | 926 | 984 | 1026 | 1031 | 1033 | 1066 | | | |
| A*31012 | 961 | 984 | 1026 | 1031 | 1033 | 1066 | | | |
| A*3105 | 961 | 984 | 1026 | 1031 | 1033 | | | | |
| A*3102 | 961 | 984 | 1026 | 1031 | 1033 | 1066 | | | |
| A*7401 | 926 | 984 | 1026 | 1031 | 1033 | 1066 | | | |
| A*7402 | 926 | 984 | 1026 | 1031 | 1033 | 1066 | | | |
| A*7403 | 926 | 982 | 984 | 1026 | 1031 | 1033 | 1066 | | |
| A*7405 | 926 | 960 | 984 | 1026 | 1031 | 1033 | 1066 | | |
| A*3202 | 926 | 984 | 1026 | 1031 | 1033 | 1066 | | | |
| A*3201 | 926 | 984 | 1026 | 1031 | 1033 | 1066 | | | |
| A*3206 | 926 | 984 | 1026 | 1031 | 1033 | 1066 | | | |
| A*3205 | 926 | 950 | 984 | 1026 | 1031 | 1033 | 1066 | | |
| A*0245 | 984 | 1066 | | | | | | | |
| A*0204 | 984 | 1066 | | | | | | | |
| A*02171 | 984 | 1004 | 1066 | | | | | | |
| A*02172 | 984 | 1004 | 1066 | | | | | | |
| A*7404 | 926 | 984 | 1026 | 1031 | 1033 | 1066 | | | |
| A*2408 | 909 | 984 | 1004 | 1027 | 1031 | | | | |
| A*02012 | 984 | 1027 | 1066 | | | | | | |
| A*02013 | 984 | 1027 | 1035 | 1066 | | | | | |
| A*02014 | 984 | 1027 | 1066 | | | | | | |
| A*02015 | 984 | 1027 | 1066 | | | | | | |
| A*0209 | 984 | 1027 | 1066 | | | | | | |
| A*0224 | 984 | 1066 | | | | | | | |
| A*0236 | 984 | 1027 | | | | | | | |
| A*02016 | 984 | | 1027 | 1066 | | | | | |
| A*0207 | 984 | 1005 | 1027 | 1066 | | | | | |
| A*0218 | 984 | 1005 | 1027 | 1066 | | | | | |
| A*0225 | 984 | 1027 | 1035 | 1066 | | | | | |
| A*0226 | 984 | 1027 | 1066 | | | | | | |
| A*0230 | 909 | 984 | 1027 | 1066 | | | | | |
| A*0231 | 927 | 984 | 1066 | | | | | | |
| A*0233 | 984 | 1005 | 1027 | 1066 | | | | | |
| A*0234 | 959 | 984 | 1027 | 1066 | | | | | |
| A*0239 | 984 | 1004 | 1027 | 1066 | | | | | |
| A*0240 | 984 | 1027 | 1066 | | | | | | |
| A*0211 | 984 | 1027 | 1066 | | | | | | |
| A*02201 | 984 | 1027 | 1066 | | | | | | |
| A*02202 | 984 | 1027 | 1066 | | | | | | |
| A*0229 | 984 | 1027 | 1066 | | | | | | |
| A*0235 | 959 | 984 | 1027 | 1066 | | | | | |
| A*02011 | 961 | 984 | 1027 | 1066 | | | | | |
| A*0242 | 984 | 1027 | 1066 | | | | | | |
| A*0216 | 984 | 1027 | 1066 | | | | | | |
| A*0206 | 984 | 1027 | 1066 | | | | | | |
| A*0241 | 984 | 1027 | 1066 | | | | | | |
| A*0210 | 984 | 1004 | 1027 | 1066 | | | | | |
| A*0221 | 924 | 984 | 1027 | 1066 | | | | | |
| A*0244 | 984 | 1027 | 1066 | | | | | | |
| A*0228 | 984 | 1027 | 1066 | | | | | | |
| A*0214 | 984 | 1027 | 1066 | | | | | | |
| A*0205 | 984 | 1027 | 1066 | | | | | | |
| A*0208 | 984 | 1027 | 1066 | | | | | | |
| A*0202 | 984 | 1027 | 1066 | | | | | | |
| A*0247 | 962 | 984 | 1027 | 1066 | | | | | |
| A*0203 | 984 | 1027 | 1066 | | | | | | |
| A*0222 | 984 | 1027 | 1066 | | | | | | |
| A*0212 | 984 | 1027 | 1066 | | | | | | |
| A*0213 | 984 | 1027 | 1066 | | | | | | |
| A*0219 | 984 | 1027 | | | | | | | |
| A*0227 | 984 | 1027 | 1066 | | | | | | |
| A*0237 | 984 | 1027 | | | | | | | |
| A*0238 | 984 | 1027 | 1035 | 1066 | | | | | |
| A*2607 | 1031 | 1033 | 1035 | 1066 | | | | | |
| A*3007 | 919 | 984 | 1011 | 1031 | 1033 | 1066 | | | |
| A*8001 | 1031 | | | | | | | | |
| A*2404 | 984 | 1004 | 1027 | 1027 | 1031 | | | | |
| A*2301 | 984 | 1004 | 1027 | 1027 | 1031 | 1033 | | | |
| A*2303 | 984 | 1004 | 1027 | 1026 | 1031 | 1033 | | | |
| A*2304 | 984 | 1004 | 1027 | 1031 | 1033 | 1066 | | | |
| A*2305 | 911 | 984 | 1004 | 1027 | 1031 | 1033 | | | |
| A*2306 | 983 | 984 | 1004 | 1027 | 1031 | 1033 | | | |
| A*2413 | 984 | 1004 | 1027 | 1031 | | | | | |
| A*2418 | 984 | 1004 | 1027 | 1031 | 1056 | 1066 | | | |
| A*2302 | 984 | 1004 | 1027 | 1031 | 1033 | | | | |
| A*2406 | 984 | 1004 | 1027 | 1031 | | | | | |
| A*2422 | 984 | 1004 | 1027 | 1031 | 1066 | | | | |
| A*2402101 | 984 | 1004 | 1027 | 1031 | | | | | |
| A*2402102L | 984 | 1004 | 1027 | 1031 | | | | | |
| A*24031 | 984 | 1004 | 1027 | 1031 | 1066 | | | | |
| A*24022 | 984 | 1004 | 1027 | | | | | | |
| A*2405 | 984 | 1004 | 1027 | 1031 | 1033 | | | | |
| A*2407 | 959 | 984 | 1004 | 1027 | 1031 | | | | |
| A*2415 | 984 | 1027 | 1031 | | | | | | |
| A*2417 | 984 | 1004 | 1027 | 1027 | 1031 | | | | |
| A*2420 | 909 | 984 | 1004 | 1027 | 1031 | | | | |
| A*2421 | 984 | 1004 | 1027 | 1031 | | | | | |
| A*2425 | 911 | 984 | 1004 | 1027 | 1031 | | | | |
| A*2426 | 984 | 1004 | 1027 | 1010 | 1031 | | | | |
| A*2427 | 984 | 1004 | 1027 | 1031 | | | | | |
| A*2429 | 984 | 1004 | 1027 | 1031 | | | | | |
| A*2431 | 948 | 984 | 1004 | 1027 | 1031 | | | | |
| A*24032 | 984 | 1004 | 1027 | 1031 | 1066 | 1070 | | | |
| A*2423 | 984 | 1004 | 1027 | 1031 | | | | | |
| A*2410 | 984 | 1004 | 1027 | 1031 | 1066 | | | | |
| A*2430 | 984 | 1004 | 1027 | 1031 | | | | | |
| A*2416 | 984 | 1026 | 1031 | 1033 | 1066 | | | | |
| A*2419 | 959 | 1004 | 1027 | 1031 | | | | | |
| A*2428 | 984 | 1004 | 1027 | 1031 | | | | | |
| A*6810 | 959 | 984 | 1027 | 1032 | 1066 | | | | |
| A*6814 | 959 | 984 | 1027 | 1032 | 1066 | | | | |
| A*0246 | 984 | 1066 | | | | | | | |
| A*0248 | 984 | 1066 | | | | | | | |
| A*2414 | 984 | 1027 | 1031 | | | | | | |
| A*3402 | 949 | 959 | 1031 | 1033 | 1035 | 1066 | | | |
| A*3403 | 949 | 959 | 1031 | 1033 | 1066 | | | | |
| A*2424 | 949 | 959 | 984 | 1004 | 1027 | 1031 | 1033 | | |
| A*3301 | 949 | 961 | 984 | 1026 | 1031 | 1033 | 1066 | 1068 | |
| A*3303 | 949 | 961 | 984 | 1026 | 1031 | 1033 | 1066 | | |
| A*3304 | 949 | 961 | 984 | 1026 | 1028 | 1031 | 1033 | 1066 | 1068 |
| A*3305 | 933 | 949 | 961 | 984 | 1026 | 1031 | 1033 | 1066 | 1068 |
| A*3306 | 932 | 949 | 961 | 984 | 1026 | 1031 | 1033 | 1066 | |
| A*68011 | 949 | 959 | 984 | 1027 | 1032 | 1066 | | | |
| A*6804 | 949 | 961 | 984 | 1027 | 1032 | 1066 | | | |
| A*6813 | 959 | 984 | 1027 | 1032 | 1066 | | | | |
| A*6808 | 949 | 959 | 984 | 1027 | 1032 | 1066 | | | |
| A*68012 | 949 | 959 | 984 | 1027 | 1032 | 1066 | | | |
| A*6812 | 949 | 959 | 984 | 1027 | 1066 | | | | |
| A*6816 | 949 | 959 | 984 | 1027 | 1032 | 1037 | 1066 | | |
| A*6817 | 949 | 959 | 984 | 1025 | 1027 | 1032 | 1066 | | |
| A*6819 | 949 | 959 | 984 | 1027 | 1032 | 1056 | 1066 | | |
| A*68031 | 949 | 984 | 1027 | 1032 | 1066 | | | | |
| A*68032 | 908 | 949 | 984 | 1027 | 1032 | 1066 | | | |
| A*6806 | 949 | 959 | 984 | 1011 | 1027 | 1032 | 1066 | | |
| A*6807 | 949 | 959 | 984 | 1027 | 1032 | 1066 | | | |
| A*6805 | 949 | 1027 | 1032 | 1066 | | | | | |
| A*6809 | 949 | 959 | 984 | 1027 | 1032 | 1066 | | | |
| A*2501 | 949 | 1031 | 1033 | 1035 | 1066 | | | | |
| A*2502 | 949 | 959 | 1031 | 1033 | 1035 | 1066 | | | |
| A*2503 | 949 | 1031 | 1033 | 1035 | 1066 | | | | |
| A*2601 | 949 | 1031 | 1033 | 1035 | 1066 | | | | |
| A*2610 | 949 | 1027 | 1031 | 1033 | 1035 | 1066 | | | |
| A*2612 | 949 | 1031 | 1033 | 1035 | 1066 | | | | |
| A*2613 | 949 | 959 | 1031 | 1033 | 1035 | 1066 | | | |
| A*2614 | 949 | 1031 | 1033 | 1066 | | | | | |
| A*2615 | 925 | 949 | 1031 | 1033 | 1035 | 1066 | | | |
| A*2617 | 949 | 985 | 1031 | 1033 | 1035 | 1066 | | | |
| A*2608 | 949 | 1031 | 1033 | 1035 | 1066 | | | | |
| A*2616 | 949 | 1031 | 1033 | 1035 | 1066 | | | | |
| A*2602 | 949 | 1031 | 1033 | 1035 | 1066 | | | | |
| A*2604 | 949 | 1031 | 1033 | 1035 | 1066 | | | | |
| A*2609 | 949 | 1031 | 1033 | 1035 | 1066 | | | | |
| A*2605 | 949 | 1031 | 1033 | 1035 | 1066 | | | | |
| A*2603 | 949 | 1031 | 1033 | 1035 | 1066 | | | | |
| A*2606 | 949 | 1031 | 1033 | 1035 | 1066 | | | | |
| A*6601 | 949 | 959 | 1031 | 1033 | 1035 | 1066 | | | |
| A*6604 | 949 | 959 | 1031 | 1033 | 1035 | 1066 | 1068 | | |
| A*3401 | 949 | 959 | 1031 | 1033 | 1035 | 1066 | | | |
| A*6602 | 949 | 959 | 984 | 1031 | 1033 | 1035 | 1066 | | |
| A*6603 | 949 | 984 | 1031 | 1033 | 1035 | 1066 | | | |
| A*6802 | 949 | 959 | 984 | 1027 | 1032 | 1066 | | | |
| A*6815 | 949 | 984 | 1027 | 1032 | 1066 | | | | |
| A*6901 | 949 | 959 | 984 | 1027 | 1032 | 1066 | | | |
| A*2901 | 926 | 948 | 959 | 984 | 1006 | 1026 | 1031 | 1033 | 1066 |
| A*2902 | 926 | 948 | 959 | 984 | 1026 | 1031 | 1033 | 1066 | |
| A*2903 | 926 | 948 | 959 | 984 | 1026 | 1031 | 1033 | | |
| A*2904 | 926 | 948 | 959 | 984 | 1026 | 1031 | 1033 | 1066 | |
| A*4301 | 948 | 1066 | | | | | | | |

TABLE 30
Correspondence of the patchworks and SEQ ID Nos for A

| | |
|---|---|
| patchwork | SEQ ID NO. |

| | | | | | | |
|---|---|---|---|---|---|---|
| 1A | 912 | | | | | |
| 2A | 913 | 917 | | | | |
| 3A | 914 | 918 | | | | |
| 4A | 915 | | | | | |
| 5A | 916 | | | | | |
| 6A | 921 | | | | | |
| 7A | 922 | | | | | |
| 8A | 923 | | | | | |
| 9A | 934 | 939 | | | | |
| 10A | 935 | 950 | | | | |
| 11A | 936 | | | | | |
| 12A | 937 | | | | | |
| 13A | 938 | | | | | |
| 14A | 941 | | | | | |
| 15A | 942 | 946 | | | | |
| 16A | 943 | | | | | |
| 17A | 944 | 947 | | | | |
| 18A | 945 | | | | | |
| 19A | 952 | | | | | |
| 20A | 953 | | | | | |
| 21A | 954 | 958 | | | | |
| 22A | 955 | | | | | |
| 23A | 956 | | | | | |
| 24A | 957 | | | | | |
| 25A | 963 | 967 | 969 | 971 | | |
| 26A | 964 | 968 | 970 | | | |
| 27A | 965 | | | | | |
| 28A | 966 | | | | | |
| 29A | 972 | 976 | | | | |
| 30A | 973 | 977 | 979 | 981 | | |
| 31A | 974 | 978 | 980 | | | |
| 32A | 975 | | | | | |
| 33A | 986 | 989 | 991 | | | |
| 34A | 987 | | | | | |
| 35A | 988 | 990 | | | | |
| 36A | 992 | | | | | |
| 37A | 993 | 995 | 997 | 999 | | |
| 38A | 994 | 996 | 998 | 1000 | 1001 | 1002 |
| 39A | 1012 | 1020 | | | | |
| 40A | 1013 | 1021 | | | | |
| 41A | 1014 | | | | | |
| 42A | 1015 | 1022 | | | | |
| 43A | 1016 | | | | | |
| 44A | 1017 | 1023 | | | | |
| 45A | 1018 | 1024 | | | | |
| 46A | 1019 | | | | | |
| 47A | 1049 | 1054 | | | | |
| 48A | 1050 | | | | | |
| 49A | 1051 | | | | | |
| 50A | 1052 | | | | | |
| 51A | 1053 | | | | | |
| 52A | 1058 | 1063 | | | | |
| 53A | 1059 | | | | | |
| 54A | 1060 | 1064 | 1065 | | | |
| 55A | 1061 | | | | | |
| 56A | 1062 | | | | | |
| 57A | 950 | 951 | | | | |
| 58A | 1007 | 1008 | | | | |
| 59A | 1030 | 1041 | | | | |
| 60A | 1036 | 1042 | 1046 | 1048 | | |
| 61A | 1038 | 1043 | 1047 | | | |
| 62A | 1039 | 1044 | | | | |
| 63A | 1040 | 1045 | | | | |
| 64A | 1067 | 1071 | | | | |

TABLE 31
Correspondence of the patchwork regions and satellite regions for types of B

| | patchwork region | | | | | | | | | | | | | | | | | | |
|---|---|---|---|---|---|---|---|---|---|---|---|---|---|---|---|---|---|---|---|
| B*35011 | 2B | 7B | 12B | 15B | 20B | 24B | 29B | 38B | 43B | 47B | 51B | 53B | 54B | 58B | 59B | 62B | | | |
| B*3524 | 2B | 7B | 12B | 15B | 20B | 24B | 29B | 38B | 43B | 47B | 51B | 53B | 54B | 62B | | | | | |
| B*3529 | 2B | 7B | 12B | 15B | 20B | 24B | 29B | 38B | 43B | 47B | 51B | 53B | 58B | 59B | 62B | | | | |
| B*3511 | 2B | 7B | 12B | 15B | 20B | 24B | 29B | 38B | 43B | 47B | 61B | 53B | 54B | 58B | 59B | 62B | | | |
| B*3514 | 2B | 7B | 12B | 15B | 20B | 24B | 29B | 38B | 43B | 47B | 51B | 53B | 54B | 58B | 59B | 62B | | | |
| B*3521 | 2B | 7B | 12B | 15B | 20B | 24B | 29B | 38B | 43B | 47B | 51B | 53B | 54B | 58B | 59B | 62B | | | |
| B*35012 | 2B | 7B | 12B | 15B | 20B | 24B | 29B | 38B | 43B | 47B | 51B | 53B | 54B | 58B | 59B | 62B | | | |
| B*3505 | 2B | 7B | 12B | 15B | 20B | 24B | 28B | 38B | 43B | 47B | 51B | 53B | 54B | 59B | 62B | | | | |
| B*3507 | 2B | 7B | 12B | 15B | 20B | 24B | 29B | 38B | 43B | 47B | 51B | 53B | 54B | 58B | 59B | 62B | | | |
| B*3510 | 2B | 7B | 12B | 15B | 20B | 24B | 29B | 38B | 43B | 47B | 51B | 52B | 53B | 54B | 58B | 59B | 62B | | |
| B*3523 | 2B | 7B | 12B | 15B | 20B | 24B | 29B | 38B | 43B | 47B | 51B | 53B | 54B | 59B | 62B | | | | |
| B*3515 | 2B | 7B | 12B | 15B | 20B | 24B | 29B | 38B | 43B | 45B | 51B | 53B | 54B | 58B | 59B | 62B | | | |
| B*3508 | 2B | 7B | 12B | 15B | 20B | 24B | 29B | 38B | 41B | 47B | 51B | 53B | 54B | 58B | 59B | 62B | | | |
| B*1522 | 2B | 7B | 12B | 15B | 20B | 24B | 29B | 38B | 44B | 47B | 51B | 53B | 54B | 62B | 64B | | | | |
| B*1559 | 2B | 7B | 12B | 15B | 20B | 24B | 29B | 34B | 44B | 47B | 51B | 53B | 54B | 61B | 62B | 64B | | | |
| B*35091 | 2B | 7B | 12B | 15B | 20B | 24B | 29B | 34B | 43B | 47B | 51B | 53B | 54B | 58B | 59B | 61B | 62B | | |
| B*35092 | 2B | 7B | 12B | 15B | 20B | 24B | 29B | 34B | 43B | 47B | 51B | 53B | 54B | 58B | 59B | 61B | | | |
| B*3504 | 2B | 7B | 12B | 15B | 20B | 24B | 29B | 34B | 43B | 47B | 51B | 53B | 54B | 58B | 59B | 61B | 62B | | |
| B*3502 | 2B | 7B | 12B | 15B | 20B | 24B | 29B | 34B | 43B | 47B | 51B | 53B | 54B | 58B | 59B | 61B | 62B | | |
| B*3512 | 2B | 7B | 12B | 15B | 20B | 24B | 29B | 34B | 43B | 47B | 51B | 53B | 54B | 61B | 62B | | | | |
| B*3518 | 2B | 7B | 12B | 15B | 20B | 24B | 29B | 34B | 44B | 47B | 51B | 53B | 54B | 58B | 59B | 61B | | | |
| B*3503 | 2B | 7B | 12B | 15B | 20B | 24B | 29B | 40B | 43B | 47B | 51B | 53B | 54B | 58B | 59B | 62B | | | |
| B*3506 | 2B | 7B | 12B | 15B | 20B | 24B | 29B | 40B | 43B | 47B | 51B | 53B | 54B | 58B | 59B | 61B | 62B | | |
| B*3513 | 2B | 7B | 12B | 15B | 20B | 24B | 29B | 40B | 43B | 47B | 51B | 53B | 54B | 58B | 59B | 62B | | | |
| B*3516 | 2B | 7B | 12B | 15B | 20B | 24B | 28B | 38B | 43B | 47B | 51B | 53B | 54B | 62B | | | | | |
| B*3517 | 2B | 7B | 12B | 15B | 20B | 24B | 28B | 38B | 43B | 47B | 51B | 53B | 54B | 62B | | | | | |
| B*3530 | 2B | 7B | 12B | 15B | 20B | 24B | 28B | 38B | 43B | 47B | 51B | 53B | 54B | 62B | | | | | |
| B*3522 | 2B | 7B | 12B | 15B | 20B | 24B | 28B | 34B | 41B | 47B | 51B | 53B | 54B | 61B | 62B | | | | |
| B*3531 | 2B | 7B | 12B | 15B | 20B | 24B | 28B | 34B | 43B | 45B | 51B | 53B | 54B | 61B | | | | | |
| B*3532 | 2B | 7B | 12B | 15B | 20B | 24B | 27B | 38B | 43B | 47B | 51B | 53B | 54B | 62B | | | | | |
| B*3535 | 2B | 7B | 12B | 15B | 20B | 24B | 27B | 38B | 43B | 46B | 51B | 53B | 54B | 58B | 59B | 62B | 63B | | |
| B*3534 | 2B | 7B | 12B | 15B | 20B | 24B | 27B | 34B | 43B | 47B | 51B | 53B | 54B | 58B | 59B | 62B | | | |
| B*3533 | 2B | 7B | 12B | 15B | 20B | 24B | 27B | 40B | 43B | 45B | 51B | 53B | 54B | 58B | 59B | 62B | | | |
| B*3536 | 2B | 7B | 12B | 15B | 20B | 24B | 27B | 40B | 43B | 47B | 51B | 53B | 54B | 58B | 59B | 62B | | | |
| B*3537 | 2B | 7B | 12B | 15B | 20B | 24B | 30B | 38B | 43B | 47B | 51B | 53B | 54B | 58B | 59B | 62B | | | |
| B*78021 | 2B | 7B | 12B | 15B | 20B | 24B | 30B | 34B | 43B | 47B | 51B | 53B | 54B | 61B | 62B | | | | |
| B*7804 | 2B | 7B | 12B | 15B | 20B | 24B | 308 | 34B | 43B | 47B | 51B | 53B | 54B | 61B | 62B | | | | |
| B*5305 | 2B | 7B | 12B | 15B | 20B | 26B | 29B | 38B | 43B | 47B | 51B | 53B | 54B | 56B | 58B | 59B | 62B | | |
| B*3527 | 2B | 7B | 12B | 15B | 21B | 24B | 29B | 38B | 43B | 47B | 51B | 53B | 54B | 58B | 59B | 62B | | | |
| B*5304 | 2B | 7B | 12B | 15B | 21B | 26B | 29B | 40B | 43B | 47B | 51B | 53B | 54B | 56B | 57B | 58B | 62B | | |
| B*5307 | 2B | 7B | 12B | 15B | 21B | 26B | 29B | 40B | 43B | 47B | 51B | 53B | 54B | 56B | 57B | 60B | 61B | 62B | |
| B*5301 | 2B | 7B | 12B | 15B | 21B | 26B | 29B | 38B | 43B | 47B | 51B | 53B | 54B | 56B | 57B | 58B | 59B | 62B | |
| B*5302 | 2B | 7B | 12B | 15B | 21B | 26B | 29B | 38B | 43B | 47B | 51B | 53B | 54B | 56B | 57B | 58B | 59B | 62B | |
| B*51012 | 2B | 7B | 12B | 15B | 21B | 26B | 30B | 34B | 43B | 47B | 51B | 53B | 54B | 56B | 57B | 58B | 61B | 62B | |
| B*51022 | 2B | 7B | 12B | 15B | 21B | 26B | 30B | 34B | 43B | 47B | 51B | 53B | 54B | 56B | 57B | 58B | 61B | 62B | |
| B*5105 | 2B | 7B | 12B | 15B | 21B | 26B | 30B | 34B | 41B | 47B | 518 | 53B | 54B | 56B | 57B | 58B | 61B | 62B | |
| B*5115 | 2B | 7B | 12B | 15B | 21B | 26B | 30B | 35B | 43B | 47B | 51B | 53B | 54B | 56B | 57B | 61B | 62B | | |
| B*5303 | 2B | 7B | 12B | 15B | 22B | 25B | 29B | 38B | 43B | 47B | 51B | 53B | 54B | 57B | 58B | 59B | 62B | | |
| B*5801 | 2B | 7B | 12B | 18B | 21B | 26B | 29B | 38B | 43B | 47B | 51B | 52B | 54B | 56B | 57B | 58B | 59B | 62B | |
| B*5804 | 2B | 7B | 12B | 18B | 21B | 26B | 29B | 36B | 43B | 47B | 51B | 52B | 54B | 56B | 57B | 58B | 59B | 62B | |
| B*5805 | 2B | 7B | 12B | 18B | 21B | 26B | 29B | 38B | 43B | 46B | 51B | 52B | 54B | 56B | 57B | 58B | 59B | 62B | |
| B*5802 | 2B | 7B | 12B | 18B | 21B | 26B | 31B | 38B | 43B | 47B | 51B | 52B | 54B | 56B | 578 | 58B | 59B | 62B | |
| B*5806 | 2B | 7B | 12B | 18B | 21B | 26B | 31B | 38B | 43B | 47B | 51B | 52B | 54B | 56B | 57B | 58B | 59B | 62B | |
| B*3520 | 2B | 7B | 12B | 16B | 20B | 24B | 29B | 38B | 43B | 47B | 51B | 53B | 59B | 62B | | | | | |
| B*3528 | 2B | 7B | 12B | 16B | 20B | 24B | 29B | 38B | 43B | 47B | 51B | 52B | 53B | 54B | 58B | 59b | 62B | | |
| B*7805 | 2B | 7B | 12B | 16B | 20B | 24B | 30B | 34B | 43B | 47B | 51B | 52B | 53B | 54B | 61B | 62B | | | |
| B*52011 | 2B | 7B | 12B | 16B | 21B | 26B | 30B | 34B | 43B | 47B | 51B | 52B | 53B | 54B | 56B | 57B | 58B | 61B | 62B |
| B*52013 | 2B | 7B | 12B | 16B | 21B | 26B | 30B | 34B | 43B | 47B | 52B | 53B | 54B | 56B | 57B | 58B | 61B | 62B | |
| B*3526 | 2B | 7B | 9B | 17B | 20B | 24B | 29B | 38B | 43B | 47B | 51B | 53B | 54B | 58B | 59B | 62B | | | |
| B*5504 | 2B | 7B | 9B | 14B | 20B | 24B | 28B | 34B | 43B | 46B | 50B | 61B | | | | | | | |
| B*5501 | 2B | 7B | 9B | 14B | 20B | 24B | 30B | 35B | 43B | 46B | 50B | 61B | 62B | | | | | | |
| B*5502 | 2B | 7B | 9B | 14B | 20B | 24B | 30B | 35B | 43B | 46B | 50B | 61B | 62B | | | | | | |
| B*5503 | 2B | 7B | 9B | 14B | 20B | 24B | 30B | 35B | 43B | 46B | 50B | 55B | 61B | 62B | | | | | |
| B*5505 | 2B | 7B | 9B | 14B | 20B | 24B | 30B | 35B | 43B | 46B | 50B | 61B | 62B | | | | | | |
| B*5601 | 2B | 7B | 9B | 14B | 20B | 24B | 30B | 35B | 43B | 47B | 50B | 61B | 62B | | | | | | |
| B*5503 | 2B | 7B | 9B | 14B | 20B | 24B | 30B | 35B | 41B | 45B | 50B | 61B | 62B | | | | | | |
| B*5603 | 2B | 7B | 9B | 14B | 20B | 24B | 29B | 38B | 44B | 47B | 62B | | | | | | | | |
| B*5508 | 2B | 7B | 9B | 14B | 20B | 24B | 29B | 34B | 43B | 47B | 50B | 61B | | | | | | | |
| B*5602 | 2B | 7B | 9B | 14B | 20B | 24B | 29B | 35B | 43B | 47B | 50B | 61B | 62B | | | | | | |
| B*5604 | 2B | 7B | 9B | 14B | 20B | 24B | 29B | 35B | 43B | 47B | 50B | 61B | 62B | | | | | | |
| B*5607 | 2B | 7B | 9B | 14B | 21B | 25B | 30B | 35B | 43B | 47B | 508 | 57B | 56B | 61B | 62B | | | | |
| B*5901 | 2B | 7B | 9B | 15B | 21B | 26B | 30B | 35B | 43B | 46B | 50B | 53B | 54B | 56B | 57B | 58B | 59B | 61B | 62B |
| B*4601 | 2B | 7B | 11B | 14B | 20B | 24B | 29B | 38B | 44B | 47B | 52B | 55B | 62B | | | | | | |
| B*4602 | 2B | 7B | 11B | 14B | 20B | 24B | 29B | 38B | 44B | 47B | 52B | 55B | 62B | | | | | | |
| B*15111 | 2B | 7B | 11B | 14B | 20B | 24B | 29B | 38B | 44B | 47B | 53B | 54B | 62B | 64B | | | | | |
| B*15112 | 2B | 7B | 11B | 14B | 20B | 24B | 29B | 38B | 44B | 47B | 53B | 54B | 62B | 64B | | | | | |
| B*5701 | 2B | 7B | 11B | 18B | 21B | 26B | 33B | 38B | 43B | 47B | 52B | 54B | 56B | 57B | 62B | | | | |
| B*5704 | 2B | 7B | 11B | 18B | 21B | 26B | 33B | 36B | 41B | 47B | 52B | 54B | 56B | 57B | 60B | 62B | | | |
| B*5702 | 2B | 7B | 11B | 18B | 21B | 26B | 33B | 34B | 41B | 47B | 52B | 54B | 56B | 57B | 61B | 62B | | | |
| B*57031 | 2B | 7B | 11B | 13B | 21B | 26B | 33B | 34B | 43B | 47B | 52B | 54B | 56B | 57B | 61B | 62B | | | |
| B*57032 | 2B | 7B | 11B | 18B | 21B | 26B | 33B | 34B | 43B | 47B | 52B | 54B | 56B | 57B | 61B | | | | |
| B*5705 | 2B | 7B | 11B | 18B | 21B | 26B | 29B | 34B | 41B | 47B | 52B | 54B | 56B | 57B | 60B | 61B | 62B | | |
| B*1516 | 2B | 7B | 11B | 18B | 21B | 26B | 29B | 38B | 43B | 47B | 50B | 52B | 54B | 56B | 57B | 58B | 59B | 62B | 64B |
| B*1517 | 2B | 7B | 11B | 18B | 21B | 26B | 29B | 36B | 43B | 47B | 52B | 54B | 56B | 57B | 62B | 64B | | | |
| B*5706 | 2B | 7B | 11B | 18B | 21B | 26B | 30B | 38B | 43B | 47B | 52B | 54B | 56B | 57B | 62B | | | | |
| B*5707 | 2B | 7B | 11B | 16B | 21B | 26B | 30B | 34B | 43B | 47B | 52B | 54B | 56B | 57B | 61B | 62B | | | |
| B*1508 | 2B | 7B | 11B | 15B | 20B | 24B | 29B | 38B | 44B | 47B | 53B | 54B | 62B | 64B | | | | | |
| B*1556 | 2B | 7B | 11B | 15B | 20B | 24B | 29B | 38B | 44B | 47B | 53B | 54B | 62B | 64B | | | | | |
| B*1521 | 2B | 7B | 11B | 17B | 20B | 24B | 29B | 38B | 43B | 47B | 53B | 54B | 58B | 60B | 62B | 64B | | | |
| B*1544 | 2B | 7B | 11B | 17B | 20B | 24B | 29B | 38B | 43B | 46B | 53B | 54B | 58B | 60B | 62B | 64B | | | |
| B*1513 | 2B | 7B | 11B | 16B | 21B | 26B | 29B | 38B | 43B | 47B | 53B | 54B | 56B | 57B | 58B | 60B | 62B | 64B | |
| B*1524 | 2B | 7B | 11B | 16B | 21B | 26B | 29B | 38B | 44B | 47B | 52B | 53B | 54B | 56B | 57B | 62B | 64B | | |
| B*1536 | 2B | 7B | 11B | 16B | 21B | 25B | 29B | 38B | 43B | 47B | 52B | 53B | 54B | 56B | 57B | 58B | 60B | 62B | 64B |
| B*1557 | 2B | 7B | 11B | 16B | 21B | 24B | 29B | 38B | 44B | 47B | 52B | 53B | 54B | 55B | 62B | 64B | | | |
| B*1504 | 2B | 7B | 11B | 16B | 20B | 24B | 30B | 38B | 44B | 47B | 52B | 53B | 54B | 58B | 62B | 64B | | | |
| B*1535 | 2B | 7B | 11B | 16B | 20B | 24B | 30B | 38B | 44B | 47B | 52B | 53B | 54B | 62B | 64B | | | | |
| B*1542 | 2B | 7B | 11B | 16B | 20B | 24B | 30B | 35B | 43B | 46B | 52B | 53B | 54B | 58B | 59B | 61B | 64B | | |
| B*1530 | 2B | 7B | 11B | 16B | 20B | 24B | 29B | 34B | 44B | 47B | 52B | 53B | 54B | 61B | 62B | 64B | | | |
| B*1563 | 2B | 7B | 11B | 16B | 20B | 24B | 29B | 34B | 44B | 47B | 52B | 53B | 54B | 62B | 64B | | | | |
| B*1548 | 2B | 7B | 11B | 16B | 20B | 24B | 29B | 34B | 43B | 47B | 52B | 53B | 54B | 61B | 63B | 64B | | | |
| B*4021 | 2B | 7B | 11B | 16B | 20B | 24B | 29B | 34B | 43B | 45B | 53B | 54B | 61B | | | | | | |
| B*1527 | 2B | 7B | 11B | 16B | 20B | 24B | 29B | 38B | 44B | 47B | 53B | 54B | 62B | 64B | | | | | |
| B*1532 | 2B | 7B | 11B | 16B | 20B | 24B | 29B | 38B | 44B | 47B | 52B | 53B | 54B | 62B | 64B | | | | |
| B*1515 | 2B | 7B | 11B | 16B | 20B | 24B | 29B | 38B | 44B | 47B | 53B | 54B | 62B | 64B | | | | | |
| B*1501101 | 2B | 7B | 11B | 16B | 20B | 24B | 29B | 38B | 44B | 47B | 52B | 53B | 54B | 62B | 64B | | | | |
| B*1512 | 2B | 7B | 11B | 16B | 20B | 24B | 29B | 38B | 44B | 47B | 52B | 53B | 54B | 62B | 64B | | | | |
| B*1519 | 2B | 7B | 11B | 16B | 20B | 24B | 29B | 38B | 44B | 47B | 52B | 53B | 54B | 62B | 64B | | | | |
| B*15012 | 2B | 7B | 11B | 16B | 20B | 24B | 29B | 38B | 44B | 47B | 52B | 53B | 54B | 62B | 64B | | | | |
| B*15013 | 2B | 7B | 11B | 16B | 20B | 24B | 29B | 38B | 44B | 47B | 52B | 53B | 54B | 62B | 64B | | | | |
| B*15014 | 2B | 7B | 11B | 16B | 20B | 24B | 29B | 38B | 44B | 47B | 50B | 52B | 53B | 54B | 62B | 64B | | | |
| B*1514 | 2B | 7B | 11B | 16B | 20B | 24B | 29B | 38B | 44B | 47B | 52B | 53B | 54B | 62B | 64B | | | | |
| B*1528 | 2B | 7B | 11B | 16B | 20B | 24B | 29B | 38B | 44B | 47B | 53B | 54B | 62B | 64B | | | | | |
| B*1533 | 2B | 7B | 11B | 16B | 20B | 24B | 29B | 38B | 44B | 47B | 52B | 53B | 54B | 64B | | | | | |
| B*1534 | 2B | 7B | 11B | 16B | 20B | 24B | 29B | 38B | 44B | 47B | 52B | 53B | 64B | 59B | 62B | 64B | | | |
| B*1538 | 2B | 7B | 11B | 16B | 20B | 24B | 29B | 38B | 44B | 47B | 52B | 53B | 54B | 62B | 64B | | | | |
| B*1560 | 2B | 7B | 11B | 16B | 20B | 24B | 29B | 38B | 44B | 47B | 52B | 53B | 54B | 62B | 64B | | | | |
| B*1550 | 2B | 7B | 11B | 16B | 20B | 24B | 29B | 38B | 44B | 46B | 52B | 53B | 54B | 62B | 64B | | | | |
| B*1525 | 2B | 7B | 11B | 16B | 20B | 24B | 29B | 38B | 43B | 47B | 52B | 53B | 54B | 58B | 60B | 62B | 64B | | |
| B*1539 | 2B | 7B | 11B | 16B | 20B | 24B | 29B | 38B | 43B | 47B | 52B | 53B | 54B | 62B | 64B | | | | |
| B*1502 | 2B | 7B | 11B | 16B | 20B | 24B | 29B | 38B | 43B | 47B | 53B | 54B | 58B | 60B | 62B | 64B | | | |
| B*1505 | 2B | 7B | 11B | 16B | 20B | 24B | 29B | 38B | 43B | 47B | 52B | 53B | 54B | 62B | 64B | | | | |
| B*1531 | 2B | 7B | 11B | 16B | 20B | 24B | 29B | 38B | 43B | 47B | 53B | 54B | 60B | 62B | 64B | | | | |
| B*1506 | 2B | 7B | 11B | 16B | 20B | 24B | 29B | 38B | 43B | 47B | 52B | 53B | 54B | 60B | 62B | 64B | | | |
| B*1520 | 2B | 7B | 11B | 16B | 20B | 24B | 29B | 38B | 43B | 47B | 52B | 53B | 54B | 58B | 59B | 64B | | | |
| B*1540 | 2B | 7B | 11B | 16B | 20B | 24B | 29B | 38B | 43B | 45B | 52B | 53B | 54B | 52B | 64B | | | | |
| B*1558 | 2B | 7B | 11B | 16B | 20B | 24B | 29B | 40B | 44B | 47B | 52B | 53B | 54B | 61B | 62B | 64B | | | |
| B*1507 | 2B | 7B | 11B | 16B | 20B | 24B | 28B | 38B | 44B | 47B | 52B | 53B | 54B | 62B | 64B | | | | |
| B*1555 | 2B | 7B | 11B | 16B | 20B | 24B | 28B | 38B | 43B | 47B | 53B | 54B | 60B | 62B | 64B | | | | |
| B*1545 | 2B | 7B | 11B | 16B | 20B | 24B | 28B | 34B | 44B | 47B | 52B | 53B | 54B | 62B | 64B | | | | |
| B*1543 | 2B | 7B | 11B | 16B | 22B | 25B | 29B | 38B | 44B | 47B | 52B | 53B | 54B | 57B | 62B | 64B | | | |
| B*3519 | 2B | 7B | 13B | 15B | 20B | 24B | 29B | 38B | 43B | 47B | 53B | 54B | 58B | 59B | 62B | | | | |
| B*4010 | 2B | 7B | 13B | 16B | 20B | 24B | 29B | 34B | 43B | 45B | 50B | 52B | 53B | 54B | 61B | | | | |
| B*1546 | 2B | 7B | 13B | 16B | 20B | 24B | 29B | 38B | 44B | 47B | 52B | 53B | 54B | 62B | 64B | | | | |
| B*5401 | 2B | 7B | 10B | 14B | 20B | 24B | 30B | 35B | 43B | 46B | 50B | 61B | 62B | | | | | | |
| B*5507 | 2B | 7B | 10B | 14B | 20B | 24B | 30B | 35B | 43B | 46B | 50B | 61B | 62B | | | | | | |
| B*40011 | 2B | 6B | 13B | 16B | 20B | 24B | 29B | 34B | 43B | 45B | 49B | 50B | 52B | 53B | 54B | 61B | | | |
| B*40012 | 2B | 6B | 13B | 16B | 20B | 24B | 29B | 34B | 43B | 45B | 49B | 50B | 52B | 53B | 54B | 61B | | | |
| B*4033 | 2B | 6B | 13B | 16B | 20B | 24B | 29B | 34B | 43B | 45B | 49B | 50B | 52B | 53B | 54B | | | | |
| B*4014 | 2B | 6B | 13B | 16B | 20B | 24B | 29B | 34B | 43B | 45B | 49B | 50B | 52B | 53B | 54B | 61B | 62B | | |
| B*4025 | 2B | 6B | 13B | 16B | 20B | 24B | 29B | 34B | 43B | 45B | 49B | 50B | 53B | 54B | 61B | | | | |
| B*4023 | 2B | 6B | 13B | 16B | 20B | 24B | 29B | 34B | 41B | 45B | 49B | 50B | 52B | 53B | 54B | 61B | | | |
| B*4101 | 2B | 6B | 13B | 16B | 20B | 24B | 29B | 34B | 42B | 46B | 49B | 50B | 52B | 53B | 54B | 61B | 62B | | |
| B*4103 | 2B | 6B | 13B | 16B | 20B | 24B | 29B | 34B | 42B | 46B | 49B | 50B | 52B | 53B | 54B | 61B | 62B | | |
| B*4105 | 2B | 6B | 13B | 16B | 20B | 27B | 29B | 34B | 42B | 46B | 49B | 50B | 52B | 53B | 54B | 61B | 62B | | |
| B*4502 | 2B | 6B | 13B | 16B | 20B | 24B | 29B | 40B | 42B | 47B | 49B | 50B | 52B | 53B | 54B | 58B | 59B | 60B | 61B |
| B*4409 | 2B | 6B | 13B | 16B | 20B | 24B | 29B | 36B | 42B | 47B | 50B | 52B | 53B | 54B | 60B | 62B | | | |
| B*4702 | 2B | 6B | 13B | 16B | 20B | 24B | 29B | 36B | 43B | 45B | 50B | 52B | 53B | 54B | | | | | |
| B*4703 | 2B | 6B | 13B | 16B | 20B | 24B | 29B | 36B | 43B | 45B | 50B | 52B | 53B | 54B | 57B | | | | |
| B*4501 | 2B | 6B | 13B | 16B | 20B | 24B | 29B | 35B | 42B | 47B | 49B | 50B | 52B | 53B | 54B | 58B | 59B | 60B | 61B |
| B*4504 | 2B | 6B | 13B | 16B | 20B | 24B | 29B | 35B | 42B | 47B | 49B | 50B | 52B | 53B | 54B | 58B | 59B | 60B | 61B |
| B*4503 | 2B | 6B | 13B | 16B | 20B | 24B | 29B | 35B | 42B | 47B | 49B | 50B | 52B | 53B | 54B | 58B | 59B | 60B | 61B |
| B*5001 | 2B | 6B | 13B | 16B | 20B | 24B | 29B | 35B | 43B | 47B | 49B | 50B | 52B | 53B | 54B | 58B | 59B | 60B | 61B |
| B*5002 | 2B | 6B | 13B | 16B | 20B | 24B | 29B | 35B | 43B | 47B | 49B | 50B | 52B | 53B | 54B | 58B | 59B | 59B | 60B |
| B*5004 | 2B | 6B | 13B | 16B | 20B | 24B | 29B | 35B | 43B | 47B | 49B | 50B | 52B | 53B | 54B | 60B | 61B | 62B | |
| B*1553 | 2B | 6B | 13B | 16B | 20B | 24B | 29B | 38B | 44B | 47B | 50B | 52B | 53B | 54B | 62B | 64B | | | |
| B*4031 | 2B | 6B | 13B | 16B | 20B | 24B | 28B | 34B | 43B | 45B | 49B | 50B | 52B | 53B | 54B | | | | |
| B*4015 | 2B | 6B | 13B | 16B | 20B | 24B | 28B | 34B | 41B | 45B | 49B | 50B | 52B | 53B | 54B | 61B | | | |
| B*4016 | 2B | 6B | 13B | 16B | 20B | 24B | 28B | 34B | 41B | 45B | 49B | 50B | 52B | 53B | 54B | 61B | | | |
| B*4032 | 2B | 6B | 13B | 16B | 20B | 24B | 28B | 34B | 41B | 45B | 49B | 50B | 52B | 53B | 54B | | | | |
| B*4102 | 2B | 6B | 13B | 16B | 20B | 24B | 28B | 34B | 42B | 46B | 49B | 50B | 52B | 53B | 54B | 61B | 62B | | |
| B*4104 | 2B | 6B | 13B | 16B | 20B | 24B | 28B | 34B | 42B | 46B | 49B | 50B | 52B | 53B | 54B | 59B | 62B | | |
| B*4415 | 2B | 6B | 13B | 16B | 20B | 25B | 29B | 35B | 42B | 47B | 49B | 50B | 52B | 53B | 54B | 56B | 57B | 58B | 59B |
| B*4030 | 2B | 6B | 13B | 16B | 20B | 24B | 33B | 34B | 43B | 45B | 49B | 50B | 52B | 53B | 54B | 61B | | | |
| B*4034 | 2B | 6B | 13B | 16B | 20B | 24B | 33B | 34B | 43B | 45B | 49B | 50B | 52B | 53B | 54B | 61B | | | |
| B*44021 | 2B | 6B | 13B | 16B | 21B | 25B | 29B | 36B | 42B | 47B | 50B | 52B | 53B | 54B | 56B | 57B | 60B | 62B | |
| B*4414 | 2B | 6B | 13B | 16B | 21B | 25B | 29B | 36B | 42B | 47B | 50B | 52B | 53B | 54B | 56B | 57B | 61B | 62B | |
| B*4411 | 2B | 6B | 13B | 16B | 21B | 25B | 29B | 36B | 42B | 47B | 50B | 52B | 53B | 54B | 57B | 60B | 62B | | |
| B*4412 | 2B | 6B | 13B | 16B | 21B | 25B | 29B | 36B | 42B | 47B | 50B | 53B | 54B | 56B | 57B | 60B | 62B | | |
| B*4424 | 2B | 6B | 13B | 168 | 21B | 25B | 29B | 36B | 42B | 47B | 50B | 52B | 53B | 54B | 56B | 57B | 60B | 62B | |
| B*4416 | 2B | 6B | 13B | 16B | 21B | 25B | 29B | 36B | 42B | 45B | 50B | 52B | 53B | 54B | 56B | 57B | 60B | 62B | |
| B*4421 | 2B | 6B | 13B | 16B | 21B | 25B | 29B | 36B | 42B | 45B | 50B | 52B | 53B | 54B | 56B | 57B | 60B | 62B | |
| B*44031 | 2B | 6B | 13B | 16B | 21B | 25B | 29B | 36B | 43B | 47B | 50B | 52B | 53B | 54B | 56B | 57B | 60B | 62B | |
| B*44032 | 2B | 6B | 13B | 16B | 21B | 25B | 29B | 36B | 43B | 47B | 50B | 52B | 53B | 54B | 56B | 57B | 58B | 60B | 62B |
| B*4407 | 2B | 6B | 13B | 16B | 21B | 25B | 29B | 36B | 43B | 47B | 50b | 52B | 53B | 54B | 54B | 57B | 58B | 60B | 62B |
| B*4413 | 2B | 6B | 13B | 16B | 21B | 25B | 29B | 36B | 43B | 47B | 50b | 52B | 53B | 54B | 56B | 57B | 60B | 62B | |
| B*4404 | 2B | 6B | 13B | 16B | 21B | 25B | 29B | 36B | 41B | 46B | 50B | 52B | 53B | 54B | 56B | 57B | 60B | 62B | |
| B*4410 | 2B | 6B | 13B | 16B | 21B | 25B | 29B | 35B | 43B | 47B | 50B | 52B | 53B | 54B | 56B | 57B | 59B | 61B | 62B |
| B*4902 | 2B | 6B | 13B | 16B | 21B | 25B | 29B | 35B | 43B | 47B | 49B | 50B | 52B | 53B | 54B | 56B | 57B | 58B | 59B |
| B*4417 | 2B | 6B | 13B | 16B | 21B | 25B | 29B | 38B | 42B | 47B | 50B | 52B | 53B | 54B | 56B | 57B | 62B | | |
| B*4420 | 2B | 6B | 13B | 16B | 21B | 25B | 30B | 36B | 42B | 47B | 50B | 52B | 53B | 54B | 56B | 57B | 58B | 60B | 62B |
| B*4418 | 2B | 6B | 13B | 16B | 21B | 26B | 29B | 35B | 42B | 47B | 49B | 50B | 52B | 53B | 54B | 56B | 57B | 58B | 59B |
| B*4901 | 2B | 6B | 13B | 16B | 21B | 26B | 29B | 35B | 43B | 47B | 49B | 50B | 52B | 53B | 54B | 56B | 57B | 58B | 59B |
| B*4701 | 2B | 6B | 13B | 16B | 22B | 25B | 29B | 36B | 43B | 45B | 50B | 52B | 53B | 54B | 57B | | | | |
| B*4007 | 2B | 6B | 13B | 15B | 20B | 24B | 29B | 34B | 43B | 45B | 49B | 50B | 52B | 53B | 54B | 61B | | | |
| B*44022 | 2B | 6B | 13B | 19B | 21B | 25B | 29B | 36B | 42B | 47B | 50B | 52B | 53B | 54B | 56B | 57B | 60B | 62B | |
| B*4408 | 2B | 6B | 11B | 16B | 21B | 25B | 29B | 36B | 42B | 47B | 50B | 52B | 53B | 54B | 56B | 57B | 60B | 62B | |
| B*1301 | 2B | 6B | 11B | 16B | 21B | 25B | 29B | 35B | 43B | 45B | 51B | 52B | 53B | 54B | 56B | 57B | 58B | 59B | 61B |
| B*1306 | 2B | 6B | 11B | 16B | 21B | 25B | 29B | 35B | 43B | 46B | 51B | 52B | 53B | 54B | 56B | 57B | 58B | 59B | 61B |
| B*1302 | 2B | 6B | 11B | 16B | 21B | 25B | 30B | 35B | 43B | 45B | 51B | 52B | 53B | 54B | 56B | 57B | 58B | 59B | 61B |
| B*1303 | 2B | 6B | 11B | 16B | 21B | 25B | 30B | 35B | 43B | 47B | 51B | 52B | 53B | 54B | 56B | 57B | 58B | 59B | 61B |
| B*1304 | 2B | 6B | 11B | 16B | 21B | 25B | 30B | 38B | 43B | 47B | 51B | 52B | 53B | 54B | 56B | 57B | 58B | 59B | 62B |
| B*0802 | 2B | 5B | 9B | 15B | 21B | 25B | 28B | 34B | 42B | 46B | 508 | 53B | 56B | 61B | 62B | | | | |
| B*0806 | 2B | 5B | 9B | 15B | 21B | 24B | 28B | 34B | 41B | 46B | 50B | 53B | 61B | | | | | | |
| B*0803 | 2B | 5B | 9B | 15B | 21B | 26B | 28B | 34B | 42B | 46B | 50B | 53B | 54B | 56B | 57B | 61B | 62B | | |
| B*3525 | 2B | 5B | 9B | 15B | 20B | 24B | 29B | 38B | 43B | 47B | 50B | 53B | 54B | 58B | 59B | 62B | | | |
| B*0812 | 2B | 5B | 9B | 15B | 20B | 24B | 29B | 34B | 42B | 46B | 50B | 53B | 61B | 62B | | | | | |
| B*0801 | 2B | 5B | 9B | 15B | 20B | 24B | 28B | 34B | 42B | 46B | 50B | 53B | 61B | 62B | | | | | |
| B*0807 | 2B | 5B | 9B | 15B | 20B | 24B | 28B | 34B | 42B | 46B | 50B | 53B | 62B | | | | | | |
| B*0805 | 2B | 5B | 9B | 15B | 20B | 24B | 28B | 34B | 42B | 46B | 50B | 53B | 61B | 62B | | | | | |
| B*0810 | 2B | 5B | 9B | 15B | 20B | 24B | 28B | 34B | 42B | 46B | 50B | 53B | 61B | 62B | | | | | |
| B*0811 | 2B | 5B | 9B | 15B | 20B | 24B | 28B | 34B | 42B | 48B | 50B | 53B | 61B | 62B | | | | | |
| B*0813 | 2B | 5B | 9B | 15B | 20B | 24B | 28B | 34B | 43B | 46B | 50B | 53B | 61B | 62B | | | | | |
| B*0809 | 2B | 5B | 9B | 15B | 20B | 24B | 30B | 34B | 42B | 46B | 50B | 53B | 58B | 61B | 62B | | | | |
| B*1529 | 2B | 5B | 9B | 15B | 20B | 24B | 29B | 38B | 43B | 47B | 50B | 53B | 54B | 62B | 64B | | | | |
| B*0804 | 2B | 5B | 9B | 16B | 20B | 24B | 28B | 34B | 42B | 46B | 50B | 53B | 62B | | | | | | |
| B*4012 | 2B | 5B | 9B | 16B | 20B | 24B | 29B | 34B | 43B | 45B | 50B | 52B | 53B | 54B | 61B | | | | |
| B*1503 | 2B | 5B | 9B | 16B | 20B | 24B | 29B | 38B | 43B | 47B | 50B | 52B | 53B | 54B | 62B | 64B | | | |
| B*1564 | 2B | 5B | 9B | 16B | 20B | 24B | 29B | 38B | 43B | 47B | 50B | 53B | 54B | 62B | 64B | | | | |
| B*1561 | 2B | 5B | 9B | 16B | 20B | 24B | 29B | 38B | 43B | 47B | 50B | 52B | 53B | 62B | 64B | | | | |
| B*1562 | 2B | 5B | 9B | 16B | 20B | 24B | 29B | 38B | 43B | 47B | 50B | 52B | 53B | 54B | 58B | 59B | 62B | 64B | |
| B*1547 | 2B | 5B | 9B | 16B | 20B | 24B | 29B | 38B | 41B | 45B | 50B | 52B | 53B | 54B | 62B | 64B | | | |
| B*1549 | 2B | 5B | 9B | 16B | 20B | 24B | 29B | 38B | 41B | 45B | 50B | 52B | 536 | 54B | 62B | 64B | | | |
| B*1554 | 2B | 5B | 9B | 16B | 20B | 24B | 29B | 38B | 44B | 47B | 50B | 52B | 53B | 54B | 62B | 64B | | | |
| B*3904 | 2B | 5B | 9B | 17B | 20B | 24B | 29B | 40B | 43B | 46B | 50B | 53B | 61B | 62B | 63B | | | | |
| B*1509 | 2B | 5B | 9B | 17B | 20B | 24B | 29B | 34B | 43B | 47B | 50B | 53B | 54B | 61B | 62B | 64B | | | |
| B*1510 | 2B | 5B | 9B | 17B | 20B | 24B | 29B | 34B | 43B | 47B | 50B | 53B | 54B | 62B | 64B | | | | |
| B*1537 | 2B | 5B | 9B | 17B | 20B | 24B | 29B | 34B | 43B | 47B | 50B | 53B | 54B | 62B | 64B | | | | |
| B*1518 | 2B | 5B | 9B | 17B | 20B | 24B | 29B | 38B | 43B | 47B | 50B | 53B | 54B | 62B | 64B | | | | |
| B*1551 | 2B | 5B | 9B | 17B | 20B | 24B | 29B | 38B | 42B | 47B | 50B | 53B | 54B | 62B | 64B | | | | |
| B*1552 | 2B | 5B | 9B | 17B | 20B | 24B | 29B | 38B | 43B | 45B | 50B | 53B | 54B | 62B | 64B | | | | |
| B*1523 | 2B | 5B | 9B | 17B | 21B | 26B | 29B | 38B | 43B | 47B | 50B | 53B | 54B | 56B | 57B | 62B | 64B | | |
| B*8301 | 2B | 5B | 9B | 14B | 20B | 24B | 29B | 36B | 42B | 47B | 60B | 62B | | | | | | | |
| B*5606 | 2B | 8B | 12B | 14B | 20B | 24B | 30B | 34B | 43B | 47B | 51B | 61B | 62B | | | | | | |
| B*7801 | 2B | 8B | 12B | 15B | 20B | 24B | 30B | 34B | 43B | 47B | 51B | 53B | 61B | 62B | | | | | |
| B*78022 | 2B | 8B | 12B | 15B | 20B | 24B | 30B | 34B | 43B | 47B | 51B | 53B | 54B | 61B | 62B | | | | |
| B*5306 | 2B | 8B | 12B | 15B | 21B | 26B | 29B | 38B | 43B | 47B | 51B | 53B | 54B | 56B | 57B | 58B | 59B | 62B | |
| B*4406 | 2B | 8B | 12B | 15B | 21B | 26B | 29B | 36B | 42B | 47B | 51B | 53B | 54B | 56B | 57B | 60B | 62B | | |
| B*5104 | 2B | 8B | 12B | 15B | 21B | 26B | 29B | 34B | 43B | 47B | 51B | 53B | 54B | 56B | 57B | 58B | 61B | 62B | |
| B*5106 | 2B | 8B | 12B | 15B | 21B | 26B | 29B | 34B | 43B | 47B | 51B | 53B | 54B | 56B | 57B | 61B | 62B | | |
| B*51131 | 2B | 8B | 12B | 15B | 21B | 26B | 30B | 40B | 43B | 47B | 51B | 53B | 54B | 56B | 57B | 58B | 61B | 62B | |
| B*51132 | 2B | 8B | 12B | 15B | 21B | 26B | 30B | 40B | 43B | 47B | 51B | 53B | 54B | 56B | 57B | 58B | 61B | 62B | |
| B*5116 | 2B | 8B | 12B | 15B | 21B | 26B | 30B | 34B | 43B | 45B | 51B | 53B | 54B | 56B | 57B | 58B | 61B | 62B | |
| B*5110 | 2B | 8B | 12B | 15B | 21B | 26B | 30B | 34B | 43B | 45B | 51B | 53B | 54B | 56B | 57B | 58B | 61B | | |
| B*5121 | 2B | 8B | 12B | 15B | 21B | 26B | 30B | 34B | 43B | 46B | 51B | 53B | 54B | 56B | 57B | 58B | 61B | 62B | |
| B*51011 | 2B | 8B | 12B | 15B | 21B | 26B | 30B | 34B | 43B | 47B | 51B | 53B | 54B | 56B | 57B | 58B | 61B | 62B | |
| B*51021 | 2B | 8B | 12B | 15B | 21B | 26B | 30B | 34B | 43B | 47B | 51B | 53B | 54B | 56B | 57B | 58B | 61B | 62B | |
| B*51014 | 2B | 8B | 12B | 15B | 21B | 26B | 30B | 34B | 43B | 47B | 51B | 53B | 54B | 56B | 57B | 58B | 61B | 62B | |
| B*5124 | 2B | 8B | 12B | 15B | 21B | 26B | 30B | 34B | 43B | 47B | 51B | 53B | 54B | 56B | 57B | 58B | 61B | 62B | |
| B*51013 | 2B | 8B | 12B | 15B | 21B | 26B | 30B | 34B | 43B | 47B | 51B | 53B | 54B | 56B | 57B | 58B | 61B | 62B | |
| B*5109 | 2B | 8B | 12B | 15B | 21B | 26B | 30B | 34B | 43B | 47B | 51B | 53B | 54B | 56B | 57B | 58B | 61B | 62B | |
| B*5103 | 2B | 8B | 12B | 15B | 21B | 26B | 30B | 34B | 43B | 47B | 51B | 53B | 54B | 56B | 57B | 58B | 61B | 62B | |
| B*5123 | 2B | 8B | 12B | 15B | 21B | 26B | 30B | 34B | 43B | 47B | 51B | 53B | 54B | 56B | 57B | 58B | 61B | 62B | |
| B*5119 | 2B | 8B | 12B | 15B | 21B | 26B | 30B | 34B | 43B | 47B | 51B | 53B | 54B | 56B | 57B | 58B | 61B | 62B | 63B |
| B*5114 | 2B | 8B | 12B | 15B | 21B | 26B | 30B | 34B | 43B | 47B | 51B | 53B | 54B | 56B | 57B | 58B | 61B | 62B | |
| B*5117 | 2B | 8B | 12B | 15B | 21B | 26B | 30B | 34B | 43B | 47B | 51B | 53B | 54B | 56B | 57B | 58B | 61B | 62B | |
| B*5118 | 2B | 8B | 12B | 15B | 21B | 26B | 30B | 34B | 43B | 47B | 51B | 53B | 54B | 56B | 57B | 58B | 61B | 62B | |
| B*5112 | 2B | 8B | 12B | 15B | 21B | 26B | 30B | 34B | 43B | 47B | 51B | 53B | 54B | 56B | 57B | 58B | 61B | 62B | |
| B*5108 | 2B | 8B | 12B | 15B | 21B | 26B | 30B | 34B | 42B | 47B | 51B | 53B | 54B | 56B | 57B | 58B | 61B | 62B | |
| B*5120 | 2B | 8B | 12B | 15B | 21B | 26B | 30B | 34B | 42B | 47B | 51B | 53B | 56B | 57B | 57B | 61B | 62B | | |
| B*5107 | 2B | 8B | 12B | 16B | 21B | 26B | 30B | 34B | 43B | 47B | 51B | 53B | 54B | 56B | 57B | 58B | 61B | 62B | |
| B*52012 | 2B | 8B | 12B | 16B | 21B | 26B | 30B | 34B | 43B | 47B | 51B | 52B | 53B | 54B | 56B | 57B | 58B | 61B | 62B |
| B*5203 | 2B | 8B | 12B | 16B | 21B | 26B | 30B | 34B | 43B | 47B | 51B | 52B | 53B | 54B | 56B | 57B | 58B | 61B | 62B |
| B*4903 | 2B | 8B | 12B | 16B | 21B | 26B | 29B | 35B | 43B | 47B | 49B | 51B | 52B | 54B | 56B | 57B | 58B | 58B | 60B |
| B*1804 | 2B | 8B | 12B | 16B | 20B | 24B | 29B | 38B | 43B | 46B | 51B | 53B | 54B | 62B | 64B | | | | |
| B*5122 | 2B | 8B | 12B | 17B | 21B | 26B | 30B | 34B | 43B | 47B | 51B | 53B | 56B | 54B | 58B | 61B | 62B | | |
| B*7803 | 2B | 8B | 12B | 17B | 20B | 24B | 30B | 34B | 43B | 47B | 51B | 53B | 61B | 62B | | | | | |
| B*5605 | 2B | 8B | 9B | 14B | 20B | 24B | 30B | 34B | 43B | 47B | 50B | 61B | 62B | | | | | | |
| B*5202 | 2B | 8B | 11B | 16B | 21B | 26B | 30B | 34B | 43B | 47B | 52B | 53B | 54B | 56B | 57B | 58B | 61B | 62B | |
| B*4020 | 1B | 6B | 13B | 16B | 20B | 24B | 29B | 38B | 43B | 45B | 49B | 50B | 52B | 53B | 54B | | | | |
| B*4004 | 1B | 6B | 13B | 16B | 20B | 24B | 29B | 34B | 43B | 45B | 49B | 50B | 52B | 53B | 54B | 61B | | | |
| B*4011 | 1B | 6B | 13B | 16B | 20B | 24B | 29B | 34B | 43B | 45B | 49B | 50B | 52B | 53B | 54B | 61B | | | |
| B*4026 | 1B | 6B | 13B | 16B | 20B | 24B | 29B | 34B | 43B | 47B | 49B | 50B | 52B | 53B | 54B | 61B | 62B | | |
| B*4028 | 1B | 6B | 13B | 16B | 20B | 24B | 29B | 34B | 43B | 47B | 49B | 50B | 52B | 53B | 54B | 61B | 62B | | |
| B*4003 | 1B | 6B | 13B | 16B | 20B | 24B | 28B | 38B | 43B | 45B | 49B | 50B | 52B | 53B | 54B | | | | |
| B*4005 | 1B | 6B | 13B | 16B | 20B | 24B | 28B | 34B | 43B | 45B | 49B | 50B | 52B | 53B | 54B | 61B | | | |
| B*4018 | 1B | 6B | 13B | 16B | 20B | 24B | 28B | 34B | 43B | 45B | 49B | 50B | 52B | 53B | 54B | | | | |
| B*4002 | 1B | 6B | 13B | 16B | 20B | 24B | 28B | 34B | 43B | 45B | 49B | 50B | 52B | 53B | 54B | 61B | | | |
| B*4009 | 1B | 6B | 13B | 16B | 20B | 24B | 28B | 34B | 43B | 45B | 49B | 50B | 52B | 53B | 54B | 60B | | | |
| B*4024 | 1B | 6B | 13B | 16B | 20B | 24B | 28B | 34B | 43B | 45B | 49B | 50B | 52B | 53B | 54B | 59B | | | |
| B*4029 | 1B | 6B | 13B | 16B | 20B | 24B | 28B | 34B | 43B | 45B | 49B | 50B | 53B | 54B | 61B | | | | |
| B*4027 | 1B | 6B | 13B | 16B | 20B | 24B | 28B | 39B | 43B | 45B | 49B | 50B | 52B | 53B | 54B | 61B | | | |
| B*4006 | 1B | 6B | 13B | 16B | 20B | 24B | 30B | 34B | 43B | 45B | 49B | 50B | 52B | 53B | 54B | 61B | | | |
| B*4019 | 1B | 6B | 13B | 16B | 21B | 26B | 28B | 34B | 43B | 45B | 49B | 50B | 52B | 53B | 54B | 56B | 57B | 61B | |
| B*4008 | 1B | 6B | 13B | 15B | 20B | 24B | 28B | 34B | 43B | 45B | 49B | 50B | 53B | 54B | 61B | | | | |
| B*4013 | 1B | 6B | 13B | 15B | 21B | 26B | 26B | 34B | 43B | 45B | 49B | 50B | 52B | 53B | 54B | 56B | 57B | 61B | |
| B*7301 | 1B | 6B | 9B | 17B | 23B | 24B | 30B | 40B | 43B | 45B | 49B | 55B | 60B | 61B | | | | | |
| B*2701 | 1B | 6B | 9B | 17B | 21B | 25B | 32B | 36B | 43B | 45B | 50B | 52B | 54B | 56B | 57B | 58B | 64B | | |
| B*2702 | 1B | 6B | 9B | 17B | 21B | 26B | 32B | 36B | 43B | 45B | 50B | 52B | 56B | 57B | 58B | 64B | | | |
| B*2707 | 1B | 6B | 9B | 17B | 22B | 25B | 28B | 34B | 43B | 45B | 50B | 52B | 57B | 61B | 64B | | | | |
| B*27052 | 1B | 6B | 9B | 17B | 22B | 25B | 32B | 36B | 43B | 45B | 50B | 52B | 57B | 58B | 64B | | | | |
| B*27054 | 1B | 6B | 9B | 17B | 22B | 25B | 32B | 36B | 43B | 45B | 50B | 52B | 57B | 58B | 64B | | | | |
| B*2713 | 1B | 6B | 9B | 17B | 22B | 25B | 32B | 36B | 43B | 45B | 50B | 52B | 57B | 58B | 64B | | | | |
| B*2716 | 1B | 6B | 9B | 17B | 22B | 25B | 32B | 36B | 43B | 45B | 50B | 53B | 52B | 57B | 58B | 64B | | | |
| B*2710 | 1B | 6B | 9B | 17B | 22B | 25B | 32B | 36B | 43B | 45B | 50B | 52B | 57B | 58B | 64B | | | | |
| B*2703 | 1B | 6B | 9B | 17B | 22B | 25B | 32B | 36B | 43B | 45B | 50B | 52B | 57B | 58B | 64B | | | | |
| B*2717 | 1B | 6B | 9B | 17B | 22B | 25B | 32B | 36B | 43B | 45B | 50B | 52B | 57B | 58B | 64B | | | | |
| B*2709 | 1B | 6B | 9B | 17B | 22B | 25B | 32B | 37B | 43B | 45B | 50B | 52B | 57B | 58B | 64B | | | | |
| B*2714 | 1B | 6B | 9B | 17B | 22B | 25B | 30B | 36B | 43B | 45B | 50B | 52B | 57B | 58B | 59B | 64B | | | |
| B*2719 | 1B | 6B | 9B | 17B | 22B | 25B | 29B | 36B | 43B | 45B | 50B | 52B | 57B | 64B | | | | | |
| B*2704 | 1B | 6B | 9B | 17B | 20B | 25B | 32B | 36B | 43B | 45B | 50B | 52B | 57B | 58B | 64B | | | | |
| B*2715 | 1B | 6B | 9B | 17B | 20B | 25B | 32B | 36B | 43B | 46B | 50B | 52B | 57B | 58B | 64B | | | | |
| B*2706 | 1B | 6B | 9B | 17B | 20B | 25B | 32B | 34B | 43B | 45B | 50B | 52B | 57B | 58B | 60B | 64B | | | |
| B*2722 | 1B | 6B | 9B | 17B | 20B | 25B | 32B | 34B | 43B | 45B | 50B | 52B | 57B | 58B | 60B | 64B | | | |
| B*2720 | 1B | 6B | 9B | 17B | 20B | 25B | 32B | 34B | 43B | 45B | 50B | 52B | 57B | 61B | 64B | | | | |
| B*2711 | 1B | 6B | 9B | 17B | 20B | 25B | 28B | 34B | 43B | 45B | 50B | 52B | 57B | 61B | 64B | | | | |
| B*2721 | 1B | 6B | 9B | 17B | 20B | 25B | 29B | 34B | 43B | 45B | 50B | 52B | 57B | 60B | 64B | | | | |
| B*2708 | 1B | 6B | 9B | 17B | 20B | 24B | 32B | 36B | 43B | 45B | 50B | 52B | 58B | 64B | | | | | |
| B*2712 | 1B | 6B | 9B | 17B | 20B | 24B | 32B | 36B | 43B | 45B | 50B | 52B | 53B | 58B | 64B | | | | |
| B*2718 | 1B | 6B | 9B | 16B | 20B | 24B | 32B | 36B | 43B | 45B | 50B | 52B | 53B | 54B | 58B | 64B | | | |
| B*2723 | 1B | 6B | 9B | 15B | 20B | 25B | 32B | 36B | 43B | 45B | 50B | 53B | 54B | 57B | 58B | 64B | | | |
| B*3803 | 1B | 5B | 9B | 16B | 20B | 25B | 29B | 40B | 43B | 46B | 50B | 53B | 52B | 56B | 57B | 61B | 62B | 63B | |
| B*4802 | 1B | 5B | 9B | 16B | 20B | 24B | 29B | 38B | 43B | 47B | 52B | 53B | 54B | 58B | 59B | 62B | | | |
| B*4801 | 1B | 5B | 9B | 16B | 20B | 24B | 28B | 34B | 43B | 45B | 52B | 53B | 54B | 61B | | | | | |
| B*4804 | 1B | 5B | 9B | 16B | 20B | 24B | 28B | 34B | 43B | 45B | 52B | 53B | 54B | 61B | | | | | |
| B*4807 | 1B | 5B | 9B | 16B | 20B | 24B | 28B | 40B | 43B | 45B | 52B | 53B | 54B | 61B | | | | | |
| B*4803 | 1B | 5B | 9B | 16B | 20B | 24B | 29B | 34B | 43B | 45B | 52B | 53B | 54B | 61B | | | | | |
| B*39021 | 1B | 5B | 9B | 16B | 20B | 24B | 29B | 40B | 43B | 46B | 50B | 53B | 52B | 61B | 62B | 63B | | | |
| B*39022 | 1B | 5B | 9B | 16B | 20B | 24B | 29B | 40B | 43B | 46B | 50B | 52B | 53B | 61B | 62B | 63B | | | |
| B*3913 | 1B | 5B | 9B | 16B | 20B | 24B | 29B | 40B | 43B | 46B | 50B | 52B | 53B | 54B | 61B | 62B | 63B | | |
| B*3923 | 1B | 5B | 9B | 16B | 20B | 24B | 29B | 40B | 43B | 46B | 50B | 52B | 53B | 616 | 61B | 63B | | | |
| B*3908 | 1B | 5B | 9B | 16B | 20B | 24B | 29B | 40B | 41B | 46B | 50B | 52B | 53B | 54B | 61B | 62B | 63B | | |
| B*3914 | 1B | 5B | 9B | 17B | 20B | 24B | 28B | 34B | 43B | 46B | 50B | 53B | 61B | 62B | 63B | | | | |
| B*3903 | 1B | 5B | 9B | 17B | 20B | 24B | 28B | 40B | 43B | 46B | 50B | 53B | 61B | 62B | 63B | | | | |
| B*3924 | 1B | 5B | 9B | 17B | 20B | 24B | 28B | 40B | 43B | 46B | 50B | 53B | 61B | 62B | 63B | | | | |
| B*39011 | 1B | 5B | 9B | 17B | 20B | 24B | 29B | 40B | 43B | 46B | 50B | 53B | 61B | 62B | 63B | | | | |
| B*39013 | 1B | 5B | 9B | 17B | 20B | 24B | 29B | 40B | 43B | 46B | 50B | 53B | 61B | 62B | 63B | | | | |
| B*3915 | 1B | 5B | 9B | 17B | 20B | 24B | 29B | 40B | 43B | 46B | 50B | 53B | 62B | 63B | | | | | |
| B*3909 | 1B | 5B | 9B | 17B | 20B | 24B | 29B | 40B | 43B | 46B | 50B | 53B | 61B | 62B | 63B | | | | |
| B*39014 | 1B | 5B | 9B | 17B | 20B | 24B | 29B | 40B | 43B | 46B | 50B | 53B | 54B | 61B | 62B | 63B | | | |
| B*3905 | 1B | 5B | 9B | 17B | 20B | 24B | 29B | 40B | 43B | 46B | 50B | 53B | 61B | 62B | 63B | | | | |
| B*3922 | 1B | 5B | 9B | 17B | 20B | 24B | 29B | 40B | 43B | 46B | 50B | 52B | 53B | 61B | 62B | 63B | | | |
| B*3918 | 1B | 5B | 9B | 17B | 20B | 24B | 29B | 40B | 44B | 46B | 50B | 53B | 61B | 62B | 63B | | | | |
| B*3911 | 1B | 5B | 9B | 17B | 20B | 24B | 29B | 40B | 41B | 46B | 50B | 53B | 54B | 61B | 62B | 63B | | | |
| B*39061 | 1B | 5B | 9B | 17B | 20B | 24B | 30B | 40B | 43B | 46B | 50B | 53B | 61B | 62B | 63B | | | | |
| B*39062 | 1B | 5B | 9B | 17B | 20B | 24B | 30B | 40B | 43B | 46B | 50B | 53B | 58B | 61B | 62B | 63B | | | |
| B*4201 | 1B | 5B | 9B | 14B | 20B | 24B | 28B | 34B | 42B | 46B | 61B | 62B | | | | | | | |
| B*4202 | 1B | 5B | 9B | 14B | 20B | 24B | 28B | 34B | 42B | 46B | 49B | 61B | 62B | | | | | | |
| B*0719 | 1B | 5B | 9B | 14B | 20B | 24B | 28B | 34B | 42B | 46B | 64B | | | | | | | | |
| B*0704 | 1B | 5B | 9B | 14B | 20B | 24B | 28B | 34B | 42B | 45B | | | | | | | | | |
| B*0725 | 1B | 5B | 9B | 14B | 20B | 24B | 28B | 34B | 42B | 45B | | | | | | | | | |
| B*8101 | 1B | 5B | 9B | 14B | 20B | 24B | 28B | 34B | 43B | 45B | 61B | | | | | | | | |
| B*07021 | 1B | 5B | 9B | 14B | 20B | 24B | 28B | 34B | 41B | 45B | | | | | | | | | |
| B*07022 | 1B | 5B | 9B | 14B | 20B | 24B | 26B | 34B | 41B | 45B | | | | | | | | | |
| B*0723 | 1B | 5B | 9B | 14B | 20B | 24B | 28B | 34B | 41B | 45B | | | | | | | | | |
| B*0705 | 1B | 5B | 9B | 14B | 20B | 24B | 28B | 34B | 41B | 45B | 61B | | | | | | | | |
| B*0703 | 1B | 5B | 9B | 14B | 20B | 24B | 28B | 34B | 41B | 45B | 53B | | | | | | | | |
| B*07023 | 1B | 5B | 9B | 14B | 20B | 24B | 28B | 34B | 41B | 45B | 50B | | | | | | | | |
| B*0715 | 1B | 5B | 9B | 14B | 20B | 24B | 28B | 34B | 41B | 45B | 55B | | | | | | | | |
| B*0716 | 1B | 5B | 9B | 14B | 20B | 24B | 28B | 34B | 41B | 45B | 53B | 54B | | | | | | | |
| B*0721 | 1B | 5B | 9B | 14B | 20B | 24B | 28B | 34B | 41B | 45B | | | | | | | | | |
| B*0722 | 1B | 5B | 9B | 14B | 20B | 24B | 28B | 34B | 41B | 45B | | | | | | | | | |
| B*0720 | 1B | 5B | 9B | 14B | 20B | 24B | 28B | 34B | 41B | 47B | | | | | | | | | |
| B*0724 | 1B | 5B | 9B | 14B | 20B | 24B | 28B | 34B | 41B | 47B | | | | | | | | | |
| B*0709 | 1B | 5B | 9B | 14B | 20B | 24B | 28B | 38B | 41B | 45B | | | | | | | | | |
| B*0717 | 1B | 5B | 9B | 14B | 20B | 24B | 28B | 38B | 41B | 45B | 60B | | | | | | | | |
| B*3917 | 1B | 5B | 9B | 14B | 20B | 24B | 29B | 35B | 43B | 46B | 50B | 53B | 61B | 60B | 62B | 63B | | | |
| B*3910 | 1B | 5B | 9B | 14B | 20B | 24B | 29B | 40B | 43B | 46B | 50B | 53B | 61B | 62B | 63B | | | | |
| B*67012 | 1B | 5B | 9B | 14B | 20B | 24B | 29B | 40B | 43B | 46B | 50B | 61B | 62B | 63B | | | | | |
| B*3916 | 1B | 5B | 9B | 14B | 20B | 24B | 29B | 40B | 43B | 46B | 50B | 53B | 61B | 62B | 63B | | | | |
| B*67011 | 1B | 5B | 9B | 14B | 20B | 24B | 29B | 40B | 43B | 46B | 50B | 61B | 62B | 63B | | | | | |
| B*0706 | 1B | 5B | 9B | 14B | 20B | 24B | 29B | 34B | 41B | 45B | 61B | | | | | | | | |
| B*0707 | 1B | 5B | 9B | 14B | 20B | 24B | 29B | 34B | 41B | 45B | | | | | | | | | |
| B*0712 | 1B | 5B | 9B | 14B | 20B | 24B | 29B | 34B | 41B | 45B | 58B | 59B | | | | | | | |
| B*0714 | 1B | 5B | 9B | 14B | 20B | 24B | 29B | 34B | 41B | 45B | 60B | | | | | | | | |
| B*0718 | 1B | 5B | 9B | 14B | 20B | 24B | 29B | 34B | 41B | 45B | 58B | | | | | | | | |
| B*5510 | 1B | 5B | 9B | 14B | 20B | 24B | 30B | 35B | 43B | 46B | 50B | 51B | 62B | | | | | | |
| B*3920 | 1B | 5B | 9B | 14B | 21B | 24B | 29B | 40B | 43B | 46B | 50B | 53B | 54B | 61B | 62B | 63B | | | |
| B*0711 | 1B | 5B | 9B | 14B | 21B | 24B | 28B | 38B | 41B | 45B | | | | | | | | | |
| B*3801 | 1B | 5B | 9B | 17B | 21B | 26B | 29B | 40B | 43B | 46B | 50B | 53B | 54B | 56B | 57B | 61B | 62B | 63B | |
| B*38021 | 1B | 5B | 9B | 17B | 21B | 25B | 29B | 40B | 43B | 46B | 50B | 53B | 54B | 56B | 57B | 61B | 62B | 63B | |
| B*38022 | 1B | 5B | 9B | 17B | 21B | 25B | 29B | 40B | 43B | 46B | 50B | 53B | 54B | 56B | 57B | 61B | 62B | 63B | |
| B*3804 | 1B | 5B | 9B | 17B | 21B | 25B | 29B | 40B | 43B | 46B | 50B | 52B | 53B | 54B | 56B | 57B | 61B | 62B | 63B |
| B*4806 | 1B | 5B | 9B | 15B | 20B | 24B | 28B | 34B | 43B | 45B | 53B | 54B | 61B | | | | | | |
| B*0708 | 1B | 5B | 9B | 15B | 20B | 24B | 28B | 34B | 41B | 45B | 53B | | | | | | | | |
| B*0710 | 1B | 5B | 9B | 17B | 20B | 24B | 28B | 34B | 41B | 45B | | | | | | | | | |
| B*1401 | 1B | 5B | 9B | 17B | 20B | 24B | 31B | 40B | 43B | 46B | 50B | 53B | 58B | 60B | 61B | 62B | 64B | | |
| B*6702 | 1B | 5B | 10B | 14B | 20B | 24B | 29B | 40B | 43B | 46B | 50B | 52B | 55B | 61B | 62B | 63B | | | |
| B*0713 | 1B | 5B | 10B | 14B | 20B | 24B | 28B | 34B | 41B | 45B | 52B | 55B | | | | | | | |
| B*3919 | 1B | 5B | 12B | 17B | 20B | 24B | 29B | 40B | 43B | 46B | 50B | 51B | 53B | 61B | 62B | 63B | | | |
| B*3806 | 1B | 5B | 12B | 15B | 21B | 26B | 29B | 40B | 43B | 46B | 50B | 51B | 53B | 54B | 56B | 57B | 61B | 62B | 63B |
| B*3807 | 1B | 5B | 12B | 15B | 21B | 26B | 29B | 40B | 43B | 46B | 50B | 51B | 53B | 54B | 56B | 57B | 61B | 62B | 63B |
| B*1807 | 1B | 5B | 12B | 15B | 20B | 24B | 29B | 38B | 43B | 46B | 51B | 53B | 54B | 62B | 64B | | | | |
| B*3701 | 1B | 5B | 12B | 16B | 22B | 25B | 29B | 40B | 42B | 46B | 49B | 51B | 52B | 54B | 57B | 60B | 61B | 62B | |
| B*3704 | 1B | 5B | 12B | 16B | 22B | 25B | 29B | 40B | 42B | 46B | 49B | 51B | 52B | 54B | 57B | 60B | 61B | 62B | |
| B*3702 | 1B | 5B | 12B | 16B | 22B | 25B | 32B | 36B | 43B | 45B | 49B | 51B | 52B | 54B | 57B | | | | |
| B*3705 | 1B | 5B | 12B | 16B | 22B | 24B | 29B | 40B | 42B | 46B | 49B | 51B | 52B | 54B | 60B | 61B | 62B | | |
| B*1802 | 1B | 5B | 12B | 16B | 20B | 24B | 32B | 38B | 43B | 46B | 51B | 53B | 54B | 58B | 62B | 64B | | | |
| B*1801 | 1B | 5B | 12B | 16B | 20B | 24B | 29B | 38B | 43B | 46B | 51B | 53B | 54B | 62B | 64B | | | | |
| B*1803 | 1B | 5B | 12B | 16B | 20B | 24B | 29B | 38B | 43B | 46B | 51B | 53B | 62B | 64B | | | | | |
| B*1811 | 1B | 5B | 12B | 16B | 20B | 24B | 29B | 38B | 43B | 46B | 51B | 53B | 54B | 62B | 64B | | | | |
| B*1805 | 1B | 5B | 12B | 16B | 20B | 24B | 29B | 38B | 43B | 46B | 51B | 53B | 54B | 62B | 64B | | | | |
| B*1806 | 1B | 5B | 12B | 16B | 20B | 24B | 29B | 38B | 43B | 46B | 51B | 53B | 54B | 55B | 62B | 64B | | | |
| B*1808 | 1B | 5B | 12B | 16B | 20B | 24B | 29B | 38B | 43B | 46B | 51B | 53B | 54B | 62B | 64B | | | | |
| B*1812 | 1B | 5B | 12B | 16B | 20B | 24B | 29B | 38B | 43B | 46B | 51B | 52B | 54B | 62B | 64B | | | | |
| B*1810 | 1B | 5B | 12B | 16B | 20B | 24B | 29B | 38B | 43B | 45B | 51B | 53B | 54B | 62B | 64B | | | | |
| B*1813 | 1B | 5B | 12B | 16B | 20B | 24B | 29B | 38B | 41B | 46B | 51B | 53B | 54B | 62B | 64B | | | | |
| B*1809 | 1B | 5B | 12B | 16B | 21B | 25B | 29B | 38B | 43B | 46B | 51B | 53B | 54B | 56B | 57B | 62B | 64B | | |
| B*4805 | 1B | 8B | 9B | 16B | 20B | 24B | 28B | 34B | 41B | 45B | 52B | 53B | 54B | 61B | | | | | |
| B*5402 | 1B | 7B | 10B | 14B | 20B | 24B | 30B | 35B | 43B | 46B | 49B | 50B | 61B | 62B | | | | | |
| B*3907 | * | * | 9B | 17B | 20B | 24B | 29B | 38B | 43B | 46B | 53B | 54B | 62B | | | | | | |
| B*27053 | * | * | 9B | 17B | 22B | 25B | 32B | 36B | 43B | 45B | 50B | 52B | 57B | 58B | | | | | |
| B*4405 | * | * | * | 16B | 21B | 25B | 29B | 34B | 42B | 47B | 52B | 53B | 54B | 56B | 57B | 60B | 62B | | |
| B*3805 | 3B | 5B | 9B | 17B | 21B | 26B | 29B | 40B | 43B | 46B | 50B | 53B | 54B | 56B | 57B | 61B | 62B | 63B | |
| B*3912 | 3B | 5B | 9B | 17B | 20B | 24B | 29B | 40B | 43B | 46B | 50B | 61B | 62B | 63B | | | | | |
| B*1402 | 3B | 5B | 9B | 17B | 20B | 24B | 31B | 40B | 43B | 46B | 50B | 53B | 58B | 60B | 61B | 62B | 64B | | |
| B*1404 | 3B | 5B | 9B | 17B | 20B | 24B | 31B | 40B | 43B | 46B | 50B | 58B | 60B | 61B | 62B | 64B | | | |
| B*1403 | 3B | 5B | 9B | 17B | 20B | 24B | 31B | 40B | 41B | 46B | 50B | 53B | 58B | 60B | 61B | 62B | 64B | | |
| B*1405 | 3B | 5B | 9B | 17B | 20B | 24B | 28B | 40B | 43B | 46B | 50B | 53B | 60B | 61B | 62B | 64B | | | |
| B*14061 | 3B | 5B | 9B | 17B | 20B | 24B | 29B | 40B | 43B | 46B | 50B | 53B | 60B | 61B | 62B | 64B | | | |
| B*14062 | 3B | 5B | 9B | 17B | 20B | 24B | 29B | 40B | 43B | 46B | 50B | 53B | 58B | 60B | 61B | 62B | 64B | | |
| B*4422 | 3B | 6B | 13B | 16B | 21B | 25B | 29B | 36B | 42B | 47B | 50B | 52B | 53B | 54B | 56B | 57B | 60B | 62B | |
| B*8201 | 4B | 5B | 9B | 14B | 20B | 24B | 29B | 35B | 42B | 47B | 50B | 61B | 62B | | | | | | |
| B*8202 | 4B | 5B | 9B | 14B | 20B | 24B | 29B | 35B | 42B | 47B | 50B | 61B | 62B | | | | | | |

| | |
|---|---|
| patchwork region | satellite region |

| | | | | | | | | | | | | | |
|---|---|---|---|---|---|---|---|---|---|---|---|---|---|
| B*35011 | | | | 1184 | 1201 | 1232 | 1242 | 1267 | 1272 | 1285 | | | |
| B*3524 | | | | 1184 | 1201 | 1232 | 1242 | 1267 | 1272 | 1285 | 1293 | | |
| B*3529 | | | | 1184 | 1201 | 1232 | 1242 | 1267 | 1272 | 1285 | | | |
| B*3511 | | | | 1184 | 1201 | 1232 | 1242 | 1267 | 1272 | | | | |
| B*3514 | | | | 1184 | 1201 | 1232 | 1242 | 1267 | 1272 | | | | |
| B*3521 | | | | 1184 | 1201 | 1232 | 1242 | 1267 | 1272 | 1293 | | | |
| B*35012 | | | | 1184 | 1201 | 1232 | 1242 | 1267 | 1272 | 1285 | 1288 | | |
| B*3505 | | | | 1184 | 1201 | 1242 | 1267 | 1272 | 1285 | | | | |
| B*3507 | | | | 1172 | 1184 | 1201 | 1232 | 1242 | 1267 | 1272 | 1285 | | |
| B*3510 | | | | 1184 | 1201 | 1232 | 1242 | 1267 | 1272 | 1285 | | | |
| B*3523 | | | | 1184 | 1201 | 1232 | 1239 | 1242 | 1267 | 1272 | 1285 | | |
| B*3515 | | | | 1184 | 1201 | 1232 | 1242 | 1267 | 1272 | 1285 | | | |
| B*3508 | | | | 1184 | 1201 | 1232 | 1242 | 1267 | 1272 | 1285 | | | |
| B*1522 | | | | 1184 | 1201 | 1267 | 1297 | | | | | | |
| B*1559 | | | | 1184 | 1201 | 1267 | 1297 | | | | | | |
| B*35091 | | | | 1184 | 1201 | 1232 | 1242 | 1272 | 1285 | | | | |
| B*35092 | | | | 1184 | 1201 | 1232 | 1242 | 1285 | | | | | |
| B*3504 | | | | 1184 | 1201 | 1232 | 1242 | 1267 | 1272 | 1285 | | | |
| B*3502 | | | | 1184 | 1201 | 1232 | 1242 | 1244 | 1267 | 1272 | 1285 | | |
| B*3512 | | | | 1184 | 1201 | 1232 | 1267 | 1272 | 1285 | | | | |
| B*3518 | | | | 1184 | 1201 | 1232 | 1242 | 1285 | | | | | |
| B*3503 | | | | 1184 | 1201 | 1232 | 1242 | 1267 | 1272 | 1285 | | | |
| B*3506 | | | | 1184 | 1201 | 1232 | 1242 | 1267 | 1272 | 1285 | | | |
| B*3513 | | | | 1184 | 53B | 1201 | 1232 | 1242 | 1267 | 1272 | 1285 | | |
| B*3516 | | | | 1184 | 53B | 1201 | 1232 | 1242 | 1267 | 1272 | 1285 | | |
| B*3517 | | | | 1184 | 1201 | 1232 | 1242 | 1267 | 1272 | 1285 | | | |
| B*3530 | | | | 1184 | 1201 | 1232 | 1267 | 1272 | 1285 | | | | |
| B*3522 | | | | 1184 | 1201 | 1267 | 1272 | 1285 | | | | | |
| B*3531 | | | | 1184 | 1201 | 1285 | | | | | | | |
| B*3532 | | | | 1184 | 1201 | 1242 | 1267 | 1272 | 1285 | | | | |
| B*3535 | | | | 1184 | 1201 | 1232 | 1242 | 1267 | 1272 | 1285 | | | |
| B*3534 | | | | 1184 | 1201 | 1232 | 1242 | 1267 | 1272 | 1285 | | | |
| B*3533 | | | | 1184 | 1201 | 1232 | 1242 | 1267 | 1272 | 1285 | | | |
| B*3536 | | | | 1184 | 1201 | 1232 | 1242 | 1267 | 1272 | 1285 | | | |
| B*3537 | | | | 1184 | 1201 | 1233 | 1242 | 1267 | 1272 | 1285 | | | |
| B*78021 | | | | 1184 | 1201 | 1233 | 1263 | 1267 | 1272 | 1293 | | | |
| B*7804 | | | | 1184 | 1201 | 1233 | 1263 | 1267 | 1272 | 1285 | | | |
| B*5305 | | | | 1184 | 1201 | 1232 | 1242 | 1267 | 1272 | 1285 | | | |
| B*3527 | | | | 1184 | 1201 | 1232 | 1242 | 1267 | 1272 | 1285 | | | |
| B*5304 | | | | 1184 | 1201 | 1232 | 1242 | 1267 | 1272 | 1285 | | | |
| B*5307 | | | | 1184 | 1201 | 1234 | 1267 | 1272 | 1285 | | | | |
| B*5301 | | | | 1184 | 1201 | 1232 | 1242 | 1267 | 1272 | 1285 | | | |
| B*5302 | | | | 1184 | 1201 | 1232 | 1242 | 1267 | 1272 | 1285 | 1293 | | |
| B*51012 | | | | 1184 | 1201 | 1233 | 1263 | 1267 | 1272 | 1293 | | | |
| B*51022 | | | | 1184 | 1201 | 1233 | 1263 | 1267 | 1272 | 1293 | | | |
| B*5105 | | | | 1184 | 1201 | 1233 | 1263 | 1267 | 1272 | 1285 | 1293 | | |
| B*5115 | | | | 1184 | 1201 | 1233 | 1267 | 1272 | 1285 | | | | |
| B*5303 | | | | 1184 | 1201 | 1232 | 1242 | 1267 | 1272 | 1285 | | | |
| B*5801 | | | | 1184 | 1191 | 1197 | 1199 | 1204 | 1232 | 1242 | 1267 | 1272 | 1285 |
| B*5804 | | | | 1184 | 1192 | 1197 | 1199 | 1204 | 1232 | 1242 | 1267 | 1272 | 1285 |
| B*5805 | | | | 1184 | 1191 | 1197 | 1199 | 1204 | 1232 | 1242 | 1267 | 1272 | 1286 |
| B*5802 | | | | 1184 | 1191 | 1197 | 1199 | 1204 | 1242 | 1267 | 1272 | 1285 | |
| B*5806 | | | | 1184 | 1191 | 1197 | 1199 | 1204 | 1242 | 1267 | 1272 | | |
| B*3520 | | | | 1184 | 1232 | 1242 | 1267 | 1272 | 1285 | | | | |
| B*3528 | | | | 1184 | 1201 | 1232 | 1242 | 1267 | 1272 | 1285 | | | |
| B*7805 | | | | 1184 | 1201 | 1233 | 1263 | 1267 | 1272 | 1293 | | | |
| B*52011 | | | | 1184 | 1201 | 1233 | 1263 | 1267 | 1272 | 1293 | | | |
| B*52013 | | | | 1184 | 1201 | 1233 | 1263 | 1267 | 1272 | 1293 | | | |
| B*3526 | | | | 1184 | 1189 | 1201 | 1232 | 1242 | 1267 | 1272 | 1285 | | |
| B*5504 | | | | 1285 | | | | | | | | | |
| B*5501 | | | | 1233 | 1267 | 1272 | | | | | | | |
| B*5502 | | | | 1233 | 1267 | 1272 | 1285 | | | | | | |
| B*5503 | | | | 1233 | 1267 | 1272 | | | | | | | |
| B*5505 | | | | 1187 | 1233 | 1267 | 1272 | | | | | | |
| B*5601 | | | | 1233 | 1267 | 1272 | 1285 | | | | | | |
| B*5509 | | | | 1233 | 1267 | 1272 | | | | | | | |
| B*5603 | | | | 1267 | | | | | | | | | |
| B*5508 | | | | 1285 | | | | | | | | | |
| B*5602 | | | | 1267 | 1272 | 1285 | | | | | | | |
| B*5604 | | | | 1267 | 1272 | 1285 | | | | | | | |
| B*5607 | | | | 1233 | 1267 | 1272 | 1285 | | | | | | |
| B*5901 | | | | 1201 | 1233 | 1267 | 1272 | 1285 | | | | | |
| B*4601 | | | | 1181 | 1184 | 1202 | 1267 | | | | | | |
| B*4602 | | | | 1181 | 1184 | 1202 | 1209 | 1267 | | | | | |
| B*15111 | | | | 1181 | 1184 | 1201 | 1267 | 1297 | | | | | |
| B*15112 | | | | 1181 | 184 | 1182 | 1201 | 1267 | 1297 | | | | |
| B*5701 | | | | 1181 | 184 | 1191 | 1197 | 1199 | 1204 | 1232 | 1267 | 1285 | |
| B*5704 | | | | 1181 | 184 | 1191 | 1197 | 1199 | 1204 | 1232 | 1267 | 1285 | |
| B*5702 | | | | 1181 | 184 | 1191 | 1197 | 1199 | 1204 | 1232 | 1267 | 1285 | |
| B*57031 | | | | 1181 | 184 | 1191 | 1197 | 1199 | 1204 | 1232 | 1267 | 1285 | |
| B*57032 | | | | 1181 | 184 | 1191 | 1197 | 1199 | 1204 | 1232 | 1267 | 1271 | 1285 |
| B*5705 | | | | 1181 | 184 | 1191 | 1197 | 1199 | 1204 | 1232 | 1267 | 1285 | |
| B*1516 | | | | 1181 | 184 | 1199 | 1204 | 1233 | 1267 | 1297 | | | |
| B*1517 | | | | 1181 | 1184 | 1199 | 1204 | 1259 | 1267 | 1297 | | | |
| B*5706 | | | | 1181 | 1184 | 1191 | 1197 | 1199 | 1204 | 1232 | 1267 | 1285 | |
| B*5707 | | | | 1181 | 1184 | 1191 | 1197 | 1199 | 1204 | 1232 | 1267 | 1285 | 1292 |
| B*1508 | | | | 1181 | 1184 | 1201 | 1267 | 1297 | | | | | |
| B*1556 | | | | 1181 | 1184 | 1201 | 1267 | 1297 | | | | | |
| B*1521 | | | | 1181 | 1184 | 1201 | 1232 | 1267 | 1297 | | | | |
| B*1544 | | | | 1181 | 1184 | 1201 | 1232 | 1267 | 1297 | | | | |
| B*1513 | | | | 1181 | 1184 | 1201 | 1232 | 1267 | 1297 | | | | |
| B*1524 | | | | 1181 | 1184 | 1201 | 1267 | 1297 | | | | | |
| B*1536 | | | | 1181 | 1184 | 1201 | 1232 | 1267 | 1297 | | | | |
| B*1557 | | | | 1181 | 1184 | 1201 | 1267 | 1297 | | | | | |
| B*1504 | | | | 1181 | 1184 | 1201 | 1233 | 1267 | 1297 | | | | |
| B*1535 | | | | 1181 | 1184 | 1201 | 1267 | 1297 | | | | | |
| B*1542 | | | | 1181 | 1184 | 1201 | 1233 | 1267 | 1297 | | | | |
| B*1530 | | | | 1181 | 1184 | 1201 | 1267 | 1297 | | | | | |
| B*1563 | | | | 1181 | 1184 | 1201 | 1267 | 1297 | | | | | |
| B*1548 | | | | 1181 | 1184 | 1201 | 1267 | 1285 | 1297 | | | | |
| B*4021 | | | | 1181 | 1184 | 1201 | 1285 | 1292 | | | | | |
| B*1527 | | | | 1181 | 1164 | 1201 | 1239 | 1267 | 1297 | | | | |
| B*1532 | | | | 1181 | 1184 | 1201 | 1267 | 1297 | | | | | |
| B*1515 | | | | 1131 | 1184 | 1201 | 1267 | 1297 | | | | | |
| B*1501101 | | | | 1181 | 1184 | 1201 | 1267 | 1297 | | | | | |
| B*1512 | | | | 1181 | 1184 | 1201 | 1267 | 1290 | 1297 | | | | |
| B*1519 | | | | 1181 | 1184 | 1201 | 1267 | 1290 | 1297 | | | | |
| B*15012 | | | | 1181 | 1184 | 1201 | 1263 | 1267 | 1297 | | | | |
| B*15013 | | | | 1181 | 1184 | 1201 | 1266 | 1267 | 1297 | | | | |
| B*15014 | | | | 1176 | 1181 | 1184 | 1201 | 1267 | 1297 | | | | |
| B*1514 | | | | 1181 | 1184 | 1201 | 1267 | 1292 | 1297 | | | | |
| B*1528 | | | | 1181 | 1184 | 1196 | 1201 | 1267 | 1297 | | | | |
| B*1533 | | | | 1181 | 1184 | 1201 | 1270 | 1297 | | | | | |
| B*1534 | | | | 1181 | 1184 | 1201 | 1267 | 1297 | | | | | |
| B*1538 | | | | 1181 | 1184 | 1201 | 1267 | 1293 | 1297 | | | | |
| B*1560 | | | | 1177 | 1181 | 1184 | 1201 | 1267 | 1297 | | | | |
| B*1550 | | | | 1181 | 1184 | 1201 | 1267 | 1297 | | | | | |
| B*1525 | | | | 1181 | 1184 | 1201 | 1232 | 1267 | 1297 | | | | |
| B*1539 | | | | 1181 | 1184 | 1201 | 1267 | 1297 | | | | | |
| B*1502 | | | | 1181 | 1184 | 1201 | 1232 | 1267 | 1297 | | | | |
| B*1505 | | | | 1181 | 1184 | 1201 | 1267 | 1285 | 1297 | | | | |
| B*1531 | | | | 1181 | 1184 | 1201 | 1267 | 1285 | 1297 | | | | |
| B*1506 | | | | 1181 | 1184 | 1201 | 1239 | 1267 | 1297 | | | | |
| B*1520 | | | | 1181 | 1184 | 1201 | 1232 | 1242 | 1267 | 1285 | 1297 | | |
| B*1540 | | | | 1181 | 1184 | 1201 | 1267 | 1297 | | | | | |
| B*1558 | | | | 1181 | 1184 | 1201 | 1267 | 1297 | | | | | |
| B*1507 | | | | 1181 | 1184 | 1201 | 1267 | 1297 | | | | | |
| B*1555 | | | | 1181 | 1184 | 1201 | 1267 | 1285 | 1297 | | | | |
| B*1545 | | | | 1181 | 1184 | 1201 | 1267 | 1297 | | | | | |
| B*1543 | | | | 1181 | 1184 | 1201 | 1267 | 1297 | | | | | |
| B*3519 | | | | 1184 | 1201 | 1232 | 1242 | 1267 | 1272 | 1285 | | | |
| B*4010 | | | | 1178 | 1184 | 1201 | 1285 | | | | | | |
| B*1546 | | | | 1178 | 1184 | 1201 | 1267 | 1297 | | | | | |
| B*5401 | | | | 1176 | 1185 | 1233 | 1267 | 1272 | 1285 | | | | |
| B*5507 | | | | 1233 | 1267 | 1272 | 1285 | | | | | | |
| B*40011 | | | | 1178 | 1184 | 1201 | 1285 | | | | | | |
| B*40012 | | | | 1178 | 1184 | 1201 | 1285 | | | | | | |
| B*4033 | | | | 1178 | 1184 | 1201 | 1285 | 1292 | | | | | |
| B*4014 | | | | 1178 | 1184 | 1201 | 1272 | 1285 | | | | | |
| B*4025 | | | | 1178 | 1184 | 1201 | 1285 | 1292 | | | | | |
| B*4023 | | | | 1178 | 1184 | 1201 | 1292 | | | | | | |
| B*4101 | | | | 1178 | 1184 | 1201 | 1233 | 1272 | 1285 | 1292 | | | |
| B*4103 | | | | 1173 | 1178 | 1184 | 1201 | 1272 | 1285 | 1292 | | | |
| B*4105 | | | | 1178 | 1184 | 1201 | 1233 | 1272 | 1285 | 1292 | | | |
| B*4502 | 62B | | | 1178 | 1184 | 1201 | 1233 | 1242 | 1267 | 1272 | 1285 | 1292 | |
| B*4409 | | | | 1178 | 1184 | 1201 | 1232 | 1267 | 1272 | 1285 | 1292 | | |
| B*4702 | | | | 1178 | 1184 | 1201 | 1239 | 1267 | 1285 | | | | |
| B*4703 | | | | 1178 | 1184 | 1201 | 1239 | 1267 | 1285 | | | | |
| B*4501 | 62B | | | 1178 | 1184 | 1201 | 1233 | 1242 | 1267 | 1272 | 1285 | 1292 | |
| B*4504 | 62B | | | 1178 | 1184 | 1201 | 1233 | 1242 | 1267 | 1272 | 1285 | | |
| B*4503 | 62B | | | 1178 | 1184 | 1201 | 1233 | 1242 | 1262 | 1267 | 1272 | 1285 | 1292 |
| B*5001 | 62B | | | 1178 | 1184 | 1201 | 1233 | 1242 | 1267 | 1272 | | | |
| B*5002 | 61B | 62B | | 1178 | 1184 | 1201 | 1233 | 1242 | 1267 | 1272 | 1292 | | |
| B*5004 | | | | 1178 | 1184 | 1201 | 1233 | 1242 | 1267 | 1272 | | | |
| B*1553 | | | | 1178 | 1184 | 1201 | 1267 | 1297 | | | | | |
| B*4031 | | | | 1178 | 1184 | 1201 | 1285 | 1292 | | | | | |
| B*4015 | | | | 1178 | 1184 | 1201 | 1293 | | | | | | |
| B*4016 | | | | 1178 | 1184 | 1201 | | | | | | | |
| B*4032 | | | | 1178 | 1184 | 1201 | 1292 | | | | | | |
| B*4102 | | | | 1178 | 1184 | 1201 | 1272 | 1285 | 1292 | | | | |
| B*4104 | | | | 1178 | 1184 | 1201 | 1242 | 1272 | 1285 | 1292 | | | |
| B*4415 | 60B | 61B | 62B | 1178 | 1184 | 1201 | 1233 | 1242 | 1267 | 1272 | 1285 | 1292 | |
| B*4030 | | | | 1178 | 1184 | 1201 | 1232 | 1285 | 1292 | | | | |
| B*4034 | | | | 1178 | 1184 | 1201 | 1232 | 1264 | 1285 | 1292 | | | |
| B*44021 | | | | 1178 | 1184 | 1201 | 1232 | 1267 | 1272 | 1285 | 1292 | | |
| B*4414 | | | | 1178 | 1184 | 1201 | 1232 | 1267 | 1272 | 1285 | 1292 | | |
| B*4411 | | | | 1178 | 1184 | 1201 | 1227 | 1232 | 1267 | 1272 | 1285 | 1292 | |
| B*4412 | | | | 1178 | 1184 | 1201 | 1232 | 1267 | 1272 | 1285 | 1292 | | |
| B*4424 | | | | 1178 | 1185 | 1201 | 1232 | 1267 | 1272 | 1285 | 1292 | | |
| B*4416 | | | | 1178 | 1184 | 1201 | 1232 | 1267 | 1272 | 1285 | | | |
| B*4421 | | | | 1178 | 1184 | 1201 | 1232 | 1267 | 1272 | 1285 | 1292 | | |
| B*44031 | | | | 1178 | 1184 | 1201 | 1232 | 1267 | 1272 | 1285 | 1292 | | |
| B*44032 | | | | 1178 | 1184 | 1201 | 1232 | 1267 | 1272 | 1285 | 1292 | | |
| B*4407 | | | | 1184 | 1201 | 1232 | 1267 | 1272 | 1285 | 1292 | | | |
| B*4413 | | | | 1178 | 1184 | 1190 | 1201 | 1232 | 1267 | 1272 | 1285 | 1292 | |
| B*4404 | | | | 1176 | 1184 | 1201 | 1232 | 1267 | 1272 | 1285 | 1292 | | |
| B*4410 | | | | 1178 | 1184 | 1201 | 1232 | 1239 | 1242 | 1267 | 1272 | 1285 | 1292 |
| B*4902 | 60B | 61B | 62B | 1178 | 1184 | 1201 | 1233 | 1242 | 1267 | 1272 | | | |
| B*4417 | | | | 1178 | 1184 | 1201 | 1232 | 1267 | 1272 | 1285 | 1292 | | |
| B*4420 | | | | 1178 | 1184 | 1201 | 1233 | 1267 | 1272 | 1285 | 1292 | | |
| B*4418 | 60B | 61B | 62B | 1178 | 1184 | 1201 | 1233 | 1242 | 1267 | 1272 | 1285 | 1292 | |
| B*4901 | 60B | 61B | 62B | 1178 | 1184 | 1201 | 1233 | 1242 | 1267 | 1272 | 1285 | | |
| B*4701 | | | | 1178 | 1184 | 1201 | 1239 | 1267 | 1285 | | | | |
| B*4007 | | | | 1178 | 1184 | 1201 | 1285 | | | | | | |
| B*44022 | | | | 1178 | 1184 | 1201 | 1232 | 1267 | 1272 | 1285 | 1292 | | |
| B*4408 | | | | 1181 | 1184 | 1201 | 1232 | 1267 | 1272 | 1285 | 1292 | | |
| B*1301 | 62B | 64B | | 1178 | 1184 | 1201 | 1232 | 1267 | 1272 | 1285 | 1297 | | |
| B*1306 | 62B | 64B | | 1178 | 1184 | 1201 | 1232 | 1267 | 1272 | 1285 | 1297 | | |
| B*1302 | 62B | 64B | | 1178 | 1184 | 1201 | 1233 | 1267 | 1272 | 1285 | 1297 | | |
| B*1303 | 62B | 64B | | 1178 | 1184 | 1201 | 1233 | 1267 | 1272 | 1285 | 1297 | | |
| B*1304 | 64B | | | 1178 | 1184 | 1201 | 1233 | 1267 | 1285 | 1297 | | | |
| B*0802 | | | | 1171 | 1201 | 1272 | 1285 | 1296 | | | | | |
| B*0806 | | | | 1171 | 1201 | 1296 | | | | | | | |
| B*0803 | | | | 1171 | 1201 | 1272 | 1285 | 1296 | | | | | |
| B*3525 | | | | 1201 | 1232 | 1242 | 1267 | 1272 | 1285 | | | | |
| B*0812 | | | | 1171 | 1201 | 1272 | 1285 | 1296 | | | | | |
| B*0801 | | | | 1171 | 1201 | 1272 | 1285 | 1296 | | | | | |
| B*0807 | | | | 171 | 1201 | 1272 | 1285 | 1296 | | | | | |
| B*0805 | | | | 171 | 1198 | 1201 | 1272 | 1285 | 1296 | | | | |
| B*0810 | | | | 171 | 1194 | 1201 | 1272 | 1285 | 1296 | | | | |
| B*0811 | | | | 171 | 1201 | 1272 | 1285 | 1296 | | | | | |
| B*0813 | | | | 171 | 1201 | 1272 | 1285 | 1296 | | | | | |
| B*0809 | | | | 171 | 1201 | 1272 | 1285 | 1296 | | | | | |
| B*1529 | | | | 1201 | 1267 | 1297 | | | | | | | |
| B*0804 | | | | 1171 | 1201 | 1272 | 1285 | 1296 | | | | | |
| B*4012 | | | | 1184 | 1201 | 1285 | | | | | | | |
| B*1503 | | | | 1201 | 1267 | 1297 | | | | | | | |
| B*1564 | | | | 1201 | 1267 | 1297 | | | | | | | |
| B*1561 | | | | 1201 | 1267 | 1297 | | | | | | | |
| B*1562 | | | | 1201 | 1232 | 1242 | 1297 | | | | | | |
| B*1547 | | | | 1201 | 1267 | 1297 | | | | | | | |
| B*1549 | | | | 1201 | 1206 | 1267 | 1297 | | | | | | |
| B*1554 | | | | 1201 | 1267 | 1297 | | | | | | | |
| B*3904 | | | | 1189 | 1201 | 1267 | 1272 | 1285 | | | | | |
| B*1509 | | | | 1201 | 1263 | 1267 | 1297 | | | | | | |
| B*1510 | | | | 1201 | 1267 | 1297 | | | | | | | |
| B*1537 | | | | 1201 | 1267 | 1293 | 1297 | | | | | | |
| B*1518 | | | | 1201 | 1267 | 1297 | | | | | | | |
| B*1551 | | | | 1201 | 1267 | 1285 | 1297 | | | | | | |
| B*1552 | | | | 1201 | 1267 | 1285 | 1297 | | | | | | |
| B*1523 | | | | 1201 | 1267 | 1297 | | | | | | | |
| B*8301 | | | | 1232 | 1267 | 1272 | 1285 | 1292 | | | | | |
| B*5606 | | | | 1184 | 1233 | 1263 | 1267 | 1272 | 1293 | | | | |
| B*7801 | | | | 1184 | 1201 | 1233 | 1263 | 1267 | 1272 | 1293 | | | |
| B*78022 | | | | 1184 | 1201 | 1233 | 1263 | 1267 | 1272 | 1293 | | | |
| B*5306 | | | | 1184 | 1201 | 1232 | 1242 | 1267 | 1272 | 1293 | | | |
| B*4406 | | | | 1184 | 1201 | 1232 | 1267 | 1272 | 1285 | 1292 | | | |
| B*5104 | | | | 1184 | 1201 | 1232 | 1263 | 1267 | 1272 | | | | |
| B*5106 | | | | 1184 | 1201 | 1263 | 1267 | 1272 | 1293 | | | | |
| B*51131 | | | | 1184 | 1201 | 1233 | 1267 | 1272 | 1293 | | | | |
| B*51132 | | | | 1184 | 1201 | 1233 | 1263 | 1267 | 1272 | 1293 | | | |
| B*5116 | | | | 1184 | 1201 | 1233 | 1263 | 1267 | 1272 | 1293 | | | |
| B*5110 | | | | 1184 | 1201 | 1233 | 1265 | | | | | | |
| B*5121 | | | | 1184 | 1201 | 1233 | 1263 | 1267 | 1272 | 1293 | | | |
| B*51011 | | | | 1184 | 1201 | 1233 | 1263 | 1267 | 1272 | 1293 | | | |
| B*51021 | | | | 1184 | 1201 | 1233 | 1263 | 1267 | 1272 | | | | |
| B*51014 | | | | 1184 | 1201 | 1233 | 1267 | 1272 | 1293 | | | | |
| B*5124 | | | | 1184 | 1201 | 1233 | 1272 | 1293 | | | | | |
| B*51013 | | | | 1201 | 1233 | 1263 | 1267 | 1272 | 1293 | | | | |
| B*5109 | | | | 1184 | 1201 | 1233 | 1263 | 1267 | 1272 | 1285 | 1293 | | |
| B*5103 | | | | 1184 | 1201 | 1233 | 1263 | 1267 | 1272 | 1293 | | | |
| B*5123 | | | | 1184 | 1201 | 1233 | 1263 | 1267 | 1272 | 1292 | | | |
| B*5119 | | | | 1184 | 1201 | 1233 | 1263 | 1267 | 1272 | 1285 | 1293 | | |
| B*5114 | | | | 1184 | 1201 | 1233 | 1264 | 1263 | 1267 | 1272 | 1293 | | |
| B*5117 | | | | 1184 | 1201 | 1233 | 1243 | 1263 | 1267 | 1272 | 1293 | | |
| B*5118 | | | | 1184 | 1201 | 1233 | 1256 | 1263 | 1267 | 1272 | 1293 | | |
| B*5112 | | | | 1184 | 1201 | 1229 | 1233 | 1263 | 1267 | 1272 | 1293 | | |
| B*5108 | | | | 1184 | 1201 | 1233 | 1263 | 1267 | 1272 | 1285 | 1293 | | |
| B*5120 | | | | 1184 | 1201 | 1233 | 1263 | 1267 | 1272 | 1285 | 1293 | | |
| B*5107 | | | | 1184 | 1201 | 1233 | 1263 | 1267 | 1272 | 1293 | | | |
| B*52012 | | | | 1184 | 1201 | 1233 | 1263 | 1267 | 1272 | 1293 | | | |
| B*5203 | | | | 1184 | 1201 | 1233 | 1263 | 1267 | 1272 | 1285 | | | |
| B*4903 | 61B | 62B | | 1184 | 1201 | 1233 | 1242 | 1267 | 1272 | | | | |
| B*1804 | | | | 1201 | 1267 | 1272 | 1285 | 1293 | 1297 | | | | |
| B*5122 | | | | 1184 | 1201 | 1233 | 1263 | 1267 | 1272 | 1293 | | | |
| B*7803 | | | | 1184 | 1201 | 1233 | 1263 | 1267 | 1272 | 1293 | | | |
| B*5605 | | | | 1233 | 1263 | 1267 | 1272 | 1293 | | | | | |
| B*5202 | | | | 1181 | 1184 | 1201 | 1233 | 1263 | 1267 | 1272 | 1293 | | |
| B*4020 | | | | 1178 | 1184 | 1201 | 1285 | | | | | | |
| B*4004 | | | | 1178 | 1184 | 1201 | 1232 | 1285 | | | | | |
| B*4011 | | | | 1178 | 1184 | 1201 | 1285 | | | | | | |
| B*4026 | | | | 1178 | 1184 | 1201 | 1263 | 1267 | 1292 | | | | |
| B*4028 | | | | 1178 | 1184 | 1201 | 1232 | 1263 | 1267 | 1272 | 1292 | 1293 | |
| B*4003 | | | | 1178 | 1184 | 1201 | 1285 | | | | | | |
| B*4005 | | | | 1178 | 1184 | 1201 | | | | | | | |
| B*4018 | | | | 1178 | 1184 | 1201 | 1285 | | | | | | |
| B*4002 | | | | 1178 | 1184 | 1201 | 1285 | | | | | | |
| B*4009 | | | | 1178 | 1184 | 1201 | 1285 | | | | | | |
| B*4024 | | | | 1178 | 1184 | 1201 | 1242 | 1285 | 1292 | | | | |
| B*4029 | | | | 1178 | 1184 | 1201 | 1193 | 1285 | 1292 | | | | |
| B*4027 | | | | 1178 | 1184 | 1201 | 1285 | 1292 | | | | | |
| B*4006 | | | | 1178 | 1184 | 1201 | 1233 | 1285 | | | | | |
| B*4019 | | | | 1178 | 1184 | 1201 | 1285 | | | | | | |
| B*4008 | | | | 1178 | 1184 | 1201 | 1285 | | | | | | |
| B*4013 | | | | 1178 | 1184 | 1201 | 1285 | | | | | | |
| B*7301 | | | | 1203 | 1233 | 1285 | 1293 | | | | | | |
| B*2701 | | | | 1171 | 1203 | 1259 | 1267 | 1285 | 1297 | | | | |
| B*2702 | | | | 1171 | 1203 | 1259 | 1267 | 1285 | 1297 | | | | |
| B*2707 | | | | 1171 | 1203 | 1285 | 1297 | | | | | | |
| B*27052 | | | | 1171 | 1203 | 1259 | 1267 | 1285 | 1297 | | | | |
| B*27054 | | | | 1171 | 1203 | 1259 | 1267 | 1285 | 1297 | | | | |
| B*2713 | | | | 1171 | 1203 | 1259 | 1267 | 1285 | 1297 | | | | |
| B*2716 | | | | 1171 | 1201 | 1259 | 1267 | 1285 | 1297 | | | | |
| B*2710 | | | | 1171 | 1203 | 1259 | 1267 | 1297 | | | | | |
| B*2703 | | | | 1171 | 1203 | 1259 | 1267 | 1285 | 1297 | | | | |
| B*2717 | | | | 1171 | 1203 | 1259 | 1267 | 1285 | 1297 | | | | |
| B*2709 | | | | 1171 | 1203 | 1259 | 1267 | 1285 | 1297 | | | | |
| B*2714 | | | | 1171 | 1203 | 1233 | 1259 | 1267 | 1285 | 1297 | | | |
| B*2719 | | | | 1171 | 1203 | 1232 | 1259 | 1267 | 1285 | 1297 | | | |
| B*2704 | | | | 1171 | 1203 | 1259 | 1267 | 1297 | | | | | |
| B*2715 | | | | 1171 | 1203 | 1259 | 1267 | 1297 | | | | | |
| B*2706 | | | | 1171 | 1203 | 1267 | 1297 | | | | | | |
| B*2722 | | | | 1171 | 1203 | 1267 | 1297 | | | | | | |
| B*2720 | | | | 1171 | 1203 | 1297 | | | | | | | |
| B*2711 | | | | 1171 | 1203 | 1265 | 1297 | | | | | | |
| B*2721 | | | | 1171 | 1203 | 1267 | 1297 | | | | | | |
| B*2708 | | | | 1171 | 1203 | 1259 | 1267 | 1285 | 1297 | | | | |
| B*2712 | | | | 1171 | 1201 | 1259 | 1267 | 1285 | 1297 | | | | |
| B*2718 | | | | 1171 | 1201 | 1259 | 1267 | 1297 | | | | | |
| B*2723 | | | | 1171 | 1201 | 1259 | 1267 | 1285 | 1297 | | | | |
| B*3803 | | | | 1189 | 1201 | 1267 | 1272 | 1285 | | | | | |
| B*4802 | | | | 1201 | 1232 | 1242 | 1267 | 1272 | 1285 | | | | |
| B*4801 | | | | 1201 | 1285 | | | | | | | | |
| B*4804 | | | | 1201 | 1285 | | | | | | | | |
| B*4807 | | | | 1201 | 1285 | | | | | | | | |
| B*4803 | | | | 1201 | 1285 | | | | | | | | |
| B*39021 | | | | 1189 | 1201 | 1267 | 1272 | 1285 | | | | | |
| B*39022 | | | | 1201 | 1267 | 1272 | 1285 | | | | | | |
| B*3913 | | | | 1201 | 1267 | 1272 | 1285 | | | | | | |
| B*3923 | | | | 1201 | 1267 | 1272 | 1284 | 1285 | | | | | |
| B*3908 | | | | 1189 | 1201 | 1267 | 1272 | 1285 | | | | | |
| B*3914 | | | | 1189 | 1201 | 1267 | 1272 | 1285 | | | | | |
| B*3903 | | | | 1189 | 1201 | 1267 | 1272 | 1285 | | | | | |
| B*3924 | | | | 1189 | 1201 | 1236 | 1267 | 1272 | 1285 | | | | |
| B*39011 | | | | 1189 | 1201 | 1267 | 1272 | 1285 | | | | | |
| B*39013 | | | | 1189 | 1201 | 1267 | 1272 | 1285 | | | | | |
| B*3915 | | | | 1189 | 1201 | 1267 | 1272 | 1285 | | | | | |
| B*3909 | | | | 1189 | 1201 | 1240 | 1267 | 1272 | 1285 | | | | |
| B*39014 | | | | 1186 | 1189 | 1201 | 1267 | 1272 | 1285 | | | | |
| B*3905 | | | | 1189 | 1201 | 1267 | 1272 | 1285 | | | | | |
| B*3922 | | | | 1189 | 1201 | 1267 | 1272 | 1285 | | | | | |
| B*3918 | | | | 1189 | 1201 | 1267 | | | | | | | |
| B*3911 | | | | 1189 | 1201 | 1267 | 1272 | 1285 | | | | | |
| B*39061 | | | | 1189 | 1201 | 1233 | 1267 | 1272 | 1285 | | | | |
| B*39062 | | | | 1189 | 1201 | 1233 | 1267 | 1272 | 1285 | | | | |
| B*4201 | | | | 1272 | 1285 | 1292 | | | | | | | |
| B*4202 | | | | 1272 | 1285 | 1292 | | | | | | | |
| B*0719 | | | | | | | | | | | | | |
| B*0704 | | | | | | | | | | | | | |
| B*0725 | | | | 1285 | | | | | | | | | |
| B*8101 | | | | 1285 | | | | | | | | | |
| B*07021 | | | | | | | | | | | | | |
| B*07022 | | | | | | | | | | | | | |
| B*0723 | | | | 1230 | | | | | | | | | |
| B*0705 | | | | | | | | | | | | | |
| B*0703 | | | | 1201 | | | | | | | | | |
| B*07023 | | | | | | | | | | | | | |
| B*0715 | | | | 1207 | | | | | | | | | |
| B*0716 | | | | 1201 | | | | | | | | | |
| B*0721 | | | | 1174 | | | | | | | | | |
| B*0722 | | | | 1269 | | | | | | | | | |
| B*0720 | | | | 1292 | | | | | | | | | |
| B*0724 | | | | | | | | | | | | | |
| B*0709 | | | | | | | | | | | | | |
| B*0717 | | | | | | | | | | | | | |
| B*3917 | | | | 1201 | 1267 | 1272 | 1285 | | | | | | |
| B*3910 | | | | 1201 | 1267 | 1272 | 1285 | | | | | | |
| B*67012 | | | | 1267 | 1272 | 1285 | | | | | | | |
| B*3916 | | | | 1201 | 1235 | 1267 | 1272 | 1285 | | | | | |
| B*67011 | | | | 1189 | 1267 | 1272 | 1285 | | | | | | |
| B*0706 | | | | | | | | | | | | | |
| B*0707 | | | | | | | | | | | | | |
| B*0712 | | | | 1232 | 1242 | | | | | | | | |
| B*0714 | | | | 1232 | | | | | | | | | |
| B*0718 | | | | 1232 | | | | | | | | | |
| B*5510 | | | | 1233 | 1267 | 1272 | 1285 | | | | | | |
| B*3920 | | | | 1201 | 1267 | 1272 | 1285 | | | | | | |
| B*0711 | | | | | | | | | | | | | |
| B*3801 | | | | 1189 | 1201 | 1267 | 1272 | 1285 | | | | | |
| B*38021 | | | | 1189 | 1201 | 1267 | 1272 | 1265 | | | | | |
| B*38022 | | | | 1189 | 1201 | 1267 | 1272 | 1285 | | | | | |
| B*3804 | | | | 1201 | 1267 | 1272 | 1285 | | | | | | |
| B*4806 | | | | 1201 | | | | | | | | | |
| B*0708 | | | | 1201 | | | | | | | | | |
| B*0710 | | | | | | | | | | | | | |
| B*1401 | | | | 1189 | 1201 | 1267 | 1272 | 1293 | 1297 | | | | |
| B*6702 | | | | 1185 | 1202 | 1207 | 1267 | 1272 | 1285 | | | | |
| B*0713 | | | | 1207 | | | | | | | | | |
| B*3919 | | | | 1184 | 1201 | 1267 | 1272 | 1285 | | | | | |
| B*3806 | | | | 1184 | 1201 | 1267 | 1272 | 1285 | | | | | |
| B*3807 | | | | 1184 | 1188 | 1189 | 1201 | 1267 | 1272 | 1285 | | | |
| B*1807 | | | | 1171 | 1201 | 1267 | 1272 | 1285 | 1293 | 1297 | | | |
| B*3701 | | | | 1201 | 1234 | 1240 | 1267 | 1272 | 1285 | | | | |
| B*3704 | | | | 1201 | 1234 | 1240 | 1267 | 1272 | 1285 | 1293 | | | |
| B*3702 | | | | 1201 | 1267 | 1285 | | | | | | | |
| B*3705 | | | | 1201 | 1234 | 1240 | 1267 | 1272 | 1285 | | | | |
| B*1802 | | | | 1171 | 1201 | 1267 | 1272 | 1285 | 1293 | 1297 | | | |
| B*1801 | | | | 1171 | 1201 | 1267 | 1272 | 1285 | 1293 | 1297 | | | |
| B*1803 | | | | 1171 | 1201 | 1267 | 1272 | 1285 | 1293 | 1297 | | | |
| B*1811 | | | | 1171 | 1201 | 1267 | 1272 | 1285 | 1297 | | | | |
| B*1805 | | | | 1171 | 1201 | 1265 | 1267 | 1272 | 1285 | 1293 | 1297 | | |
| B*1806 | | | | 1171 | 1201 | 1267 | 1272 | 1285 | 1293 | 1297 | | | |
| B*1808 | | | | 1171 | 1201 | 1237 | 1267 | 1272 | 1285 | 1293 | 1297 | | |
| B*1812 | | | | 1171 | 1201 | 1267 | 1272 | 1285 | 1293 | 1297 | | | |
| B*1810 | | | | 1171 | 1201 | 1267 | 1272 | 1285 | 1297 | | | | |
| B*1813 | | | | 1171 | 1201 | 1267 | 1272 | 1285 | 1293 | 1297 | | | |
| B*1809 | | | | 1171 | 1201 | 1267 | 1272 | 1285 | 1293 | 1297 | | | |
| B*4805 | | | | 1201 | | | | | | | | | |
| B*5402 | | | | 1176 | 1185 | 1233 | 1267 | 1272 | 1285 | | | | |
| B*3907 | | | | 1189 | 1201 | 1267 | 1272 | 1285 | 65B | | | | |
| B*27053 | | | | 1203 | 1259 | 1267 | 1285 | | | | | | |
| B*4405 | | | | 1184 | 1201 | 1232 | 1267 | 1272 | 1285 | 1292 | | | |
| B*3805 | | | | 1189 | 1201 | 1267 | 1272 | 1285 | | | | | |
| B*3912 | | | | 1171 | 1189 | 1201 | 1267 | 1272 | 1285 | | | | |
| B*1402 | | | | 1189 | 1201 | 1267 | 1272 | 1293 | 1297 | | | | |
| B*1404 | | | | 1189 | 1199 | 1201 | 1267 | 1272 | 1293 | 1297 | | | |
| B*1403 | | | | 1189 | 1201 | 1267 | 1272 | 1293 | 1297 | | | | |
| B*1405 | | | | 1189 | 1201 | 1267 | 1272 | 1293 | 1297 | | | | |
| B*14061 | | | | 1189 | 1201 | 1267 | 1272 | 1293 | 1297 | | | | |
| B*14062 | | | | 1189 | 1201 | 1267 | 1272 | 1293 | 1297 | | | | |
| B*4422 | | | | 1178 | 1184 | 1201 | 1232 | 1267 | 1272 | 1285 | 1292 | | |
| B*8201 | | | | 1267 | 1272 | 1285 | 1292 | | | | | | |
| B*8202 | | | | 1267 | 1272 | 1285 | 1292 | | | | | | |

TABLE 32
Correspondence of the patchworks and SEQ ID Nos for B

| patchwork | SEQ ID NO: | | | | | | | | | |
|---|---|---|---|---|---|---|---|---|---|---|
| 1B | 1080 | | | | | | | | | |
| 2B | 1081 | | | | | | | | | |
| 3B | 1082 | | | | | | | | | |
| 4B | 1083 | | | | | | | | | |
| 5B | 1084 | | | | | | | | | |
| 6B | 1085 | 1088 | | | | | | | | |
| 7B | 1086 | | | | | | | | | |
| 8B | 1087 | | | | | | | | | |
| 9B | 1089 | 1094 | | | | | | | | |
| 10B | 1090 | | | | | | | | | |
| 11B | 1091 | 1095 | | | | | | | | |
| 12B | 1092 | 1096 | | | | | | | | |
| 13B | 1093 | | | | | | | | | |
| 14B | 1097 | 1103 | 1106 | | | | | | | |
| 15B | 1098 | 1104 | | | | | | | | |
| 16B | 1099 | | | | | | | | | |
| 17B | 1100 | 1105 | 1107 | 1108 | | | | | | |
| 18B | 1101 | | | | | | | | | |
| 19B | 1102 | | | | | | | | | |
| 20B | 1109 | 1113 | 1115 | | | | | | | |
| 21B | 1110 | 1114 | | | | | | | | |
| 22B | 1111 | | | | | | | | | |
| 23B | 1112 | | | | | | | | | |
| 24B | 1116 | 1120 | | | | | | | | |
| 25B | 1117 | 1121 | 1122 | | | | | | | |
| 26B | 1118 | | | | | | | | | |
| 27B | 1119 | | | | | | | | | |
| 28B | 1123 | 1129 | 1133 | | | | | | | |
| 29B | 1124 | 1130 | 1134 | 1135 | 1136 | 1137 | 1138 | 1139 | 1140 | 1141 |
| 30B | 1125 | 1131 | | | | | | | | |
| 31B | 1126 | | | | | | | | | |
| 32B | 1127 | 1132 | | | | | | | | |
| 33B | 1128 | | | | | | | | | |
| 34B | 1142 | 1149 | 1153 | 1156 | 1157 | | | | | |
| 35B | 1143 | 1150 | 1154 | | | | | | | |
| 36B | 1144 | 1151 | | | | | | | | |
| 37B | 1145 | | | | | | | | | |
| 38B | 1146 | | | | | | | | | |
| 39B | 1147 | | | | | | | | | |
| 40B | 1148 | 1152 | 1155 | | | | | | | |
| 41B | 1158 | 1162 | | | | | | | | |
| 42B | 1159 | | | | | | | | | |
| 43B | 1160 | 1163 | 1164 | | | | | | | |
| 44B | 1161 | | | | | | | | | |
| 45B | 1165 | | | | | | | | | |
| 46B | 1166 | | | | | | | | | |
| 47B | 1167 | 1169 | | | | | | | | |
| 48B | 1168 | | | | | | | | | |
| 49B | 1170 | 1179 | | | | | | | | |
| 50B | 1175 | 1180 | | | | | | | | |
| 51B | 1183 | 1212 | 1218 | 1222 | | | | | | |
| 52B | 1195 | 1213 | 1219 | 1223 | 1226 | | | | | |
| 53B | 1206 | 1215 | | | | | | | | |
| 54B | 1209 | 1216 | 1220 | 1224 | | | | | | |
| 55B | 1211 | 1217 | 1221 | 1225 | | | | | | |
| 56B | 1227 | 1246 | | | | | | | | |
| 57B | 1229 | 1247 | 1251 | 1253 | 1255 | 1256 | | | | |
| 58B | 1239 | 1249 | 1252 | 1254 | | | | | | |
| 59B | 1242 | 1250 | | | | | | | | |
| 60B | 1259 | 1274 | 1277 | | | | | | | |
| 61B | 1261 | 1275 | 1278 | 1279 | 1280 | 1281 | 1282 | 1284 | | |
| 62B | 1269 | 1276 | | | | | | | | |
| 63B | 1288 | 1295 | | | | | | | | |
| 64B | 1296 | 1299 | | | | | | | | |

TABLE 33
Correspondence of Primers

| | SEQ ID NOS. | SEQ ID NOS. |
|---|---|---|
| Primers for DRB1 | 590, 591, 591 | 593 |
| Primers for DPB1 | 448, 449 | 450-455 |
| Primers for DQB1 | 504, 505 | 506 |
| Primers for A | 899, 900, 903-905 | 901, 902, 906, 907 |
| Primers for B | 1073, 1075-1077 | 1074, 1078, 1079 |

[References]

(1) Kobayashi, T. et al.: The significance of HLA-DRB1 matching in clinical renal transplantation, Transplantation, 54: 238, 1992

(2) Olerup, O. et al.: HLA-DR typing by PCR amplification with sequence-specific primers (PCR-SSP) in 2 hours: An alternative to serological DR typing in clinical practice including donor-recipient matching in cadaveric transplantation, Tissue Antigen, 39: 225, 1992

(3) Inoko, H. et al.: PCR-RFLP, Handbook of HLA typing techniques (edited by Hui, K. M. & Bidwell, J. L.), CRC press, Florida, U.S.A., 1993, p.9

(4) Erlich, H. et al.: HLA-DR, -DQ and -DP typing using PCR amplification and immobilized probes, European Journal of Immunogenetics, 18: 33, 1991

(5) Carrington, M. et al.: Typing of HLA-DQA1 and DQB1 using DNA single-strand conformation polymorphism, Hum. Immunol., 33: 208, 1992

Industrial Applicability

According to the present invention, there are provided a kit and method that are suitable for processing of a large number of specimens and enable a large number of high accuracy typing tests by one test for one specimen, and oligonucleotides and primers used therefor.

What is claimed is:

1. A typing kit for determining HLA genotype of a test specimen by hybridization between a nucleic acid sequence derived from the test specimen and oligonucleotides, which comprises a substrate on which the oligonucleotides are immobilized through covalent bonds, wherein the oligonucleotides are 10-24 nucleotide length and are derived from sequences of a group of genes belonging to HLA class I or class II antigen on a human genome and each of the oligonucleotides includes polymorphism of each gene as alloantigen in the sequence.

2. The typing kit according to claim 1, wherein a surface of the substrate is coated with carbodiimide groups or isocyanate groups and the covalent bonds are formed through reactions of the carbodiimide groups or isocyanate groups and linkers added to ends of the oligonucleotides.

3. The typing kit according to claim 1, wherein the linker is an amino group, or a compound having an amino group or a homopolymer of thymidine residues at an end of the compound.

4. The typing kit according to claim 1, wherein the oligonucleotides are immobilized in an area on a surface of the substrate having a size of 10-1,000 μm in diameter.

5. The typing kit according to claim 1, wherein the oligonucleotides consist of DNA or a peptide nucleic acid.

6. The typing kit according to claim 1, wherein class I antigen is an antigen controlled by any of gene loci coding for HLA-A, HLA-B, HLA-C, HLA-D, HLA-E, HLA-F and HLA-G and class II antigen is an antigen controlled by any of gene loci coding for HLA-DQ, HLA-DR or HLA-DP.

7. The typing kit according to claim 1 or 6, wherein HLA-DQ is an antigen derived from any of DQA1, DQA2, DQB1 and DQB2 gene loci.

8. The typing kit according to claim 1 or 6, wherein HLA-DR is an antigen derived from any of DRA, DRB1, DRB3, DRB4 and DRB5 gene loci.

9. The typing kit according to claim 1 or 6, wherein HLA-DP is an antigen derived from any of DPA1, DPA2, DPB1 and DPB2 gene loci.

10. The typing kit according to claim 1 or 6, wherein the oligonucleotides contain at least one of the nucleic acid sequences of SEQ ID NOS: 1-397, 456-503, 507-589, 594-898, 908-1072 or 1080-1298.

11. The typing kit according to any one of claims 1, 6 and 7, which is for determining HLA-DQA1 genotype, and wherein the oligonucleotides contain at least one of the nucleic acid sequences of SEQ ID NOS: 1-54.

12. The typing kit according to any one of claims 1, 6 and 7, which is for determining HLA-DQB1 genotype, and wherein the oligonucleotides contain at least one of the nucleic acid sequences of SEQ ID NOS: 55-140 or 507-589.

13. The typing kit according to any one of claims 1, 6 and 8, which is for determining HLA-DRA genotype, and wherein the oligonucleotides contain at least one of the nucleic acid sequences of SEQ ID NOS: 141-144.

14. The typing kit according to any one of claims 1, 6 and 8, which is for determining genotypes of HLA-DRB1, HLA-DRB3, HLA-DRB4, HLA-DRB5, HLA-DRB6 or HLA-DRB7, and wherein the oligonucleotides contain at least one of the nucleic acid sequences of SEQ ID NOS: 145-323 or 594-853.

15. The typing kit according to any one of claims 1, 6 and 9, which is for determining HLA-DPA1 genotype, and wherein the oligonucleotides contain at least one of the nucleic acid sequences of SEQ ID NOS: 324-348.

16. The typing kit according to any one of claims 1, 6 and 9, which is for determining HLA-DPB1 genotype, and wherein the oligonucleotides contain at least one of the nucleic acid sequences of SEQ ID NOS: 349-397 or 456-503.

17. The typing kit according to claim 10, wherein at least one of the oligonucleotides is replaced with an oligonucleotide of 8-24 nucleotide length obtained by extending or shortening any of the nucleic acid sequences of SEQ ID NOS: 1-397, 456-503, 507-589, 594-898, 908-1072 or 1080-1298 for a gene sequence on the genome at 5′ or 3′ end, or at both ends without eliminating or changing nucleotides associated with the gene polymorphism to have optimized binding affinity for the hybridization.

18. The typing kit according to any one of claims 1, 2, 4, 5 and 10-17, wherein at least one of the oligonucleotides is an oligonucleotide whose binding affinity for the hybridization is reduced by replacing an arbitrary nucleotide not involved in the gene polymorphism with a spacer compound.

19. The typing kit according to claim 18, wherein the spacer compound has a nucleic acid frame that does not have complementary binding property with any kind of nucleotides.

20. PCR primers for excising and amplifying a nucleic acid sequence associated with gene polymorphism of HLA class I antigen or class II antigen of a test specimen.

21. The PCR primers according to claim 20, which is for low accuracy genotyping, and wherein a combination of 5′ primer and 3′ primer corresponding to HLA-DQ consists of at least one of (SEQ ID NOS: 398 and 400) and (SEQ ID NOS: 399 and 400), and a combination of 5′ primer and 3′ primer corresponding to HLA-DR consists of at least one of (SEQ ID NOS: 401 and 403) and (SEQ ID NOS: 402 and 403).

22. The PCR primers according to claim 20, which is for high accuracy genotyping, and wherein a combination of 5′ primer and 3′ primer corresponding to HLA-DQB1 consists of at least one of (SEQ ID NOS: 404 and 406), (SEQ ID NOS: 405 and 406), (SEQ ID NOS: 407 and 409), (SEQ ID NOS: 408 and 409), (SEQ ID NOS: 410 and 412) and (SEQ ID NOS: 411 and 412), and a combination of 5′ primer and 3′ primer corresponding to HLA-DRB1 consists of at least one of (SEQ ID NOS: 413 and 417), (SEQ ID NOS: 414 and 417), (SEQ ID NOS: 415 and 417) and (SEQ ID NOS: 416 and 417).

23. The PCR primers according to claim 20, which is for high accuracy genotyping, and wherein a combination of 5′ primer and 3′ primer corresponding to HLA-DQA1 consists of at least one of (SEQ ID NOS: 418 and 420) and (SEQ ID NOS: 419 and 420), a combination of 5′ primer and 3′ primer corresponding to-HLA-DQB1 is (SEQ ID NOS: 421 and 422), a combination of 5′ primer and 3′ primer corresponding to HLA-DRA is (SEQ ID NOS: 423 and 424), a combination of 5′ primer and 3′ primer corresponding to HLA-DRB2 consists of at least one of (SEQ ID NOS: 425 and 428), (SEQ ID NOS: 426 and 428) and (SEQ ID NOS: 427 and 428), a combination of 5′ primer and 3′ primer corresponding to HLA-DRB3 consists of at least one of (SEQ ID NOS: 429 and 431) and (SEQ ID NOS: 430 and 431), a combination of 5′ primer and 3′ primer corresponding to HLA-DRB4 consists of at least one of (SEQ ID NOS: 432 and 433) and (SEQ ID NOS: 434 and 435), a combination of 5′ primer and 3′ primer corresponding to HLA-DRB5 is (SEQ ID NOS: 436 and 437), a combination of 5′ primer and 3′ primer corresponding to HLA-DRB6 consists of at least one of (SEQ ID NOS: 438 and 439) and (SEQ ID NOS: 439 and 440), and a combination of 5′ primer and 3′ primer corresponding to HLA-DRB7 is (SEQ ID NOS: 441 and 442), a combination of 5′ primer and 3′ primer corresponding to HLA-DPA1 is (SEQ ID NOS: 443 and 444) and a combination of 5′ primer and 3′ primer corresponding to HLA-DPB1 consists of at least one of (SEQ ID NOS: 445 and 446) and (SEQ ID NOS: 445 and 447).

24. A method for determining HLA genotype of a test specimen, which comprises allowing hybridization of the oligonucleotides on the substrate of the typing kit according to any one of claims 1-16 with a nucleic acid sequence derived from the specimen and detecting occurrence of hybridization of the oligonucleotides and the nucleic acid sequence derived from the specimen.

25. The method for determining HLA genotype of a test specimen according to claim 24, comprising the steps of:
carrying out low accuracy typing of HLA genotype of the specimen by performing first PCR amplification using the primers for low accuracy genotyping according to claim 21 and a nucleic acid sequence derived from the specimen as a template, allowing hybridization of the amplification product with the oligonucleotides contained in the typing kit according to any one of claims 1-16, and detecting occurrence of hybridization of the nucleic acid sequence derived from the specimen and each of the oligonucleotides; and
carrying out high accuracy HLA genotyping of the specimen by performing second PCR amplification based on the above determination result using primers appropriately selected from the primers for high accuracy genotyping according to claim 22 and a nucleic acid sequence derived from the specimen as a template, allowing hybridization of the amplification product with the oligonucleotides contained in the typing kit according to any one of claims 1-16, and detecting occurrence of hybridization of the nucleic acid sequence derived from the specimen and each of the oligonucleotides.

26. The method for determining HLA genotype of a test specimen according to claim 24, wherein PCR amplification is performed by using a nucleic acid sequence derived from the test specimen as a template and the primers for high accuracy genotyping according to claim 24 (22 or 23?) and the amplification product is hybridized with each of the oligonucleotides contained in the typing kit according to any one of claims 1-16.

27. The method for determining HLA genotype of a test specimen according to claim 24, which comprises preparing alignment of gene sequences of each HLA, and setting a nucleotide sequence in which at least two nucleotide polymorphisms or sequence polymorphisms involving the HLA typing are observed in a nucleotide sequence consisting of 1-10 nucleotides as a patchwork segment, and/or setting a nucleotide sequence in which at least one nucleotide polymorphism or sequence polymorphism is observed as a satellite segment, and
finding the patchworks and/or the satellites in gene sequences of all of the HLA genes, performing typing by combination of the patchworks and/or the satellites and judging whether each of HLA typing of the test specimen is homozygote or heterozygote together with determining the types of HLA.

28. The method for determining HLA genotype of a test specimen according to claim 27, wherein a pseudogene of DRB1 is included in the HLA types to be determined.

29. The typing kit according to claim 1 or 6, wherein the oligonucleotides comprises at least one of the nucleotide sequences of SEQ ID NOS: 908-1071, and the kit is for determining HLA-A genotype.

30. The typing kit according to claim 1 or 6, wherein the oligonucleotides comprises at least one of the nucleotide sequences of SEQ ID NOS: 1080-1298, and the kit is for determining HLA-DRA genotype.


SEQUENCE LISTING (1298 sequences)

SEQ ID NO: 1 - DNA, 18 bp, Artificial Sequence
  Description of Artificial Sequencecapture
ctgaccacgttgcctctt

SEQ ID NO: 2 - DNA, 18 bp, Artificial Sequence
  Description of Artificial Sequencecapture
ctgaccatgttgcctctt

SEQ ID NO: 3 - DNA, 18 bp, Artificial Sequence
  Description of Artificial Sequencecapture
ctgaccacgtcgcctctt

SEQ ID NO: 4 - DNA, 18 bp, Artificial Sequence
  Description of Artificial Sequencecapture
gcctcttgtggtgtaaac

SEQ ID NO: 5 - DNA, 18 bp, Artificial Sequence
  Description of Artificial Sequencecapture
gcctcttacggtgtaaac

SEQ ID NO: 6 - DNA, 18 bp, Artificial Sequence
  Description of Artificial Sequencecapture
gcctcttatggtgtaaac

SEQ ID NO: 7 - DNA, 16 bp, Artificial Sequence
  Description of Artificial Sequencecapture
ccagttttacggtccc

SEQ ID NO: 8 - DNA, 16 bp, Artificial Sequence
  Description of Artificial Sequencecapture
ccagtcttacggtccc

SEQ ID NO: 9 - DNA, 16 bp, Artificial Sequence
  Description of Artificial Sequencecapture
ccagtcttatggtccc

SEQ ID NO: 10 - DNA, 18 bp, Artificial Sequence
  Description of Artificial Sequencecapture
ttacggtccctctggcca

SEQ ID NO: 11 - DNA, 18 bp, Artificial Sequence
  Description of Artificial Sequencecapture
ttacggtctctctggcca

SEQ ID NO: 12 - DNA, 17 bp, Artificial Sequence
  Description of Artificial Sequencecapture
cctctggccagtacacc

SEQ ID NO: 13 - DNA, 17 bp, Artificial Sequence
  Description of Artificial Sequencecapture
cctctgggcagtacacc

SEQ ID NO: 14 - DNA, 16 bp, Artificial Sequence
  Description of Artificial Sequencecapture
ggccagtacacccatg

SEQ ID NO: 15 - DNA, 16 bp, Artificial Sequence
  Description of Artificial Sequencecapture
ggccagttcacccatg

SEQ ID NO: 16 - DNA, 18 bp, Artificial Sequence
  Description of Artificial Sequencecapture
cagtacacccatgaattt

SEQ ID NO: 17 - DNA, 18 bp, Artificial Sequence
  Description of Artificial Sequencecapture
cagtacagccatgaattt

SEQ ID NO: 18 - DNA, 18 bp, Artificial Sequence
  Description of Artificial Sequencecapture
atggagatgaggagttct

SEQ ID NO: 19 - DNA, 18 bp, Artificial Sequence
  Description of Artificial Sequencecapture
atggagacgaggagttct

SEQ ID NO: 20 - DNA, 18 bp, Artificial Sequence
  Description of Artificial Sequencecapture
atggagatgagcagttct

SEQ ID NO: 21 - DNA, 18 bp, Artificial Sequence
  Description of Artificial Sequencecapture
atggagacgagcagttct

SEQ ID NO: 22 - DNA, 18 bp, Artificial Sequence
  Description of Artificial Sequencecapture
agttctacgtggacctgg

SEQ ID NO: 23 - DNA, 18 bp, Artificial Sequence
  Description of Artificial Sequencecapture
agttctatgtggacctgg

SEQ ID NO: 24 - DNA, 15 bp, Artificial Sequence
  Description of Artificial Sequencecapture
ctggagaggaaggag

SEQ ID NO: 25 - DNA, 15 bp, Artificial Sequence
  Description of Artificial Sequencecapture
ctggggaggaaggag

SEQ ID NO: 26 - DNA, 15 bp, Artificial Sequence
  Description of Artificial Sequencecapture
ctggagaagaaggag

SEQ ID NO: 27 - DNA, 17 bp, Artificial Sequence
  Description of Artificial Sequencecapture
agactgcctggcggtgg

SEQ ID NO: 28 - DNA, 17 bp, Artificial Sequence
  Description of Artificial Sequencecapture
agactgtctggaagttg

SEQ ID NO: 29 - DNA, 17 bp, Artificial Sequence
  Description of Artificial Sequencecapture
agactgtctggcagttg

SEQ ID NO: 30 - DNA, 18 bp, Artificial Sequence
  Description of Artificial Sequencecapture
actgtctggtgtttgcct

SEQ ID NO: 31 - DNA, 18 bp, Artificial Sequence
  Description of Artificial Sequencecapture
gagttcagcaaatttgga

SEQ ID NO: 32 - DNA, 18 bp, Artificial Sequence
  Description of Artificial Sequencecapture
ctgttccacagacttaga

SEQ ID NO: 33 - DNA, 18 bp, Artificial Sequence
  Description of Artificial Sequencecapture
ctgttccgcagatttaga

SEQ ID NO: 34 - DNA, 18 bp, Artificial Sequence
  Description of Artificial Sequencecapture
gttctcagacaatttaga

SEQ ID NO: 35 - DNA, 18 bp, Artificial Sequence
  Description of Artificial Sequencecapture
aaatttggaggttttgac

SEQ ID NO: 36 - DNA, 18 bp, Artificial Sequence
  Description of Artificial Sequencecapture
agatttagaagatttgac

SEQ ID NO: 37 - DNA, 18 bp, Artificial Sequence
  Description of Artificial Sequencecapture
cacagacttagatttgac

SEQ ID NO: 38 - DNA, 18 bp, Artificial Sequence
  Description of Artificial Sequencecapture
agacaatttagatttgac

SEQ ID NO: 39 - DNA, 18 bp, Artificial Sequence
  Description of Artificial Sequencecapture
tttgacccgcagggtgca

SEQ ID NO: 40 - DNA, 18 bp, Artificial Sequence
  Description of Artificial Sequencecapture
tttgaccggcaatttgca

SEQ ID NO: 41 - DNA, 18 bp, Artificial Sequence
  Description of Artificial Sequencecapture
tttgacccgcaatttgca

SEQ ID NO: 42 - DNA, 18 bp, Artificial Sequence
  Description of Artificial Sequencecapture
catggctgtggcaaaaca

SEQ ID NO: 43 - DNA, 18 bp, Artificial Sequence
  Description of Artificial Sequencecapture
catcgctgtgctaaaaca

SEQ ID NO: 44 - DNA, 18 bp, Artificial Sequence
  Description of Artificial Sequencecapture
catcgctgtgacaaaaca

SEQ ID NO: 45 - DNA, 18 bp, Artificial Sequence
  Description of Artificial Sequencecapture
catcgctgtcctaaaaca

SEQ ID NO: 46 - DNA, 18 bp, Artificial Sequence
  Description of Artificial Sequencecapture
catcgccgtgacaaaaca

SEQ ID NO: 47 - DNA, 18 bp, Artificial Sequence
  Description of Artificial Sequencecapture
acttgaacatcatgatta

SEQ ID NO: 48 - DNA, 18 bp, Artificial Sequence
  Description of Artificial Sequencecapture
acttgaacatcctgatta

SEQ ID NO: 49 - DNA, 18 bp, Artificial Sequence
  Description of Artificial Sequencecapture
acttgaacatcgtgatta

SEQ ID NO: 50 - DNA, 18 bp, Artificial Sequence
  Description of Artificial Sequencecapture
acttgaacagtctgatta

SEQ ID NO: 51 - DNA, 18 bp, Artificial Sequence
  Description of Artificial Sequencecapture
ttaaacgctacaactcta

SEQ ID NO: 52 - DNA, 18 bp, Artificial Sequence
  Description of Artificial Sequencecapture
ttaaacgctccaactcta

SEQ ID NO: 53 - DNA, 18 bp, Artificial Sequence
  Description of Artificial Sequencecapture
aactctaccgctgctacc

SEQ ID NO: 54 - DNA, 18 bp, Artificial Sequence
  Description of Artificial Sequencecapture
aactctactgctgctacc

SEQ ID NO: 55 - DNA, 18 bp, Artificial Sequence
  Description of Artificial Sequencecapture
gcgggatcttgcagagga

SEQ ID NO: 56 - DNA, 18 bp, Artificial Sequence
  Description of Artificial Sequencecapture
gcgggatcctgcagagga

SEQ ID NO: 57 - DNA, 18 bp, Artificial Sequence
  Description of Artificial Sequencecapture
gcacgaccttgcagcggc

SEQ ID NO: 58 - DNA, 18 bp, Artificial Sequence
  Description of Artificial Sequencecapture
ggagaggaaacgggcggc

SEQ ID NO: 59 - DNA, 18 bp, Artificial Sequence
  Description of Artificial Sequencecapture
tcggaatggccaggagga

SEQ ID NO: 60 - DNA, 18 bp, Artificial Sequence
  Description of Artificial Sequencecapture
ggagacagctggcgttgt

SEQ ID NO: 61 - DNA, 18 bp, Artificial Sequence
  Description of Artificial Sequencecapture
attaggaatggtgactgg

SEQ ID NO: 62 - DNA, 18 bp, Artificial Sequence
  Description of Artificial Sequencecapture
atttcgtgtaccagttta

SEQ ID NO: 63 - DNA, 18 bp, Artificial Sequence
  Description of Artificial Sequencecapture
atttcgtgctccagttta

SEQ ID NO: 64 - DNA, 18 bp, Artificial Sequence
  Description of Artificial Sequencecapture
atttcgtgttccagttta

SEQ ID NO: 65 - DNA, 18 bp, Artificial Sequence
  Description of Artificial Sequencecapture
tttaagggcctgtgctac

SEQ ID NO: 66 - DNA, 18 bp, Artificial Sequence
  Description of Artificial Sequencecapture
tttaaggccatgtgctac

SEQ ID NO: 67 - DNA, 18 bp, Artificial Sequence
  Description of Artificial Sequencecapture
tttaagggcatgtgctac

SEQ ID NO: 68 - DNA, 18 bp, Artificial Sequence
  Description of Artificial Sequencecapture
ttcaccaacgggacggag

SEQ ID NO: 69 - DNA, 18 bp, Artificial Sequence
  Description of Artificial Sequencecapture
ttcaccaatgggacggag

SEQ ID NO: 70 - DNA, 18 bp, Artificial Sequence
  Description of Artificial Sequencecapture
cgggacggagcgcgtgcg

SEQ ID NO: 71 - DNA, 18 bp, Artificial Sequence
  Description of Artificial Sequencecapture
cgggacagagcgcgtgcg

SEQ ID NO: 72 - DNA, 18 bp, Artificial Sequence
  Description of Artificial Sequencecapture
cgggaccgagcgcgtgcg

SEQ ID NO: 73 - DNA, 18 bp, Artificial Sequence
  Description of Artificial Sequencecapture
cgggaccgagctcgtgcg

SEQ ID NO: 74 - DNA, 18 bp, Artificial Sequence
  Description of Artificial Sequencecapture
gtgcggggtgtgaccaga

SEQ ID NO: 75 - DNA, 18 bp, Artificial Sequence
  Description of Artificial Sequencecapture
gtgcgttatgtgaccaga

SEQ ID NO: 76 - DNA, 18 bp, Artificial Sequence
  Description of Artificial Sequencecapture
gtgcgtcttgtgaccaga

SEQ ID NO: 77 - DNA, 18 bp, Artificial Sequence
  Description of Artificial Sequencecapture
gtgcgtcttgtaaccaga

SEQ ID NO: 78 - DNA, 18 bp, Artificial Sequence
  Description of Artificial Sequencecapture
gtgcgtcttgtgagcaga

SEQ ID NO: 79 - DNA, 18 bp, Artificial Sequence
  Description of Artificial Sequencecapture
accagacacatctataac

SEQ ID NO: 80 - DNA, 18 bp, Artificial Sequence
  Description of Artificial Sequencecapture
accagatacatctataac

SEQ ID NO: 81 - DNA, 18 bp, Artificial Sequence
  Description of Artificial Sequencecapture
agcagaagcatctataac

SEQ ID NO: 82 - DNA, 18 bp, Artificial Sequence
  Description of Artificial Sequencecapture
ccgagaggagtacgtgcg

SEQ ID NO: 83 - DNA, 18 bp, Artificial Sequence
  Description of Artificial Sequencecapture
ccgagaagagtacgtgcg

SEQ ID NO: 84 - DNA, 18 bp, Artificial Sequence
  Description of Artificial Sequencecapture
ccgagaggaggacgtgcg

SEQ ID NO: 85 - DNA, 18 bp, Artificial Sequence
  Description of Artificial Sequencecapture
ccgagaagagatcgtgcg

SEQ ID NO: 86 - DNA, 18 bp, Artificial Sequence
  Description of Artificial Sequencecapture
ggagtacgtgcgcttcga

SEQ ID NO: 87 - DNA, 18 bp, Artificial Sequence
  Description of Artificial Sequencecapture
ggagtacgcgcgcttcga

SEQ ID NO: 88 - DNA, 18 bp, Artificial Sequence
  Description of Artificial Sequencecapture
ggagtacgcacgcttcga

SEQ ID NO: 89 - DNA, 18 bp, Artificial Sequence
  Description of Artificial Sequencecapture
gacgtgggggtgtaccgg

SEQ ID NO: 90 - DNA, 18 bp, Artificial Sequence
  Description of Artificial Sequencecapture
gacgtgggggagttccgg

SEQ ID NO: 91 - DNA, 18 bp, Artificial Sequence
  Description of Artificial Sequencecapture
gacgtggaggtgtaccgg

SEQ ID NO: 92 - DNA, 18 bp, Artificial Sequence
  Description of Artificial Sequencecapture
ggtgtaccgggcagtgac

SEQ ID NO: 93 - DNA, 18 bp, Artificial Sequence
  Description of Artificial Sequencecapture
ggtgtaccgggcggtgac

SEQ ID NO: 94 - DNA, 18 bp, Artificial Sequence
  Description of Artificial Sequencecapture
ggtgtatcgggcggtgac

SEQ ID NO: 95 - DNA, 18 bp, Artificial Sequence
  Description of Artificial Sequencecapture
ggtgtaccgcgcggtgac

SEQ ID NO: 96 - DNA, 18 bp, Artificial Sequence
  Description of Artificial Sequencecapture
ggtgtatcgggtggtgac

SEQ ID NO: 97 - DNA, 18 bp, Artificial Sequence
  Description of Artificial Sequencecapture
gcggtgacgccgcagggg

SEQ ID NO: 98 - DNA, 18 bp, Artificial Sequence
  Description of Artificial Sequencecapture
gcggtgaccccgcagggg

SEQ ID NO: 99 - DNA, 18 bp, Artificial Sequence
  Description of Artificial Sequencecapture
cgccgcaggggcggcctg

SEQ ID NO: 100 - DNA, 18 bp, Artificial Sequence
  Description of Artificial Sequencecapture
cgctgctggggctgcctg

SEQ ID NO: 101 - DNA, 18 bp, Artificial Sequence
  Description of Artificial Sequencecapture
cgccgctggggccgcctg

SEQ ID NO: 102 - DNA, 18 bp, Artificial Sequence
  Description of Artificial Sequencecapture
cgccgctggggcggcttg

SEQ ID NO: 103 - DNA, 18 bp, Artificial Sequence
  Description of Artificial Sequencecapture
gcggcctgttgccgagta

SEQ ID NO: 104 - DNA, 18 bp, Artificial Sequence
  Description of Artificial Sequencecapture
gcggcctagcgccgagta

SEQ ID NO: 105 - DNA, 18 bp, Artificial Sequence
  Description of Artificial Sequencecapture
gcggcctgatgccgagta

SEQ ID NO: 106 - DNA, 18 bp, Artificial Sequence
  Description of Artificial Sequencecapture
gcggcctgacgccgagta

SEQ ID NO: 107 - DNA, 18 bp, Artificial Sequence
  Description of Artificial Sequencecapture
gcggcctgtcgccgagta

SEQ ID NO: 108 - DNA, 18 bp, Artificial Sequence
  Description of Artificial Sequencecapture
gctgcctgccgccgagta

SEQ ID NO: 109 - DNA, 18 bp, Artificial Sequence
  Description of Artificial Sequencecapture
gctgcctgacgccgagta

SEQ ID NO: 110 - DNA, 18 bp, Artificial Sequence
  Description of Artificial Sequencecapture
gccgcctgacgccgagta

SEQ ID NO: 111 - DNA, 18 bp, Artificial Sequence
  Description of Artificial Sequencecapture
gccgcctgccgccgagta

SEQ ID NO: 112 - DNA, 18 bp, Artificial Sequence
  Description of Artificial Sequencecapture
gcggcttgacgccgagta

SEQ ID NO: 113 - DNA, 18 bp, Artificial Sequence
  Description of Artificial Sequencecapture
gcggcttgacgccgagta

SEQ ID NO: 114 - DNA, 18 bp, Artificial Sequence
  Description of Artificial Sequencecapture
tactggaacagccagaag

SEQ ID NO: 115 - DNA, 18 bp, Artificial Sequence
  Description of Artificial Sequencecapture
tactggaatagccagaag

SEQ ID NO: 116 - DNA, 18 bp, Artificial Sequence
  Description of Artificial Sequencecapture
cagaaggaagtcctggag

SEQ ID NO: 117 - DNA, 18 bp, Artificial Sequence
  Description of Artificial Sequencecapture
cagaaggacatcctggag

SEQ ID NO: 118 - DNA, 18 bp, Artificial Sequence
  Description of Artificial Sequencecapture
ctggagggggcccgggcg

SEQ ID NO: 119 - DNA, 18 bp, Artificial Sequence
  Description of Artificial Sequencecapture
ctggaggaggaccgggcg

SEQ ID NO: 120 - DNA, 18 bp, Artificial Sequence
  Description of Artificial Sequencecapture
ctggagaggacccgagcg

SEQ ID NO: 121 - DNA, 18 bp, Artificial Sequence
  Description of Artificial Sequencecapture
ctggaggggacccgggcg

SEQ ID NO: 122 - DNA, 18 bp, Artificial Sequence
  Description of Artificial Sequencecapture
ctggagaggacccgggcg

SEQ ID NO: 123 - DNA, 18 bp, Artificial Sequence
  Description of Artificial Sequencecapture
ctggagaggaaacgggcg

SEQ ID NO: 124 - DNA, 18 bp, Artificial Sequence
  Description of Artificial Sequencecapture
aggacccgagcggagttg

SEQ ID NO: 125 - DNA, 18 bp, Artificial Sequence
  Description of Artificial Sequencecapture
aggacccgggcggagttg

SEQ ID NO: 126 - DNA, 18 bp, Artificial Sequence
  Description of Artificial Sequencecapture
cccgggcgtcggtggaca

SEQ ID NO: 127 - DNA, 18 bp, Artificial Sequence
  Description of Artificial Sequencecapture
cccgggcggagttggaca

SEQ ID NO: 128 - DNA, 18 bp, Artificial Sequence
  Description of Artificial Sequencecapture
cccgggcggcggtggaca

SEQ ID NO: 129 - DNA, 18 bp, Artificial Sequence
  Description of Artificial Sequencecapture
aacgggcggcggtggaca

SEQ ID NO: 130 - DNA, 18 bp, Artificial Sequence
  Description of Artificial Sequencecapture
tggacagagtgtgcagac

SEQ ID NO: 131 - DNA, 18 bp, Artificial Sequence
  Description of Artificial Sequencecapture
tggacagggtgtgcagac

SEQ ID NO: 132 - DNA, 18 bp, Artificial Sequence
  Description of Artificial Sequencecapture
tggacacggtgtgcagac

SEQ ID NO: 133 - DNA, 18 bp, Artificial Sequence
  Description of Artificial Sequencecapture
tggacaccgtatgcagac

SEQ ID NO: 134 - DNA, 18 bp, Artificial Sequence
  Description of Artificial Sequencecapture
gaggtggggtaccgcggg

SEQ ID NO: 135 - DNA, 18 bp, Artificial Sequence
  Description of Artificial Sequencecapture
gaggtggcgttccgcggg

SEQ ID NO: 136 - DNA, 18 bp, Artificial Sequence
  Description of Artificial Sequencecapture
cagttggagctccgcacg

SEQ ID NO: 137 - DNA, 18 bp, Artificial Sequence
  Description of Artificial Sequencecapture
gaggtggcgtaccgcggg

SEQ ID NO: 138 - DNA, 18 bp, Artificial Sequence
  Description of Artificial Sequencecapture
cgcgggatcctgcagagg

SEQ ID NO: 139 - DNA, 18 bp, Artificial Sequence
  Description of Artificial Sequencecapture
cgcgggatcttgcagagg

SEQ ID NO: 140 - DNA, 18 bp, Artificial Sequence
  Description of Artificial Sequencecapture
cgcacgaccttgcagcgg

SEQ ID NO: 141 - DNA, 18 bp, Artificial Sequence
  Description of Artificial Sequencecapture
gtgtgtgccctgggcctg

SEQ ID NO: 142 - DNA, 18 bp, Artificial Sequence
  Description of Artificial Sequencecapture
gtgtgtgccttgggcctg

SEQ ID NO: 143 - DNA, 18 bp, Artificial Sequence
  Description of Artificial Sequencecapture
atcaagggagtgcgcaaa

SEQ ID NO: 144 - DNA, 18 bp, Artificial Sequence
  Description of Artificial Sequencecapture
atcaagggattgcgcaaa

SEQ ID NO: 145 - DNA, 18 bp, Artificial Sequence
  Description of Artificial Sequencecapture
tcttgtggcagcttaagt

SEQ ID NO: 146 - DNA, 18 bp, Artificial Sequence
  Description of Artificial Sequencecapture
ttcttggagtactctacg

SEQ ID NO: 147 - DNA, 18 bp, Artificial Sequence
  Description of Artificial Sequencecapture
tcttggagcaggttaaac

SEQ ID NO: 148 - DNA, 18 bp, Artificial Sequence
  Description of Artificial Sequencecapture
tttcctgtggcagggtaa

SEQ ID NO: 149 - DNA, 18 bp, Artificial Sequence
  Description of Artificial Sequencecapture
cagcctaagagggagtgt

SEQ ID NO: 150 - DNA, 18 bp, Artificial Sequence
  Description of Artificial Sequencecapture
tcttgaagcaggataagt

SEQ ID NO: 151 - DNA, 18 bp, Artificial Sequence
  Description of Artificial Sequencecapture
tcttggaggagcgtaagt

SEQ ID NO: 152 - DNA, 18 bp, Artificial Sequence
  Description of Artificial Sequencecapture
ttggagctgcgtaagtct

SEQ ID NO: 153 - DNA, 18 bp, Artificial Sequence
  Description of Artificial Sequencecapture
ttggagctgtgtaagtct

SEQ ID NO: 154 - DNA, 18 bp, Artificial Sequence
  Description of Artificial Sequencecapture
ttggagctgcttaagtct

SEQ ID NO: 155 - DNA, 18 bp, Artificial Sequence
  Description of Artificial Sequencecapture
tcttggagcaggctaagt

SEQ ID NO: 156 - DNA, 18 bp, Artificial Sequence
  Description of Artificial Sequencecapture
tcttgcagcaggataagt

SEQ ID NO: 157 - DNA, 18 bp, Artificial Sequence
  Description of Artificial Sequencecapture
ctgtggcagcctaagagg

SEQ ID NO: 158 - DNA, 18 bp, Artificial Sequence
  Description of Artificial Sequencecapture
tctacgggtgagtgttat

SEQ ID NO: 159 - DNA, 18 bp, Artificial Sequence
  Description of Artificial Sequencecapture
gagtgtcaattcttcaat

SEQ ID NO: 160 - DNA, 18 bp, Artificial Sequence
  Description of Artificial Sequencecapture
agcaggttacacatgagt

SEQ ID NO: 161 - DNA, 18 bp, Artificial Sequence
  Description of Artificial Sequencecapture
agcaggttaaacatgagt

SEQ ID NO: 162 - DNA, 18 bp, Artificial Sequence
  Description of Artificial Sequencecapture
ggagcaggctacacatga

SEQ ID NO: 163 - DNA, 18 bp, Artificial Sequence
  Description of Artificial Sequencecapture
tgaagcaggataagtttg

SEQ ID NO: 164 - DNA, 18 bp, Artificial Sequence
  Description of Artificial Sequencecapture
cagggtaagtataagtgt

SEQ ID NO: 165 - DNA, 18 bp, Artificial Sequence
  Description of Artificial Sequencecapture
tggagctgcttaagtctg

SEQ ID NO: 166 - DNA, 18 bp, Artificial Sequence
  Description of Artificial Sequencecapture
gcttaagtttgaatgtca

SEQ ID NO: 167 - DNA, 18 bp, Artificial Sequence
  Description of Artificial Sequencecapture
agtactctaggggtgagt

SEQ ID NO: 168 - DNA, 18 bp, Artificial Sequence
  Description of Artificial Sequencecapture
agtactctatgggtgagt

SEQ ID NO: 169 - DNA, 18 bp, Artificial Sequence
  Description of Artificial Sequencecapture
tttcttcaacgggacgga

SEQ ID NO: 170 - DNA, 18 bp, Artificial Sequence
  Description of Artificial Sequencecapture
agcgggtgcagttcctgg

SEQ ID NO: 171 - DNA, 18 bp, Artificial Sequence
  Description of Artificial Sequencecapture
gggtgcggtacctggaca

SEQ ID NO: 172 - DNA, 18 bp, Artificial Sequence
  Description of Artificial Sequencecapture
ttactggagagacacttc

SEQ ID NO: 173 - DNA, 18 bp, Artificial Sequence
  Description of Artificial Sequencecapture
gctggaaagatgcatcta

SEQ ID NO: 174 - DNA, 18 bp, Artificial Sequence
  Description of Artificial Sequencecapture
ttcctggagagatacttc

SEQ ID NO: 175 - DNA, 18 bp, Artificial Sequence
  Description of Artificial Sequencecapture
gcggtatctgcacagagg

SEQ ID NO: 176 - DNA, 18 bp, Artificial Sequence
  Description of Artificial Sequencecapture
tggaaagactcttctata

SEQ ID NO: 177 - DNA, 18 bp, Artificial Sequence
  Description of Artificial Sequencecapture
tggaaagtctcttctata

SEQ ID NO: 178 - DNA, 18 bp, Artificial Sequence
  Description of Artificial Sequencecapture
tggacagacacttctata

SEQ ID NO: 179 - DNA, 18 bp, Artificial Sequence
  Description of Artificial Sequencecapture
cagatacttccataacca

SEQ ID NO: 180 - DNA, 18 bp, Artificial Sequence
  Description of Artificial Sequencecapture
acttctatcaccaagagg

SEQ ID NO: 181 - DNA, 18 bp, Artificial Sequence
  Description of Artificial Sequencecapture
cttctataatcaggagga

SEQ ID NO: 182 - DNA, 18 bp, Artificial Sequence
  Description of Artificial Sequencecapture
ccataaccgggaggagaa

SEQ ID NO: 183 - DNA, 18 bp, Artificial Sequence
  Description of Artificial Sequencecapture
tcaccaagaagagtacgt

SEQ ID NO: 184 - DNA, 18 bp, Artificial Sequence
  Description of Artificial Sequencecapture
gaggagaacgtgcgcttc

SEQ ID NO: 185 - DNA, 18 bp, Artificial Sequence
  Description of Artificial Sequencecapture
aagaggaggacgtgcgct

SEQ ID NO: 186 - DNA, 18 bp, Artificial Sequence
  Description of Artificial Sequencecapture
agaggaggacttgcgctt

SEQ ID NO: 187 - DNA, 18 bp, Artificial Sequence
  Description of Artificial Sequencecapture
aagaggagtccgtgcgct

SEQ ID NO: 188 - DNA, 18 bp, Artificial Sequence
  Description of Artificial Sequencecapture
gaggagttcgtgcgcttc

SEQ ID NO: 189 - DNA, 18 bp, Artificial Sequence
  Description of Artificial Sequencecapture
gaggagaacctgcgcttc

SEQ ID NO: 190 - DNA, 18 bp, Artificial Sequence
  Description of Artificial Sequencecapture
gaggagttcctgcgcttc

SEQ ID NO: 191 - DNA, 18 bp, Artificial Sequence
  Description of Artificial Sequencecapture
gaggagctcctgcgcttc

SEQ ID NO: 192 - DNA, 18 bp, Artificial Sequence
  Description of Artificial Sequencecapture
gcgacgtgagggagtacc

SEQ ID NO: 193 - DNA, 18 bp, Artificial Sequence
  Description of Artificial Sequencecapture
gtacgtgcggttcgacag

SEQ ID NO: 194 - DNA, 18 bp, Artificial Sequence
  Description of Artificial Sequencecapture
tgggggagttccgggcgg

SEQ ID NO: 195 - DNA, 18 bp, Artificial Sequence
  Description of Artificial Sequencecapture
gtaccgggtggtgacgga

SEQ ID NO: 196 - DNA, 17 bp, Artificial Sequence
  Description of Artificial Sequencecapture
cgggcggcgacggagct

SEQ ID NO: 197 - DNA, 18 bp, Artificial Sequence
  Description of Artificial Sequencecapture
gggcggtgatggagctgg

SEQ ID NO: 198 - DNA, 18 bp, Artificial Sequence
  Description of Artificial Sequencecapture
gacggagctagggcggcc

SEQ ID NO: 199 - DNA, 18 bp, Artificial Sequence
  Description of Artificial Sequencecapture
gcctagcgccgagtactg

SEQ ID NO: 200 - DNA, 18 bp, Artificial Sequence
  Description of Artificial Sequencecapture
ggggcggcctgctgcgga

SEQ ID NO: 201 - DNA, 18 bp, Artificial Sequence
  Description of Artificial Sequencecapture
ggcctgacgctgagtact

SEQ ID NO: 202 - DNA, 18 bp, Artificial Sequence
  Description of Artificial Sequencecapture
cctgtcgccgagtcctgg

SEQ ID NO: 203 - DNA, 18 bp, Artificial Sequence
  Description of Artificial Sequencecapture
tgatgcggagcactggaa

SEQ ID NO: 204 - DNA, 18 bp, Artificial Sequence
  Description of Artificial Sequencecapture
gcctgatgaggagtactg

SEQ ID NO: 205 - DNA, 18 bp, Artificial Sequence
  Description of Artificial Sequencecapture
gcctgatgctgagtactg

SEQ ID NO: 206 - DNA, 18 bp, Artificial Sequence
  Description of Artificial Sequencecapture
gcctgatgaggagtactg

SEQ ID NO: 207 - DNA, 18 bp, Artificial Sequence
  Description of Artificial Sequencecapture
cctgatgcccagtactgg

SEQ ID NO: 208 - DNA, 18 bp, Artificial Sequence
  Description of Artificial Sequencecapture
cctgatgaggagcactgg

SEQ ID NO: 209 - DNA, 18 bp, Artificial Sequence
  Description of Artificial Sequencecapture
ggcctgataccgagtact

SEQ ID NO: 210 - DNA, 18 bp, Artificial Sequence
  Description of Artificial Sequencecapture
ccagaaggacttcctgga

SEQ ID NO: 211 - DNA, 18 bp, Artificial Sequence
  Description of Artificial Sequencecapture
ccagaaggacatcctgga

SEQ ID NO: 212 - DNA, 17 bp, Artificial Sequence
  Description of Artificial Sequencecapture
ctggagcaggcgcgggc

SEQ ID NO: 213 - DNA, 17 bp, Artificial Sequence
  Description of Artificial Sequencecapture
ctggagcagaggcgggc

SEQ ID NO: 214 - DNA, 17 bp, Artificial Sequence
  Description of Artificial Sequencecapture
ctggagcggaggcgggc

SEQ ID NO: 215 - DNA, 18 bp, Artificial Sequence
  Description of Artificial Sequencecapture
ggaagacgagcgggccgc

SEQ ID NO: 216 - DNA, 18 bp, Artificial Sequence
  Description of Artificial Sequencecapture
tcctggagcagaagcggg

SEQ ID NO: 217 - DNA, 18 bp, Artificial Sequence
  Description of Artificial Sequencecapture
ctggaggacaggcggggc

SEQ ID NO: 218 - DNA, 18 bp, Artificial Sequence
  Description of Artificial Sequencecapture
tggaagacaagcgggccg

SEQ ID NO: 219 - DNA, 18 bp, Artificial Sequence
  Description of Artificial Sequencecapture
agcagaggcaggccgcgg

SEQ ID NO: 220 - DNA, 18 bp, Artificial Sequence
  Description of Artificial Sequencecapture
tggaagacaggcgcgccg

SEQ ID NO: 221 - DNA, 18 bp, Artificial Sequence
  Description of Artificial Sequencecapture
aagacagggccgccgcgg

SEQ ID NO: 222 - DNA, 18 bp, Artificial Sequence
  Description of Artificial Sequencecapture
ggcgggccctggtggaca

SEQ ID NO: 223 - DNA, 18 bp, Artificial Sequence
  Description of Artificial Sequencecapture
ggcgggccgaggtggaca

SEQ ID NO: 224 - DNA, 18 bp, Artificial Sequence
  Description of Artificial Sequencecapture
ggcgggccgtggtggaca

SEQ ID NO: 225 - DNA, 18 bp, Artificial Sequence
  Description of Artificial Sequencecapture
ggccgcgctggacaacta

SEQ ID NO: 226 - DNA, 18 bp, Artificial Sequence
  Description of Artificial Sequencecapture
agcggggccgggtggaca

SEQ ID NO: 227 - DNA, 17 bp, Artificial Sequence
  Description of Artificial Sequencecapture
gcggggccaggtggaca

SEQ ID NO: 228 - DNA, 18 bp, Artificial Sequence
  Description of Artificial Sequencecapture
gggccgccgtggacacct

SEQ ID NO: 229 - DNA, 18 bp, Artificial Sequence
  Description of Artificial Sequencecapture
tggacaactactgcagac

SEQ ID NO: 230 - DNA, 18 bp, Artificial Sequence
  Description of Artificial Sequencecapture
ggtggacaattactgcag

SEQ ID NO: 231 - DNA, 18 bp, Artificial Sequence
  Description of Artificial Sequencecapture
tggacaccgtgtgcagac

SEQ ID NO: 232 - DNA, 18 bp, Artificial Sequence
  Description of Artificial Sequencecapture
ggacacctattgcagaca

SEQ ID NO: 233 - DNA, 18 bp, Artificial Sequence
  Description of Artificial Sequencecapture
ctacggagttgtggagag

SEQ ID NO: 234 - DNA, 18 bp, Artificial Sequence
  Description of Artificial Sequencecapture
cggggttgtggagagctt

SEQ ID NO: 235 - DNA, 18 bp, Artificial Sequence
  Description of Artificial Sequencecapture
acggggttgatgagagct

SEQ ID NO: 236 - DNA, 18 bp, Artificial Sequence
  Description of Artificial Sequencecapture
tacggggctgtggagagc

SEQ ID NO: 237 - DNA, 18 bp, Artificial Sequence
  Description of Artificial Sequencecapture
aactaccgggttgtggag

SEQ ID NO: 238 - DNA, 18 bp, Artificial Sequence
  Description of Artificial Sequencecapture
acggggttgttgagagct

SEQ ID NO: 239 - DNA, 18 bp, Artificial Sequence
  Description of Artificial Sequencecapture
agcttcacggtgcagcgg

SEQ ID NO: 240 - DNA, 18 bp, Artificial Sequence
  Description of Artificial Sequencecapture
tcacagtgcaccggcgac

SEQ ID NO: 241 - DNA, 18 bp, Artificial Sequence
  Description of Artificial Sequencecapture
ggttcccggacagatact

SEQ ID NO: 242 - DNA, 18 bp, Artificial Sequence
  Description of Artificial Sequencecapture
cctgatgaggactactgg

SEQ ID NO: 243 - DNA, 18 bp, Artificial Sequence
  Description of Artificial Sequencecapture
acagaggcatctataacc

SEQ ID NO: 244 - DNA, 18 bp, Artificial Sequence
  Description of Artificial Sequencecapture
tgcggagcactggaacag

SEQ ID NO: 245 - DNA, 18 bp, Artificial Sequence
  Description of Artificial Sequencecapture
gtggagagattcacagtg

SEQ ID NO: 246 - DNA, 18 bp, Artificial Sequence
  Description of Artificial Sequencecapture
acggggttggtgagagct

SEQ ID NO: 247 - DNA, 18 bp, Artificial Sequence
  Description of Artificial Sequencecapture
ttctataaccaggaggag

SEQ ID NO: 248 - DNA, 16 bp, Artificial Sequence
  Description of Artificial Sequencecapture
ccataaccaggaggag

SEQ ID NO: 249 - DNA, 18 bp, Artificial Sequence
  Description of Artificial Sequencecapture
gaggagtacgtgcgcttc

SEQ ID NO: 250 - DNA, 18 bp, Artificial Sequence
  Description of Artificial Sequencecapture
ggcctgatgccgagtact

SEQ ID NO: 251 - DNA, 17 bp, Artificial Sequence
  Description of Artificial Sequencecapture
catggcacctctgacag

SEQ ID NO: 252 - DNA, 18 bp, Artificial Sequence
  Description of Artificial Sequencecapture
tggagctgcgtaagtctg

SEQ ID NO: 253 - DNA, 18 bp, Artificial Sequence
  Description of Artificial Sequencecapture
tggagctgtgtaagtctg

SEQ ID NO: 254 - DNA, 18 bp, Artificial Sequence
  Description of Artificial Sequencecapture
tggagctgcttaagtctg

SEQ ID NO: 255 - DNA, 18 bp, Artificial Sequence
  Description of Artificial Sequencecapture
tgcggtacctggacagat

SEQ ID NO: 256 - DNA, 18 bp, Artificial Sequence
  Description of Artificial Sequencecapture
tgcggtacctgaacagat

SEQ ID NO: 257 - DNA, 18 bp, Artificial Sequence
  Description of Artificial Sequencecapture
tgcggttcctggagagac

SEQ ID NO: 258 - DNA, 18 bp, Artificial Sequence
  Description of Artificial Sequencecapture
tggacagatacttccata

SEQ ID NO: 259 - DNA, 18 bp, Artificial Sequence
  Description of Artificial Sequencecapture
tggagagacacttccata

SEQ ID NO: 260 - DNA, 17 bp, Artificial Sequence
  Description of Artificial Sequencecapture
gaggagttcctgcgctt

SEQ ID NO: 261 - DNA, 17 bp, Artificial Sequence
  Description of Artificial Sequencecapture
gaggagttcgtgcgctt

SEQ ID NO: 262 - DNA, 17 bp, Artificial Sequence
  Description of Artificial Sequencecapture
gaggagtacgcgcgctt

SEQ ID NO: 263 - DNA, 17 bp, Artificial Sequence
  Description of Artificial Sequencecapture
gaggagtccgtgcgctt

SEQ ID NO: 264 - DNA, 17 bp, Artificial Sequence
  Description of Artificial Sequencecapture
gaggagaacgcgcgctt

SEQ ID NO: 265 - DNA, 18 bp, Artificial Sequence
  Description of Artificial Sequencecapture
ggcggtgacggagctggg

SEQ ID NO: 266 - DNA, 18 bp, Artificial Sequence
  Description of Artificial Sequencecapture
ggcggtgagggagctggg

SEQ ID NO: 267 - DNA, 16 bp, Artificial Sequence
  Description of Artificial Sequencecapture
cggcctgtcgccgagt

SEQ ID NO: 268 - DNA, 16 bp, Artificial Sequence
  Description of Artificial Sequencecapture
cggcctgttgccgagt

SEQ ID NO: 269 - DNA, 16 bp, Artificial Sequence
  Description of Artificial Sequencecapture
cggcctgatgccgagt

SEQ ID NO: 270 - DNA, 16 bp, Artificial Sequence
  Description of Artificial Sequencecapture
cggcctagcgccgagt

SEQ ID NO: 271 - DNA, 18 bp, Artificial Sequence
  Description of Artificial Sequencecapture
agcggggccgggtggaca

SEQ ID NO: 272 - DNA, 18 bp, Artificial Sequence
  Description of Artificial Sequencecapture
agcggggccaggtggaca

SEQ ID NO: 273 - DNA, 18 bp, Artificial Sequence
  Description of Artificial Sequencecapture
ggtggacaattactgcag

SEQ ID NO: 274 - DNA, 18 bp, Artificial Sequence
  Description of Artificial Sequencecapture
ggtggacaactactgcag

SEQ ID NO: 275 - DNA, 18 bp, Artificial Sequence
  Description of Artificial Sequencecapture
tactgcagacacaactac

SEQ ID NO: 276 - DNA, 18 bp, Artificial Sequence
  Description of Artificial Sequencecapture
tactgcaggcacaactac

SEQ ID NO: 277 - DNA, 17 bp, Artificial Sequence
  Description of Artificial Sequencecapture
aactacggggttggtga

SEQ ID NO: 278 - DNA, 17 bp, Artificial Sequence
  Description of Artificial Sequencecapture
aactacggagttggtga

SEQ ID NO: 279 - DNA, 18 bp, Artificial Sequence
  Description of Artificial Sequencecapture
cggggttggtgagagctt

SEQ ID NO: 280 - DNA, 18 bp, Artificial Sequence
  Description of Artificial Sequencecapture
cggggttgtggagagctt

SEQ ID NO: 281 - DNA, 18 bp, Artificial Sequence
  Description of Artificial Sequencecapture
ggtggacacctactgcag

SEQ ID NO: 282 - DNA, 18 bp, Artificial Sequence
  Description of Artificial Sequencecapture
ggtgggcacctactgcag

SEQ ID NO: 283 - DNA, 18 bp, Artificial Sequence
  Description of Artificial Sequencecapture
ggtggacaactactgcag

SEQ ID NO: 284 - DNA, 18 bp, Artificial Sequence
  Description of Artificial Sequencecapture
actgcagatacaactacg

SEQ ID NO: 285 - DNA, 18 bp, Artificial Sequence
  Description of Artificial Sequencecapture
actgcagacacaactacg

SEQ ID NO: 286 - DNA, 17 bp, Artificial Sequence
  Description of Artificial Sequencecapture
cggaacagccaggaaga

SEQ ID NO: 287 - DNA, 17 bp, Artificial Sequence
  Description of Artificial Sequencecapture
cggaacggccaggaaga

SEQ ID NO: 288 - DNA, 18 bp, Artificial Sequence
  Description of Artificial Sequencecapture
ccctctcacggtgcaatg

SEQ ID NO: 289 - DNA, 18 bp, Artificial Sequence
  Description of Artificial Sequencecapture
ccctctcacagtgcaatg

SEQ ID NO: 290 - DNA, 17 bp, Artificial Sequence
  Description of Artificial Sequencecapture
gcacagagacatctata

SEQ ID NO: 291 - DNA, 17 bp, Artificial Sequence
  Description of Artificial Sequencecapture
gcacagaggcatctata

SEQ ID NO: 292 - DNA, 18 bp, Artificial Sequence
  Description of Artificial Sequencecapture
agaggaggacttgcgctt

SEQ ID NO: 293 - DNA, 18 bp, Artificial Sequence
  Description of Artificial Sequencecapture
agaggagaacgtgcgctt

SEQ ID NO: 294 - DNA, 18 bp, Artificial Sequence
  Description of Artificial Sequencecapture
agaggaggacgtgcgctt

SEQ ID NO: 295 - DNA, 18 bp, Artificial Sequence
  Description of Artificial Sequencecapture
ccagaaggacttcctgga

SEQ ID NO: 296 - DNA, 18 bp, Artificial Sequence
  Description of Artificial Sequencecapture
ccagaaggacatcctgga

SEQ ID NO: 297 - DNA, 16 bp, Artificial Sequence
  Description of Artificial Sequencecapture
tcctggaagacaggcg

SEQ ID NO: 298 - DNA, 17 bp, Artificial Sequence
  Description of Artificial Sequencecapture
tggaagacacgcgcgcc

SEQ ID NO: 299 - DNA, 17 bp, Artificial Sequence
  Description of Artificial Sequencecapture
tcctggagcaggcgcgg

SEQ ID NO: 300 - DNA, 18 bp, Artificial Sequence
  Description of Artificial Sequencecapture
acaggcgggccgcggtgg

SEQ ID NO: 301 - DNA, 18 bp, Artificial Sequence
  Description of Artificial Sequencecapture
aggcgcgggccgcggtgg

SEQ ID NO: 302 - DNA, 18 bp, Artificial Sequence
  Description of Artificial Sequencecapture
acaggcgcgccgcggtgg

SEQ ID NO: 303 - DNA, 18 bp, Artificial Sequence
  Description of Artificial Sequencecapture
aggcgggccctggtggac

SEQ ID NO: 304 - DNA, 18 bp, Artificial Sequence
  Description of Artificial Sequencecapture
aggcgcgccgcggtggac

SEQ ID NO: 305 - DNA, 18 bp, Artificial Sequence
  Description of Artificial Sequencecapture
gcgggccgcggtggacac

SEQ ID NO: 306 - DNA, 17 bp, Artificial Sequence
  Description of Artificial Sequencecapture
acggggttggtgagagc

SEQ ID NO: 307 - DNA, 17 bp, Artificial Sequence
  Description of Artificial Sequencecapture
acggggctgtggagagc

SEQ ID NO: 308 - DNA, 17 bp, Artificial Sequence
  Description of Artificial Sequencecapture
gaacctgcccttcgaca

SEQ ID NO: 309 - DNA, 17 bp, Artificial Sequence
  Description of Artificial Sequencecapture
gaacctgcgcttcgaca

SEQ ID NO: 310 - DNA, 17 bp, Artificial Sequence
  Description of Artificial Sequencecapture
cgacagcgacgtggagg

SEQ ID NO: 311 - DNA, 17 bp, Artificial Sequence
  Description of Artificial Sequencecapture
cgacagcaacgtggagg

SEQ ID NO: 312 - DNA, 18 bp, Artificial Sequence
  Description of Artificial Sequencecapture
gaagggaatcctggagga

SEQ ID NO: 313 - DNA, 18 bp, Artificial Sequence
  Description of Artificial Sequencecapture
gaagggcatcccggagga

SEQ ID NO: 314 - DNA, 17 bp, Artificial Sequence
  Description of Artificial Sequencecapture
gaggagaatcgggacaa

SEQ ID NO: 315 - DNA, 17 bp, Artificial Sequence
  Description of Artificial Sequencecapture
gaggagaagcgggacaa

SEQ ID NO: 316 - DNA, 18 bp, Artificial Sequence
  Description of Artificial Sequencecapture
gtggacacctactgcaga

SEQ ID NO: 317 - DNA, 18 bp, Artificial Sequence
  Description of Artificial Sequencecapture
atggacgactactgcaga

SEQ ID NO: 318 - DNA, 18 bp, Artificial Sequence
  Description of Artificial Sequencecapture
gatacagttacggggttt

SEQ ID NO: 319 - DNA, 18 bp, Artificial Sequence
  Description of Artificial Sequencecapture
gatacaattacggggttt

SEQ ID NO: 320 - DNA, 18 bp, Artificial Sequence
  Description of Artificial Sequencecapture
gatacaattacagggttt

SEQ ID NO: 321 - DNA, 18 bp, Artificial Sequence
  Description of Artificial Sequencecapture
taaagatacttctataac

SEQ ID NO: 322 - DNA, 18 bp, Artificial Sequence
  Description of Artificial Sequencecapture
taaagatatttctataac

SEQ ID NO: 323 - DNA, 18 bp, Artificial Sequence
  Description of Artificial Sequencecapture
tccagacactgatgatgc

SEQ ID NO: 324 - DNA, 18 bp, Artificial Sequence
  Description of Artificial Sequencecapture
cttatgccgcgtttgtac

SEQ ID NO: 325 - DNA, 18 bp, Artificial Sequence
  Description of Artificial Sequencecapture
cttatgccatgtttgtac

SEQ ID NO: 326 - DNA, 18 bp, Artificial Sequence
  Description of Artificial Sequencecapture
tgtacagacgcatagacc

SEQ ID NO: 327 - DNA, 18 bp, Artificial Sequence
  Description of Artificial Sequencecapture
tgtacagacgcatagacc

SEQ ID NO: 328 - DNA, 18 bp, Artificial Sequence
  Description of Artificial Sequencecapture
catagaacaacaggagag

SEQ ID NO: 329 - DNA, 18 bp, Artificial Sequence
  Description of Artificial Sequencecapture
caacaggagagtttatgt

SEQ ID NO: 330 - DNA, 18 bp, Artificial Sequence
  Description of Artificial Sequencecapture
caacaggggagtttatgt

SEQ ID NO: 331 - DNA, 18 bp, Artificial Sequence
  Description of Artificial Sequencecapture
tatgtttgaatttgatga

SEQ ID NO: 332 - DNA, 18 bp, Artificial Sequence
  Description of Artificial Sequencecapture
tatgtttgagtttgatga

SEQ ID NO: 333 - DNA, 18 bp, Artificial Sequence
  Description of Artificial Sequencecapture
gaatttgatgaagatgag

SEQ ID NO: 334 - DNA, 18 bp, Artificial Sequence
  Description of Artificial Sequencecapture
gaatttgatgacgatgag

SEQ ID NO: 335 - DNA, 18 bp, Artificial Sequence
  Description of Artificial Sequencecapture
gagtttgatgatgatgag

SEQ ID NO: 336 - DNA, 18 bp, Artificial Sequence
  Description of Artificial Sequencecapture
gatgagatgttctatgtg

SEQ ID NO: 337 - DNA, 18 bp, Artificial Sequence
  Description of Artificial Sequencecapture
gatgagcagttctatgtg

SEQ ID NO: 338 - DNA, 18 bp, Artificial Sequence
  Description of Artificial Sequencecapture
atctggacaagaaggagg

SEQ ID NO: 339 - DNA, 18 bp, Artificial Sequence
  Description of Artificial Sequencecapture
atctggataaaaaggagg

SEQ ID NO: 340 - DNA, 18 bp, Artificial Sequence
  Description of Artificial Sequencecapture
atctggataagaaggagg

SEQ ID NO: 341 - DNA, 18 bp, Artificial Sequence
  Description of Artificial Sequencecapture
gtttggccaagccttttc

SEQ ID NO: 342 - DNA, 18 bp, Artificial Sequence
  Description of Artificial Sequencecapture
gtttggccgagccttttc

SEQ ID NO: 343 - DNA, 18 bp, Artificial Sequence
  Description of Artificial Sequencecapture
ttgctatattgaacaaca

SEQ ID NO: 344 - DNA, 18 bp, Artificial Sequence
  Description of Artificial Sequencecapture
ttgctatatcgaacaaca

SEQ ID NO: 345 - DNA, 18 bp, Artificial Sequence
  Description of Artificial Sequencecapture
ttgaataccttgatccag

SEQ ID NO: 346 - DNA, 18 bp, Artificial Sequence
  Description of Artificial Sequencecapture
ttgaatatcgctatccag

SEQ ID NO: 347 - DNA, 18 bp, Artificial Sequence
  Description of Artificial Sequencecapture
tcaggccaccaacggtac

SEQ ID NO: 348 - DNA, 18 bp, Artificial Sequence
  Description of Artificial Sequencecapture
tcaggccgccaatggtac

SEQ ID NO: 349 - DNA, 18 bp, Artificial Sequence
  Description of Artificial Sequencecapture
gagaattacgtgtaccag

SEQ ID NO: 350 - DNA, 18 bp, Artificial Sequence
  Description of Artificial Sequencecapture
gagaattaccttttccag

SEQ ID NO: 351 - DNA, 18 bp, Artificial Sequence
  Description of Artificial Sequencecapture
gagaattacgtgcaccag

SEQ ID NO: 352 - DNA, 18 bp, Artificial Sequence
  Description of Artificial Sequencecapture
gagaattaagtgtaccag

SEQ ID NO: 353 - DNA, 18 bp, Artificial Sequence
  Description of Artificial Sequencecapture
gagaattacgtggaccag

SEQ ID NO: 354 - DNA, 18 bp, Artificial Sequence
  Description of Artificial Sequencecapture
accagggacggcaggaat

SEQ ID NO: 355 - DNA, 18 bp, Artificial Sequence
  Description of Artificial Sequencecapture
tccagggacgacaggaat

SEQ ID NO: 356 - DNA, 18 bp, Artificial Sequence
  Description of Artificial Sequencecapture
accagttacggcaggaat

SEQ ID NO: 357 - DNA, 18 bp, Artificial Sequence
  Description of Artificial Sequencecapture
tccagggactgcaggaat

SEQ ID NO: 358 - DNA, 18 bp, Artificial Sequence
  Description of Artificial Sequencecapture
tctacaaccgggaggagt

SEQ ID NO: 359 - DNA, 18 bp, Artificial Sequence
  Description of Artificial Sequencecapture
tctacaacaggcaggagt

SEQ ID NO: 360 - DNA, 18 bp, Artificial Sequence
  Description of Artificial Sequencecapture
tctacaaccggcaggagt

SEQ ID NO: 361 - DNA, 18 bp, Artificial Sequence
  Description of Artificial Sequencecapture
aggagtacgcgcgcttcg

SEQ ID NO: 362 - DNA, 18 bp, Artificial Sequence
  Description of Artificial Sequencecapture
aggagctcgtgcgcttcg

SEQ ID NO: 363 - DNA, 18 bp, Artificial Sequence
  Description of Artificial Sequencecapture
aggagttcgcgcgcttcg

SEQ ID NO: 364 - DNA, 18 bp, Artificial Sequence
  Description of Artificial Sequencecapture
aggagttcgtgcgcttcg

SEQ ID NO: 365 - DNA, 18 bp, Artificial Sequence
  Description of Artificial Sequencecapture
gacgtgggggagttccgg

SEQ ID NO: 366 - DNA, 18 bp, Artificial Sequence
  Description of Artificial Sequencecapture
gacgtgggagagttccgg

SEQ ID NO: 367 - DNA, 18 bp, Artificial Sequence
  Description of Artificial Sequencecapture
cggcctgctgcggagtac

SEQ ID NO: 368 - DNA, 18 bp, Artificial Sequence
  Description of Artificial Sequencecapture
cggcctgatgaggagtac

SEQ ID NO: 369 - DNA, 18 bp, Artificial Sequence
  Description of Artificial Sequencecapture
cggcctgacgaggagtac

SEQ ID NO: 370 - DNA, 18 bp, Artificial Sequence
  Description of Artificial Sequencecapture
cggcctgatgaggactac

SEQ ID NO: 371 - DNA, 18 bp, Artificial Sequence
  Description of Artificial Sequencecapture
cggcctgaggcggagtac

SEQ ID NO: 372 - DNA, 18 bp, Artificial Sequence
  Description of Artificial Sequencecapture
cggcctgatgaggtgtac

SEQ ID NO: 373 - DNA, 18 bp, Artificial Sequence
  Description of Artificial Sequencecapture
tgatgaggagtactggaa

SEQ ID NO: 374 - DNA, 18 bp, Artificial Sequence
  Description of Artificial Sequencecapture
tgatgaggactactggaa

SEQ ID NO: 375 - DNA, 18 bp, Artificial Sequence
  Description of Artificial Sequencecapture
tgatgaggtgtactggaa

SEQ ID NO: 376 - DNA, 18 bp, Artificial Sequence
  Description of Artificial Sequencecapture
cagaaggacatcctggag

SEQ ID NO: 377 - DNA, 18 bp, Artificial Sequence
  Description of Artificial Sequencecapture
cagaaggacctcctggag

SEQ ID NO: 378 - DNA, 18 bp, Artificial Sequence
  Description of Artificial Sequencecapture
cagaaggacttcctggag

SEQ ID NO: 379 - DNA, 18 bp, Artificial Sequence
  Description of Artificial Sequencecapture
cagaaggacaacctggag

SEQ ID NO: 380 - DNA, 18 bp, Artificial Sequence
  Description of Artificial Sequencecapture
cagaaggacctcctgtag

SEQ ID NO: 381 - DNA, 18 bp, Artificial Sequence
  Description of Artificial Sequencecapture
gacctcctgtaggagaag

SEQ ID NO: 382 - DNA, 18 bp, Artificial Sequence
  Description of Artificial Sequencecapture
ctggaggagaagcgggca

SEQ ID NO: 383 - DNA, 18 bp, Artificial Sequence
  Description of Artificial Sequencecapture
ctggaggaggagcgggca

SEQ ID NO: 384 - DNA, 18 bp, Artificial Sequence
  Description of Artificial Sequencecapture
ctggaggagaggcgggca

SEQ ID NO: 385 - DNA, 18 bp, Artificial Sequence
  Description of Artificial Sequencecapture
ctgtaggagaagcgggca

SEQ ID NO: 386 - DNA, 18 bp, Artificial Sequence
  Description of Artificial Sequencecapture
agcgggcagtgccggaca

SEQ ID NO: 387 - DNA, 18 bp, Artificial Sequence
  Description of Artificial Sequencecapture
agcgggcattgccggaca

SEQ ID NO: 388 - DNA, 18 bp, Artificial Sequence
  Description of Artificial Sequencecapture
gcgggcagtgctggacag

SEQ ID NO: 389 - DNA, 18 bp, Artificial Sequence
  Description of Artificial Sequencecapture
gcgggcagtgctggacag

SEQ ID NO: 390 - DNA, 18 bp, Artificial Sequence
  Description of Artificial Sequencecapture
ggacagggtatgcagaca

SEQ ID NO: 391 - DNA, 18 bp, Artificial Sequence
  Description of Artificial Sequencecapture
ggacaggatgtgcagaca

SEQ ID NO: 392 - DNA, 18 bp, Artificial Sequence
  Description of Artificial Sequencecapture
ggacaggatatgcagaca

SEQ ID NO: 393 - DNA, 18 bp, Artificial Sequence
  Description of Artificial Sequencecapture
acgagctggacgaggccg

SEQ ID NO: 394 - DNA, 18 bp, Artificial Sequence
  Description of Artificial Sequencecapture
acgagctgggcgggccca

SEQ ID NO: 395 - DNA, 18 bp, Artificial Sequence
  Description of Artificial Sequencecapture
acgagctggtcgggccca

SEQ ID NO: 396 - DNA, 18 bp, Artificial Sequence
  Description of Artificial Sequencecapture
gaatgctacgcgtttaat

SEQ ID NO: 397 - DNA, 18 bp, Artificial Sequence
  Description of Artificial Sequencecapture
gaatgctacccgtttaat

SEQ ID NO: 398 - DNA, 23 bp, Artificial Sequence
  Description of Artificial Sequenceprimer
gagtactggaatagccagaagga

SEQ ID NO: 399 - DNA, 23 bp, Artificial Sequence
  Description of Artificial Sequenceprimer
gagtactggaacagccagaagga

SEQ ID NO: 400 - DNA, 18 bp, Artificial Sequence
  Description of Artificial Sequenceprimer
ggcgacgacgctcacctc

SEQ ID NO: 401 - DNA, 18 bp, Artificial Sequence
  Description of Artificial Sequenceprimer
ttcgtgtccccacagcac

SEQ ID NO: 402 - DNA, 18 bp, Artificial Sequence
  Description of Artificial Sequenceprimer
ttcgtgtccccccagcac

SEQ ID NO: 403 - DNA, 16 bp, Artificial Sequence
  Description of Artificial Sequenceprimer
gcacccgctccgtccc

SEQ ID NO: 404 - DNA, 21 bp, Artificial Sequence
  Description of Artificial Sequenceprimer
gattccccgcagaggatttcg

SEQ ID NO: 405 - DNA, 21 bp, Artificial Sequence
  Description of Artificial Sequenceprimer
gctcacctctcctctgcaaga

SEQ ID NO: 406 - DNA, 20 bp, Artificial Sequence
  Description of Artificial Sequenceprimer
ctcacctctcctctgcagga

SEQ ID NO: 407 - DNA, 20 bp, Artificial Sequence
  Description of Artificial Sequenceprimer
gctacttcaccaacgggacc

SEQ ID NO: 408 - DNA, 20 bp, Artificial Sequence
  Description of Artificial Sequenceprimer
gctacttcaccaacgggacg

SEQ ID NO: 409 - DNA, 19 bp, Artificial Sequence
  Description of Artificial Sequenceprimer
tcgtgcggagctccaactg

SEQ ID NO: 410 - DNA, 23 bp, Artificial Sequence
  Description of Artificial Sequenceprimer
tcggtgacagatttctatccagc

SEQ ID NO: 411 - DNA, 24 bp, Artificial Sequence
  Description of Artificial Sequenceprimer
ctcagtgacagatttctatccagc

SEQ ID NO: 412 - DNA, 21 bp, Artificial Sequence
  Description of Artificial Sequenceprimer
agcatcaccaggatctggaag

SEQ ID NO: 413 - DNA, 21 bp, Artificial Sequence
  Description of Artificial Sequenceprimer
tccccacagcacgtttcttgt

SEQ ID NO: 414 - DNA, 23 bp, Artificial Sequence
  Description of Artificial Sequenceprimer
gcacgtttcttggagtactctac

SEQ ID NO: 415 - DNA, 26 bp, Artificial Sequence
  Description of Artificial Sequenceprimer
cacgtttcttggagcaggttaaacat

SEQ ID NO: 416 - DNA, 19 bp, Artificial Sequence
  Description of Artificial Sequenceprimer
gtccccacagcacgtttcc

SEQ ID NO: 417 - DNA, 17 bp, Artificial Sequence
  Description of Artificial Sequenceprimer
tcacctcgccgctgcac

SEQ ID NO: 418 - DNA, 29 bp, Artificial Sequence
  Description of Artificial Sequenceprimer
gcttgtcatcttcactcatcagctgacca

SEQ ID NO: 419 - DNA, 25 bp, Artificial Sequence
  Description of Artificial Sequenceprimer
gtcaccttcactcgtcagctgacca

SEQ ID NO: 420 - DNA, 26 bp, Artificial Sequence
  Description of Artificial Sequenceprimer
ggtggacacataccattggtagcagc

SEQ ID NO: 421 - DNA, 25 bp, Artificial Sequence
  Description of Artificial Sequenceprimer
ctgattccccgcagaggatttcgtg

SEQ ID NO: 422 - DNA, 20 bp, Artificial Sequence
  Description of Artificial Sequenceprimer
ggggcgacgacgctcacctc

SEQ ID NO: 423 - DNA, 23 bp, Artificial Sequence
  Description of Artificial Sequenceprimer
gatgctccaagccctctcccaga

SEQ ID NO: 424 - DNA, 23 bp, Artificial Sequence
  Description of Artificial Sequenceprimer
ttacagaggccccctgcgttctg

SEQ ID NO: 425 - DNA, 25 bp, Artificial Sequence
  Description of Artificial Sequenceprimer
cgttcttgtccccccagcacgtttc

SEQ ID NO: 426 - DNA, 25 bp, Artificial Sequence
  Description of Artificial Sequenceprimer
cgtgtccccacagcacgtttcctgt

SEQ ID NO: 427 - DNA, 24 bp, Artificial Sequence
  Description of Artificial Sequenceprimer
ttcgtgtccccacagcacgtttct

SEQ ID NO: 428 - DNA, 20 bp, Artificial Sequence
  Description of Artificial Sequenceprimer
cgctcacctcgccgctgcac

SEQ ID NO: 429 - DNA, 24 bp, Artificial Sequence
  Description of Artificial Sequenceprimer
acccgcagcacgtttcttggagct

SEQ ID NO: 430 - DNA, 23 bp, Artificial Sequence
  Description of Artificial Sequenceprimer
cccgcagcacgtttctcggagct

SEQ ID NO: 431 - DNA, 23 bp, Artificial Sequence
  Description of Artificial Sequenceprimer
tcgccgctgcactgtgaagctct

SEQ ID NO: 432 - DNA, 24 bp, Artificial Sequence
  Description of Artificial Sequenceprimer
agtacgcgcgctacaacagtgacc

SEQ ID NO: 433 - DNA, 18 bp, Artificial Sequence
  Description of Artificial Sequenceprimer
ggacccaggccccgccct

SEQ ID NO: 434 - DNA, 34 bp, Artificial Sequence
  Description of Artificial Sequenceprimer
tcattcccactaccttttttttctcctagtccaa

SEQ ID NO: 435 - DNA, 32 bp, Artificial Sequence
  Description of Artificial Sequenceprimer
tttaggaagtcagaaagctgctaactccattg

SEQ ID NO: 436 - DNA, 20 bp, Artificial Sequence
  Description of Artificial Sequenceprimer
ggagcgggtgcggttcctgc

SEQ ID NO: 437 - DNA, 23 bp, Artificial Sequence
  Description of Artificial Sequenceprimer
tcgccgctgcactgtgaagctct

SEQ ID NO: 438 - DNA, 33 bp, Artificial Sequence
  Description of Artificial Sequenceprimer
ctaagtgtgagtgtcatatcttcaatgggatga

SEQ ID NO: 439 - DNA, 28 bp, Artificial Sequence
  Description of Artificial Sequenceprimer
gctgcactgtgaagctctcaaaaaaccc

SEQ ID NO: 440 - DNA, 27 bp, Artificial Sequence
  Description of Artificial Sequenceprimer
gctgcactgtgaagctctcaaaaaccc

SEQ ID NO: 441 - DNA, 25 bp, Artificial Sequence
  Description of Artificial Sequenceprimer
caatgggacggagcggtacctgtaa

SEQ ID NO: 442 - DNA, 23 bp, Artificial Sequence
  Description of Artificial Sequenceprimer
cggaactcccccatgtcgctgtt

SEQ ID NO: 443 - DNA, 26 bp, Artificial Sequence
  Description of Artificial Sequenceprimer
cagcggaccatgtgtcaacttatgcc

SEQ ID NO: 444 - DNA, 27 bp, Artificial Sequence
  Description of Artificial Sequenceprimer
ggctacagaggaagaggcaaagatagg

SEQ ID NO: 445 - DNA, 21 bp, Artificial Sequence
  Description of Artificial Sequenceprimer
gccccctccccgcagagaatt

SEQ ID NO: 446 - DNA, 20 bp, Artificial Sequence
  Description of Artificial Sequenceprimer
ctcggcgctgcagggtcacg

SEQ ID NO: 447 - DNA, 20 bp, Artificial Sequence
  Description of Artificial Sequenceprimer
ctcggcgctgcagggtcatg

SEQ ID NO: 448 - DNA, 19 bp, Artificial Sequence
  Description of Artificial Sequenceprimer
ctccccgcagagaattacg

SEQ ID NO: 449 - DNA, 19 bp, Artificial Sequence
  Description of Artificial Sequenceprimer
ctccccgcagagaattacc

SEQ ID NO: 450 - DNA, 20 bp, Artificial Sequence
  Description of Artificial Sequenceprimer
caaagccctcactacctcgg

SEQ ID NO: 451 - DNA, 19 bp, Artificial Sequence
  Description of Artificial Sequenceprimer
caaagccctactacctcgg

SEQ ID NO: 452 - DNA, 20 bp, Artificial Sequence
  Description of Artificial Sequenceprimer
caaagccctcactacctcgt

SEQ ID NO: 453 - DNA, 19 bp, Artificial Sequence
  Description of Artificial Sequenceprimer
caaagccctactacctcgt

SEQ ID NO: 454 - DNA, 20 bp, Artificial Sequence
  Description of Artificial Sequenceprimer
caaagccctcactacctcga

SEQ ID NO: 455 - DNA, 19 bp, Artificial Sequence
  Description of Artificial Sequenceprimer
caaagccctactacctcga

SEQ ID NO: 456 - DNA, 17 bp, Artificial Sequence
  Description of Artificial Sequencecapture
attacgtgcaccagtta

SEQ ID NO: 457 - DNA, 17 bp, Artificial Sequence
  Description of Artificial Sequencecapture
attacgtggaccagtta

SEQ ID NO: 458 - DNA, 16 bp, Artificial Sequence
  Description of Artificial Sequencecapture
gtaccagggacggcag

SEQ ID NO: 459 - DNA, 16 bp, Artificial Sequence
  Description of Artificial Sequencecapture
gtaccagttacggcag

SEQ ID NO: 460 - DNA, 16 bp, Artificial Sequence
  Description of Artificial Sequencecapture
tttccagggacggcag

SEQ ID NO: 461 - DNA, 16 bp, Artificial Sequence
  Description of Artificial Sequencecapture
gcaccagttacggcag

SEQ ID NO: 462 - DNA, 16 bp, Artificial Sequence
  Description of Artificial Sequencecapture
tttccagggactgcag

SEQ ID NO: 463 - DNA, 16 bp, Artificial Sequence
  Description of Artificial Sequencecapture
ggaccagttacggcag

SEQ ID NO: 464 - DNA, 17 bp, Artificial Sequence
  Description of Artificial Sequencecapture
ccagggactgcaggaat

SEQ ID NO: 465 - DNA, 18 bp, Artificial Sequence
  Description of Artificial Sequencecapture
aatgctacccgtttaatg

SEQ ID NO: 466 - DNA, 18 bp, Artificial Sequence
  Description of Artificial Sequencecapture
ctacaacaggcaggagta

SEQ ID NO: 467 - DNA, 18 bp, Artificial Sequence
  Description of Artificial Sequencecapture
tacaaccggcaggagtac

SEQ ID NO: 468 - DNA, 17 bp, Artificial Sequence
  Description of Artificial Sequencecapture
ggaggagttcgcgcgct

SEQ ID NO: 469 - DNA, 18 bp, Artificial Sequence
  Description of Artificial Sequencecapture
gaggagtacgcgcgcttg

SEQ ID NO: 470 - DNA, 18 bp, Artificial Sequence
  Description of Artificial Sequencecapture
gaggagttcgtgcgcttg

SEQ ID NO: 471 - DNA, 18 bp, Artificial Sequence
  Description of Artificial Sequencecapture
ggaggagctcgtgcgctt

SEQ ID NO: 472 - DNA, 18 bp, Artificial Sequence
  Description of Artificial Sequencecapture
caggagtacgcgcgcttg

SEQ ID NO: 473 - DNA, 17 bp, Artificial Sequence
  Description of Artificial Sequencecapture
gacgtgggagagttccg

SEQ ID NO: 474 - DNA, 18 bp, Artificial Sequence
  Description of Artificial Sequencecapture
cggcctgctgcggagtac

SEQ ID NO: 475 - DNA, 18 bp, Artificial Sequence
  Description of Artificial Sequencecapture
cggcctgatgaggagtac

SEQ ID NO: 476 - DNA, 18 bp, Artificial Sequence
  Description of Artificial Sequencecapture
cggcctgacgaggagtac

SEQ ID NO: 477 - DNA, 18 bp, Artificial Sequence
  Description of Artificial Sequencecapture
gcggcctgaggcggagta

SEQ ID NO: 478 - DNA, 18 bp, Artificial Sequence
  Description of Artificial Sequencecapture
cggcctgaggaggagtac

SEQ ID NO: 479 - DNA, 18 bp, Artificial Sequence
  Description of Artificial Sequencecapture
cggcctgatgaggactac

SEQ ID NO: 480 - DNA, 18 bp, Artificial Sequence
  Description of Artificial Sequencecapture
cggcctgatgaggtgtac

SEQ ID NO: 481 - DNA, 17 bp, Artificial Sequence
  Description of Artificial Sequencecapture
tgatgaggtgtactgga

SEQ ID NO: 482 - DNA, 17 bp, Artificial Sequence
  Description of Artificial Sequencecapture
gatgaggactactggaa

SEQ ID NO: 483 - DNA, 17 bp, Artificial Sequence
  Description of Artificial Sequencecapture
agaaggacatcctggag

SEQ ID NO: 484 - DNA, 17 bp, Artificial Sequence
  Description of Artificial Sequencecapture
agaaggacctcctggag

SEQ ID NO: 485 - DNA, 17 bp, Artificial Sequence
  Description of Artificial Sequencecapture
agaaggacttcctggag

SEQ ID NO: 486 - DNA, 17 bp, Artificial Sequence
  Description of Artificial Sequencecapture
agaaggacctcctgtag

SEQ ID NO: 487 - DNA, 17 bp, Artificial Sequence
  Description of Artificial Sequencecapture
gaaggacaacctggagg

SEQ ID NO: 488 - DNA, 17 bp, Artificial Sequence
  Description of Artificial Sequencecapture
acctcctgtaggagaag

SEQ ID NO: 489 - DNA, 17 bp, Artificial Sequence
  Description of Artificial Sequencecapture
tggaggagaagcgggca

SEQ ID NO: 490 - DNA, 17 bp, Artificial Sequence
  Description of Artificial Sequencecapture
tggaggaggagcgggca

SEQ ID NO: 491 - DNA, 17 bp, Artificial Sequence
  Description of Artificial Sequencecapture
ggaggagaggcgggcag

SEQ ID NO: 492 - DNA, 17 bp, Artificial Sequence
  Description of Artificial Sequencecapture
tgtaggagaagcgggca

SEQ ID NO: 493 - DNA, 17 bp, Artificial Sequence
  Description of Artificial Sequencecapture
agcgggcattgccggac

SEQ ID NO: 494 - DNA, 17 bp, Artificial Sequence
  Description of Artificial Sequencecapture
gcttgtgctggacaggg

SEQ ID NO: 495 - DNA, 17 bp, Artificial Sequence
  Description of Artificial Sequencecapture
ccggacagagtatgcag

SEQ ID NO: 496 - DNA, 19 bp, Artificial Sequence
  Description of Artificial Sequencecapture
cggacagggtatgcagaca

SEQ ID NO: 497 - DNA, 19 bp, Artificial Sequence
  Description of Artificial Sequencecapture
cggacaggatgtgcagaca

SEQ ID NO: 498 - DNA, 19 bp, Artificial Sequence
  Description of Artificial Sequencecapture
tggacagggtatgcagaca

SEQ ID NO: 499 - DNA, 17 bp, Artificial Sequence
  Description of Artificial Sequencecapture
cggacaggatatgcaga

SEQ ID NO: 500 - DNA, 17 bp, Artificial Sequence
  Description of Artificial Sequencecapture
cgagctggacgaggccg

SEQ ID NO: 501 - DNA, 17 bp, Artificial Sequence
  Description of Artificial Sequencecapture
cgagctgggcgggccca

SEQ ID NO: 502 - DNA, 17 bp, Artificial Sequence
  Description of Artificial Sequencecapture
cgagctggtcgggccca

SEQ ID NO: 503 - DNA, 17 bp, Artificial Sequence
  Description of Artificial Sequencecapture
gtgaccctacagcgccg

SEQ ID NO: 504 - DNA, 21 bp, Artificial Sequence
  Description of Artificial Sequenceprimer
gattcctcgcagaggatttcg

SEQ ID NO: 505 - DNA, 21 bp, Artificial Sequence
  Description of Artificial Sequenceprimer
gattccccgcagaggatttcg

SEQ ID NO: 506 - DNA, 17 bp, Artificial Sequence
  Description of Artificial Sequenceprimer
gcgacgacgctcacctc

SEQ ID NO: 507 - DNA, 18 bp, Artificial Sequence
  Description of Artificial Sequencecapture
atttcgtgtaccagttta

SEQ ID NO: 508 - DNA, 18 bp, Artificial Sequence
  Description of Artificial Sequencecapture
atttcgtgttccagttta

SEQ ID NO: 509 - DNA, 18 bp, Artificial Sequence
  Description of Artificial Sequencecapture
atttcgtgctccagttta

SEQ ID NO: 510 - DNA, 17 bp, Artificial Sequence
  Description of Artificial Sequencecapture
tttaagggcctgtgcta

SEQ ID NO: 511 - DNA, 17 bp, Artificial Sequence
  Description of Artificial Sequencecapture
ttaagggcatgtgctac

SEQ ID NO: 512 - DNA, 17 bp, Artificial Sequence
  Description of Artificial Sequencecapture
tttaaggccatgtgcta

SEQ ID NO: 513 - DNA, 17 bp, Artificial Sequence
  Description of Artificial Sequencecapture
ttcaccaatgggacgga

SEQ ID NO: 514 - DNA, 17 bp, Artificial Sequence
  Description of Artificial Sequencecapture
aacgggacagagcgcgt

SEQ ID NO: 515 - DNA, 17 bp, Artificial Sequence
  Description of Artificial Sequencecapture
aacgggaccgagcgcgt

SEQ ID NO: 516 - DNA, 17 bp, Artificial Sequence
  Description of Artificial Sequencecapture
gaccgagctcgtgcggg

SEQ ID NO: 517 - DNA, 17 bp, Artificial Sequence
  Description of Artificial Sequencecapture
gcgtgcggggtgtgacc

SEQ ID NO: 518 - DNA, 17 bp, Artificial Sequence
  Description of Artificial Sequencecapture
gcgtgcgtcttgtgacc

SEQ ID NO: 519 - DNA, 17 bp, Artificial Sequence
  Description of Artificial Sequencecapture
gcgtgcgttatgtgacc

SEQ ID NO: 520 - DNA, 17 bp, Artificial Sequence
  Description of Artificial Sequencecapture
gcgtgcgtcttgtgacc

SEQ ID NO: 521 - DNA, 17 bp, Artificial Sequence
  Description of Artificial Sequencecapture
gcgtgcgtcttgtgagc

SEQ ID NO: 522 - DNA, 17 bp, Artificial Sequence
  Description of Artificial Sequencecapture
tcgtgcggggtgtgacc

SEQ ID NO: 523 - DNA, 17 bp, Artificial Sequence
  Description of Artificial Sequencecapture
gcgtgcgtcttgtaacc

SEQ ID NO: 524 - DNA, 17 bp, Artificial Sequence
  Description of Artificial Sequencecapture
cgtcttgtaaccagata

SEQ ID NO: 525 - DNA, 18 bp, Artificial Sequence
  Description of Artificial Sequencecapture
tcttgtgagcagaagcat

SEQ ID NO: 526 - DNA, 17 bp, Artificial Sequence
  Description of Artificial Sequencecapture
taaccagatacatctat

SEQ ID NO: 527 - DNA, 18 bp, Artificial Sequence
  Description of Artificial Sequencecapture
tgagcagaagcatctata

SEQ ID NO: 528 - DNA, 17 bp, Artificial Sequence
  Description of Artificial Sequencecapture
aaccgagaagagtacgt

SEQ ID NO: 529 - DNA, 18 bp, Artificial Sequence
  Description of Artificial Sequencecapture
gagaagagatcgtgcgct

SEQ ID NO: 530 - DNA, 16 bp, Artificial Sequence
  Description of Artificial Sequencecapture
gagaggagacgtgcgc

SEQ ID NO: 531 - DNA, 17 bp, Artificial Sequence
  Description of Artificial Sequencecapture
tgaccagatacatctat

SEQ ID NO: 532 - DNA, 17 bp, Artificial Sequence
  Description of Artificial Sequencecapture
aaccgagaagagatcgt

SEQ ID NO: 533 - DNA, 16 bp, Artificial Sequence
  Description of Artificial Sequencecapture
gagtacgtgcgcttcg

SEQ ID NO: 534 - DNA, 16 bp, Artificial Sequence
  Description of Artificial Sequencecapture
gagtacgcacgcttcg

SEQ ID NO: 535 - DNA, 17 bp, Artificial Sequence
  Description of Artificial Sequencecapture
ggagtacgcgcgcttcg

SEQ ID NO: 536 - DNA, 16 bp, Artificial Sequence
  Description of Artificial Sequencecapture
gagatcgtgcgcttcg

SEQ ID NO: 537 - DNA, 16 bp, Artificial Sequence
  Description of Artificial Sequencecapture
gaggacgtgcgcttcg

SEQ ID NO: 538 - DNA, 16 bp, Artificial Sequence
  Description of Artificial Sequencecapture
cgacgtggaggtgtac

SEQ ID NO: 539 - DNA, 17 bp, Artificial Sequence
  Description of Artificial Sequencecapture
cgtgggggagttccggg

SEQ ID NO: 540 - DNA, 16 bp, Artificial Sequence
  Description of Artificial Sequencecapture
ggggtgtaccgggcag

SEQ ID NO: 541 - DNA, 16 bp, Artificial Sequence
  Description of Artificial Sequencecapture
ggggtgtatcgggcgg

SEQ ID NO: 542 - DNA, 16 bp, Artificial Sequence
  Description of Artificial Sequencecapture
ggggagttccgggcgg

SEQ ID NO: 543 - DNA, 16 bp, Artificial Sequence
  Description of Artificial Sequencecapture
ggggtgtaccgggcgg

SEQ ID NO: 544 - DNA, 16 bp, Artificial Sequence
  Description of Artificial Sequencecapture
gaggtgtaccgggcgg

SEQ ID NO: 545 - DNA, 16 bp, Artificial Sequence
  Description of Artificial Sequencecapture
ggggtgtatcgggtgg

SEQ ID NO: 546 - DNA, 16 bp, Artificial Sequence
  Description of Artificial Sequencecapture
ggggtgtatcgggcgg

SEQ ID NO: 547 - DNA, 16 bp, Artificial Sequence
  Description of Artificial Sequencecapture
ggggtgtaccgcgcgg

SEQ ID NO: 548 - DNA, 17 bp, Artificial Sequence
  Description of Artificial Sequencecapture
gtgtaccgcgcggtgac

SEQ ID NO: 549 - DNA, 17 bp, Artificial Sequence
  Description of Artificial Sequencecapture
taccgggcagtgacgcc

SEQ ID NO: 550 - DNA, 16 bp, Artificial Sequence
  Description of Artificial Sequencecapture
tatcgggtggtgacgc

SEQ ID NO: 551 - DNA, 17 bp, Artificial Sequence
  Description of Artificial Sequencecapture
gcggtgaccccgcaggg

SEQ ID NO: 552 - DNA, 17 bp, Artificial Sequence
  Description of Artificial Sequencecapture
ggtgacgctgcaggggc

SEQ ID NO: 553 - DNA, 17 bp, Artificial Sequence
  Description of Artificial Sequencecapture
gacgctgctggggctgc

SEQ ID NO: 554 - DNA, 17 bp, Artificial Sequence
  Description of Artificial Sequencecapture
gctggggctgcctgccg

SEQ ID NO: 555 - DNA, 17 bp, Artificial Sequence
  Description of Artificial Sequencecapture
gctggggccgcctgacg

SEQ ID NO: 556 - DNA, 17 bp, Artificial Sequence
  Description of Artificial Sequencecapture
ggggcggcttgacgccg

SEQ ID NO: 557 - DNA, 17 bp, Artificial Sequence
  Description of Artificial Sequencecapture
gctggggccgcctgccg

SEQ ID NO: 558 - DNA, 17 bp, Artificial Sequence
  Description of Artificial Sequencecapture
gctggggctgcctgacg

SEQ ID NO: 559 - DNA, 17 bp, Artificial Sequence
  Description of Artificial Sequencecapture
gcggcctgttgccgagt

SEQ ID NO: 560 - DNA, 17 bp, Artificial Sequence
  Description of Artificial Sequencecapture
gcggcctagcgccgagt

SEQ ID NO: 561 - DNA, 17 bp, Artificial Sequence
  Description of Artificial Sequencecapture
gcggcctgacgccgagt

SEQ ID NO: 562 - DNA, 17 bp, Artificial Sequence
  Description of Artificial Sequencecapture
gctgcctgccgccgagt

SEQ ID NO: 563 - DNA, 17 bp, Artificial Sequence
  Description of Artificial Sequencecapture
gcggcctgatgccgagt

SEQ ID NO: 564 - DNA, 17 bp, Artificial Sequence
  Description of Artificial Sequencecapture
gcggcctgtcgccgagt

SEQ ID NO: 565 - DNA, 17 bp, Artificial Sequence
  Description of Artificial Sequencecapture
gctgcctgacgccgagt

SEQ ID NO: 566 - DNA, 17 bp, Artificial Sequence
  Description of Artificial Sequencecapture
gccgcctgccgccgagt

SEQ ID NO: 567 - DNA, 17 bp, Artificial Sequence
  Description of Artificial Sequencecapture
gcggcctgatgccgaga

SEQ ID NO: 568 - DNA, 17 bp, Artificial Sequence
  Description of Artificial Sequencecapture
gccgcctgacgccgagt

SEQ ID NO: 569 - DNA, 17 bp, Artificial Sequence
  Description of Artificial Sequencecapture
gcggcttgacgccgagt

SEQ ID NO: 570 - DNA, 17 bp, Artificial Sequence
  Description of Artificial Sequencecapture
atgccgagaactggaac

SEQ ID NO: 571 - DNA, 17 bp, Artificial Sequence
  Description of Artificial Sequencecapture
tactggaatagccagaa

SEQ ID NO: 572 - DNA, 16 bp, Artificial Sequence
  Description of Artificial Sequencecapture
agaaggaagtcctgga

SEQ ID NO: 573 - DNA, 16 bp, Artificial Sequence
  Description of Artificial Sequencecapture
agaaggacatcctgga

SEQ ID NO: 574 - DNA, 18 bp, Artificial Sequence
  Description of Artificial Sequencecapture
ctggagggggcccgggcg

SEQ ID NO: 575 - DNA, 18 bp, Artificial Sequence
  Description of Artificial Sequencecapture
ctggaggaggaccgggcg

SEQ ID NO: 576 - DNA, 18 bp, Artificial Sequence
  Description of Artificial Sequencecapture
ctggagaggaaacgggcg

SEQ ID NO: 577 - DNA, 18 bp, Artificial Sequence
  Description of Artificial Sequencecapture
ctggagaggacccgggcg

SEQ ID NO: 578 - DNA, 18 bp, Artificial Sequence
  Description of Artificial Sequencecapture
ctggaggggacccgggcg

SEQ ID NO: 579 - DNA, 18 bp, Artificial Sequence
  Description of Artificial Sequencecapture
ctggagaggacccgagcg

SEQ ID NO: 580 - DNA, 17 bp, Artificial Sequence
  Description of Artificial Sequencecapture
aggacccgagcggagtt

SEQ ID NO: 581 - DNA, 17 bp, Artificial Sequence
  Description of Artificial Sequencecapture
aacgggcggcggtggac

SEQ ID NO: 582 - DNA, 17 bp, Artificial Sequence
  Description of Artificial Sequencecapture
cgggcggagttggacac

SEQ ID NO: 583 - DNA, 17 bp, Artificial Sequence
  Description of Artificial Sequencecapture
gttggacacggtgtgca

SEQ ID NO: 584 - DNA, 17 bp, Artificial Sequence
  Description of Artificial Sequencecapture
gtggacagagtgtgcag

SEQ ID NO: 585 - DNA, 17 bp, Artificial Sequence
  Description of Artificial Sequencecapture
gacaccgtatgcagaca

SEQ ID NO: 586 - DNA, 18 bp, Artificial Sequence
  Description of Artificial Sequencecapture
gaggtggcgtaccgcggg

SEQ ID NO: 587 - DNA, 18 bp, Artificial Sequence
  Description of Artificial Sequencecapture
cagttggagctccgcacg

SEQ ID NO: 588 - DNA, 17 bp, Artificial Sequence
  Description of Artificial Sequencecapture
ggtggcgttccgcggga

SEQ ID NO: 589 - DNA, 17 bp, Artificial Sequence
  Description of Artificial Sequencecapture
ccaggtggggtaccgcg

SEQ ID NO: 590 - DNA, 19 bp, Artificial Sequence
  Description of Artificial Sequenceprimer
tgtccccacagcacgtttc

SEQ ID NO: 591 - DNA, 18 bp, Artificial Sequence
  Description of Artificial Sequenceprimer
gtccccccagcacgtttc

SEQ ID NO: 592 - DNA, 18 bp, Artificial Sequence
  Description of Artificial Sequenceprimer
gcccccacagcacgtttc

SEQ ID NO: 593 - DNA, 18 bp, Artificial Sequence
  Description of Artificial Sequenceprimer
cgccgctgcactgtgaag

SEQ ID NO: 594 - DNA, 18 bp, Artificial Sequence
  Description of Artificial Sequencecapture
tggagctgcttaagtctg

SEQ ID NO: 595 - DNA, 17 bp, Artificial Sequence
  Description of Artificial Sequencecapture
cagggtaaatataagtg

SEQ ID NO: 596 - DNA, 16 bp, Artificial Sequence
  Description of Artificial Sequencecapture
gcaggttaaacatgag

SEQ ID NO: 597 - DNA, 18 bp, Artificial Sequence
  Description of Artificial Sequencecapture
actctacgggtgagtgtt

SEQ ID NO: 598 - DNA, 18 bp, Artificial Sequence
  Description of Artificial Sequencecapture
actctaggggtgagtgtt

SEQ ID NO: 599 - DNA, 18 bp, Artificial Sequence
  Description of Artificial Sequencecapture
actctatgggtgagtgtt

SEQ ID NO: 600 - DNA, 17 bp, Artificial Sequence
  Description of Artificial Sequencecapture
ttgaatgtcatttcttc

SEQ ID NO: 601 - DNA, 17 bp, Artificial Sequence
  Description of Artificial Sequencecapture
gtgagtgttatttcttc

SEQ ID NO: 602 - DNA, 17 bp, Artificial Sequence
  Description of Artificial Sequencecapture
gagtgtcaattcttcaa

SEQ ID NO: 603 - DNA, 17 bp, Artificial Sequence
  Description of Artificial Sequencecapture
ctgagtgtcatttcttc

SEQ ID NO: 604 - DNA, 17 bp, Artificial Sequence
  Description of Artificial Sequencecapture
atgagtgtcatttcttc

SEQ ID NO: 605 - DNA, 17 bp, Artificial Sequence
  Description of Artificial Sequencecapture
ataagtgtcatttcttc

SEQ ID NO: 606 - DNA, 17 bp, Artificial Sequence
  Description of Artificial Sequencecapture
ttgagtgtcatttcttc

SEQ ID NO: 607 - DNA, 17 bp, Artificial Sequence
  Description of Artificial Sequencecapture
gtgagtgttatttcttc

SEQ ID NO: 608 - DNA, 17 bp, Artificial Sequence
  Description of Artificial Sequencecapture
gcgggtgcagttcctgg

SEQ ID NO: 609 - DNA, 19 bp, Artificial Sequence
  Description of Artificial Sequencecapture
gggtgcggttgctggaaag

SEQ ID NO: 610 - DNA, 17 bp, Artificial Sequence
  Description of Artificial Sequencecapture
gtgcggttcctggagag

SEQ ID NO: 611 - DNA, 17 bp, Artificial Sequence
  Description of Artificial Sequencecapture
gtgcggtacctggacag

SEQ ID NO: 612 - DNA, 17 bp, Artificial Sequence
  Description of Artificial Sequencecapture
gtgcggtatctgcacag

SEQ ID NO: 613 - DNA, 17 bp, Artificial Sequence
  Description of Artificial Sequencecapture
gtgcggttactggagag

SEQ ID NO: 614 - DNA, 17 bp, Artificial Sequence
  Description of Artificial Sequencecapture
gtgcggttcctggacag

SEQ ID NO: 615 - DNA, 17 bp, Artificial Sequence
  Description of Artificial Sequencecapture
gtgcagttcctggaaag

SEQ ID NO: 616 - DNA, 17 bp, Artificial Sequence
  Description of Artificial Sequencecapture
gtgcagttcctggacag

SEQ ID NO: 617 - DNA, 17 bp, Artificial Sequence
  Description of Artificial Sequencecapture
gtgcggttcccggacag

SEQ ID NO: 618 - DNA, 17 bp, Artificial Sequence
  Description of Artificial Sequencecapture
gcggttcccggacagat

SEQ ID NO: 619 - DNA, 17 bp, Artificial Sequence
  Description of Artificial Sequencecapture
gttgctggaaagatgca

SEQ ID NO: 620 - DNA, 17 bp, Artificial Sequence
  Description of Artificial Sequencecapture
gtacctggacagatact

SEQ ID NO: 621 - DNA, 17 bp, Artificial Sequence
  Description of Artificial Sequencecapture
gttcctggagagatact

SEQ ID NO: 622 - DNA, 17 bp, Artificial Sequence
  Description of Artificial Sequencecapture
gtatctgcacagaggca

SEQ ID NO: 623 - DNA, 17 bp, Artificial Sequence
  Description of Artificial Sequencecapture
gttcctggaaagactct

SEQ ID NO: 624 - DNA, 17 bp, Artificial Sequence
  Description of Artificial Sequencecapture
gttcctggacagatact

SEQ ID NO: 625 - DNA, 17 bp, Artificial Sequence
  Description of Artificial Sequencecapture
gttactggagagacact

SEQ ID NO: 626 - DNA, 17 bp, Artificial Sequence
  Description of Artificial Sequencecapture
gttcctggaaagtctct

SEQ ID NO: 627 - DNA, 17 bp, Artificial Sequence
  Description of Artificial Sequencecapture
gtacctggacagatact

SEQ ID NO: 628 - DNA, 17 bp, Artificial Sequence
  Description of Artificial Sequencecapture
gttgctggaaagacgcg

SEQ ID NO: 629 - DNA, 17 bp, Artificial Sequence
  Description of Artificial Sequencecapture
gttcctggacagacact

SEQ ID NO: 630 - DNA, 17 bp, Artificial Sequence
  Description of Artificial Sequencecapture
gttcccggacagatact

SEQ ID NO: 631 - DNA, 17 bp, Artificial Sequence
  Description of Artificial Sequencecapture
ctggaaagtctcttcta

SEQ ID NO: 632 - DNA, 17 bp, Artificial Sequence
  Description of Artificial Sequencecapture
ggacagacacttctata

SEQ ID NO: 633 - DNA, 16 bp, Artificial Sequence
  Description of Artificial Sequencecapture
gatgcatctataacca

SEQ ID NO: 634 - DNA, 16 bp, Artificial Sequence
  Description of Artificial Sequencecapture
ataaccaagaggagtc

SEQ ID NO: 635 - DNA, 16 bp, Artificial Sequence
  Description of Artificial Sequencecapture
gatacttccataacca

SEQ ID NO: 636 - DNA, 16 bp, Artificial Sequence
  Description of Artificial Sequencecapture
ataaccaggaggagaa

SEQ ID NO: 637 - DNA, 16 bp, Artificial Sequence
  Description of Artificial Sequencecapture
gactcttctataacca

SEQ ID NO: 638 - DNA, 16 bp, Artificial Sequence
  Description of Artificial Sequencecapture
ataaccaggaggagtt

SEQ ID NO: 639 - DNA, 17 bp, Artificial Sequence
  Description of Artificial Sequencecapture
acttctatcaccaagag

SEQ ID NO: 640 - DNA, 16 bp, Artificial Sequence
  Description of Artificial Sequencecapture
ataaccgggaggagaa

SEQ ID NO: 641 - DNA, 16 bp, Artificial Sequence
  Description of Artificial Sequencecapture
gacgcgtccataacca

SEQ ID NO: 642 - DNA, 16 bp, Artificial Sequence
  Description of Artificial Sequencecapture
ataaccaagaggagta

SEQ ID NO: 643 - DNA, 16 bp, Artificial Sequence
  Description of Artificial Sequencecapture
gaggcatctataacca

SEQ ID NO: 644 - DNA, 16 bp, Artificial Sequence
  Description of Artificial Sequencecapture
ataaccaagaggagta

SEQ ID NO: 645 - DNA, 16 bp, Artificial Sequence
  Description of Artificial Sequencecapture
gacacttccataacca

SEQ ID NO: 646 - DNA, 16 bp, Artificial Sequence
  Description of Artificial Sequencecapture
ataaccaggaggagtc

SEQ ID NO: 647 - DNA, 16 bp, Artificial Sequence
  Description of Artificial Sequencecapture
gatacttctataacca

SEQ ID NO: 648 - DNA, 16 bp, Artificial Sequence
  Description of Artificial Sequencecapture
ataaccaggaggagta

SEQ ID NO: 649 - DNA, 17 bp, Artificial Sequence
  Description of Artificial Sequencecapture
acttctatcaccaagaa

SEQ ID NO: 650 - DNA, 16 bp, Artificial Sequence
  Description of Artificial Sequencecapture
gatacttctataacca

SEQ ID NO: 651 - DNA, 16 bp, Artificial Sequence
  Description of Artificial Sequencecapture
ataaccaagaggagga

SEQ ID NO: 652 - DNA, 16 bp, Artificial Sequence
  Description of Artificial Sequencecapture
ataaccaggaggagtt

SEQ ID NO: 653 - DNA, 16 bp, Artificial Sequence
  Description of Artificial Sequencecapture
gatacttctataacca

SEQ ID NO: 654 - DNA, 16 bp, Artificial Sequence
  Description of Artificial Sequencecapture
ataaccaggaggagtc

SEQ ID NO: 655 - DNA, 16 bp, Artificial Sequence
  Description of Artificial Sequencecapture
ataaccaagaggagtt

SEQ ID NO: 656 - DNA, 16 bp, Artificial Sequence
  Description of Artificial Sequencecapture
ataaccaggaggagct

SEQ ID NO: 657 - DNA, 16 bp, Artificial Sequence
  Description of Artificial Sequencecapture
gacacttctataacca

SEQ ID NO: 658 - DNA, 16 bp, Artificial Sequence
  Description of Artificial Sequencecapture
ataaccaggaggagtc

SEQ ID NO: 659 - DNA, 16 bp, Artificial Sequence
  Description of Artificial Sequencecapture
ataaccaagaggagaa

SEQ ID NO: 660 - DNA, 16 bp, Artificial Sequence
  Description of Artificial Sequencecapture
ataaccaggaggagtt

SEQ ID NO: 661 - DNA, 16 bp, Artificial Sequence
  Description of Artificial Sequencecapture
gatacttctataacca

SEQ ID NO: 662 - DNA, 16 bp, Artificial Sequence
  Description of Artificial Sequencecapture
ataaccaggaggagaa

SEQ ID NO: 663 - DNA, 16 bp, Artificial Sequence
  Description of Artificial Sequencecapture
ataaccaagaggagtc

SEQ ID NO: 664 - DNA, 17 bp, Artificial Sequence
  Description of Artificial Sequencecapture
aggagaacctgcgcttc

SEQ ID NO: 665 - DNA, 17 bp, Artificial Sequence
  Description of Artificial Sequencecapture
caccaagaagagtacgt

SEQ ID NO: 666 - DNA, 17 bp, Artificial Sequence
  Description of Artificial Sequencecapture
aggaggacttgcgcttc

SEQ ID NO: 667 - DNA, 16 bp, Artificial Sequence
  Description of Artificial Sequencecapture
agaggagtccgtgcgc

SEQ ID NO: 668 - DNA, 16 bp, Artificial Sequence
  Description of Artificial Sequencecapture
agaggagaacgtgcgc

SEQ ID NO: 669 - DNA, 16 bp, Artificial Sequence
  Description of Artificial Sequencecapture
agaggagtacgtgcgc

SEQ ID NO: 670 - DNA, 16 bp, Artificial Sequence
  Description of Artificial Sequencecapture
agaggagttcgtgcgc

SEQ ID NO: 671 - DNA, 16 bp, Artificial Sequence
  Description of Artificial Sequencecapture
agaggaggacgtgcgc

SEQ ID NO: 672 - DNA, 16 bp, Artificial Sequence
  Description of Artificial Sequencecapture
ggaggagctcctgcgc

SEQ ID NO: 673 - DNA, 16 bp, Artificial Sequence
  Description of Artificial Sequencecapture
ggaggagtccgtgcgc

SEQ ID NO: 674 - DNA, 16 bp, Artificial Sequence
  Description of Artificial Sequencecapture
ggaggagaacgtgcgc

SEQ ID NO: 675 - DNA, 16 bp, Artificial Sequence
  Description of Artificial Sequencecapture
agaagagtacgtgcgc

SEQ ID NO: 676 - DNA, 16 bp, Artificial Sequence
  Description of Artificial Sequencecapture
ggaggagttcgtgcgc

SEQ ID NO: 677 - DNA, 16 bp, Artificial Sequence
  Description of Artificial Sequencecapture
agaggaggacttgcgc

SEQ ID NO: 678 - DNA, 16 bp, Artificial Sequence
  Description of Artificial Sequencecapture
ggaggagaacctgcgc

SEQ ID NO: 679 - DNA, 16 bp, Artificial Sequence
  Description of Artificial Sequencecapture
agaggagtacgtgcgg

SEQ ID NO: 680 - DNA, 16 bp, Artificial Sequence
  Description of Artificial Sequencecapture
ggaggagttcctgcgc

SEQ ID NO: 681 - DNA, 16 bp, Artificial Sequence
  Description of Artificial Sequencecapture
agaggagtacgtgcac

SEQ ID NO: 682 - DNA, 16 bp, Artificial Sequence
  Description of Artificial Sequencecapture
agaggagtacgcgcgc

SEQ ID NO: 683 - DNA, 16 bp, Artificial Sequence
  Description of Artificial Sequencecapture
ggaggagtacgtgcgc

SEQ ID NO: 684 - DNA, 17 bp, Artificial Sequence
  Description of Artificial Sequencecapture
gtccgtgcacttcgaca

SEQ ID NO: 685 - DNA, 17 bp, Artificial Sequence
  Description of Artificial Sequencecapture
tccgtgcggttcgacag

SEQ ID NO: 686 - DNA, 17 bp, Artificial Sequence
  Description of Artificial Sequencecapture
gcgacgtgcgggagtac

SEQ ID NO: 687 - DNA, 17 bp, Artificial Sequence
  Description of Artificial Sequencecapture
ggagttcctggcggtga

SEQ ID NO: 688 - DNA, 17 bp, Artificial Sequence
  Description of Artificial Sequencecapture
gcgacgtgagggagtac

SEQ ID NO: 689 - DNA, 17 bp, Artificial Sequence
  Description of Artificial Sequencecapture
gggggagtaccgggcgg

SEQ ID NO: 690 - DNA, 17 bp, Artificial Sequence
  Description of Artificial Sequencecapture
gggggagttccgggcgg

SEQ ID NO: 691 - DNA, 17 bp, Artificial Sequence
  Description of Artificial Sequencecapture
gagggagtaccgggcgg

SEQ ID NO: 692 - DNA, 17 bp, Artificial Sequence
  Description of Artificial Sequencecapture
ggggagttctgggcggt

SEQ ID NO: 693 - DNA, 17 bp, Artificial Sequence
  Description of Artificial Sequencecapture
gggggagtaccgggtgg

SEQ ID NO: 694 - DNA, 17 bp, Artificial Sequence
  Description of Artificial Sequencecapture
gggggagttccaggcgg

SEQ ID NO: 695 - DNA, 17 bp, Artificial Sequence
  Description of Artificial Sequencecapture
gcgggagtaccgggcgg

SEQ ID NO: 696 - DNA, 17 bp, Artificial Sequence
  Description of Artificial Sequencecapture
ggagttccaggcggtga

SEQ ID NO: 697 - DNA, 17 bp, Artificial Sequence
  Description of Artificial Sequencecapture
gtaccgggtggtgacgg

SEQ ID NO: 698 - DNA, 17 bp, Artificial Sequence
  Description of Artificial Sequencecapture
ccgggcggcgacggagc

SEQ ID NO: 699 - DNA, 17 bp, Artificial Sequence
  Description of Artificial Sequencecapture
ggcggtgagggagctgg

SEQ ID NO: 700 - DNA, 17 bp, Artificial Sequence
  Description of Artificial Sequencecapture
ggcggtgatggagctgg

SEQ ID NO: 701 - DNA, 20 bp, Artificial Sequence
  Description of Artificial Sequencecapture
gcctgatgccgagtactgga

SEQ ID NO: 702 - DNA, 17 bp, Artificial Sequence
  Description of Artificial Sequencecapture
cggcctagcgccgagta

SEQ ID NO: 703 - DNA, 20 bp, Artificial Sequence
  Description of Artificial Sequencecapture
gcctgacgctgagtactgga

SEQ ID NO: 704 - DNA, 16 bp, Artificial Sequence
  Description of Artificial Sequencecapture
cggcctgtcgccgagt

SEQ ID NO: 705 - DNA, 17 bp, Artificial Sequence
  Description of Artificial Sequencecapture
gccgagtcctggaacag

SEQ ID NO: 706 - DNA, 20 bp, Artificial Sequence
  Description of Artificial Sequencecapture
gcctgacgcggagcactgga

SEQ ID NO: 707 - DNA, 17 bp, Artificial Sequence
  Description of Artificial Sequencecapture
cctgatgctgagtactg

SEQ ID NO: 708 - DNA, 17 bp, Artificial Sequence
  Description of Artificial Sequencecapture
gcggcctgttgccgagt

SEQ ID NO: 709 - DNA, 17 bp, Artificial Sequence
  Description of Artificial Sequencecapture
tgccgagtctgccgagt

SEQ ID NO: 710 - DNA, 17 bp, Artificial Sequence
  Description of Artificial Sequencecapture
gcggcctatcgccgagt

SEQ ID NO: 711 - DNA, 17 bp, Artificial Sequence
  Description of Artificial Sequencecapture
ggcctgataccgagtac

SEQ ID NO: 712 - DNA, 19 bp, Artificial Sequence
  Description of Artificial Sequencecapture
tgatgcggagcactggaac

SEQ ID NO: 713 - DNA, 20 bp, Artificial Sequence
  Description of Artificial Sequencecapture
gcctgatgaggagtactgga

SEQ ID NO: 714 - DNA, 17 bp, Artificial Sequence
  Description of Artificial Sequencecapture
ctgatgcccagtactgg

SEQ ID NO: 715 - DNA, 17 bp, Artificial Sequence
  Description of Artificial Sequencecapture
tgatgaggggtactgga

SEQ ID NO: 716 - DNA, 17 bp, Artificial Sequence
  Description of Artificial Sequencecapture
gatgaggactactggaa

SEQ ID NO: 717 - DNA, 17 bp, Artificial Sequence
  Description of Artificial Sequencecapture
tgatgcggagcactgga

SEQ ID NO: 718 - DNA, 17 bp, Artificial Sequence
  Description of Artificial Sequencecapture
atgaggagcactggaac

SEQ ID NO: 719 - DNA, 17 bp, Artificial Sequence
  Description of Artificial Sequencecapture
cctgtcgccgagtcctg

SEQ ID NO: 720 - DNA, 17 bp, Artificial Sequence
  Description of Artificial Sequencecapture
tgatgcggagcactgga

SEQ ID NO: 721 - DNA, 17 bp, Artificial Sequence
  Description of Artificial Sequencecapture
gcctgatgaggagtact

SEQ ID NO: 722 - DNA, 15 bp, Artificial Sequence
  Description of Artificial Sequencecapture
cctgatgagggtact

SEQ ID NO: 723 - DNA, 16 bp, Artificial Sequence
  Description of Artificial Sequencecapture
cctgatgaggactact

SEQ ID NO: 724 - DNA, 16 bp, Artificial Sequence
  Description of Artificial Sequencecapture
cctgatgaggagcact

SEQ ID NO: 725 - DNA, 17 bp, Artificial Sequence
  Description of Artificial Sequencecapture
gccagaagaacatcctg

SEQ ID NO: 726 - DNA, 17 bp, Artificial Sequence
  Description of Artificial Sequencecapture
agaaggacctcctggag

SEQ ID NO: 727 - DNA, 17 bp, Artificial Sequence
  Description of Artificial Sequencecapture
agaaggacatcctggag

SEQ ID NO: 728 - DNA, 17 bp, Artificial Sequence
  Description of Artificial Sequencecapture
agaaggacttcctggag

SEQ ID NO: 729 - DNA, 17 bp, Artificial Sequence
  Description of Artificial Sequencecapture
agaaggacctcctggaa

SEQ ID NO: 730 - DNA, 17 bp, Artificial Sequence
  Description of Artificial Sequencecapture
agaaggacatcctggaa

SEQ ID NO: 731 - DNA, 17 bp, Artificial Sequence
  Description of Artificial Sequencecapture
agaaggacttcctggaa

SEQ ID NO: 732 - DNA, 16 bp, Artificial Sequence
  Description of Artificial Sequencecapture
agaagaacatcctgga

SEQ ID NO: 733 - DNA, 19 bp, Artificial Sequence
  Description of Artificial Sequencecapture
cctggagcagaggcgggcc

SEQ ID NO: 734 - DNA, 19 bp, Artificial Sequence
  Description of Artificial Sequencecapture
cctggaagacgagcgggcc

SEQ ID NO: 735 - DNA, 18 bp, Artificial Sequence
  Description of Artificial Sequencecapture
cctggaagacaagcgggc

SEQ ID NO: 736 - DNA, 17 bp, Artificial Sequence
  Description of Artificial Sequencecapture
cctggaagacaggcggg

SEQ ID NO: 737 - DNA, 16 bp, Artificial Sequence
  Description of Artificial Sequencecapture
cctggagcgaggcggg

SEQ ID NO: 738 - DNA, 16 bp, Artificial Sequence
  Description of Artificial Sequencecapture
ggagcagaagcgggcc

SEQ ID NO: 739 - DNA, 17 bp, Artificial Sequence
  Description of Artificial Sequencecapture
tggagcaggcgcgggcc

SEQ ID NO: 740 - DNA, 17 bp, Artificial Sequence
  Description of Artificial Sequencecapture
cctggaggacaggcggg

SEQ ID NO: 741 - DNA, 19 bp, Artificial Sequence
  Description of Artificial Sequencecapture
cctggagcagaggcaggcc

SEQ ID NO: 742 - DNA, 18 bp, Artificial Sequence
  Description of Artificial Sequencecapture
cctggaagacgagcgcgc

SEQ ID NO: 743 - DNA, 16 bp, Artificial Sequence
  Description of Artificial Sequencecapture
cctggaagacagggcc

SEQ ID NO: 744 - DNA, 16 bp, Artificial Sequence
  Description of Artificial Sequencecapture
cctggaagacaggcgc

SEQ ID NO: 745 - DNA, 19 bp, Artificial Sequence
  Description of Artificial Sequencecapture
cctggagcggaggcgtgcc

SEQ ID NO: 746 - DNA, 17 bp, Artificial Sequence
  Description of Artificial Sequencecapture
ggagcagaagcggggcc

SEQ ID NO: 747 - DNA, 19 bp, Artificial Sequence
  Description of Artificial Sequencecapture
cctggagcggaggcgcgcc

SEQ ID NO: 748 - DNA, 17 bp, Artificial Sequence
  Description of Artificial Sequencecapture
cagaggcgggccgcggt

SEQ ID NO: 749 - DNA, 17 bp, Artificial Sequence
  Description of Artificial Sequencecapture
cggaggcgcgccgcggt

SEQ ID NO: 750 - DNA, 17 bp, Artificial Sequence
  Description of Artificial Sequencecapture
gacagggccgccgcggt

SEQ ID NO: 751 - DNA, 17 bp, Artificial Sequence
  Description of Artificial Sequencecapture
gcagaggcaggccgcgg

SEQ ID NO: 752 - DNA, 17 bp, Artificial Sequence
  Description of Artificial Sequencecapture
cagaggcgtgccgcggt

SEQ ID NO: 753 - DNA, 17 bp, Artificial Sequence
  Description of Artificial Sequencecapture
cagaggcgcgccgcggt

SEQ ID NO: 754 - DNA, 17 bp, Artificial Sequence
  Description of Artificial Sequencecapture
cagaggcgggccgccgt

SEQ ID NO: 755 - DNA, 17 bp, Artificial Sequence
  Description of Artificial Sequencecapture
gacaggcgcgccgcggt

SEQ ID NO: 756 - DNA, 17 bp, Artificial Sequence
  Description of Artificial Sequencecapture
gacgagcgggccgcggt

SEQ ID NO: 757 - DNA, 17 bp, Artificial Sequence
  Description of Artificial Sequencecapture
gacaggcgcgccgcggt

SEQ ID NO: 758 - DNA, 17 bp, Artificial Sequence
  Description of Artificial Sequencecapture
caggcgcgggccgcggt

SEQ ID NO: 759 - DNA, 17 bp, Artificial Sequence
  Description of Artificial Sequencecapture
cagaagcggggccgggt

SEQ ID NO: 760 - DNA, 17 bp, Artificial Sequence
  Description of Artificial Sequencecapture
cagaagcgggccgcggt

SEQ ID NO: 761 - DNA, 17 bp, Artificial Sequence
  Description of Artificial Sequencecapture
cagaggcgggccgaggt

SEQ ID NO: 762 - DNA, 17 bp, Artificial Sequence
  Description of Artificial Sequencecapture
gacaggcgggccctggt

SEQ ID NO: 763 - DNA, 17 bp, Artificial Sequence
  Description of Artificial Sequencecapture
gacaggcgggccgcggt

SEQ ID NO: 764 - DNA, 17 bp, Artificial Sequence
  Description of Artificial Sequencecapture
cggaggcgggccgcggt

SEQ ID NO: 765 - DNA, 17 bp, Artificial Sequence
  Description of Artificial Sequencecapture
cagaggcgggccctggt

SEQ ID NO: 766 - DNA, 17 bp, Artificial Sequence
  Description of Artificial Sequencecapture
gacaggcggggccaggt

SEQ ID NO: 767 - DNA, 17 bp, Artificial Sequence
  Description of Artificial Sequencecapture
cggaggcgggccgaggt

SEQ ID NO: 768 - DNA, 17 bp, Artificial Sequence
  Description of Artificial Sequencecapture
gacaggcgggccgtggt

SEQ ID NO: 769 - DNA, 17 bp, Artificial Sequence
  Description of Artificial Sequencecapture
gacgagcgcgccgcggt

SEQ ID NO: 770 - DNA, 17 bp, Artificial Sequence
  Description of Artificial Sequencecapture
gacaagcgggccgcggt

SEQ ID NO: 771 - DNA, 15 bp, Artificial Sequence
  Description of Artificial Sequencecapture
cgggccgcggtggac

SEQ ID NO: 772 - DNA, 14 bp, Artificial Sequence
  Description of Artificial Sequencecapture
cgggccctggtgga

SEQ ID NO: 773 - DNA, 14 bp, Artificial Sequence
  Description of Artificial Sequencecapture
cgggccgaggtgga

SEQ ID NO: 774 - DNA, 15 bp, Artificial Sequence
  Description of Artificial Sequencecapture
cggggccgggtggac

SEQ ID NO: 775 - DNA, 15 bp, Artificial Sequence
  Description of Artificial Sequencecapture
cggggccaggtggac

SEQ ID NO: 776 - DNA, 15 bp, Artificial Sequence
  Description of Artificial Sequencecapture
cgggccgtggtggac

SEQ ID NO: 777 - DNA, 17 bp, Artificial Sequence
  Description of Artificial Sequencecapture
cgggccgccgtggacac

SEQ ID NO: 778 - DNA, 15 bp, Artificial Sequence
  Description of Artificial Sequencecapture
caggccgcggtggac

SEQ ID NO: 779 - DNA, 15 bp, Artificial Sequence
  Description of Artificial Sequencecapture
cgtgccgcggtggac

SEQ ID NO: 780 - DNA, 15 bp, Artificial Sequence
  Description of Artificial Sequencecapture
cgcgccgcggtggac

SEQ ID NO: 781 - DNA, 15 bp, Artificial Sequence
  Description of Artificial Sequencecapture
gccgccgcggtggac

SEQ ID NO: 782 - DNA, 18 bp, Artificial Sequence
  Description of Artificial Sequencecapture
ggtggacaactactgcag

SEQ ID NO: 783 - DNA, 19 bp, Artificial Sequence
  Description of Artificial Sequencecapture
ggtggacaattactgcaga

SEQ ID NO: 784 - DNA, 17 bp, Artificial Sequence
  Description of Artificial Sequencecapture
gacacctactgcagaca

SEQ ID NO: 785 - DNA, 17 bp, Artificial Sequence
  Description of Artificial Sequencecapture
gacacctattgcagaca

SEQ ID NO: 786 - DNA, 18 bp, Artificial Sequence
  Description of Artificial Sequencecapture
ggacaccgtgtgcagaca

SEQ ID NO: 787 - DNA, 17 bp, Artificial Sequence
  Description of Artificial Sequencecapture
gacaattactgcagaca

SEQ ID NO: 788 - DNA, 17 bp, Artificial Sequence
  Description of Artificial Sequencecapture
gacaactactgcagaca

SEQ ID NO: 789 - DNA, 17 bp, Artificial Sequence
  Description of Artificial Sequencecapture
acaactaccgggttgtg

SEQ ID NO: 790 - DNA, 17 bp, Artificial Sequence
  Description of Artificial Sequencecapture
aactacggagttgtgga

SEQ ID NO: 791 - DNA, 17 bp, Artificial Sequence
  Description of Artificial Sequencecapture
ctacggggctgtggaga

SEQ ID NO: 792 - DNA, 16 bp, Artificial Sequence
  Description of Artificial Sequencecapture
ggggttggtgagagct

SEQ ID NO: 793 - DNA, 16 bp, Artificial Sequence
  Description of Artificial Sequencecapture
ggggttgtggagagct

SEQ ID NO: 794 - DNA, 16 bp, Artificial Sequence
  Description of Artificial Sequencecapture
ggggttgatgagagct

SEQ ID NO: 795 - DNA, 16 bp, Artificial Sequence
  Description of Artificial Sequencecapture
ggggctgtggagagct

SEQ ID NO: 796 - DNA, 16 bp, Artificial Sequence
  Description of Artificial Sequencecapture
ggggttgtggagagat

SEQ ID NO: 797 - DNA, 16 bp, Artificial Sequence
  Description of Artificial Sequencecapture
cgggttgtggagagct

SEQ ID NO: 798 - DNA, 16 bp, Artificial Sequence
  Description of Artificial Sequencecapture
ggagttgtggagagct

SEQ ID NO: 799 - DNA, 17 bp, Artificial Sequence
  Description of Artificial Sequencecapture
gtggagagattcacagt

SEQ ID NO: 800 - DNA, 18 bp, Artificial Sequence
  Description of Artificial Sequencecapture
ggtgcggctcctggagag

SEQ ID NO: 801 - DNA, 18 bp, Artificial Sequence
  Description of Artificial Sequencecapture
agtgtggaacctgatcag

SEQ ID NO: 802 - DNA, 17 bp, Artificial Sequence
  Description of Artificial Sequencecapture
gagcctgatcagataca

SEQ ID NO: 803 - DNA, 18 bp, Artificial Sequence
  Description of Artificial Sequencecapture
ggtgcggttcctgcacag

SEQ ID NO: 804 - DNA, 18 bp, Artificial Sequence
  Description of Artificial Sequencecapture
ggtgcggtacctggagag

SEQ ID NO: 805 - DNA, 18 bp, Artificial Sequence
  Description of Artificial Sequencecapture
ggtgcggtacctgaacag

SEQ ID NO: 806 - DNA, 18 bp, Artificial Sequence
  Description of Artificial Sequencecapture
gtacctgaacagatactt

SEQ ID NO: 807 - DNA, 18 bp, Artificial Sequence
  Description of Artificial Sequencecapture
gaacctgatcagatacat

SEQ ID NO: 808 - DNA, 19 bp, Artificial Sequence
  Description of Artificial Sequencecapture
ggagcctgatcagatacat

SEQ ID NO: 809 - DNA, 19 bp, Artificial Sequence
  Description of Artificial Sequencecapture
ggttcctgcacagagacat

SEQ ID NO: 810 - DNA, 18 bp, Artificial Sequence
  Description of Artificial Sequencecapture
gtacctggagagatactt

SEQ ID NO: 811 - DNA, 19 bp, Artificial Sequence
  Description of Artificial Sequencecapture
ggttcctgcacagaggcat

SEQ ID NO: 812 - DNA, 18 bp, Artificial Sequence
  Description of Artificial Sequencecapture
gttcctggagagacactt

SEQ ID NO: 813 - DNA, 18 bp, Artificial Sequence
  Description of Artificial Sequencecapture
gctcctggagagacactt

SEQ ID NO: 814 - DNA, 17 bp, Artificial Sequence
  Description of Artificial Sequencecapture
ggcacgatgcatctata

SEQ ID NO: 815 - DNA, 17 bp, Artificial Sequence
  Description of Artificial Sequencecapture
gatcagatacatctata

SEQ ID NO: 816 - DNA, 18 bp, Artificial Sequence
  Description of Artificial Sequencecapture
ggcacgatgaatctataa

SEQ ID NO: 817 - DNA, 19 bp, Artificial Sequence
  Description of Artificial Sequencecapture
ggagagacacttccataac

SEQ ID NO: 818 - DNA, 19 bp, Artificial Sequence
  Description of Artificial Sequencecapture
ggacagatacttccataac

SEQ ID NO: 819 - DNA, 19 bp, Artificial Sequence
  Description of Artificial Sequencecapture
ggagagatacttccataac

SEQ ID NO: 820 - DNA, 19 bp, Artificial Sequence
  Description of Artificial Sequencecapture
gaacagatacttccataac

SEQ ID NO: 821 - DNA, 16 bp, Artificial Sequence
  Description of Artificial Sequencecapture
gatacatctataacca

SEQ ID NO: 822 - DNA, 16 bp, Artificial Sequence
  Description of Artificial Sequencecapture
gagacatctataacca

SEQ ID NO: 823 - DNA, 16 bp, Artificial Sequence
  Description of Artificial Sequencecapture
gatacttccataacca

SEQ ID NO: 824 - DNA, 16 bp, Artificial Sequence
  Description of Artificial Sequencecapture
gacacttccataacca

SEQ ID NO: 825 - DNA, 16 bp, Artificial Sequence
  Description of Artificial Sequencecapture
ataaccaagaggagta

SEQ ID NO: 826 - DNA, 16 bp, Artificial Sequence
  Description of Artificial Sequencecapture
ataaccaagaggagga

SEQ ID NO: 827 - DNA, 16 bp, Artificial Sequence
  Description of Artificial Sequencecapture
ataaccaggaggagtc

SEQ ID NO: 828 - DNA, 16 bp, Artificial Sequence
  Description of Artificial Sequencecapture
ataaccaggaggagaa

SEQ ID NO: 829 - DNA, 16 bp, Artificial Sequence
  Description of Artificial Sequencecapture
ataaccaggaggagta

SEQ ID NO: 830 - DNA, 16 bp, Artificial Sequence
  Description of Artificial Sequencecapture
agaggaggacttgcgc

SEQ ID NO: 831 - DNA, 19 bp, Artificial Sequence
  Description of Artificial Sequencecapture
aggaggagtacgcgcgctt

SEQ ID NO: 832 - DNA, 16 bp, Artificial Sequence
  Description of Artificial Sequencecapture
ggagttcctgcgcttc

SEQ ID NO: 833 - DNA, 17 bp, Artificial Sequence
  Description of Artificial Sequencecapture
aggaggacttgcgcttc

SEQ ID NO: 834 - DNA, 17 bp, Artificial Sequence
  Description of Artificial Sequencecapture
ggagtacgcgcgcttcg

SEQ ID NO: 835 - DNA, 18 bp, Artificial Sequence
  Description of Artificial Sequencecapture
aggagaacgcgcgcttcg

SEQ ID NO: 836 - DNA, 17 bp, Artificial Sequence
  Description of Artificial Sequencecapture
ggcggtgagggagctgg

SEQ ID NO: 837 - DNA, 17 bp, Artificial Sequence
  Description of Artificial Sequencecapture
gcggcctgttgccgagt

SEQ ID NO: 838 - DNA, 15 bp, Artificial Sequence
  Description of Artificial Sequencecapture
gccgagtcctggaac

SEQ ID NO: 839 - DNA, 19 bp, Artificial Sequence
  Description of Artificial Sequencecapture
tcctggaagacacgcgcgc

SEQ ID NO: 840 - DNA, 17 bp, Artificial Sequence
  Description of Artificial Sequencecapture
gacacgcgcgccgcggt

SEQ ID NO: 841 - DNA, 17 bp, Artificial Sequence
  Description of Artificial Sequencecapture
cagaagcggggccaggt

SEQ ID NO: 842 - DNA, 14 bp, Artificial Sequence
  Description of Artificial Sequencecapture
cgggccctggtgga

SEQ ID NO: 843 - DNA, 17 bp, Artificial Sequence
  Description of Artificial Sequencecapture
cgaggtgggcacctact

SEQ ID NO: 844 - DNA, 17 bp, Artificial Sequence
  Description of Artificial Sequencecapture
ggtggacaactactgca

SEQ ID NO: 845 - DNA, 17 bp, Artificial Sequence
  Description of Artificial Sequencecapture
tactgcaggcacaacta

SEQ ID NO: 846 - DNA, 17 bp, Artificial Sequence
  Description of Artificial Sequencecapture
actgcagatacaactac

SEQ ID NO: 847 - DNA, 16 bp, Artificial Sequence
  Description of Artificial Sequencecapture
aactacggagttggtg

SEQ ID NO: 848 - DNA, 17 bp, Artificial Sequence
  Description of Artificial Sequencecapture
ctacggggctgtggaga

SEQ ID NO: 849 - DNA, 17 bp, Artificial Sequence
  Description of Artificial Sequencecapture
attgcagatacaactac

SEQ ID NO: 850 - DNA, 18 bp, Artificial Sequence
  Description of Artificial Sequencecapture
cggagttggtgagagctt

SEQ ID NO: 851 - DNA, 18 bp, Artificial Sequence
  Description of Artificial Sequencecapture
cggcgttgtggagagctt

SEQ ID NO: 852 - DNA, 17 bp, Artificial Sequence
  Description of Artificial Sequencecapture
acgtttctcggagctgc

SEQ ID NO: 853 - DNA, 17 bp, Artificial Sequence
  Description of Artificial Sequencecapture
tggagctgtgtaagtct

SEQ ID NO: 854 - DNA, 17 bp, Artificial Sequence
  Description of Artificial Sequencecapture
gtgagtgtcatatcttc

SEQ ID NO: 855 - DNA, 17 bp, Artificial Sequence
  Description of Artificial Sequencecapture
ctgagtgtcatttcttc

SEQ ID NO: 856 - DNA, 17 bp, Artificial Sequence
  Description of Artificial Sequencecapture
acgagtgctatttctgc

SEQ ID NO: 857 - DNA, 17 bp, Artificial Sequence
  Description of Artificial Sequencecapture
gtgcagtacctgaacag

SEQ ID NO: 858 - DNA, 17 bp, Artificial Sequence
  Description of Artificial Sequencecapture
gagcggtacctgtaaag

SEQ ID NO: 859 - DNA, 17 bp, Artificial Sequence
  Description of Artificial Sequencecapture
atgcggtttgtgcagag

SEQ ID NO: 860 - DNA, 16 bp, Artificial Sequence
  Description of Artificial Sequencecapture
tacctgtaaagatact

SEQ ID NO: 861 - DNA, 16 bp, Artificial Sequence
  Description of Artificial Sequencecapture
tacctgtaaagatatt

SEQ ID NO: 862 - DNA, 17 bp, Artificial Sequence
  Description of Artificial Sequencecapture
gtacctgaacagataca

SEQ ID NO: 863 - DNA, 17 bp, Artificial Sequence
  Description of Artificial Sequencecapture
gtttgtgcagagactcg

SEQ ID NO: 864 - DNA, 17 bp, Artificial Sequence
  Description of Artificial Sequencecapture
taaagatacttctataa

SEQ ID NO: 865 - DNA, 17 bp, Artificial Sequence
  Description of Artificial Sequencecapture
taaagatatttctataa

SEQ ID NO: 866 - DNA, 25 bp, Artificial Sequence
  Description of Artificial Sequencecapture
gatacatccataaacgggaggagaa

SEQ ID NO: 867 - DNA, 25 bp, Artificial Sequence
  Description of Artificial Sequencecapture
gatacttctataaccaagaggagta

SEQ ID NO: 868 - DNA, 25 bp, Artificial Sequence
  Description of Artificial Sequencecapture
gatatttctataaccaagaggagta

SEQ ID NO: 869 - DNA, 24 bp, Artificial Sequence
  Description of Artificial Sequencecapture
gactcgtccacaccggaaggagta

SEQ ID NO: 870 - DNA, 16 bp, Artificial Sequence
  Description of Artificial Sequencecapture
ggaggagaacctgccc

SEQ ID NO: 871 - DNA, 17 bp, Artificial Sequence
  Description of Artificial Sequencecapture
gaaggagtatgcgcgct

SEQ ID NO: 872 - DNA, 17 bp, Artificial Sequence
  Description of Artificial Sequencecapture
gaacctgcccttcgaca

SEQ ID NO: 873 - DNA, 17 bp, Artificial Sequence
  Description of Artificial Sequencecapture
ggaggagttccaggcag

SEQ ID NO: 874 - DNA, 17 bp, Artificial Sequence
  Description of Artificial Sequencecapture
cagaaagttccgggcgg

SEQ ID NO: 875 - DNA, 20 bp, Artificial Sequence
  Description of Artificial Sequencecapture
gcctgtcgcagagaactgga

SEQ ID NO: 876 - DNA, 20 bp, Artificial Sequence
  Description of Artificial Sequencecapture
agaagagtccaggaatgcaa

SEQ ID NO: 877 - DNA, 18 bp, Artificial Sequence
  Description of Artificial Sequencecapture
cagaagaacctcttgggc

SEQ ID NO: 878 - DNA, 18 bp, Artificial Sequence
  Description of Artificial Sequencecapture
cagaagggaatcctggag

SEQ ID NO: 879 - DNA, 18 bp, Artificial Sequence
  Description of Artificial Sequencecapture
cagaagggcatcccggag

SEQ ID NO: 880 - DNA, 19 bp, Artificial Sequence
  Description of Artificial Sequencecapture
cctggaggagaatcgggac

SEQ ID NO: 881 - DNA, 19 bp, Artificial Sequence
  Description of Artificial Sequencecapture
cccggaggagaagcgggac

SEQ ID NO: 882 - DNA, 19 bp, Artificial Sequence
  Description of Artificial Sequencecapture
cttgggctgcttgcggggt

SEQ ID NO: 883 - DNA, 17 bp, Artificial Sequence
  Description of Artificial Sequencecapture
gagaatcgggacaaggt

SEQ ID NO: 884 - DNA, 17 bp, Artificial Sequence
  Description of Artificial Sequencecapture
gagaagcgggacaagat

SEQ ID NO: 885 - DNA, 17 bp, Artificial Sequence
  Description of Artificial Sequencecapture
cagaagcgggccgaagt

SEQ ID NO: 886 - DNA, 17 bp, Artificial Sequence
  Description of Artificial Sequencecapture
tgcttgcggggtctgtt

SEQ ID NO: 887 - DNA, 15 bp, Artificial Sequence
  Description of Artificial Sequencecapture
cgggacaaggtggac

SEQ ID NO: 888 - DNA, 15 bp, Artificial Sequence
  Description of Artificial Sequencecapture
cgggacaagatggac

SEQ ID NO: 889 - DNA, 15 bp, Artificial Sequence
  Description of Artificial Sequencecapture
cgggccgaagtggac

SEQ ID NO: 890 - DNA, 15 bp, Artificial Sequence
  Description of Artificial Sequencecapture
cggggtctgttggac

SEQ ID NO: 891 - DNA, 17 bp, Artificial Sequence
  Description of Artificial Sequencecapture
gacacctactgcagata

SEQ ID NO: 892 - DNA, 17 bp, Artificial Sequence
  Description of Artificial Sequencecapture
gacgactactgcagata

SEQ ID NO: 893 - DNA, 17 bp, Artificial Sequence
  Description of Artificial Sequencecapture
gacaactaatgcagaca

SEQ ID NO: 894 - DNA, 17 bp, Artificial Sequence
  Description of Artificial Sequencecapture
acaattacagggttttt

SEQ ID NO: 895 - DNA, 16 bp, Artificial Sequence
  Description of Artificial Sequencecapture
ggggtttttgagagct

SEQ ID NO: 896 - DNA, 16 bp, Artificial Sequence
  Description of Artificial Sequencecapture
ggggttttttgagagc

SEQ ID NO: 897 - DNA, 16 bp, Artificial Sequence
  Description of Artificial Sequencecapture
tacagggttttttgag

SEQ ID NO: 898 - DNA, 16 bp, Artificial Sequence
  Description of Artificial Sequencecapture
ggggttgtggagagat

SEQ ID NO: 899 - DNA, 21 bp, Artificial Sequence
  Description of Artificial Sequenceprimer
cagccactcctcgtccccagg

SEQ ID NO: 900 - DNA, 21 bp, Artificial Sequence
  Description of Artificial Sequenceprimer
cagccactcctcgcccccagg

SEQ ID NO: 901 - DNA, 21 bp, Artificial Sequence
  Description of Artificial Sequenceprimer
gggatgaggggtcgtgacctg

SEQ ID NO: 902 - DNA, 21 bp, Artificial Sequence
  Description of Artificial Sequenceprimer
gggatgagaggtcgtgacctg

SEQ ID NO: 903 - DNA, 20 bp, Artificial Sequence
  Description of Artificial Sequenceprimer
ggtcggggccaggttctcac

SEQ ID NO: 904 - DNA, 20 bp, Artificial Sequence
  Description of Artificial Sequenceprimer
ggtccgggccaggttctcac

SEQ ID NO: 905 - DNA, 20 bp, Artificial Sequence
  Description of Artificial Sequenceprimer
ggtcgaggccaggttctcac

SEQ ID NO: 906 - DNA, 20 bp, Artificial Sequence
  Description of Artificial Sequenceprimer
ccgtggcccctggtacccgt

SEQ ID NO: 907 - DNA, 20 bp, Artificial Sequence
  Description of Artificial Sequenceprimer
ccgtggcccctggtacccgc

SEQ ID NO: 908 - DNA, 17 bp, Artificial Sequence
  Description of Artificial Sequencecapture
ccaggctctcactccat

SEQ ID NO: 909 - DNA, 17 bp, Artificial Sequence
  Description of Artificial Sequencecapture
ggctctcagtccatgag

SEQ ID NO: 910 - DNA, 16 bp, Artificial Sequence
  Description of Artificial Sequencecapture
gctcccaatccatgag

SEQ ID NO: 911 - DNA, 17 bp, Artificial Sequence
  Description of Artificial Sequencecapture
catgaggtgtttctcca

SEQ ID NO: 912 - DNA, 18 bp, Artificial Sequence
  Description of Artificial Sequencecapture
tatttcttcacatccgtg

SEQ ID NO: 913 - DNA, 17 bp, Artificial Sequence
  Description of Artificial Sequencecapture
atttctacacctccgtg

SEQ ID NO: 914 - DNA, 17 bp, Artificial Sequence
  Description of Artificial Sequencecapture
gtatttctccacatccg

SEQ ID NO: 915 - DNA, 16 bp, Artificial Sequence
  Description of Artificial Sequencecapture
gtatttcaccacatcc

SEQ ID NO: 916 - DNA, 17 bp, Artificial Sequence
  Description of Artificial Sequencecapture
atttctacacttccgtg

SEQ ID NO: 917 - DNA, 17 bp, Artificial Sequence
  Description of Artificial Sequencecapture
atttctacacctccatg

SEQ ID NO: 918 - DNA, 15 bp, Artificial Sequence
  Description of Artificial Sequencecapture
ggttctccacatccg

SEQ ID NO: 919 - DNA, 17 bp, Artificial Sequence
  Description of Artificial Sequencecapture
ggcagtggagagccccg

SEQ ID NO: 920 - DNA, 16 bp, Artificial Sequence
  Description of Artificial Sequencecapture
gcagtgggaagccccg

SEQ ID NO: 921 - DNA, 17 bp, Artificial Sequence
  Description of Artificial Sequencecapture
cttcatcgccgtgggct

SEQ ID NO: 922 - DNA, 17 bp, Artificial Sequence
  Description of Artificial Sequencecapture
ttcatcgcagtgggcta

SEQ ID NO: 923 - DNA, 17 bp, Artificial Sequence
  Description of Artificial Sequencecapture
cttcatctcagtgggct

SEQ ID NO: 924 - DNA, 17 bp, Artificial Sequence
  Description of Artificial Sequencecapture
acgtggacaacacgcag

SEQ ID NO: 925 - DNA, 17 bp, Artificial Sequence
  Description of Artificial Sequencecapture
gtgcggttggacagcga

SEQ ID NO: 926 - DNA, 17 bp, Artificial Sequence
  Description of Artificial Sequencecapture
gtgcggtttgacagcga

SEQ ID NO: 927 - DNA, 17 bp, Artificial Sequence
  Description of Artificial Sequencecapture
cgacgccgggagccaga

SEQ ID NO: 928 - DNA, 17 bp, Artificial Sequence
  Description of Artificial Sequencecapture
gcgagccagaagatgga

SEQ ID NO: 929 - DNA, 17 bp, Artificial Sequence
  Description of Artificial Sequencecapture
gcgagccagaggatgga

SEQ ID NO: 930 - DNA, 17 bp, Artificial Sequence
  Description of Artificial Sequencecapture
gcgagccggaggatgga

SEQ ID NO: 931 - DNA, 17 bp, Artificial Sequence
  Description of Artificial Sequencecapture
gggagccagaggatgga

SEQ ID NO: 932 - DNA, 17 bp, Artificial Sequence
  Description of Artificial Sequencecapture
ccgtggatggagcagga

SEQ ID NO: 933 - DNA, 17 bp, Artificial Sequence
  Description of Artificial Sequencecapture
gatagagcgggaggggc

SEQ ID NO: 934 - DNA, 17 bp, Artificial Sequence
  Description of Artificial Sequencecapture
gcaggaggggccggagt

SEQ ID NO: 935 - DNA, 17 bp, Artificial Sequence
  Description of Artificial Sequencecapture
agcaggagaggcctgag

SEQ ID NO: 936 - DNA, 17 bp, Artificial Sequence
  Description of Artificial Sequencecapture
caggagggtccggagta

SEQ ID NO: 937 - DNA, 17 bp, Artificial Sequence
  Description of Artificial Sequencecapture
gcaggagagtccggagt

SEQ ID NO: 938 - DNA, 17 bp, Artificial Sequence
  Description of Artificial Sequencecapture
gcaggaggagccggagt

SEQ ID NO: 939 - DNA, 16 bp, Artificial Sequence
  Description of Artificial Sequencecapture
cgggaggggccggagt

SEQ ID NO: 940 - DNA, 17 bp, Artificial Sequence
  Description of Artificial Sequencecapture
gagaggcctgagtattg

SEQ ID NO: 941 - DNA, 17 bp, Artificial Sequence
  Description of Artificial Sequencecapture
ttgggaccaggagacac

SEQ ID NO: 942 - DNA, 16 bp, Artificial Sequence
  Description of Artificial Sequencecapture
ttgggacggggagaca

SEQ ID NO: 943 - DNA, 18 bp, Artificial Sequence
  Description of Artificial Sequencecapture
tattgggacgaggagaca

SEQ ID NO: 944 - DNA, 17 bp, Artificial Sequence
  Description of Artificial Sequencecapture
ttgggaccggaacacac

SEQ ID NO: 945 - DNA, 17 bp, Artificial Sequence
  Description of Artificial Sequencecapture
ttgggacctgcagacac

SEQ ID NO: 946 - DNA, 16 bp, Artificial Sequence
  Description of Artificial Sequencecapture
gggacgggcagacagg

SEQ ID NO: 947 - DNA, 19 bp, Artificial Sequence
  Description of Artificial Sequencecapture
ttgggacgggaagacacgg

SEQ ID NO: 948 - DNA, 17 bp, Artificial Sequence
  Description of Artificial Sequencecapture
gggacgggcagacaggg

SEQ ID NO: 949 - DNA, 17 bp, Artificial Sequence
  Description of Artificial Sequencecapture
gaccggaacacacggaa

SEQ ID NO: 950 - DNA, 15 bp, Artificial Sequence
  Description of Artificial Sequencecapture
aggagacagggaaag

SEQ ID NO: 951 - DNA, 18 bp, Artificial Sequence
  Description of Artificial Sequencecapture
gggcagacagggaaagtg

SEQ ID NO: 952 - DNA, 16 bp, Artificial Sequence
  Description of Artificial Sequencecapture
cacggaatatgaaggc

SEQ ID NO: 953 - DNA, 17 bp, Artificial Sequence
  Description of Artificial Sequencecapture
cacggaatgtgaaggcc

SEQ ID NO: 954 - DNA, 16 bp, Artificial Sequence
  Description of Artificial Sequencecapture
cacggaaagtgaaggc

SEQ ID NO: 955 - DNA, 16 bp, Artificial Sequence
  Description of Artificial Sequencecapture
cacggaacgtgaaggc

SEQ ID NO: 956 - DNA, 18 bp, Artificial Sequence
  Description of Artificial Sequencecapture
gacacggcaagtgaaggc

SEQ ID NO: 957 - DNA, 18 bp, Artificial Sequence
  Description of Artificial Sequencecapture
gacacggcaagtgaaggc

SEQ ID NO: 958 - DNA, 16 bp, Artificial Sequence
  Description of Artificial Sequencecapture
cagggaaagtgaaggc

SEQ ID NO: 959 - DNA, 12 bp, Artificial Sequence
  Description of Artificial Sequencecapture
gcccagtcacag

SEQ ID NO: 960 - DNA, 17 bp, Artificial Sequence
  Description of Artificial Sequencecapture
agtcacaggctgaccga

SEQ ID NO: 961 - DNA, 17 bp, Artificial Sequence
  Description of Artificial Sequencecapture
ctcacagattcaccgag

SEQ ID NO: 962 - DNA, 17 bp, Artificial Sequence
  Description of Artificial Sequencecapture
ctcacagagtcaccgag

SEQ ID NO: 963 - DNA, 17 bp, Artificial Sequence
  Description of Artificial Sequencecapture
acagactgaccgagcga

SEQ ID NO: 964 - DNA, 17 bp, Artificial Sequence
  Description of Artificial Sequencecapture
cacagactcaccgagtg

SEQ ID NO: 965 - DNA, 17 bp, Artificial Sequence
  Description of Artificial Sequencecapture
acagactcatcgagtgg

SEQ ID NO: 966 - DNA, 17 bp, Artificial Sequence
  Description of Artificial Sequencecapture
cacagactaaccgagcg

SEQ ID NO: 967 - DNA, 17 bp, Artificial Sequence
  Description of Artificial Sequencecapture
acagactgaccgagaga

SEQ ID NO: 968 - DNA, 17 bp, Artificial Sequence
  Description of Artificial Sequencecapture
cacagagtcaccgagtg

SEQ ID NO: 969 - DNA, 17 bp, Artificial Sequence
  Description of Artificial Sequencecapture
acagattgaccgagtgg

SEQ ID NO: 970 - DNA, 17 bp, Artificial Sequence
  Description of Artificial Sequencecapture
cacagactcaccgagag

SEQ ID NO: 971 - DNA, 17 bp, Artificial Sequence
  Description of Artificial Sequencecapture
cacaggctgaccgagtg

SEQ ID NO: 972 - DNA, 18 bp, Artificial Sequence
  Description of Artificial Sequencecapture
gaccgagcgaacctgggg

SEQ ID NO: 973 - DNA, 17 bp, Artificial Sequence
  Description of Artificial Sequencecapture
tgaccgagagaacctgg

SEQ ID NO: 974 - DNA, 17 bp, Artificial Sequence
  Description of Artificial Sequencecapture
gaccgagtggacctggg

SEQ ID NO: 975 - DNA, 16 bp, Artificial Sequence
  Description of Artificial Sequencecapture
gaccgagagagcctgc

SEQ ID NO: 976 - DNA, 18 bp, Artificial Sequence
  Description of Artificial Sequencecapture
aaccgagagaacctgggg

SEQ ID NO: 977 - DNA, 17 bp, Artificial Sequence
  Description of Artificial Sequencecapture
tcaccgagagaacctgc

SEQ ID NO: 978 - DNA, 17 bp, Artificial Sequence
  Description of Artificial Sequencecapture
catcgagtggacctggg

SEQ ID NO: 979 - DNA, 17 bp, Artificial Sequence
  Description of Artificial Sequencecapture
tgaccgagagaacctgc

SEQ ID NO: 980 - DNA, 17 bp, Artificial Sequence
  Description of Artificial Sequencecapture
caccgagtggacctggg

SEQ ID NO: 981 - DNA, 17 bp, Artificial Sequence
  Description of Artificial Sequencecapture
gaccgagtggacctggc

SEQ ID NO: 982 - DNA, 18 bp, Artificial Sequence
  Description of Artificial Sequencecapture
ggacctggcgaccctgcg

SEQ ID NO: 983 - DNA, 17 bp, Artificial Sequence
  Description of Artificial Sequencecapture
actacaacgagagcgag

SEQ ID NO: 984 - DNA, 17 bp, Artificial Sequence
  Description of Artificial Sequencecapture
gagcgaggccggtgagt

SEQ ID NO: 985 - DNA, 17 bp, Artificial Sequence
  Description of Artificial Sequencecapture
ggccaggtactcacacc

SEQ ID NO: 986 - DNA, 17 bp, Artificial Sequence
  Description of Artificial Sequencecapture
tcacaccatccagataa

SEQ ID NO: 987 - DNA, 17 bp, Artificial Sequence
  Description of Artificial Sequencecapture
tcacaccgtccagagga

SEQ ID NO: 988 - DNA, 17 bp, Artificial Sequence
  Description of Artificial Sequencecapture
tcacaccctccagagga

SEQ ID NO: 989 - DNA, 17 bp, Artificial Sequence
  Description of Artificial Sequencecapture
tcacaccatccagatga

SEQ ID NO: 990 - DNA, 16 bp, Artificial Sequence
  Description of Artificial Sequencecapture
tcacaccctccagatg

SEQ ID NO: 991 - DNA, 16 bp, Artificial Sequence
  Description of Artificial Sequencecapture
tcacaccatccagagg

SEQ ID NO: 992 - DNA, 17 bp, Artificial Sequence
  Description of Artificial Sequencecapture
catccagataatgtatg

SEQ ID NO: 993 - DNA, 17 bp, Artificial Sequence
  Description of Artificial Sequencecapture
atccagatgatgtatgg

SEQ ID NO: 994 - DNA, 18 bp, Artificial Sequence
  Description of Artificial Sequencecapture
catccagaggatgtatgg

SEQ ID NO: 995 - DNA, 18 bp, Artificial Sequence
  Description of Artificial Sequencecapture
cgtccagatgatgtatgg

SEQ ID NO: 996 - DNA, 18 bp, Artificial Sequence
  Description of Artificial Sequencecapture
cgtccagaggatgtatgg

SEQ ID NO: 997 - DNA, 17 bp, Artificial Sequence
  Description of Artificial Sequencecapture
ctccagatgatgtttgg

SEQ ID NO: 998 - DNA, 18 bp, Artificial Sequence
  Description of Artificial Sequencecapture
cctccagaggatgtatgg

SEQ ID NO: 999 - DNA, 17 bp, Artificial Sequence
  Description of Artificial Sequencecapture
ctccagatgatgtttgg

SEQ ID NO: 1000 - DNA, 18 bp, Artificial Sequence
  Description of Artificial Sequencecapture
cgtccagaggatgtttgg

SEQ ID NO: 1001 - DNA, 18 bp, Artificial Sequence
  Description of Artificial Sequencecapture
cgtccagaggatgtctgg

SEQ ID NO: 1002 - DNA, 18 bp, Artificial Sequence
  Description of Artificial Sequencecapture
cgtccagaggatgtgtgg

SEQ ID NO: 1003 - DNA, 17 bp, Artificial Sequence
  Description of Artificial Sequencecapture
gaggatgtgtggctgcg

SEQ ID NO: 1004 - DNA, 17 bp, Artificial Sequence
  Description of Artificial Sequencecapture
gaggatgtttggctgcg

SEQ ID NO: 1005 - DNA, 17 bp, Artificial Sequence
  Description of Artificial Sequencecapture
gaggatgtctggctgcg

SEQ ID NO: 1006 - DNA, 17 bp, Artificial Sequence
  Description of Artificial Sequencecapture
atggctgccacgtgggg

SEQ ID NO: 1007 - DNA, 17 bp, Artificial Sequence
  Description of Artificial Sequencecapture
acgtggggtcggacggg

SEQ ID NO: 1008 - DNA, 17 bp, Artificial Sequence
  Description of Artificial Sequencecapture
acgtggggtcggactgg

SEQ ID NO: 1009 - DNA, 17 bp, Artificial Sequence
  Description of Artificial Sequencecapture
gggcgcttactccgcgg

SEQ ID NO: 1010 - DNA, 17 bp, Artificial Sequence
  Description of Artificial Sequencecapture
cttcctccacgggtacc

SEQ ID NO: 1011 - DNA, 17 bp, Artificial Sequence
  Description of Artificial Sequencecapture
cgcgggtatgaacagca

SEQ ID NO: 1012 - DNA, 18 bp, Artificial Sequence
  Description of Artificial Sequencecapture
cgggtaccaccagtacgc

SEQ ID NO: 1013 - DNA, 18 bp, Artificial Sequence
  Description of Artificial Sequencecapture
cgggtaccaccagtacgc

SEQ ID NO: 1014 - DNA, 17 bp, Artificial Sequence
  Description of Artificial Sequencecapture
cgggtaccagcaggacg

SEQ ID NO: 1015 - DNA, 18 bp, Artificial Sequence
  Description of Artificial Sequencecapture
cgggtaccagcagaacgc

SEQ ID NO: 1016 - DNA, 17 bp, Artificial Sequence
  Description of Artificial Sequencecapture
ggtaccagcgggacgct

SEQ ID NO: 1017 - DNA, 17 bp, Artificial Sequence
  Description of Artificial Sequencecapture
cgggtatgaacagcacg

SEQ ID NO: 1018 - DNA, 17 bp, Artificial Sequence
  Description of Artificial Sequencecapture
ggtaccggcaggacgcc

SEQ ID NO: 1019 - DNA, 17 bp, Artificial Sequence
  Description of Artificial Sequencecapture
accggcagcacgcctac

SEQ ID NO: 1020 - DNA, 17 bp, Artificial Sequence
  Description of Artificial Sequencecapture
accaccagtacgcctac

SEQ ID NO: 1021 - DNA, 17 bp, Artificial Sequence
  Description of Artificial Sequencecapture
accagcagtacgcctac

SEQ ID NO: 1022 - DNA, 17 bp, Artificial Sequence
  Description of Artificial Sequencecapture
accagcagaacgcttac

SEQ ID NO: 1023 - DNA, 17 bp, Artificial Sequence
  Description of Artificial Sequencecapture
atgaacagcacgcctac

SEQ ID NO: 1024 - DNA, 17 bp, Artificial Sequence
  Description of Artificial Sequencecapture
ggtaccggcaggacgct

SEQ ID NO: 1025 - DNA, 17 bp, Artificial Sequence
  Description of Artificial Sequencecapture
ccggcaggtcgcctacg

SEQ ID NO: 1026 - DNA, 17 bp, Artificial Sequence
  Description of Artificial Sequencecapture
acatcgccttgaacgag

SEQ ID NO: 1027 - DNA, 17 bp, Artificial Sequence
  Description of Artificial Sequencecapture
gccctgaaagaggacct

SEQ ID NO: 1028 - DNA, 17 bp, Artificial Sequence
  Description of Artificial Sequencecapture
aggacctgagctcctgg

SEQ ID NO: 1029 - DNA, 17 bp, Artificial Sequence
  Description of Artificial Sequencecapture
accgcggcagacatggc

SEQ ID NO: 1030 - DNA, 17 bp, Artificial Sequence
  Description of Artificial Sequencecapture
ggcggacaaggcagctc

SEQ ID NO: 1031 - DNA, 17 bp, Artificial Sequence
  Description of Artificial Sequencecapture
gacatggcggctcagat

SEQ ID NO: 1032 - DNA, 18 bp, Artificial Sequence
  Description of Artificial Sequencecapture
agctcagaccaccaagca

SEQ ID NO: 1033 - DNA, 17 bp, Artificial Sequence
  Description of Artificial Sequencecapture
agatcacccagcgcaag

SEQ ID NO: 1034 - DNA, 17 bp, Artificial Sequence
  Description of Artificial Sequencecapture
agatcaccgagcgcaag

SEQ ID NO: 1035 - DNA, 17 bp, Artificial Sequence
  Description of Artificial Sequencecapture
agtgggagacggcccat

SEQ ID NO: 1036 - DNA, 17 bp, Artificial Sequence
  Description of Artificial Sequencecapture
ggcggcccgtgtggcgg

SEQ ID NO: 1037 - DNA, 17 bp, Artificial Sequence
  Description of Artificial Sequencecapture
ggcggcccttgtggcgg

SEQ ID NO: 1038 - DNA, 17 bp, Artificial Sequence
  Description of Artificial Sequencecapture
ggcccatgtggcggagc

SEQ ID NO: 1039 - DNA, 17 bp, Artificial Sequence
  Description of Artificial Sequencecapture
ggcccatgaggcggagc

SEQ ID NO: 1040 - DNA, 17 bp, Artificial Sequence
  Description of Artificial Sequencecapture
ggcccgttgggcggagc

SEQ ID NO: 1041 - DNA, 17 bp, Artificial Sequence
  Description of Artificial Sequencecapture
ggcggacaaggcggctc

SEQ ID NO: 1042 - DNA, 17 bp, Artificial Sequence
  Description of Artificial Sequencecapture
ggcggcccgtgaggcgg

SEQ ID NO: 1043 - DNA, 17 bp, Artificial Sequence
  Description of Artificial Sequencecapture
ggcccgtgtggcggagc

SEQ ID NO: 1044 - DNA, 17 bp, Artificial Sequence
  Description of Artificial Sequencecapture
ggcccgtgaggcggagc

SEQ ID NO: 1045 - DNA, 17 bp, Artificial Sequence
  Description of Artificial Sequencecapture
ggcccgtcgggcggagc

SEQ ID NO: 1046 - DNA, 17 bp, Artificial Sequence
  Description of Artificial Sequencecapture
ggcggcccgtcgggcgg

SEQ ID NO: 1047 - DNA, 17 bp, Artificial Sequence
  Description of Artificial Sequencecapture
ggcccttgtggcggagc

SEQ ID NO: 1048 - DNA, 17 bp, Artificial Sequence
  Description of Artificial Sequencecapture
ggcggcccgttgggcgg

SEQ ID NO: 1049 - DNA, 17 bp, Artificial Sequence
  Description of Artificial Sequencecapture
ggagcagcggagagtct

SEQ ID NO: 1050 - DNA, 17 bp, Artificial Sequence
  Description of Artificial Sequencecapture
ggagcagttgagagcct

SEQ ID NO: 1051 - DNA, 18 bp, Artificial Sequence
  Description of Artificial Sequencecapture
cggagcagtggagagcct

SEQ ID NO: 1052 - DNA, 17 bp, Artificial Sequence
  Description of Artificial Sequencecapture
ggagcagcagagagcct

SEQ ID NO: 1053 - DNA, 17 bp, Artificial Sequence
  Description of Artificial Sequencecapture
ggagcagctgagagcct

SEQ ID NO: 1054 - DNA, 17 bp, Artificial Sequence
  Description of Artificial Sequencecapture
ggagcagcggagagcct

SEQ ID NO: 1055 - DNA, 17 bp, Artificial Sequence
  Description of Artificial Sequencecapture
cctacctgcagggccgg

SEQ ID NO: 1056 - DNA, 17 bp, Artificial Sequence
  Description of Artificial Sequencecapture
tacctggatggcacgtg

SEQ ID NO: 1057 - DNA, 17 bp, Artificial Sequence
  Description of Artificial Sequencecapture
cctggatgccacgtgcg

SEQ ID NO: 1058 - DNA, 17 bp, Artificial Sequence
  Description of Artificial Sequencecapture
ggagggccggtgcgtgg

SEQ ID NO: 1059 - DNA, 17 bp, Artificial Sequence
  Description of Artificial Sequencecapture
tggagggctggtgcgtg

SEQ ID NO: 1060 - DNA, 18 bp, Artificial Sequence
  Description of Artificial Sequencecapture
tggagggcacgtgcgtgg

SEQ ID NO: 1061 - DNA, 18 bp, Artificial Sequence
  Description of Artificial Sequencecapture
tggagggcgagtgcgtgg

SEQ ID NO: 1062 - DNA, 17 bp, Artificial Sequence
  Description of Artificial Sequencecapture
ggagggcctgtgcgtgg

SEQ ID NO: 1063 - DNA, 17 bp, Artificial Sequence
  Description of Artificial Sequencecapture
gcagggccggtgcgtgg

SEQ ID NO: 1064 - DNA, 18 bp, Artificial Sequence
  Description of Artificial Sequencecapture
tggatggcacgtgcgtgg

SEQ ID NO: 1065 - DNA, 18 bp, Artificial Sequence
  Description of Artificial Sequencecapture
tggatgccacgtgcgtgg

SEQ ID NO: 1066 - DNA, 16 bp, Artificial Sequence
  Description of Artificial Sequencecapture
gcgtggagtggctccg

SEQ ID NO: 1067 - DNA, 17 bp, Artificial Sequence
  Description of Artificial Sequencecapture
gcgtggagtggctccgc

SEQ ID NO: 1068 - DNA, 17 bp, Artificial Sequence
  Description of Artificial Sequencecapture
tccgcagacacctggag

SEQ ID NO: 1069 - DNA, 14 bp, Artificial Sequence
  Description of Artificial Sequencecapture
tggagaaccggaag

SEQ ID NO: 1070 - DNA, 17 bp, Artificial Sequence
  Description of Artificial Sequencecapture
cagcgcactggtgagtg

SEQ ID NO: 1071 - DNA, 17 bp, Artificial Sequence
  Description of Artificial Sequencecapture
gcgtggactggctccgc

SEQ ID NO: 1072 - DNA, 19 bp, Artificial Sequence
  Description of Artificial Sequencecapture
cctggagaagcggcggccc

SEQ ID NO: 1073 - DNA, 19 bp, Artificial Sequence
  Description of Artificial Sequenceprimer
gcccctcctcgcccccagg

SEQ ID NO: 1074 - DNA, 18 bp, Artificial Sequence
  Description of Artificial Sequenceprimer
ggccggggtcactcaccg

SEQ ID NO: 1075 - DNA, 18 bp, Artificial Sequence
  Description of Artificial Sequenceprimer
ggccggggccagggtctc

SEQ ID NO: 1076 - DNA, 18 bp, Artificial Sequence
  Description of Artificial Sequenceprimer
gggcggggccagggtctc

SEQ ID NO: 1077 - DNA, 18 bp, Artificial Sequence
  Description of Artificial Sequenceprimer
ggcctgggccagggtctc

SEQ ID NO: 1078 - DNA, 20 bp, Artificial Sequence
  Description of Artificial Sequenceprimer
ccccactgcccctggtacca

SEQ ID NO: 1079 - DNA, 20 bp, Artificial Sequence
  Description of Artificial Sequenceprimer
ccccactgcccctggtaccc

SEQ ID NO: 1080 - DNA, 17 bp, Artificial Sequence
  Description of Artificial Sequencecapture
acacctccgtgtcccgg

SEQ ID NO: 1081 - DNA, 17 bp, Artificial Sequence
  Description of Artificial Sequencecapture
acaccgccatgtcccgg

SEQ ID NO: 1082 - DNA, 17 bp, Artificial Sequence
  Description of Artificial Sequencecapture
tctacaccgccgtgtcc

SEQ ID NO: 1083 - DNA, 20 bp, Artificial Sequence
  Description of Artificial Sequencecapture
ctacaccgctatgtcccggc

SEQ ID NO: 1084 - DNA, 18 bp, Artificial Sequence
  Description of Artificial Sequencecapture
gcttcatctcagtgggct

SEQ ID NO: 1085 - DNA, 17 bp, Artificial Sequence
  Description of Artificial Sequencecapture
cttcatcaccgtgggct

SEQ ID NO: 1086 - DNA, 17 bp, Artificial Sequence
  Description of Artificial Sequencecapture
gcttcatcgcagtgggc

SEQ ID NO: 1087 - DNA, 16 bp, Artificial Sequence
  Description of Artificial Sequencecapture
gcttcattgcagtggg

SEQ ID NO: 1088 - DNA, 17 bp, Artificial Sequence
  Description of Artificial Sequencecapture
ttcatctccgtgggcta

SEQ ID NO: 1089 - DNA, 18 bp, Artificial Sequence
  Description of Artificial Sequencecapture
gtccgagagaggagccgc

SEQ ID NO: 1090 - DNA, 17 bp, Artificial Sequence
  Description of Artificial Sequencecapture
tccgagaggggagccgc

SEQ ID NO: 1091 - DNA, 17 bp, Artificial Sequence
  Description of Artificial Sequencecapture
gtccgaggatggcgccc

SEQ ID NO: 1092 - DNA, 17 bp, Artificial Sequence
  Description of Artificial Sequencecapture
gtccgaggacggagccc

SEQ ID NO: 1093 - DNA, 17 bp, Artificial Sequence
  Description of Artificial Sequencecapture
gtccgaggaaggagccg

SEQ ID NO: 1094 - DNA, 18 bp, Artificial Sequence
  Description of Artificial Sequencecapture
gtccgagagaggagcccc

SEQ ID NO: 1095 - DNA, 17 bp, Artificial Sequence
  Description of Artificial Sequencecapture
gtccgaggatggctccc

SEQ ID NO: 1096 - DNA, 17 bp, Artificial Sequence
  Description of Artificial Sequencecapture
gtccgaggacggagccc

SEQ ID NO: 1097 - DNA, 17 bp, Artificial Sequence
  Description of Artificial Sequencecapture
acagatctacaaggccc

SEQ ID NO: 1098 - DNA, 17 bp, Artificial Sequence
  Description of Artificial Sequencecapture
acagatcttcaagacca

SEQ ID NO: 1099 - DNA, 17 bp, Artificial Sequence
  Description of Artificial Sequencecapture
acagatctccaagacca

SEQ ID NO: 1100 - DNA, 17 bp, Artificial Sequence
  Description of Artificial Sequencecapture
acagatctgcaaggccc

SEQ ID NO: 1101 - DNA, 17 bp, Artificial Sequence
  Description of Artificial Sequencecapture
acggaacatgaaggcct

SEQ ID NO: 1102 - DNA, 17 bp, Artificial Sequence
  Description of Artificial Sequencecapture
acagatctcgaagacca

SEQ ID NO: 1103 - DNA, 17 bp, Artificial Sequence
  Description of Artificial Sequencecapture
acagatctacaagacca

SEQ ID NO: 1104 - DNA, 17 bp, Artificial Sequence
  Description of Artificial Sequencecapture
acagaccttcaagacca

SEQ ID NO: 1105 - DNA, 16 bp, Artificial Sequence
  Description of Artificial Sequencecapture
acagatctgcaagacc

SEQ ID NO: 1106 - DNA, 17 bp, Artificial Sequence
  Description of Artificial Sequencecapture
acagaagtacaagcgcc

SEQ ID NO: 1107 - DNA, 17 bp, Artificial Sequence
  Description of Artificial Sequencecapture
acagaactgcaagacca

SEQ ID NO: 1108 - DNA, 17 bp, Artificial Sequence
  Description of Artificial Sequencecapture
acagatctgcaaggcca

SEQ ID NO: 1109 - DNA, 17 bp, Artificial Sequence
  Description of Artificial Sequencecapture
ccgagagagcctgcgga

SEQ ID NO: 1110 - DNA, 17 bp, Artificial Sequence
  Description of Artificial Sequencecapture
ccgagagaacctgcgga

SEQ ID NO: 1111 - DNA, 17 bp, Artificial Sequence
  Description of Artificial Sequencecapture
ccgagaggacctgcgga

SEQ ID NO: 1112 - DNA, 16 bp, Artificial Sequence
  Description of Artificial Sequencecapture
ccgagtgggcctgcgg

SEQ ID NO: 1113 - DNA, 17 bp, Artificial Sequence
  Description of Artificial Sequencecapture
ccgagtgagcctgcgga

SEQ ID NO: 1114 - DNA, 17 bp, Artificial Sequence
  Description of Artificial Sequencecapture
ccgagaggacctgcgca

SEQ ID NO: 1115 - DNA, 17 bp, Artificial Sequence
  Description of Artificial Sequencecapture
ccgagagagcctgcgca

SEQ ID NO: 1116 - DNA, 17 bp, Artificial Sequence
  Description of Artificial Sequencecapture
cctgcggaacctgcgcg

SEQ ID NO: 1117 - DNA, 19 bp, Artificial Sequence
  Description of Artificial Sequencecapture
gacctgcggaccctgctcc

SEQ ID NO: 1118 - DNA, 17 bp, Artificial Sequence
  Description of Artificial Sequencecapture
cctgcggatcgcgctcc

SEQ ID NO: 1119 - DNA, 17 bp, Artificial Sequence
  Description of Artificial Sequencecapture
ctgcggaagctgcgcgg

SEQ ID NO: 1120 - DNA, 17 bp, Artificial Sequence
  Description of Artificial Sequencecapture
cctgcggaacctgctcc

SEQ ID NO: 1121 - DNA, 17 bp, Artificial Sequence
  Description of Artificial Sequencecapture
cctgcgcaccgcgctcc

SEQ ID NO: 1122 - DNA, 17 bp, Artificial Sequence
  Description of Artificial Sequencecapture
cctgcgcaccccgctcc

SEQ ID NO: 1123 - DNA, 17 bp, Artificial Sequence
  Description of Artificial Sequencecapture
cctccagagcatgtacg

SEQ ID NO: 1124 - DNA, 17 bp, Artificial Sequence
  Description of Artificial Sequencecapture
ctccagaggatgtacgg

SEQ ID NO: 1125 - DNA, 17 bp, Artificial Sequence
  Description of Artificial Sequencecapture
ctccagacgatgtacgg

SEQ ID NO: 1126 - DNA, 17 bp, Artificial Sequence
  Description of Artificial Sequencecapture
cctccagtggatgtatg

SEQ ID NO: 1127 - DNA, 17 bp, Artificial Sequence
  Description of Artificial Sequencecapture
ctccagaatatgtacgg

SEQ ID NO: 1128 - DNA, 17 bp, Artificial Sequence
  Description of Artificial Sequencecapture
catccaggtgatgtatg

SEQ ID NO: 1129 - DNA, 18 bp, Artificial Sequence
  Description of Artificial Sequencecapture
catccagagcatgtacgg

SEQ ID NO: 1130 - DNA, 17 bp, Artificial Sequence
  Description of Artificial Sequencecapture
atccagaggatgtatgg

SEQ ID NO: 1131 - DNA, 16 bp, Artificial Sequence
  Description of Artificial Sequencecapture
tggcagacgatgtatg

SEQ ID NO: 1132 - DNA, 17 bp, Artificial Sequence
  Description of Artificial Sequencecapture
ctccagaatatgtatgg

SEQ ID NO: 1133 - DNA, 17 bp, Artificial Sequence
  Description of Artificial Sequencecapture
cctccagagcacgtacg

SEQ ID NO: 1134 - DNA, 17 bp, Artificial Sequence
  Description of Artificial Sequencecapture
atccagaggatgtacgg

SEQ ID NO: 1135 - DNA, 17 bp, Artificial Sequence
  Description of Artificial Sequencecapture
ctccagaggatgtatgg

SEQ ID NO: 1136 - DNA, 17 bp, Artificial Sequence
  Description of Artificial Sequencecapture
ctccagaggatgtttgg

SEQ ID NO: 1137 - DNA, 17 bp, Artificial Sequence
  Description of Artificial Sequencecapture
tggcagaggatgtatgg

SEQ ID NO: 1138 - DNA, 17 bp, Artificial Sequence
  Description of Artificial Sequencecapture
ctccagaggatgtctgg

SEQ ID NO: 1139 - DNA, 17 bp, Artificial Sequence
  Description of Artificial Sequencecapture
ctccagaggatgtgtgg

SEQ ID NO: 1140 - DNA, 17 bp, Artificial Sequence
  Description of Artificial Sequencecapture
atccagaggatgtctgg

SEQ ID NO: 1141 - DNA, 17 bp, Artificial Sequence
  Description of Artificial Sequencecapture
ctccacaggatgtacgg

SEQ ID NO: 1142 - DNA, 17 bp, Artificial Sequence
  Description of Artificial Sequencecapture
tgaccagtacgcctacg

SEQ ID NO: 1143 - DNA, 16 bp, Artificial Sequence
  Description of Artificial Sequencecapture
aaccagttagcctacg

SEQ ID NO: 1144 - DNA, 17 bp, Artificial Sequence
  Description of Artificial Sequencecapture
accaccaggacgcctac

SEQ ID NO: 1145 - DNA, 17 bp, Artificial Sequence
  Description of Artificial Sequencecapture
accaccagcacgcctac

SEQ ID NO: 1146 - DNA, 17 bp, Artificial Sequence
  Description of Artificial Sequencecapture
tgaccagtccgcctacg

SEQ ID NO: 1147 - DNA, 17 bp, Artificial Sequence
  Description of Artificial Sequencecapture
ataaccagaacgcctac

SEQ ID NO: 1148 - DNA, 17 bp, Artificial Sequence
  Description of Artificial Sequencecapture
taaccagttcgcctacg

SEQ ID NO: 1149 - DNA, 17 bp, Artificial Sequence
  Description of Artificial Sequencecapture
taaccagtacgcctacg

SEQ ID NO: 1150 - DNA, 17 bp, Artificial Sequence
  Description of Artificial Sequencecapture
taaccagttagcctacg

SEQ ID NO: 1151 - DNA, 17 bp, Artificial Sequence
  Description of Artificial Sequencecapture
atgaccaggacgcctac

SEQ ID NO: 1152 - DNA, 17 bp, Artificial Sequence
  Description of Artificial Sequencecapture
tgaccagttcgcctacg

SEQ ID NO: 1153 - DNA, 17 bp, Artificial Sequence
  Description of Artificial Sequencecapture
taaacagtacgcctacg

SEQ ID NO: 1154 - DNA, 16 bp, Artificial Sequence
  Description of Artificial Sequencecapture
aaccggttagcctacg

SEQ ID NO: 1155 - DNA, 17 bp, Artificial Sequence
  Description of Artificial Sequencecapture
taaccagttcgcctacg

SEQ ID NO: 1156 - DNA, 16 bp, Artificial Sequence
  Description of Artificial Sequencecapture
aaccagtacgcctacg

SEQ ID NO: 1157 - DNA, 16 bp, Artificial Sequence
  Description of Artificial Sequencecapture
aaccagtacgcctacg

SEQ ID NO: 1158 - DNA, 17 bp, Artificial Sequence
  Description of Artificial Sequencecapture
ggagcagcggagagcct

SEQ ID NO: 1159 - DNA, 17 bp, Artificial Sequence
  Description of Artificial Sequencecapture
ggagcaggacagagcct

SEQ ID NO: 1160 - DNA, 17 bp, Artificial Sequence
  Description of Artificial Sequencecapture
ggagcagctgagagcct

SEQ ID NO: 1161 - DNA, 17 bp, Artificial Sequence
  Description of Artificial Sequencecapture
cggagcagtggagagcc

SEQ ID NO: 1162 - DNA, 17 bp, Artificial Sequence
  Description of Artificial Sequencecapture
ggagcagcggagaacct

SEQ ID NO: 1163 - DNA, 17 bp, Artificial Sequence
  Description of Artificial Sequencecapture
ggagcagctgagaacct

SEQ ID NO: 1164 - DNA, 17 bp, Artificial Sequence
  Description of Artificial Sequencecapture
ggagcagctgagagctt

SEQ ID NO: 1165 - DNA, 17 bp, Artificial Sequence
  Description of Artificial Sequencecapture
ggagggcgagtgcgtgg

SEQ ID NO: 1166 - DNA, 17 bp, Artificial Sequence
  Description of Artificial Sequencecapture
ggagggcacgtgcgtgg

SEQ ID NO: 1167 - DNA, 17 bp, Artificial Sequence
  Description of Artificial Sequencecapture
ggagggcctgtgcgtgg

SEQ ID NO: 1168 - DNA, 17 bp, Artificial Sequence
  Description of Artificial Sequencecapture
ggagggcgcgtgcgtgg

SEQ ID NO: 1169 - DNA, 17 bp, Artificial Sequence
  Description of Artificial Sequencecapture
ggaggacctgtgcgtgg

SEQ ID NO: 1170 - DNA, 17 bp, Artificial Sequence
  Description of Artificial Sequencecapture
ggtatttccacacctcc

SEQ ID NO: 1171 - DNA, 17 bp, Artificial Sequence
  Description of Artificial Sequencecapture
ggtatttcgacaccgcc

SEQ ID NO: 1172 - DNA, 17 bp, Artificial Sequence
  Description of Artificial Sequencecapture
ccggcccgtccgcgggg

SEQ ID NO: 1173 - DNA, 17 bp, Artificial Sequence
  Description of Artificial Sequencecapture
ggccgcggagagccccg

SEQ ID NO: 1174 - DNA, 17 bp, Artificial Sequence
  Description of Artificial Sequencecapture
cggggagctccgcttca

SEQ ID NO: 1175 - DNA, 16 bp, Artificial Sequence
  Description of Artificial Sequencecapture
acgacacgcagttcgt

SEQ ID NO: 1176 - DNA, 17 bp, Artificial Sequence
  Description of Artificial Sequencecapture
agttcgtgcggttcgac

SEQ ID NO: 1177 - DNA, 17 bp, Artificial Sequence
  Description of Artificial Sequencecapture
tgaggttcaacagcgac

SEQ ID NO: 1178 - DNA, 17 bp, Artificial Sequence
  Description of Artificial Sequencecapture
gcgacgccacgagtccg

SEQ ID NO: 1179 - DNA, 17 bp, Artificial Sequence
  Description of Artificial Sequencecapture
ggtatttccacaccgcc

SEQ ID NO: 1180 - DNA, 16 bp, Artificial Sequence
  Description of Artificial Sequencecapture
acgacacgctgttcgt

SEQ ID NO: 1181 - DNA, 18 bp, Artificial Sequence
  Description of Artificial Sequencecapture
aggatggcgccccgggcg

SEQ ID NO: 1182 - DNA, 18 bp, Artificial Sequence
  Description of Artificial Sequencecapture
aggatggctccccgggcg

SEQ ID NO: 1183 - DNA, 17 bp, Artificial Sequence
  Description of Artificial Sequencecapture
atggctccccgggcgcc

SEQ ID NO: 1184 - DNA, 17 bp, Artificial Sequence
  Description of Artificial Sequencecapture
cgggcgccatggataga

SEQ ID NO: 1185 - DNA, 17 bp, Artificial Sequence
  Description of Artificial Sequencecapture
gccgtgggtggagcagg

SEQ ID NO: 1186 - DNA, 17 bp, Artificial Sequence
  Description of Artificial Sequencecapture
gaggggccagaatattg

SEQ ID NO: 1187 - DNA, 17 bp, Artificial Sequence
  Description of Artificial Sequencecapture
ggggccggcgtattggg

SEQ ID NO: 1188 - DNA, 17 bp, Artificial Sequence
  Description of Artificial Sequencecapture
gggccgggatattggga

SEQ ID NO: 1189 - DNA, 16 bp, Artificial Sequence
  Description of Artificial Sequencecapture
ggccggaatattggga

SEQ ID NO: 1190 - DNA, 17 bp, Artificial Sequence
  Description of Artificial Sequencecapture
tattgggagcgggagac

SEQ ID NO: 1191 - DNA, 17 bp, Artificial Sequence
  Description of Artificial Sequencecapture
attgggacggggagaca

SEQ ID NO: 1192 - DNA, 17 bp, Artificial Sequence
  Description of Artificial Sequencecapture
attgggacgaggagaca

SEQ ID NO: 1193 - DNA, 17 bp, Artificial Sequence
  Description of Artificial Sequencecapture
ggagccggagacacaga

SEQ ID NO: 1194 - DNA, 17 bp, Artificial Sequence
  Description of Artificial Sequencecapture
gggaccgggacacacag

SEQ ID NO: 1195 - DNA, 17 bp, Artificial Sequence
  Description of Artificial Sequencecapture
ggaccgggagacacaga

SEQ ID NO: 1196 - DNA, 17 bp, Artificial Sequence
  Description of Artificial Sequencecapture
ccgggagatacagatct

SEQ ID NO: 1197 - DNA, 17 bp, Artificial Sequence
  Description of Artificial Sequencecapture
ggagacacggaacatga

SEQ ID NO: 1198 - DNA, 17 bp, Artificial Sequence
  Description of Artificial Sequencecapture
cacacagaccttcaaga

SEQ ID NO: 1199 - DNA, 17 bp, Artificial Sequence
  Description of Artificial Sequencecapture
cacacagaactgcaaga

SEQ ID NO: 1200 - DNA, 17 bp, Artificial Sequence
  Description of Artificial Sequencecapture
gacacggaacatgaagg

SEQ ID NO: 1201 - DNA, 17 bp, Artificial Sequence
  Description of Artificial Sequencecapture
tctacaagaccaacaca

SEQ ID NO: 1202 - DNA, 18 bp, Artificial Sequence
  Description of Artificial Sequencecapture
aagtacaagcgccaggca

SEQ ID NO: 1203 - DNA, 17 bp, Artificial Sequence
  Description of Artificial Sequencecapture
gcaaggccaaggcacag

SEQ ID NO: 1204 - DNA, 18 bp, Artificial Sequence
  Description of Artificial Sequencecapture
aaggcctccgcgcagact

SEQ ID NO: 1205 - DNA, 18 bp, Artificial Sequence
  Description of Artificial Sequencecapture
caagaccaacacacagac

SEQ ID NO: 1206 - DNA, 17 bp, Artificial Sequence
  Description of Artificial Sequencecapture
caacacacggacttacc

SEQ ID NO: 1207 - DNA, 17 bp, Artificial Sequence
  Description of Artificial Sequencecapture
aggcacaggctgaccga

SEQ ID NO: 1208 - DNA, 17 bp, Artificial Sequence
  Description of Artificial Sequencecapture
cacagacttaccgagag

SEQ ID NO: 1209 - DNA, 17 bp, Artificial Sequence
  Description of Artificial Sequencecapture
acagactggccgagtga

SEQ ID NO: 1210 - DNA, 17 bp, Artificial Sequence
  Description of Artificial Sequencecapture
tgaccgagtgagcctgc

SEQ ID NO: 1211 - DNA, 17 bp, Artificial Sequence
  Description of Artificial Sequencecapture
atggcgccccgggcgcc

SEQ ID NO: 1212 - DNA, 17 bp, Artificial Sequence
  Description of Artificial Sequencecapture
ggaccgggagacacgga

SEQ ID NO: 1213 - DNA, 17 bp, Artificial Sequence
  Description of Artificial Sequencecapture
gacacggaacatgaagg

SEQ ID NO: 1214 - DNA, 18 bp, Artificial Sequence
  Description of Artificial Sequencecapture
caagaccaacacacggac

SEQ ID NO: 1215 - DNA, 17 bp, Artificial Sequence
  Description of Artificial Sequencecapture
cgcagacttaccgagag

SEQ ID NO: 1216 - DNA, 17 bp, Artificial Sequence
  Description of Artificial Sequencecapture
ttaccgagtgaacctgc

SEQ ID NO: 1217 - DNA, 17 bp, Artificial Sequence
  Description of Artificial Sequencecapture
acggagccccgggcgcc

SEQ ID NO: 1218 - DNA, 17 bp, Artificial Sequence
  Description of Artificial Sequencecapture
ggaccgggagatacaga

SEQ ID NO: 1219 - DNA, 17 bp, Artificial Sequence
  Description of Artificial Sequencecapture
cacggacttaccgagag

SEQ ID NO: 1220 - DNA, 17 bp, Artificial Sequence
  Description of Artificial Sequencecapture
ttaccgagtgagcctgc

SEQ ID NO: 1221 - DNA, 17 bp, Artificial Sequence
  Description of Artificial Sequencecapture
gaggagccccgggcgcc

SEQ ID NO: 1222 - DNA, 17 bp, Artificial Sequence
  Description of Artificial Sequencecapture
ggagccggagacacaga

SEQ ID NO: 1223 - DNA, 17 bp, Artificial Sequence
  Description of Artificial Sequencecapture
cacagacttaccgagtg

SEQ ID NO: 1224 - DNA, 17 bp, Artificial Sequence
  Description of Artificial Sequencecapture
tgaccgagtgggcctgc

SEQ ID NO: 1225 - DNA, 17 bp, Artificial Sequence
  Description of Artificial Sequencecapture
ggagcgggagacacaga

SEQ ID NO: 1226 - DNA, 18 bp, Artificial Sequence
  Description of Artificial Sequencecapture
ctgcggatcgcgctccgc

SEQ ID NO: 1227 - DNA, 17 bp, Artificial Sequence
  Description of Artificial Sequencecapture
gcgcaccccgctccgct

SEQ ID NO: 1228 - DNA, 17 bp, Artificial Sequence
  Description of Artificial Sequencecapture
accctgctccgctacta

SEQ ID NO: 1229 - DNA, 18 bp, Artificial Sequence
  Description of Artificial Sequencecapture
gcgctccgcgactacaac

SEQ ID NO: 1230 - DNA, 17 bp, Artificial Sequence
  Description of Artificial Sequencecapture
tgcgcggccactacaac

SEQ ID NO: 1231 - DNA, 17 bp, Artificial Sequence
  Description of Artificial Sequencecapture
tctcacatcatccagag

SEQ ID NO: 1232 - DNA, 18 bp, Artificial Sequence
  Description of Artificial Sequencecapture
ggtctcacatcatccaga

SEQ ID NO: 1233 - DNA, 17 bp, Artificial Sequence
  Description of Artificial Sequencecapture
gtctcacacttggcaga

SEQ ID NO: 1234 - DNA, 16 bp, Artificial Sequence
  Description of Artificial Sequencecapture
ctcacatcatccagag

SEQ ID NO: 1235 - DNA, 17 bp, Artificial Sequence
  Description of Artificial Sequencecapture
accctccacaggatgta

SEQ ID NO: 1236 - DNA, 17 bp, Artificial Sequence
  Description of Artificial Sequencecapture
ccagagcacgtacggct

SEQ ID NO: 1237 - DNA, 17 bp, Artificial Sequence
  Description of Artificial Sequencecapture
gaggatgtgtggctgcg

SEQ ID NO: 1238 - DNA, 17 bp, Artificial Sequence
  Description of Artificial Sequencecapture
aggatgtatggctgcga

SEQ ID NO: 1239 - DNA, 17 bp, Artificial Sequence
  Description of Artificial Sequencecapture
aggatgtttggctgcga

SEQ ID NO: 1240 - DNA, 17 bp, Artificial Sequence
  Description of Artificial Sequencecapture
aggatgtctggctgcga

SEQ ID NO: 1241 - DNA, 16 bp, Artificial Sequence
  Description of Artificial Sequencecapture
gctgcgacctggggcc

SEQ ID NO: 1242 - DNA, 16 bp, Artificial Sequence
  Description of Artificial Sequencecapture
tggggcccgacgggcg

SEQ ID NO: 1243 - DNA, 17 bp, Artificial Sequence
  Description of Artificial Sequencecapture
gacgggcgtctcctccg

SEQ ID NO: 1244 - DNA, 15 bp, Artificial Sequence
  Description of Artificial Sequencecapture
acgggcgcttcctcc

SEQ ID NO: 1245 - DNA, 18 bp, Artificial Sequence
  Description of Artificial Sequencecapture
ctgcgcaccgcgctccgc

SEQ ID NO: 1246 - DNA, 17 bp, Artificial Sequence
  Description of Artificial Sequencecapture
accgcgctccgctacta

SEQ ID NO: 1247 - DNA, 17 bp, Artificial Sequence
  Description of Artificial Sequencecapture
tctcacatcatccaggt

SEQ ID NO: 1248 - DNA, 17 bp, Artificial Sequence
  Description of Artificial Sequencecapture
acgatgtatggctgcga

SEQ ID NO: 1249 - DNA, 17 bp, Artificial Sequence
  Description of Artificial Sequencecapture
gctgcgacctggggccg

SEQ ID NO: 1250 - DNA, 17 bp, Artificial Sequence
  Description of Artificial Sequencecapture
atcgcgctccgctacta

SEQ ID NO: 1251 - DNA, 17 bp, Artificial Sequence
  Description of Artificial Sequencecapture
tggatgtatggctgcga

SEQ ID NO: 1252 - DNA, 17 bp, Artificial Sequence
  Description of Artificial Sequencecapture
accccgctccgctacta

SEQ ID NO: 1253 - DNA, 17 bp, Artificial Sequence
  Description of Artificial Sequencecapture
aatatgtatggctgcga

SEQ ID NO: 1254 - DNA, 17 bp, Artificial Sequence
  Description of Artificial Sequencecapture
aacctgctccgctacta

SEQ ID NO: 1255 - DNA, 17 bp, Artificial Sequence
  Description of Artificial Sequencecapture
atcgcgctccgcgacta

SEQ ID NO: 1256 - DNA, 16 bp, Artificial Sequence
  Description of Artificial Sequencecapture
cctcctccccgggcat

SEQ ID NO: 1257 - DNA, 17 bp, Artificial Sequence
  Description of Artificial Sequencecapture
ctccgcggttataacca

SEQ ID NO: 1258 - DNA, 17 bp, Artificial Sequence
  Description of Artificial Sequencecapture
tccgcgggtatgaccag

SEQ ID NO: 1259 - DNA, 17 bp, Artificial Sequence
  Description of Artificial Sequencecapture
tccgcgggtaccaccag

SEQ ID NO: 1260 - DNA, 17 bp, Artificial Sequence
  Description of Artificial Sequencecapture
gcgggcataaccagtac

SEQ ID NO: 1261 - DNA, 17 bp, Artificial Sequence
  Description of Artificial Sequencecapture
cgggcataaacagtacg

SEQ ID NO: 1262 - DNA, 17 bp, Artificial Sequence
  Description of Artificial Sequencecapture
gtataaccggttagcct

SEQ ID NO: 1263 - DNA, 17 bp, Artificial Sequence
  Description of Artificial Sequencecapture
gacggcaaagattacat

SEQ ID NO: 1264 - DNA, 17 bp, Artificial Sequence
  Description of Artificial Sequencecapture
gacggcaaagattacat

SEQ ID NO: 1265 - DNA, 16 bp, Artificial Sequence
  Description of Artificial Sequencecapture
ccctgaaggaggacct

SEQ ID NO: 1266 - DNA, 17 bp, Artificial Sequence
  Description of Artificial Sequencecapture
gccctgaatgaggacct

SEQ ID NO: 1267 - DNA, 17 bp, Artificial Sequence
  Description of Artificial Sequencecapture
aggacctgagctcctgg

SEQ ID NO: 1268 - DNA, 17 bp, Artificial Sequence
  Description of Artificial Sequencecapture
tggaccgcggcggacac

SEQ ID NO: 1269 - DNA, 17 bp, Artificial Sequence
  Description of Artificial Sequencecapture
ccgccgcgaacacggcg

SEQ ID NO: 1270 - DNA, 17 bp, Artificial Sequence
  Description of Artificial Sequencecapture
cgcggacaaggcggctc

SEQ ID NO: 1271 - DNA, 17 bp, Artificial Sequence
  Description of Artificial Sequencecapture
gcggacacagcggctca

SEQ ID NO: 1272 - DNA, 17 bp, Artificial Sequence
  Description of Artificial Sequencecapture
gcggacaccgcggctca

SEQ ID NO: 1273 - DNA, 17 bp, Artificial Sequence
  Description of Artificial Sequencecapture
tccgcgggtataaccag

SEQ ID NO: 1274 - DNA, 17 bp, Artificial Sequence
  Description of Artificial Sequencecapture
gcgggcataaccagtta

SEQ ID NO: 1275 - DNA, 17 bp, Artificial Sequence
  Description of Artificial Sequencecapture
tggacagcggcggacac

SEQ ID NO: 1276 - DNA, 17 bp, Artificial Sequence
  Description of Artificial Sequencecapture
tccgcgggtataaccgg

SEQ ID NO: 1277 - DNA, 17 bp, Artificial Sequence
  Description of Artificial Sequencecapture
cgcgggtataaccagtt

SEQ ID NO: 1278 - DNA, 17 bp, Artificial Sequence
  Description of Artificial Sequencecapture
gcgggcataaccagttc

SEQ ID NO: 1279 - DNA, 17 bp, Artificial Sequence
  Description of Artificial Sequencecapture
gcgggcataaccaggac

SEQ ID NO: 1280 - DNA, 17 bp, Artificial Sequence
  Description of Artificial Sequencecapture
gcgggtataaccggtta

SEQ ID NO: 1281 - DNA, 17 bp, Artificial Sequence
  Description of Artificial Sequencecapture
gcggttataaccagtac

SEQ ID NO: 1282 - DNA, 17 bp, Artificial Sequence
  Description of Artificial Sequencecapture
ccgggcataaccagtac

SEQ ID NO: 1283 - DNA, 17 bp, Artificial Sequence
  Description of Artificial Sequencecapture
gcgggtataaccagttc

SEQ ID NO: 1284 - DNA, 17 bp, Artificial Sequence
  Description of Artificial Sequencecapture
gatcacccggcgcaagt

SEQ ID NO: 1285 - DNA, 17 bp, Artificial Sequence
  Description of Artificial Sequencecapture
ggcccgtgtggcggagc

SEQ ID NO: 1286 - DNA, 17 bp, Artificial Sequence
  Description of Artificial Sequencecapture
ggcccgtgcggcggagc

SEQ ID NO: 1287 - DNA, 17 bp, Artificial Sequence
  Description of Artificial Sequencecapture
agctgagaacctacctg

SEQ ID NO: 1288 - DNA, 17 bp, Artificial Sequence
  Description of Artificial Sequencecapture
ctgagagcttacctgga

SEQ ID NO: 1289 - DNA, 17 bp, Artificial Sequence
  Description of Artificial Sequencecapture
tgcgtggattggctccg

SEQ ID NO: 1290 - DNA, 17 bp, Artificial Sequence
  Description of Artificial Sequencecapture
tgcgtggacgggctccg

SEQ ID NO: 1291 - DNA, 17 bp, Artificial Sequence
  Description of Artificial Sequencecapture
gcgtggaggggctccgc

SEQ ID NO: 1292 - DNA, 17 bp, Artificial Sequence
  Description of Artificial Sequencecapture
cgtggagtcgctccgca

SEQ ID NO: 1293 - DNA, 17 bp, Artificial Sequence
  Description of Artificial Sequencecapture
tccgcagacacctggag

SEQ ID NO: 1294 - DNA, 17 bp, Artificial Sequence
  Description of Artificial Sequencecapture
agcggagaacctacctg

SEQ ID NO: 1295 - DNA, 17 bp, Artificial Sequence
  Description of Artificial Sequencecapture
ggaaggagacgctggag

SEQ ID NO: 1296 - DNA, 17 bp, Artificial Sequence
  Description of Artificial Sequencecapture
ggaaggacacgctggag

SEQ ID NO: 1297 - DNA, 16 bp, Artificial Sequence
  Description of Artificial Sequencecapture
agacgctgcagcgcgc

SEQ ID NO: 1298 - DNA, 17 bp, Artificial Sequence
  Description of Artificial Sequencecapture
ggaaggagacgctgcag